SEQUENCE LISTING (538 sequences)

SEQ ID NO: 1 - protein, 1057 aa, Synthetic construct
MNQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 2 - protein, 1057 aa, Synthetic construct
MNQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 3 - protein, 1057 aa, Synthetic construct
MNQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKFSHRVDKKPNRQLINDTLYSTRMKDEHDYIVQTITDIYGKDNTNLKKQFNKNPEKFLMYQNDPKTFEKLSIIMKQYSDEKNPLAKYYEETGEYLTKYSKKNNGPIVKKIKLLGNKVGNHLDVTNKYENSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 4 - protein, 1057 aa, Synthetic construct
MNQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTSYLKAYFSANNMDVKVKTINGSFTNHLRKVWRFDKYRNHGYKHHAEDALIIANADFLFKENKKLQNTNKILEKPTIENNTKKVTVEKEEDYNNVFETPKLVEDIKQYRDYKFSHRVDKKPNRQLINDTLYSTRMKDEHDYIVQTITDIYGKDNTNLKKQFNKNPEKFLMYQNDPKTFEKLSIIMKQYSDEKNPLAKYYEETGEYLTKYSKKNNGPIVKKIKLLGNKVGNHLDVTNKYENSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 5 - protein, 1054 aa, Synthetic construct
MNQKFILGLDIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHNINVSSEDEDASNELSTKEQINRNNKLLKDKYVCEVQLQRLKEGQIRGEKNRFKTTDILKEIDQLLKVQKDYHNLDIDFINQYKEIVETRREYFEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGKPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 6 - protein, 1057 aa, Synthetic construct
MNQKFILGLDIGITSVGYGLIDYETKNIIDAGVRLFPEANVENNEGRRSKRGSRRLKRRRIHRLDRVKHLLAEYDLLDLTNIPKSTNPYQTRVKGLNEKLSKDELVIALLHIAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGKGSPFGWEGNIKKWFEQMMGHCTYFPEELRSVKYSYSAELFNALNDLNNLVITRDEDAKLNYGEKFQIIENVFKQKKTPNLKQIAIEIGVHETEIKGYRVNKSGTPEFTEFKLYHDLKSIVFDKSILENEAILDQIAEILTIYQDEQSIKEELNKLPEILNEQDKAEIAKLIGYNGTHRLSLKCIHLINEELWQTSRNQMEIFNYLNIKPNKVDLSEQNKIPKDMVNDFILSPVVKRTFIQSINVINKVIEKYGIPEDIIIELARENNSDDRKKFINNLQKKNEATRKRINEIIGQTGNQNAKRIVEKIRLHDQQEGKCLYSLKDIPLEDLLRNPNNYDIDHIIPRSVSFDDSMHNKVLVRREQNAKKNNQTPYQYLTSGYADIKYSVFKQHVLNLAENKDRMTKKKREYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 7 - protein, 1057 aa, Synthetic construct
MNQKFILGLDIGITSVGYGLIDYETKNIIDAGVRLFPEANVENNEGRRSKRGSRRLKRRRIHRLDRVKHLLAEYDLLDLTNIPKSTNPYQTRVKGLNEKLSKDELVIALLHIAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGKGSPFGWEGNIKKWFEQMMGHCTYFPEELRSVKYSYSAELFNALNDLNNLVITRDEDAKLNYGEKFQIIENVFKQKKTPNLKQIAIEIGVHETEIKGYRVNKSGTPEFTEFKLYHDLKSIVFDKSILENEAILDQIAEILTIYQDEQSIKEELNKLPEILNEQDKAEIAKLIGYNGTHRLSLKCIHLINEELWQTSRNQMEIFNYLNIKPNKVDLSEQNKIPKDMVNDFILSPVVKRTFIQSINVINKVIEKYGIPEDIIIELARENNSDDRKKFINNLQKKNEATRKRINEIIGQTGNQNAKRIVEKIRLHDQQEGKCLYSLKDIPLEDLLRNPNNYDIDHIIPRSVSFDDSMHNKVLVRREQNAKKNNQTPYQYLTSGYADIKYSVFKQHVLNLAENKDRMTKKKREYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETDQKDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 8 - protein, 1057 aa, Synthetic construct
MNQKFILGLDIGITSVGYGLIDYETKNIIDAGVRLFPEANVENNEGRRSKRGSRRLKRRRIHRLDRVKHLLAEYDLLDLTNIPKSTNPYQTRVKGLNEKLSKDELVIALLHIAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGKGSPFGWEGNIKKWFEQMMGHCTYFPEELRSVKYSYSAELFNALNDLNNLVITRDEDAKLNYGEKFQIIENVFKQKKTPNLKQIAIEIGVHETEIKGYRVNKSGTPEFTEFKLYHDLKSIVFDKSILENEAILDQIAEILTIYQDEQSIKEELNKLPEILNEQDKAEIAKLIGYNGTHRLSLKCIHLINEELWQTSRNQMEIFNYLNIKPNKVDLSEQNKIPKDMVNDFILSPVVKRTFIQSINVINKVIEKYGIPEDIIIELARENNSDDRKKFINNLQKKNEATRKRINEIIGQTGNQNAKRIVEKIRLHDQQEGKCLYSLKDIPLEDLLRNPNNYDIDHIIPRSVSFDDSMHNKVLVRREQNAKKNNQTPYQYLTSGYADIKYSVFKQHVLNLAENKDRMTKKKREYLLEERDINKFEVQKEFINRNLVDTRYATRELTNYLKAYFTANNMDVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 9 - protein, 1057 aa, Synthetic construct
MNQKFILGLDIGITSVGYGLIDYETKNIIDAGVRLFPEANVENNEGRRSKRGSRRLKRRRIHRLDRVKHLLAEYDLLDLTNIPKSTNPYQTRVKGLNEKLSKDELVIALLHIAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGKGSPFGWEGNIKKWFEQMMGHCTYFPEELRSVKYSYSAELFNALNDLNNLVITRDEDAKLNYGEKFQIIENVFKQKKTPNLKQIAIEIGVHETEIKGYRVNKSGTPEFTEFKLYHDLKSIVFDKSILENEAILDQIAEILTIYQDEQSIKEELNKLPEILNEQDKAEIAKLIGYNGTHRLSLKCIHLINEELWQTSRNQMEIFNYLNIKPNKVDLSEQNKIPKDMVNDFILSPVVKRTFIQSINVINKVIEKYGIPEDIIIELARENNSDDRKKFINNLQKKNEATRKRINEIIGQTGNQNAKRIVEKIRLHDQQEGKCLYSLKDIPLEDLLRNPNNYDIDHIIPRSVSFDDSMHNKVLVRREQNAKKNNQTPYQYLTSGYADIKYSVFKQHVLNLAENKDRMTKKKREYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETNQNDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 10 - protein, 1053 aa, Synthetic construct
MNQKFILGLDIGITSVGYGLIDYETKNIIDAGVRLFPEANVENNEGRRSKRGSRRLKRRRIHRLERVKSLLSEYKIISGLAPTNNQPYNIRVKGLTEQLTKDELAVALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTEFKLYHDLKSVLFDQSILENEDVLDQIAEILTIYQDKDSIKSKLTELDILLNEEDKENIAQLTGYNGTHRLSLKCIRLVLEEQWYSSRNQMEIFTHLNIKPKKINLTAANKIPKAMIDEFILSPVVKRTFIQSINVINKVIEKYGIPEDIIIELARENNSDDRKKFINNLQKKNEATRKRINEIIGQTGNQNAKRIVEKIRLHDQQEGKCLYSLESIALMDLLNNPQNYEVDHIIPRSVAFDNSIHNKVLVKQIENSKKGNRTPYQYLNSSDAKLSYNQFKQHILNLSKSKDRISKKKKDYLLEERDINKFEVQKEFINRNLVDTRYATRELTSYLKAYFSANNMDVKVKTINGSFTNHLRKVWRFDKYRNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 11 - protein, 1053 aa, Synthetic construct
MNQKFILGLDIGITSVGYGLIDYETKNIIDAGVRLFPEANVENNEGRRSKRGSRRLKRRRIHRLERVKSLLSEYKIISGLAPTNNQPYNIRVKGLTEQLTKDELAVALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTEFKLYHDLKSVLFDQSILENEDVLDQIAEILTIYQDKDSIKSKLTELDILLNEEDKENIAQLTGYNGTHRLSLKCIRLVLEEQWYSSRNQMEIFTHLNIKPKKINLTAANKIPKAMIDEFILSPVVKRTFIQSINVINKVIEKYGIPEDIIIELARENNSDDRKKFINNLQKKNEATRKRINEIIGQTGNQNAKRIVEKIRLHDQQEGKCLYSLESIALMDLLNNPQNYEVDHIIPRSVAFDNSIHNKVLVKQIENSKKGNRTPYQYLNSSDAKLSYNQFKQHILNLSKSKDRISKKKKDYLLEERDINKFEVQKEFINRNLVDTRYATRELTSYLKAYFSANNMDVKVKTINGSFTNHLRKVWRFDKYRNHGYKHHAEDALIIANADFLFKENKKLKAVNSVLEKPEIETDQKDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 12 - protein, 1053 aa, Synthetic construct
MNQKFILGLDIGITSVGYGLIDYETKNIIDAGVRLFPEANVENNEGRRSKRGSRRLKRRRIHRLERVKSLLSEYKIISGLAPTNNQPYNIRVKGLTEQLTKDELAVALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTEFKLYHDLKSVLFDQSILENEDVLDQIAEILTIYQDKDSIKSKLTELDILLNEEDKENIAQLTGYNGTHRLSLKCIRLVLEEQWYSSRNQMEIFTHLNIKPKKINLTAANKIPKAMIDEFILSPVVKRTFIQSINVINKVIEKYGIPEDIIIELARENNSDDRKKFINNLQKKNEATRKRINEIIGQTGNQNAKRIVEKIRLHDQQEGKCLYSLESIALMDLLNNPQNYEVDHIIPRSVAFDNSIHNKVLVKQIENSKKGNRTPYQYLNSSDAKLSYNQFKQHILNLSKSKDRISKKKKDYLLEERDINKFEVQKEFINRNLVDTRYATRELTSYLKAYFSANNMDVKVKTINGSFTNHLRKVWRFDKYRNHGYKHHAEDALIIANADFLFKENKKLKAVNSVLEKPEIETNQNDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 13 - DNA, 3171 bp, Synthetic construct
  source 1..3171: mol_type = other DNA
atgaaccagaaatttatcctgggtctggatattggtattaccagcgttggttatggcctgattgattacgaaaccaaaaacattattgatgccggtgttcgtctgtttccggaagcaaatgttgaaaataatgaaggtcgtcgtagcaaacgtggtagccgtcgtctgaaacgtcgtcgtattcatcgtctggaacgtgttaaactgctgctgaccgaatatgatctgattaacaaagagcagattccgaccagcaataacccgtatcagattcgtgttaaaggtctgagcgaaatcctgagcaaagatgaactggcaattgcactgctgcatctggcaaaacgccgtggcattcataatgttgatgttgcagcagataaagaagaaaccgcaagcgatagcctgagcaccaaagatcagattaacaaaaacgccaaatttctggaaagccgctatgtttgtgaactgcagaaagaacgtctggaaaatgaaggtcatgttcgtggtgttgaaaatcgctttctgacgaaagatattgtgcgtgaggccaaaaaaatcatcgatacccagatgcagtattacccggaaattgatgaaaccttcaaagagaaatatatcagcctggttgaaacccgtcgcgaatattttgaaggtcctggtcagggtagcccgtttggttggaatggtgatctgaaaaaatggtacgaaatgctgatgggtcactgtacctattttccgcaagaactgcgtagcgttaaatatgcctatagcgcagacctgtttaatgcactgaatgatctgaacaacctgattattcagcgcgataatagcgagaaactggaataccatgagaagtatcacatcatcgagaacgtgttcaagcagaaaaaaaagccgacgctgaaacaaatcgcaaaagagattggcgttaacccggaagatattaaaggttatcgtattaccaaaagcggcacaccggagtttacatcctttaaactgttccacgatctgaaaaaagtggtgaaagatcatgccatcctggatgatattgatctgctgaatcagattgcagaaatcctgaccatctatcaggataaagatagcattgttgcagaactgggtcagctggaatatctgatgagcgaagccgataaacagagcattagcgaactgaccggttataccggtacacatagcctgtcactgaaatgcatgaacatgattatcgatgaactgtggcatagcagcatgaaccagatggaagtttttacctatctgaatatgcgtccgaaaaagtatgagctgaaaggttatcagcgtattccgaccgatatgattgatgatgcaattctgagtccggttgtgaaacgcacctttattcagagcatcaacgtgatcaacaaagtgatcgagaaatatggcatccccgaagatatcattatcgaactggcacgtgaaaataactccgatgatcgcaaaaagttcatcaacaacctgcagaaaaagaatgaagcaacccgcaaacgcattaacgaaattattggtcagaccggtaatcagaatgccaaacgtattgtggaaaaaatccgtctgcatgatcagcaagagggcaaatgtctgtatagcctggaaagcattcctctggaagatctgctgaacaatccgaatcattatgaagtggatcacattattccgcgtagcgtgagctttgataattcctatcataataaagtgctggtgaaacagagcgaaaactccaaaaaatccaacctgacaccgtatcagtatttcaatagcggcaaatccaaactgagctacaaccagtttaaacagcatattctgaacctgagcaaaagccaggatcgcatcagcaagaagaagaaggagtacctgctggaagaacgcgacatcaacaaatttgaagtgcagaaagaatttatcaaccgcaacctggttgatacccgttatgcaacccgtgaactgaccaattatctgaaagcatatttcagcgccaacaacatgaacgtgaaagtgaaaacgattaacggcagctttaccgattatctgcgtaaagtgtggaaattcaaaaaagaacgcaaccacggctataaacatcatgccgaagatgccctgattattgcaaatgcagatttcctgtttaaagaaaacaaaaaactgaaagccgtcaacagcgtgctggaaaaaccggaaattgagacaaaacagctggacattcaggttgatagcgaagataattacagcgaaatgtttatcatcccgaaacaggtgcaggatatcaaagattttcgcaacttcaaatatagccaccgcgttgacaaaaaacctaatcgtcagctgattaacgataccctgtatagcacccgcaaaaaagataacagcacctatattgtgcagaccattaaagacatctacgccaaagataataccaccctgaaaaaacagttcgacaaaagcccagaaaaatttctgatgtatcagcatgatccgcgtaccttcgaaaaactggaagttattatgaaacagtatgccaacgagaaaaatccgctggccaaatatcacgaagaaaccggtgaatatctgaccaaatattccaagaagaacaacggtccgatcgttaaatccctgaaatatatcggtaataaactgggcagccatctggatgttacccatcagtttaaaagctccacaaagaagctggttaaactgtccatcaaaccgtatcgctttgatgtgtatctgaccgacaaaggctataaattcattaccatcagctatctggacgtgctgaaaaaagacaactattattatatcccggaacagaaatatgataaactgaaactgggtaaagccatcgataaaaacgccaaatttatcgccagcttctacaaaaacgacctgattaaactggatggcgagatctataaaatcatcggtgttaatagcgacacccgcaatatgattgagctggatctgccggatattcgctataaagaatattgcgaactgaacaacattaaaggcgaaccgcgtatcaaaaagaccatcggcaaaaaagtgaatagcatcgagaaactgaccaccgatgttctgggtaatgtgtttaccaatacccagtataccaaacctcagctgctgttcaaacgcggtaat

SEQ ID NO: 14 - DNA, 3171 bp, Synthetic construct
  source 1..3171: mol_type = other DNA
atgaaccagaaatttatcctgggtctggatattggtattaccagcgttggttatggcctgattgattacgaaaccaaaaacattattgatgccggtgttcgtctgtttccggaagcaaatgttgaaaataatgaaggtcgtcgtagcaaacgtggtagccgtcgtctgaaacgtcgtcgtattcatcgtctggaacgtgttaaactgctgctgaccgaatatgatctgattaacaaagagcagattccgaccagcaataacccgtatcagattcgtgttaaaggtctgagcgaaatcctgagcaaagatgaactggcaattgcactgctgcatctggcaaaacgccgtggcattcataatgttgatgttgcagcagataaagaagaaaccgcaagcgatagcctgagcaccaaagatcagattaacaaaaacgccaaatttctggaaagccgctatgtttgtgaactgcagaaagaacgtctggaaaatgaaggtcatgttcgtggtgttgaaaatcgctttctgacgaaagatattgtgcgtgaggccaaaaaaatcatcgatacccagatgcagtattacccggaaattgatgaaaccttcaaagagaaatatatcagcctggttgaaacccgtcgcgaatattttgaaggtcctggtcagggtagcccgtttggttggaatggtgatctgaaaaaatggtacgaaatgctgatgggtcactgtacctattttccgcaagaactgcgtagcgttaaatatgcctatagcgcagacctgtttaatgcactgaatgatctgaacaacctgattattcagcgcgataatagcgagaaactggaataccatgagaagtatcacatcatcgagaacgtgttcaagcagaaaaaaaagccgacgctgaaacaaatcgcaaaagagattggcgttaacccggaagatattaaaggttatcgtattaccaaaagcggcacaccggagtttacatcctttaaactgttccacgatctgaaaaaagtggtgaaagatcatgccatcctggatgatattgatctgctgaatcagattgcagaaatcctgaccatctatcaggataaagatagcattgttgcagaactgggtcagctggaatatctgatgagcgaagccgataaacagagcattagcgaactgaccggttataccggtacacatagcctgtcactgaaatgcatgaacatgattatcgatgaactgtggcatagcagcatgaaccagatggaagtttttacctatctgaatatgcgtccgaaaaagtatgagctgaaaggttatcagcgtattccgaccgatatgattgatgatgcaattctgagtccggttgtgaaacgcacctttattcagagcatcaacgtgatcaacaaagtgatcgagaaatatggcatccccgaagatatcattatcgaactggcacgtgaaaataactccgatgatcgcaaaaagttcatcaacaacctgcagaaaaagaatgaagcaacccgcaaacgcattaacgaaattattggtcagaccggtaatcagaatgccaaacgtattgtggaaaaaatccgtctgcatgatcagcaagagggcaaatgtctgtatagcctggaaagcattcctctggaagatctgctgaacaatccgaatcattatgaagtggatcacattattccgcgtagcgtgagctttgataattcctatcataataaagtgctggtgaaacagagcgaaaactccaaaaaatccaacctgacaccgtatcagtatttcaatagcggcaaatccaaactgagctacaaccagtttaaacagcatattctgaacctgagcaaaagccaggatcgcatcagcaagaagaagaaggagtacctgctggaagaacgcgatattaacaaatttgaagtgcagaaagaatttatcaaccgcaacctggttgatacccgttatgcaacccgtgaactgaccaattatctgaaagcatatttcagcgccaacaacatgaacgtgaaagtgaaaacgattaacggcagctttaccgattatctgcgtaaagtgtggaaattcaaaaaagaacgcaaccacggctataaacatcatgccgaagatgccctgattattgcaaatgcagatttcctgtttaaagaaaacaaaaaactgaaagccgtcaacagcgtgctggaaaaaccggaaattgagacaaaacagctggacattcaggttgatagcgaagataattacagcgaaatgtttatcatcccgaaacaggtgcaggatatcaaagattttcgcaacttcaaatatagccaccgcgttgacaaaaaacctaatcgtcagctgattaacgataccctgtatagcacccgcaaaaaagataacagcacctatattgtgcagaccattaaagacatctacgccaaagataataccaccctgaaaaaacagttcgacaaaagcccagaaaaatttctgatgtatcagcatgatccgcgtaccttcgaaaaactggaagttattatgaaacagtatgccaacgagaaaaatccgctggccaaatatcacgaagaaaccggtgaatatctgaccaaatattccaagaagaacaacggtccgatcgttaaatccctgaaatatatcggtaataaactgggcagccatctggatgttacccatcagtttaaaagctccacaaagaagctggttaagctgtccattaaaaactatcgcttcgatgtgtatctgaccgagaaaggttataagtttgtgaccattgcctacctgaatgtgttcaaaaaagacaactattactatattccgaaagacaaataccaagaacttaaagagaagaagaaaatcaaggacaccgatcagtttatcgccagcttctataaaaacgatctgatcaagctgaacggcgacctgtataaaatcattggtgtgaatagtgatgaccgcaacatcattgagctggattattacgacatcaaatacaaggattactgcgagatcaacaacattaaaggtgaaccgcgtatcaaaaagaccattggcaaaaaaacggaaagcatcgaaaagtttaccaccgatgttctgggtaatctgtatctgcatagtaccgaaaaagcaccgcagctgattttcaaacgcggtctg

SEQ ID NO: 15 - DNA, 3171 bp, Synthetic construct
  source 1..3171: mol_type = other DNA
atgaaccagaagttcattctcggtctggacattggcattactagcgtgggatacggcttgattgactacgagactaagaacatcatcgatgccggcgtccgcctgttcccggaagccaacgtggagaacaatgagggccggaggtcgaagagaggctcccgccgcctgaagcggcggcgaatccaccggctggagagagtgaagctgctgctcaccgaatacgacctgatcaacaaagaacagatcccgacctccaacaacccgtaccagatcagagtgaagggtctgtcagaaatcctgtccaaggacgaactggcaatcgccctgctgcacctggccaagcggcgcggaatccacaacgtggatgtggctgccgacaaggaagaaaccgcttccgactccctgagcactaaggaccagatcaacaagaacgccaagttcttggagtcccgctacgtgtgcgagctgcagaaggaacggctggaaaacgaaggtcacgtgcgcggagtggaaaaccggttcctgacaaaggacattgtgcgcgaagcgaagaagatcattgatacccaaatgcagtactaccctgaaatcgacgagactttcaaggaaaagtacatttccctggtggaaacccggcgggaatacttcgaaggccccggacagggatcgccgttcggatggaacggggacctcaagaagtggtacgagatgctgatggggcactgtacctactttccgcaagaactgcgctccgtgaagtacgcgtactccgcggatctcttcaacgcgttgaatgacctgaacaacctgatcattcagagagacaattccgaaaagctcgagtaccacgagaagtatcacatcatcgagaatgtgttcaagcagaagaagaaaccgaccctcaagcaaatcgccaaggagattggcgtcaacccagaggacatcaagggatatcggattaccaagagcggcactcccgagtttacctctttcaagctgtttcatgatctgaagaaagtcgtgaaggaccatgccattctcgacgacattgatctcctgaatcagatcgcagagatcctgactatctaccaagacaaggactcgattgtggcagagctgggtcagctcgaatacctgatgtccgaggccgacaagcagtccatctccgaactgacagggtacacggggactcatagcctgtcgctgaagtgcatgaacatgatcattgatgaactgtggcacagctccatgaaccaaatggaagtgtttacctacctcaacatgcgccctaagaagtacgaactgaaaggctaccagcgcatccccaccgacatgatcgacgacgcgatcttgtcccctgtggtcaagaggaccttcattcaatccatcaacgtgatcaacaaggtcatcgaaaagtacggaataccagaagatatcatcattgagctcgctcgggagaacaactcggatgaccggaagaagttcatcaacaatcttcagaagaagaacgaagcgactcggaaacggatcaacgagatcatcggacagaccggaaaccagaacgccaaacggattgtcgaaaagattagactgcacgaccagcaggaagggaagtgcctgtactcactcgagtcaataccgctcgaggacctgttgaacaaccctaaccactatgaagtggaccacatcatccctcggtccgtgagcttcgacaactcgtaccacaacaaagtgctcgtgaagcagtccgaaaactccaagaaatccaacctgaccccgtaccaatacttcaattcgggaaagtcgaagctgtcgtacaaccagttcaaacaacacatactcaaccttagcaaaagccaggatcgcatttccaagaagaagaaggaatacctcctcgaggaaagggacatcaacaagttcgaagtgcagaaagagttcatcaatcgcaacttggtggataccagatatgccacccgggaactcaccaactatctcaaggcctacttttccgccaacaacatgaacgtgaaggtcaagaccatcaacgggtccttcactgactacctgagaaaggtctggaagttcaagaaggaacgcaaccacggatacaagcaccacgctgaggacgctctgatcatcgccaatgccgacttcctgttcaaggaaaacaagaagctgaaagctgtcaactcagtgctggaaaagcctgaaatcgagactaagcagctggatatccaagtggactctgaggacaactacagcgagatgttcatcatccctaaacaagtgcaggatatcaaggactttcgcaacttcaagtactcacaccgggtggacaagaaaccgaatagacagctgatcaacgacacgttgtattccacccggaagaaggataactcaacctacattgtgcagactatcaaggatatctacgccaaagataacactactctgaagaaacaattcgacaagtccccagagaagttcctgatgtaccagcacgacccccgaacctttgagaagcttgaagtgatcatgaagcagtacgccaacgagaagaacccgctggccaagtaccatgaagaaaccggagaatacctgaccaagtacagcaagaagaacaacggtcccattgtcaagagcctgaagtacatcggcaacaagctgggatcccacctcgacgtgacacatcagttcaagtcgtcgactaagaagcttgtgaagctgtcaatcaagaactatagattcgacgtgtacttgaccgaaaagggatacaagttcgtgaccatagcctatctgaacgtgttcaagaaagataactactactacatccccaaggacaagtaccaggagctcaaagaaaagaagaagatcaaagacaccgaccagttcattgcctccttctacaagaacgacctgatcaaactgaacggcgacctctacaagatcattggagtgaacagcgatgacaggaacatcattgagctggactactacgacatcaagtacaaggactactgcgagatcaacaacatcaagggcgaaccccggatcaagaaaactattggaaagaaaaccgagtccattgagaagttcaccactgacgtgctgggaaacctttacctccactccaccgagaaggcaccacaactgatcttcaagcgcggcctg

SEQ ID NO: 16 - DNA, 3171 bp, Synthetic construct
  source 1..3171: mol_type = other DNA
atgaaccagaaatttatcctgggtctggatattggtattaccagcgttggttatggcctgattgattacgaaaccaaaaacattattgatgccggtgttcgtctgtttccggaagcaaatgttgaaaataatgaaggtcgtcgtagcaaacgtggtagccgtcgtctgaaacgtcgtcgtattcatcgtctggaacgtgttaaactgctgctgaccgaatatgatctgattaacaaagagcagattccgaccagcaataacccgtatcagattcgtgttaaaggtctgagcgaaatcctgagcaaagatgaactggcaattgcactgctgcatctggcaaaacgccgtggcattcataatgttgatgttgcagcagataaagaagaaaccgcaagcgatagcctgagcaccaaagatcagattaacaaaaacgccaaatttctggaaagccgctatgtttgtgaactgcagaaagaacgtctggaaaatgaaggtcatgttcgtggtgttgaaaatcgctttctgacgaaagatattgtgcgtgaggccaaaaaaatcatcgatacccagatgcagtattacccggaaattgatgaaaccttcaaagagaaatatatcagcctggttgaaacccgtcgcgaatattttgaaggtcctggtcagggtagcccgtttggttggaatggtgatctgaaaaaatggtacgaaatgctgatgggtcactgtacctattttccgcaagaactgcgtagcgttaaatatgcctatagcgcagacctgtttaatgcactgaatgatctgaacaacctgattattcagcgcgataatagcgagaaactggaataccatgagaagtatcacatcatcgagaacgtgttcaagcagaaaaaaaagccgacgctgaaacaaatcgcaaaagagattggcgttaacccggaagatattaaaggttatcgtattaccaaaagcggcacaccggagtttacatcctttaaactgttccacgatctgaaaaaagtggtgaaagatcatgccatcctggatgatattgatctgctgaatcagattgcagaaatcctgaccatctatcaggataaagatagcattgttgcagaactgggtcagctggaatatctgatgagcgaagccgataaacagagcattagcgaactgaccggttataccggtacacatagcctgtcactgaaatgcatgaacatgattatcgatgaactgtggcatagcagcatgaaccagatggaagtttttacctatctgaatatgcgtccgaaaaagtatgagctgaaaggttatcagcgtattccgaccgatatgattgatgatgcaattctgagtccggttgtgaaacgcacctttattcagagcatcaacgtgatcaacaaagtgatcgagaaatatggcatccccgaagatatcattatcgaactggcacgtgaaaataactccgatgatcgcaaaaagttcatcaacaacctgcagaaaaagaatgaagcaacccgcaaacgcattaacgaaattattggtcagaccggtaatcagaatgccaaacgtattgtggaaaaaatccgtctgcatgatcagcaagagggcaaatgtctgtatagcctggaaagcattcctctggaagatctgctgaacaatccgaatcattatgaagtggatcacattattccgcgtagcgtgagctttgataattcctatcataataaagtgctggtgaaacagagcgaaaactccaaaaaatccaacctgacaccgtatcagtatttcaatagcggcaaatccaaactgagctacaaccagtttaaacagcatattctgaacctgagcaaaagccaggatcgcatcagcaagaagaagaaggagtacctgctggaagaacgcgatattaacaaatttgaagtgcagaaagaatttatcaaccgcaacctggttgatacccgttatgcaacccgtgaactgaccaattatctgaaagcatatttcagcgccaacaacatgaacgtgaaagtgaaaacgattaacggcagctttaccgattatctgcgtaaagtgtggaaattcaaaaaagaacgcaaccacggctataaacatcatgccgaagatgccctgattattgcaaatgcagatttcctgtttaaagaaaacaaaaaactgaaagccgtcaacagcgtgctggaaaaaccggaaattgagacaaaacagctggacattcaggttgatagcgaagataattacagcgaaatgtttatcatcccgaaacaggtgcaggatatcaaagattttcgcaacttcaaattcagccaccgcgttgataaaaaaccgaatcgtcagctgattaacgataccctgtatagcacccgtatgaaagatgagcatgattatattgtgcagaccatcacggatatctatggcaaagataataccaacctgaaaaaacagttcaacaaaaacccggaaaaatttctgatgtatcagaacgatccgaaaacctttgagaaactgagcatcatcatgaaacagtacagcgacgaaaaaaacccgctggccaaatattacgaagaaaccggtgaatatctgaccaaatatagcaagaaaaacaacggtccgatcgtgaaaaagatcaaactgctgggtaataaagtgggcaatcatctggatgtgaccaacaaatatgaaaactccacgaagaagctggttaagctgtccattaaaaactatcgcttcgatgtgtatctgaccgagaaaggttataagtttgtgaccattgcctacctgaatgtgttcaaaaaagacaactattactatattccgaaagacaaataccaagaacttaaagagaagaagaaaatcaaggacaccgatcagtttatcgccagcttctataaaaacgatctgatcaagctgaacggcgacctgtataaaatcattggtgtgaatagtgatgaccgcaacatcattgagctggattattacgacatcaaatacaaggattactgcgagatcaacaacattaaaggtgaaccgcgtatcaaaaagaccattggcaaaaaaacggaaagcatcgaaaagtttaccaccgatgttctgggtaatctgtatctgcatagtaccgaaaaagcaccgcagctgattttcaaacgcggtctg

SEQ ID NO: 17 - DNA, 3171 bp, Synthetic construct
  source 1..3171: mol_type = other DNA
atgaaccagaaatttatcctgggtctggatattggtattaccagcgttggttatggcctgattgattacgaaaccaaaaacattattgatgccggtgttcgtctgtttccggaagcaaatgttgaaaataatgaaggtcgtcgtagcaaacgtggtagccgtcgtctgaaacgtcgtcgtattcatcgtctggaacgtgttaaactgctgctgaccgaatatgatctgattaacaaagagcagattccgaccagcaataacccgtatcagattcgtgttaaaggtctgagcgaaatcctgagcaaagatgaactggcaattgcactgctgcatctggcaaaacgccgtggcattcataatgttgatgttgcagcagataaagaagaaaccgcaagcgatagcctgagcaccaaagatcagattaacaaaaacgccaaatttctggaaagccgctatgtttgtgaactgcagaaagaacgtctggaaaatgaaggtcatgttcgtggtgttgaaaatcgctttctgacgaaagatattgtgcgtgaggccaaaaaaatcatcgatacccagatgcagtattacccggaaattgatgaaaccttcaaagagaaatatatcagcctggttgaaacccgtcgcgaatattttgaaggtcctggtcagggtagcccgtttggttggaatggtgatctgaaaaaatggtacgaaatgctgatgggtcactgtacctattttccgcaagaactgcgtagcgttaaatatgcctatagcgcagacctgtttaatgcactgaatgatctgaacaacctgattattcagcgcgataatagcgagaaactggaataccatgagaagtatcacatcatcgagaacgtgttcaagcagaaaaaaaagccgacgctgaaacaaatcgcaaaagagattggcgttaacccggaagatattaaaggttatcgtattaccaaaagcggcacaccggagtttacatcctttaaactgttccacgatctgaaaaaagtggtgaaagatcatgccatcctggatgatattgatctgctgaatcagattgcagaaatcctgaccatctatcaggataaagatagcattgttgcagaactgggtcagctggaatatctgatgagcgaagccgataaacagagcattagcgaactgaccggttataccggtacacatagcctgtcactgaaatgcatgaacatgattatcgatgaactgtggcatagcagcatgaaccagatggaagtttttacctatctgaatatgcgtccgaaaaagtatgagctgaaaggttatcagcgtattccgaccgatatgattgatgatgcaattctgagtccggttgtgaaacgcacctttattcagagcatcaacgtgatcaacaaagtgatcgagaaatatggcatccccgaagatatcattatcgaactggcacgtgaaaataactccgatgatcgcaaaaagttcatcaacaacctgcagaaaaagaatgaagcaacccgcaaacgcattaacgaaattattggtcagaccggtaatcagaatgccaaacgtattgtggaaaaaatccgtctgcatgatcagcaagagggcaaatgtctgtatagcctggaaagcattcctctggaagatctgctgaacaatccgaatcattatgaagtggatcacattattccgcgtagcgtgagctttgataattcctatcataataaagtgctggtgaaacagagcgaaaactccaaaaaatccaacctgacaccgtatcagtatttcaatagcggcaaatccaaactgagctacaaccagtttaaacagcatattctgaacctgagcaaaagccaggatcgcatcagcaagaagaagaaggagtacctgctggaagaacgcgatatcaacaaatttgaagtccagaaagagtttatcaaccgcaatctggttgatacccgttatgcaacccgtgaactgaccagctatctgaaagcatatttcagcgccaataacatggacgtgaaagtgaaaacaattaacggcagctttaccaaccatctgcgtaaagtttggcgctttgataaatatcgcaaccacggctataaacatcatgccgaagatgcactgattattgccaatgcagatttcctgttcaaagaaaacaaaaaactgcagaacaccaacaagatcctggaaaaaccgaccattgaaaacaacaccaaaaaagtgaccgtcgagaaagaagaggattacaacaacgtttttgaaaccccgaaactggtcgaggatattaaacagtatcgcgactataaattcagccaccgcgttgataaaaaaccgaatcgtcagctgattaacgataccctgtatagcacccgtatgaaagatgagcatgattatattgtgcagaccatcacggatatctatggcaaagataataccaacctgaaaaaacagttcaacaaaaacccggaaaaatttctgatgtatcagaacgatccgaaaacctttgagaaactgagcatcatcatgaaacagtacagcgacgaaaaaaacccgctggccaaatattacgaagaaaccggtgaatatctgaccaaatatagcaagaaaaacaacggtccgatcgtgaaaaagatcaaactgctgggtaataaagtgggcaatcatctggatgtgaccaacaaatatgaaaactccacgaagaagctggttaagctgtccattaaaaactatcgcttcgatgtgtatctgaccgagaaaggttataagtttgtgaccattgcctacctgaatgtgttcaaaaaagacaactattactatattccgaaagacaaataccaagaacttaaagagaagaagaaaatcaaggacaccgatcagtttatcgccagcttctataaaaacgatctgatcaagctgaacggcgacctgtataaaatcattggtgtgaatagtgatgaccgcaacatcattgagctggattattacgacatcaaatacaaggattactgcgagatcaacaacattaaaggtgaaccgcgtatcaaaaagaccattggcaaaaaaacggaaagcatcgaaaagtttaccaccgatgttctgggtaatctgtatctgcatagtaccgaaaaagcaccgcagctgattttcaaacgcggtctg

SEQ ID NO: 18 - DNA, 3171 bp, Synthetic construct
  source 1..3171: mol_type = other DNA
atgaaccagaagttcattctcggtctggacattggcattactagcgtgggatacggcttgattgactacgagactaagaacatcatcgatgccggcgtccgcctgttcccggaagccaacgtggagaacaatgagggccggaggtcgaagagaggctcccgccgcctgaagcggcggcgaatccaccggctggagagagtgaagctgctgctcaccgaatacgacctgatcaacaaagaacagatcccgacctccaacaacccgtaccagatcagagtgaagggtctgtcagaaatcctgtccaaggacgaactggcaatcgccctgctgcacctggccaagcggcgcggaatccacaacgtggatgtggctgccgacaaggaagaaaccgcttccgactccctgagcactaaggaccagatcaacaagaacgccaagttcttggagtcccgctacgtgtgcgagctgcagaaggaacggctggaaaacgaaggtcacgtgcgcggagtggaaaaccggttcctgacaaaggacattgtgcgcgaagcgaagaagatcattgatacccaaatgcagtactaccctgaaatcgacgagactttcaaggaaaagtacatttccctggtggaaacccggcgggaatacttcgaaggccccggacagggatcgccgttcggatggaacggggacctcaagaagtggtacgagatgctgatggggcactgtacctactttccgcaagaactgcgctccgtgaagtacgcgtactccgcggatctcttcaacgcgttgaatgacctgaacaacctgatcattcagagagacaattccgaaaagctcgagtaccacgagaagtatcacatcatcgagaatgtgttcaagcagaagaagaaaccgaccctcaagcaaatcgccaaggagattggcgtcaacccagaggacatcaagggatatcggattaccaagagcggcactcccgagtttacctctttcaagctgtttcatgatctgaagaaagtcgtgaaggaccatgccattctcgacgacattgatctcctgaatcagatcgcagagatcctgactatctaccaagacaaggactcgattgtggcagagctgggtcagctcgaatacctgatgtccgaggccgacaagcagtccatctccgaactgacagggtacacggggactcatagcctgtcgctgaagtgcatgaacatgatcattgatgaactgtggcacagctccatgaaccaaatggaagtgtttacctacctcaacatgcgccctaagaagtacgaactgaaaggctaccagcgcatccccaccgacatgatcgacgacgcgatcttgtcccctgtggtcaagaggaccttcattcaatccatcaacgtgatcaacaaggtcatcgaaaagtacggtattccagaagatatcatcattgagctcgctcgggagaacaactcggatgaccggaagaagttcatcaacaatcttcagaagaagaacgaagcgactcggaaacggatcaacgagatcatcggacagaccggaaaccagaacgccaaacggattgtcgaaaagattagactgcacgaccagcaggaagggaagtgcctgtactcactcgagtcaataccgctcgaggacctgttgaacaaccctaaccactatgaagtggaccacatcatccctcggtccgtgagcttcgacaactcgtaccacaacaaagtgctcgtgaagcagtccgaaaactccaagaaatccaacctgaccccgtaccaatacttcaattcgggaaagtcgaagctgtcgtacaaccagttcaaacaacacatactcaaccttagcaaaagccaggatcgcatttccaagaagaagaaggaatacctcctcgaggaaagggacatcaacaagttcgaagtgcagaaagagttcatcaatcgcaacttggtggataccagatatgccacccgggaactcaccagctatctcaaggcctacttttccgccaacaacatggacgtgaaggtcaagaccatcaacgggtccttcactaaccatctgagaaaggtctggcggtttgacaagtaccgcaaccacggatacaagcaccacgctgaagacgctctgatcatcgccaatgccgacttcctgttcaaggaaaacaagaagctgcagaacacgaacaagattctggaaaagcctaccattgagaacaacactaagaaggtcaccgtggagaaggaagaggactacaacaacgtgttcgaaactcctaaactggtggaggatatcaagcaataccgcgactacaagttctcacaccgggtggacaagaaaccgaatagacagctgatcaacgacacgttgtattccacccggatgaaggatgagcatgactacattgtgcagactatcaccgatatctacggaaaagataacactaacctgaagaaacaattcaacaagaacccagagaagttcctgatgtaccagaacgaccccaagacctttgagaagctttccatcatcatgaagcagtactccgacgagaagaacccgctggccaagtactacgaagaaaccggagaatacctgaccaagtacagcaagaagaacaacggtcccattgtcaagaagatcaagctgctcggcaacaaggtcggaaaccacctcgacgtgacaaacaagtacgagaactcgactaagaagcttgtgaagctgtcaatcaagaactatagattcgacgtgtacttgaccgaaaagggatacaagttcgtgaccatagcctatctgaacgtgttcaagaaagataactactactacatccccaaggacaagtaccaggagctcaaagaaaagaagaagatcaaagacaccgaccagttcattgcctccttctacaagaacgacctgatcaaactgaacggcgacctctacaagatcattggagtgaacagcgatgacaggaacatcattgagctggactactacgacatcaagtacaaggactactgcgagatcaacaacatcaagggcgaaccccggatcaagaaaactattggaaagaaaaccgagtccattgagaagttcaccactgacgtgctgggaaacctttacctccactccaccgagaaggcaccacaactgatcttcaagcgcggcctg

SEQ ID NO: 19 - DNA, 3166 bp, Synthetic construct
  source 1..3166: mol_type = other DNA
atgaaccagaaatttatcctgggtctggatattggtattaccagcgttggttatggcctgattgattacgaaaccaaaaacattattgatgccggtgttcgtctgtttccggaagcaaatgttgaaaataatgaaggtcgtcgtagcaaacgtggtagccgtcgtctgaaacgtcgtcgtattcatcgtctggaacgtgttaaaaaactgctggaagattataacctgctggatcagagccagattccgcagagcaccaatccgtatgcaattcgtgttaaaggtctgagcgaagcactgagcaaagatgaactggttattgcactgctgcatattgcaaaacgccgtggcattcataatatcaatgttagcagcgaagatgaggatgcaagcaatgaactgagcaccaaagaacaaattaaccgcaataataagctgctgaaggacaaatatgtttgcgaagttcagctgcagcgtctgaaagaaggtcagattcgcggagaaaaaaatcgctttaaaaccaccgatatcctgaaagaaattgatcagctgcttaaagtgcagaaggattatcataacctggacatcgatttcatcaaccagtacaaagaaatcgttgaaacccgtcgcgaatattttgaaggtccgggtaaaggtagcccgtatggttgggaaggtgatccgaaagcatggtatgaaaccctgatgggtcattgtacctattttccggatgaactgcgtagcgttaaatatgcctatagcgcagacctgtttaatgcactgaatgatctgaataacctggtgattcagcgtgatggtctgagcaaactggaatatcatgagaaatatcacatcatcgaaaacgtgttcaaacagaagaagaaaccgaccctgaaacaaatcgccaacgaaattaatgtgaacccggaagatattaaaggctaccgtattaccaaaagcggcaaaccggaatttacatcctttaaactgttccacgatctgaaaaaagtggtgaaagatcatgccatcctggatgatattgatctgctgaatcagattgcagaaatcctgaccatctatcaggataaagatagcattgttgcagaactgggtcagctggaatatctgatgagcgaagccgataaacagagcattagcgaactgaccggttataccggtacacatagcctgtcactgaaatgcatgaacatgattatcgatgaactgtggcatagcagcatgaaccagatggaagtttttacctatctgaatatgcgtccgaaaaagtatgagctgaaaggttatcagcgtattccgaccgatatgattgatgatgcaattctgagtccggttgtgaaacgcacctttattcagagcatcaacgtgatcaacaaagtgatcgagaaatatggcatccccgaagatatcattatcgaactggcacgtgaaaataactccgatgatcgcaaaaagttcatcaacaacctgcagaaaaagaatgaagcaacccgcaaacgcattaacgaaattattggtcagaccggtaatcagaatgccaaacgtattgtggaaaaaatccgtctgcatgatcagcaagagggtaaatgtctgtatagcctggaaagcattcctctggaagatctgctgaacaatccgaatcattatgaagtggatcacattattccgcgtagcgtgagctttgataattcctatcataataaagtgctggtgaaacagagcgaaaactccaaaaaatccaacctgacaccgtatcagtatttcaatagcggcaaatccaaactgagctacaaccagtttaaacagcatattctgaacctgagcaaaagccaggatcgcatcagcaagaagaagaaggagtacctgctggaagaacgcgatattaacaaatttgaagtgcagaaagaatttatcaaccgcaacctggttgatacccgttatgcaacccgtgaactgaccaattatctgaaagcatatttcagcgccaacaacatgaacgtgaaagtgaaaacgattaacggcagctttaccgattatctgcgtaaagtgtggaaattcaaaaaagaacgcaaccacggctataaacatcatgccgaagatgccctgattattgcaaatgcagatttcctgtttaaagaaaacaaaaaactgaaagccgtcaacagcgtgctggaaaaaccggaaattgagacaaaacagctggacattcaggttgatagcgaagataattacagcgaaatgtttatcatcccgaaacaggtgcaggatatcaaagattttcgcaacttcaaatatagccaccgcgttgacaaaaaacctaatcgtcagctgattaacgataccctgtatagcacccgcaaaaaagataacagcacctatattgtgcagaccattaaagacatctacgccaaagataataccaccctgaaaaaacagttcgacaaaagcccagaaaaatttctgatgtatcagcatgatccgcgtaccttcgaaaaactggaagttattatgaaacagtatgccaacgagaaaaatccgctggccaaatatcacgaagaaaccggtgaatatctgaccaaatattccaagaagaacaacggtccgatcgttaaatccctgaaatatatcggtaataaactgggcagccatctggatgttacccatcagtttaaaagctccacaaagaagctggttaaactgtccatcaaaccgtatcgctttgatgtgtatctgaccgacaaaggctataaattcattaccatcagctatctggacgtgctgaaaaaagacaactattattatatcccggaacagaaatatgataaactgaaactgggtaaagccatcgataaaaacgccaaatttatcgccagcttctacaaaaacgacctgattaaactggatggcgagatctataaaatcatcggtgttaatagcgacacccgcaatatgattgagctggatctgccggatattcgctataaagaatattgcgaactgaacaacattaaaggcgaaccgcgtatcaaaaagaccatcggcaaaaaagtgaatagcatcgagaaactgaccaccgatgttctgggtaatgtgtttaccaatacccagtataccaaacctcagctgctgttcaaacgcggtaatggtg

SEQ ID NO: 20 - DNA, 3162 bp, Synthetic construct
  source 1..3162: mol_type = other DNA
atgaaccaaaagttcattctggggctcgatatcggcatcacctccgtgggatatggtctgatcgactacgagactaagaacatcatcgacgctggagtgcgactgttcccggaagcgaacgtggagaacaacgaaggccgcagatccaagcgcgggtccagaaggctcaagaggcggaggatccatagactcgagagagtgaagaagctccttgaagattacaatctgttggaccagtcacagattccccaaagcaccaacccgtacgccatcagagtgaagggcctgtcagaagcactgtcgaaagatgaactggtcattgccctgctgcatattgccaaacggcgcggaatccataacatcaacgtgtcgagcgaagatgaggacgcgtccaacgaactgtcaaccaaggaacagatcaaccggaacaacaaactgctgaaggacaaatacgtctgcgaggtgcagcttcaacggctgaaagagggacagatcaggggagagaaaaaccggttcaagaccaccgacatccttaaggagatcgaccaactcctgaaagtgcagaaggactatcacaacctcgacattgattttatcaaccagtacaaggagattgtggaaactcggagggagtacttcgagggacctggaaagggatccccttatggctgggaaggggaccccaaggcttggtacgaaaccctgatgggccattgcacttactttccggatgaactccggtccgtgaagtacgcttactctgccgacctgttcaatgcactcaacgaccttaacaacttggtgatccaacgcgatggtctttccaagttggagtaccacgaaaagtaccacatcatcgagaacgtgttcaagcagaaaaagaagccaactctgaagcagattgccaacgaaattaacgtgaaccccgaggatatcaagggataccggattaccaagtccggcaaaccagagttcacctcattcaagctgtttcacgatctgaagaaggtcgtgaaggaccacgccatcctggatgacattgatcttctgaaccagattgctgagatcctgaccatctaccaggacaaggactcgattgtggccgaactgggacagctcgagtacctgatgtccgaagccgataagcagtccatcagcgaactcaccggttacaccggtacccactccttgtcccttaagtgcatgaacatgatcattgacgaactgtggcactccagcatgaaccagatggaggtgttcacctacttgaacatgcgcccgaagaagtacgagctgaagggctaccagcgcatacccacggacatgatcgacgacgccatcctctcaccggtggtcaagcgcaccttcattcaatctatcaacgtgatcaacaaggtcatcgagaagtacggcattcctgaggatatcatcatcgagctggctcgggagaacaactcagacgataggaagaagttcattaacaacctccagaaaaagaacgaggccactcgcaagcggattaatgagatcatcggtcagaccgggaaccagaacgccaagcggatcgtggaaaagattcggctccacgaccaacaggagggaaagtgtctgtactcgctggagtccattcccctggaggacctcctgaacaacccaaaccactacgaggtcgatcacataatcccccgcagcgtgtcattcgacaactcctaccataacaaggtcctcgtgaagcagtcggagaatagcaagaagtcgaacctgactccgtaccagtacttcaactccggcaaatccaagctgtcctacaatcagttcaaacagcacattctcaacctgtccaagagccaggacaggatttcgaagaagaagaaggaataccttctcgaggaacgggatatcaataagttcgaggtgcagaaggagtttatcaatagaaacctggtggacactcgctatgccacccgcgaactgaccaactacctgaaggcgtacttctccgccaacaacatgaacgtgaaggtcaaaactattaacggcagcttcaccgactatctgcgcaaggtctggaagttcaagaaggaacgcaaccacggttacaagcaccacgcggaagatgcgctgattatcgccaacgctgacttcctgttcaaggaaaacaagaagctcaaggccgtgaactcagtgctcgagaagcctgaaatcgagactaagcagctggacatccaggtcgattcggaagataactactccgaaatgttcatcatccctaagcaagtgcaggacatcaaggacttcaggaatttcaagtacagccatcgcgtggacaagaagccaaacagacagctgatcaacgatacactgtattccacccggaagaaggacaactccacctacatcgtccaaaccattaaggacatctacgcaaaggacaacaccacgcttaagaagcagttcgacaagagccccgaaaagttcctcatgtaccagcacgaccccagaaccttcgagaagcttgaagtgatcatgaagcagtacgccaacgaaaagaacccactggctaagtaccacgaggaaaccggcgaatacctgaccaagtactccaaaaagaacaacggaccgatcgtcaagtccctgaagtacattgggaacaagctcggctcgcacctcgatgtgacccaccagttcaagtcctcgaccaaaaagctcgtgaagctgtccatcaagccgtaccggttcgacgtgtacctgactgacaagggatataagttcatcaccatttcctacctcgacgtgttgaagaaggataactactactacattccggaacagaagtacgacaagctcaagctcggaaaggccatcgacaaaaatgcgaagttcatcgcgagcttctacaagaatgacttgatcaagctggatggcgaaatctacaagatcatcggggtcaactccgatacccgcaacatgattgagctggatctgcccgacattcggtacaaggaatactgcgagctgaacaacatcaagggagaaccgcggatcaagaaaaccatcggaaagaaagtgaacagcatcgagaaactgactactgacgtcctgggaaacgtgttcaccaacacacaatacaccaaaccccagctgctgtttaagcgcgggaac

SEQ ID NO: 21 - DNA, 3171 bp, Synthetic construct
  source 1..3171: mol_type = other DNA
atgaaccagaaatttatcctgggtctggatattggtattaccagcgttggttatggcctgattgattacgaaaccaaaaacattattgatgccggtgttcgtctgtttccggaagcaaatgttgaaaataatgaaggtcgtcgtagcaaacgtggtagccgtcgtctgaaacgtcgtcgtattcatcgtctggatcgtgttaaacatctgctggcagaatatgatctgctggatctgaccaatattccgaaaagcaccaatccgtatcagacccgtgttaaaggtctgaatgaaaagctgagcaaagatgaactggttattgcactgctgcatattgcaaaacgccgtggcattcataacgttgatgttgcagcagataaagaagaaaccgcaagcgatagcctgagcaccaaagatcagattaacaaaaacgccaaatttctggaaagccgctatgtttgtgaactgcagaaagaacgtctggaaaatgaaggtcatgttcgtggtgttgaaaatcgctttctgacgaaagatattgtgcgtgaggccaaaaaaatcatcgatacccagatgcagtattacccggaaattgatgaaaccttcaaagagaaatatatcagcctggttgaaacccgtcgcgaatattttgaaggtccgggtaaaggtagcccgtttggttgggaaggtaatatcaagaaatggtttgagcagatgatgggccactgtacctattttccagaagaactgcgtagcgtcaaatatagctattcagccgaactgtttaacgccctgaatgatctgaataatctggtgattacccgtgatgaagatgccaaactgaattatggtgagaaattccagatcatcgaaaacgtgttcaaacagaagaaaacaccgaacctgaaacaaatcgccattgaaattggtgtgcatgaaaccgaaatcaaaggttatcgtgtgaacaaaagcggtacaccggaatttaccgaatttaaactgtatcatgacctgaaaagcatcgtgttcgataaaagcattctggaaaatgaagccatcctggatcagattgcagaaattctgaccatctatcaggatgagcagagcattaaagaggaactgaataaactgccggaaatactgaacgaacaggataaagcagaaatcgccaaactgattggttataatggcacccatcgtctgagcctgaaatgtattcacctgattaatgaagaactgtggcagaccagccgtaatcagatggaaattttcaactacctgaacatcaaaccgaacaaagtggatctgagtgagcagaacaaaatcccgaaagatatggtgaacgactttattctgagtccggttgtgaaacgcacctttattcagagcatcaacgtgatcaacaaagtgatcgagaaatatggcatccccgaagatatcattatcgaactggcacgtgaaaataactccgatgatcgcaaaaagttcatcaacaacctgcagaaaaagaatgaagcaacccgcaaacgcattaacgaaattattggtcagaccggtaatcagaatgccaaacgtattgtggaaaaaatccgtctgcatgatcagcaagagggtaaatgtctgtatagcctgaaagatatcccgctggaagatctgctgcgcaatccgaacaattatgatatcgaccatattattccgcgaagcgtgagctttgatgatagcatgcataacaaagttctggttcgtcgcgaacagaatgccaaaaagaataatcagaccccgtatcagtatctgaccagtggttatgcagatatcaaatacagcgtgtttaagcagcatgttctgaatctggccgaaaataaagatcgcatgaccaaaaaaaagcgcgagtatctgctggaagaacgcgacattaacaaatttgaagtgcagaaagaatttatcaaccgcaacctggttgatacccgttatgcaacccgtgaactgaccaattatctgaaagcatatttcagcgccaacaacatgaacgtgaaagtgaaaacgattaacggcagctttaccgattatctgcgtaaagtgtggaaattcaaaaaagaacgcaaccacggctataaacatcatgccgaagatgccctgattattgcaaatgcagatttcctgtttaaagaaaacaaaaaactgaaagccgtcaacagcgtgctggaaaaaccggaaattgagacaaaacagctggacattcaggttgatagcgaagataattacagcgaaatgtttatcatcccgaaacaggtgcaggatatcaaagattttcgcaacttcaaatatagccaccgcgttgacaaaaaacctaatcgtcagctgattaacgataccctgtatagcacccgcaaaaaagataacagcacctatattgtgcagaccattaaagacatctacgccaaagataataccaccctgaaaaaacagttcgacaaaagcccagaaaaatttctgatgtatcagcatgatccgcgtaccttcgaaaaactggaagttattatgaaacagtatgccaacgagaaaaatccgctggccaaatatcacgaagaaaccggtgaatatctgaccaaatattccaagaagaacaacggtccgatcgttaaatccctgaaatatatcggtaataaactgggcagccatctggatgttacccatcagtttaaaagctccacaaagaagctggttaaactgtccatcaaaccgtatcgctttgatgtgtatctgaccgacaaaggctataaattcattaccatcagctatctggacgtgctgaaaaaagacaactattattatatcccggaacagaaatatgataaactgaaactgggtaaagccatcgataaaaacgccaaatttatcgccagcttctacaaaaacgacctgattaaactggatggcgagatctataaaatcatcggtgttaatagcgacacccgcaatatgattgagctggatctgccggatattcgctataaagaatattgcgaactgaacaacattaaaggcgaaccgcgtatcaaaaagaccatcggcaaaaaagtgaatagcatcgagaaactgaccaccgatgttctgggtaatgtgtttaccaatacccagtataccaaacctcagctgctgttcaaacgcggtaat

SEQ ID NO: 22 - DNA, 3171 bp, Synthetic construct
  source 1..3171: mol_type = other DNA
atgaaccaaaagttcattctggggctcgatatcggcatcacctccgtgggatatggtctgatcgactacgagactaagaacatcatcgacgctggagtgcgactgttcccggaagcgaacgtggagaacaacgaaggccgcagatccaagcgcgggtccagaaggctcaagaggcggaggatccatagactcgacagagtgaagcacctccttgccgaatacgatctgttggaccttaccaacattcccaagagcaccaacccgtaccaaaccagagtgaagggcctgaacgaaaagctgtcgaaagatgaactggtcattgccctgctgcatattgccaaacggcgcggaatccataacgtggacgtggccgctgacaaggaagagactgcgtccgactcgctgtcaaccaaggaccagatcaacaagaacgccaaattcctggaaagccgctacgtctgcgagcttcaaaaagaacggctggagaacgagggacacgtcaggggagtggagaaccggttcctgaccaaggacatcgtgcgggaagccaagaagatcatcgacacccaaatgcagtattatccggaaattgatgaaacttttaaggagaagtacatttccctggtggaaactcggagggagtacttcgagggacctggaaagggatcccctttcggctgggaagggaacattaagaagtggtttgaacagatgatgggccattgcacttactttccggaagaactccggtccgtgaagtactcatactctgccgagctgttcaatgcactcaacgaccttaacaacttggtgatcacccgcgatgaagatgccaagttgaactacggagaaaagttccagatcatcgagaacgtgttcaagcagaaaaagaccccaaatctgaagcagattgccatcgaaattggcgtgcacgagactgagatcaagggataccgggtcaacaagtccggcacgccagagttcaccgagttcaagctgtaccacgatctgaagtcgatcgtgtttgacaagtccatcctggaaaacgaagccattctggaccagattgctgagatcctgaccatctaccaggacgagcaatcgattaaggaagaactgaacaagctccccgagattctgaacgaacaggataaggccgagatcgcgaagctcattggttacaatggtacccaccgcttgtcccttaagtgcatccatctgatcaatgaggaactgtggcagaccagccggaaccagatggagatcttcaattacttgaacatcaagccgaacaaggtggacctgtccgaacagaacaagatacccaaggacatggtcaacgactttatcctctcaccggtggtcaagcgcaccttcattcaatctatcaacgtgatcaacaaggtcatcgagaagtacggcattcctgaggatatcatcatcgagctggctcgggagaacaactcagacgataggaagaagttcattaacaacctccagaaaaagaacgaggccactcgcaagcggattaatgagatcatcggtcagaccgggaaccagaacgccaagcggatcgtggaaaagattcggctccacgaccaacaggagggaaagtgtctgtactcgctgaaggacattcccctggaggacctcctgaggaacccaaacaactacgacatcgatcacataatcccccgcagcgtgtcattcgacgattccatgcataacaaggtcctcgtgcggagagagcagaatgccaagaagaacaaccagactccgtaccagtacctgacgtccggctacgcagacatcaagtactcagtgttcaaacagcacgtgctcaacctggccgagaacaaggacaggatgaccaagaagaagcgcgaataccttctcgaggaacgggatatcaataagttcgaggtgcagaaggagtttatcaatagaaacctggtggacactcgctatgccacccgcgaactgaccaactacctgaaggcgtacttctccgccaacaacatgaacgtgaaggtcaaaactattaacggcagcttcaccgactatctgcgcaaggtctggaagttcaagaaggaacgcaaccacggttacaagcaccacgcggaagatgcgctgattatcgccaacgctgacttcctgttcaaggaaaacaagaagctcaaggccgtgaactcagtgctcgagaagcctgaaatcgagactaagcagctggacatccaggtcgattcggaagataactactccgaaatgttcatcatccctaagcaagtgcaggacatcaaggacttcaggaatttcaagtacagccatcgcgtggacaagaagccaaacagacagctgatcaacgatacactgtattccacccggaagaaggacaactccacctacatcgtccaaaccattaaggacatctacgcaaaggacaacaccacgcttaagaagcagttcgacaagagccccgaaaagttcctcatgtaccagcacgaccccagaaccttcgagaagcttgaagtgatcatgaagcagtacgccaacgaaaagaacccactggctaagtaccacgaggaaaccggcgaatacctgaccaagtactccaaaaagaacaacggaccgatcgtcaagtccctgaagtacattgggaacaagctcggctcgcacctcgatgtgacccaccagttcaagtcctcgaccaaaaagctcgtgaagctgtccatcaagccgtaccggttcgacgtgtacctgactgacaagggatataagttcatcaccatttcctacctcgacgtgttgaagaaggataactactactacattccggaacagaagtacgacaagctcaagctcggaaaggccatcgacaaaaatgcgaagttcatcgcgagcttctacaagaatgacttgatcaagctggatggcgaaatctacaagatcatcggggtcaactccgatacccgcaacatgattgagctggatctgcccgacattcggtacaaggaatactgcgagctgaacaacatcaagggagaaccgcggatcaagaaaaccatcggaaagaaagtgaacagcatcgagaaactgactactgacgtcctgggaaacgtgttcaccaacacacaatacaccaaaccccagctgctgtttaagcgcgggaac

SEQ ID NO: 23 - DNA, 3171 bp, Synthetic construct
  source 1..3171: mol_type = other DNA
atgaaccagaaatttatcctgggtctggatattggtattaccagcgttggttatggcctgattgattacgaaaccaaaaacattattgatgccggtgttcgtctgtttccggaagcaaatgttgaaaataatgaaggtcgtcgtagcaaacgtggtagccgtcgtctgaaacgtcgtcgtattcatcgtctggatcgtgttaaacatctgctggcagaatatgatctgctggatctgaccaatattccgaaaagcaccaatccgtatcagacccgtgttaaaggtctgaatgaaaagctgagcaaagatgaactggttattgcactgctgcatattgcaaaacgccgtggcattcataacgttgatgttgcagcagataaagaagaaaccgcaagcgatagcctgagcaccaaagatcagattaacaaaaacgccaaatttctggaaagccgctatgtttgtgaactgcagaaagaacgtctggaaaatgaaggtcatgttcgtggtgttgaaaatcgctttctgacgaaagatattgtgcgtgaggccaaaaaaatcatcgatacccagatgcagtattacccggaaattgatgaaaccttcaaagagaaatatatcagcctggttgaaacccgtcgcgaatattttgaaggtccgggtaaaggtagcccgtttggttgggaaggtaatatcaagaaatggtttgagcagatgatgggccactgtacctattttccagaagaactgcgtagcgtcaaatatagctattcagccgaactgtttaacgccctgaatgatctgaataatctggtgattacccgtgatgaagatgccaaactgaattatggtgagaaattccagatcatcgaaaacgtgttcaaacagaagaaaacaccgaacctgaaacaaatcgccattgaaattggtgtgcatgaaaccgaaatcaaaggttatcgtgtgaacaaaagcggtacaccggaatttaccgaatttaaactgtatcatgacctgaaaagcatcgtgttcgataaaagcattctggaaaatgaagccatcctggatcagattgcagaaattctgaccatctatcaggatgagcagagcattaaagaggaactgaataaactgccggaaatactgaacgaacaggataaagcagaaatcgccaaactgattggttataatggcacccatcgtctgagcctgaaatgtattcacctgattaatgaagaactgtggcagaccagccgtaatcagatggaaattttcaactacctgaacatcaaaccgaacaaagtggatctgagtgagcagaacaaaatcccgaaagatatggtgaacgactttattctgagtccggttgtgaaacgcacctttattcagagcatcaacgtgatcaacaaagtgatcgagaaatatggcatccccgaagatatcattatcgaactggcacgtgaaaataactccgatgatcgcaaaaagttcatcaacaacctgcagaaaaagaatgaagcaacccgcaaacgcattaacgaaattattggtcagaccggtaatcagaatgccaaacgtattgtggaaaaaatccgtctgcatgatcagcaagagggtaaatgtctgtatagcctgaaagatatcccgctggaagatctgctgcgcaatccgaacaattatgatatcgaccatattattccgcgaagcgtgagctttgatgatagcatgcataacaaagttctggttcgtcgcgaacagaatgccaaaaagaataatcagaccccgtatcagtatctgaccagtggttatgcagatatcaaatacagcgtgtttaagcagcatgttctgaatctggccgaaaataaagatcgcatgaccaaaaaaaagcgcgagtatctgctggaagaacgcgacattaacaaatttgaagtgcagaaagaatttatcaaccgcaacctggttgatacccgttatgcaacccgtgaactgaccaattatctgaaagcatatttcagcgccaacaacatgaacgtgaaagtgaaaacgattaacggcagctttaccgattatctgcgtaaagtgtggaaattcaaaaaagaacgcaaccacggctataaacatcatgccgaagatgccctgattattgcaaatgcagatttcctgtttaaagaaaacaaaaaactgaaagccgtcaacagcgtgctggaaaaaccggaaattgagacagatcagaaagacattcaggttgatagcgaagataattacagcgaaatgtttatcatcccgaaacaggtgcaggatatcaaagattttcgcaacttcaaatatagccaccgcgttgacaaaaaacctaatcgtcagctgattaacgataccctgtatagcacccgcaaaaaagataacagcacctatattgtgcagaccattaaagacatctacgccaaagataataccaccctgaaaaaacagttcgacaaaagcccagaaaaatttctgatgtatcagcatgatccgcgtaccttcgaaaaactggaagttattatgaaacagtatgccaacgagaaaaatccgctggccaaatatcacgaagaaaccggtgaatatctgaccaaatattccaagaagaacaacggtccgatcgttaaatccctgaaatatatcggtaataaactgggcagccatctggatgttacccatcagtttaaaagctccacaaagaagctggttaaactgtccatcaaaccgtatcgctttgatgtgtatctgaccgacaaaggctataaattcattaccatcagctatctggacgtgctgaaaaaagacaactattattatatcccggaacagaaatatgataaactgaaactgggtaaagccatcgataaaaacgccaaatttatcgccagcttctacaaaaacgacctgattaaactggatggcgagatctataaaatcatcggtgttaatagcgacacccgcaatatgattgagctggatctgccggatattcgctataaagaatattgcgaactgaacaacattaaaggcgaaccgcgtatcaaaaagaccatcggcaaaaaagtgaatagcatcgagaaactgaccaccgatgttctgggtaatgtgtttaccaatacccagtataccaaacctcagctgctgttcaaacgcggtaat

SEQ ID NO: 24 - DNA, 3171 bp, Synthetic construct
  source 1..3171: mol_type = other DNA
atgaaccagaaatttatcctgggtctggatattggtattaccagcgttggttatggcctgattgattacgaaaccaaaaacattattgatgccggtgttcgtctgtttccggaagcaaatgttgaaaataatgaaggtcgtcgtagcaaacgtggtagccgtcgtctgaaacgtcgtcgtattcatcgtctggatcgtgttaaacatctgctggcagaatatgatctgctggatctgaccaatattccgaaaagcaccaatccgtatcagacccgtgttaaaggtctgaatgaaaagctgagcaaagatgaactggttattgcactgctgcatattgcaaaacgccgtggcattcataacgttgatgttgcagcagataaagaagaaaccgcaagcgatagcctgagcaccaaagatcagattaacaaaaacgccaaatttctggaaagccgctatgtttgtgaactgcagaaagaacgtctggaaaatgaaggtcatgttcgtggtgttgaaaatcgctttctgacgaaagatattgtgcgtgaggccaaaaaaatcatcgatacccagatgcagtattacccggaaattgatgaaaccttcaaagagaaatatatcagcctggttgaaacccgtcgcgaatattttgaaggtccgggtaaaggtagcccgtttggttgggaaggtaatatcaagaaatggtttgagcagatgatgggccactgtacctattttccagaagaactgcgtagcgtcaaatatagctattcagccgaactgtttaacgccctgaatgatctgaataatctggtgattacccgtgatgaagatgccaaactgaattatggtgagaaattccagatcatcgaaaacgtgttcaaacagaagaaaacaccgaacctgaaacaaatcgccattgaaattggtgtgcatgaaaccgaaatcaaaggttatcgtgtgaacaaaagcggtacaccggaatttaccgaatttaaactgtatcatgacctgaaaagcatcgtgttcgataaaagcattctggaaaatgaagccatcctggatcagattgcagaaattctgaccatctatcaggatgagcagagcattaaagaggaactgaataaactgccggaaatactgaacgaacaggataaagcagaaatcgccaaactgattggttataatggcacccatcgtctgagcctgaaatgtattcacctgattaatgaagaactgtggcagaccagccgtaatcagatggaaattttcaactacctgaacatcaaaccgaacaaagtggatctgagtgagcagaacaaaatcccgaaagatatggtgaacgactttattctgagtccggttgtgaaacgcacctttattcagagcatcaacgtgatcaacaaagtgatcgagaaatatggcatccccgaagatatcattatcgaactggcacgtgaaaataactccgatgatcgcaaaaagttcatcaacaacctgcagaaaaagaatgaagcaacccgcaaacgcattaacgaaattattggtcagaccggtaatcagaatgccaaacgtattgtggaaaaaatccgtctgcatgatcagcaagagggtaaatgtctgtatagcctgaaagatatcccgctggaagatctgctgcgcaatccgaacaattatgatatcgaccatattattccgcgaagcgtgagctttgatgatagcatgcataacaaagttctggttcgtcgcgaacagaatgccaaaaagaataatcagaccccgtatcagtatctgaccagtggttatgcagatatcaaatacagcgtgtttaagcagcatgttctgaatctggccgaaaataaagatcgcatgaccaaaaaaaagcgcgagtatctgctggaagaacgcgacattaacaaatttgaagtgcagaaagaatttatcaaccgcaacctggttgatacccgttatgcaacccgtgaactgaccaattatctgaaagcatatttcaccgccaacaacatggatgtgaaagtgaaaacgattaacggcagctttaccgattatctgcgtaaagtgtggaaattcaaaaaagaacgcaaccacggctataaacatcatgccgaagatgccctgattattgcaaatgcagatttcctgtttaaagaaaacaaaaaactgaaagccgtcaacagcgtgctggaaaaaccggaaattgagacaaaacagctggacattcaggttgatagcgaagataattacagcgaaatgtttatcatcccgaaacaggtgcaggatatcaaagattttcgcaacttcaaatatagccaccgcgttgacaaaaaacctaatcgtcagctgattaacgataccctgtatagcacccgcaaaaaagataacagcacctatattgtgcagaccattaaagacatctacgccaaagataataccaccctgaaaaaacagttcgacaaaagcccagaaaaatttctgatgtatcagcatgatccgcgtaccttcgaaaaactggaagttattatgaaacagtatgccaacgagaaaaatccgctggccaaatatcacgaagaaaccggtgaatatctgaccaaatattccaagaagaacaacggtccgatcgttaaatccctgaaatatatcggtaataaactgggcagccatctggatgttacccatcagtttaaaagctccacaaagaagctggttaaactgtccatcaaaccgtatcgctttgatgtgtatctgaccgacaaaggctataaattcattaccatcagctatctggacgtgctgaaaaaagacaactattattatatcccggaacagaaatatgataaactgaaactgggtaaagccatcgataaaaacgccaaatttatcgccagcttctacaaaaacgacctgattaaactggatggcgagatctataaaatcatcggtgttaatagcgacacccgcaatatgattgagctggatctgccggatattcgctataaagaatattgcgaactgaacaacattaaaggcgaaccgcgtatcaaaaagaccatcggcaaaaaagtgaatagcatcgagaaactgaccaccgatgttctgggtaatgtgtttaccaatacccagtataccaaacctcagctgctgttcaaacgcggtaat

SEQ ID NO: 25 - DNA, 3171 bp, Synthetic construct
  source 1..3171: mol_type = other DNA
atgaaccagaaatttatcctgggtctggatattggtattaccagcgttggttatggcctgattgattacgaaaccaaaaacattattgatgccggtgttcgtctgtttccggaagcaaatgttgaaaataatgaaggtcgtcgtagcaaacgtggtagccgtcgtctgaaacgtcgtcgtattcatcgtctggatcgtgttaaacatctgctggcagaatatgatctgctggatctgaccaatattccgaaaagcaccaatccgtatcagacccgtgttaaaggtctgaatgaaaagctgagcaaagatgaactggttattgcactgctgcatattgcaaaacgccgtggcattcataacgttgatgttgcagcagataaagaagaaaccgcaagcgatagcctgagcaccaaagatcagattaacaaaaacgccaaatttctggaaagccgctatgtttgtgaactgcagaaagaacgtctggaaaatgaaggtcatgttcgtggtgttgaaaatcgctttctgacgaaagatattgtgcgtgaggccaaaaaaatcatcgatacccagatgcagtattacccggaaattgatgaaaccttcaaagagaaatatatcagcctggttgaaacccgtcgcgaatattttgaaggtccgggtaaaggtagcccgtttggttgggaaggtaatatcaagaaatggtttgagcagatgatgggccactgtacctattttccagaagaactgcgtagcgtcaaatatagctattcagccgaactgtttaacgccctgaatgatctgaataatctggtgattacccgtgatgaagatgccaaactgaattatggtgagaaattccagatcatcgaaaacgtgttcaaacagaagaaaacaccgaacctgaaacaaatcgccattgaaattggtgtgcatgaaaccgaaatcaaaggttatcgtgtgaacaaaagcggtacaccggaatttaccgaatttaaactgtatcatgacctgaaaagcatcgtgttcgataaaagcattctggaaaatgaagccatcctggatcagattgcagaaattctgaccatctatcaggatgagcagagcattaaagaggaactgaataaactgccggaaatactgaacgaacaggataaagcagaaatcgccaaactgattggttataatggcacccatcgtctgagcctgaaatgtattcacctgattaatgaagaactgtggcagaccagccgtaatcagatggaaattttcaactacctgaacatcaaaccgaacaaagtggatctgagtgagcagaacaaaatcccgaaagatatggtgaacgactttattctgagtccggttgtgaaacgcacctttattcagagcatcaacgtgatcaacaaagtgatcgagaaatatggcatccccgaagatatcattatcgaactggcacgtgaaaataactccgatgatcgcaaaaagttcatcaacaacctgcagaaaaagaatgaagcaacccgcaaacgcattaacgaaattattggtcagaccggtaatcagaatgccaaacgtattgtggaaaaaatccgtctgcatgatcagcaagagggtaaatgtctgtatagcctgaaagatatcccgctggaagatctgctgcgcaatccgaacaattatgatatcgaccatattattccgcgaagcgtgagctttgatgatagcatgcataacaaagttctggttcgtcgcgaacagaatgccaaaaagaataatcagaccccgtatcagtatctgaccagtggttatgcagatatcaaatacagcgtgtttaagcagcatgttctgaatctggccgaaaataaagatcgcatgaccaaaaaaaagcgcgagtatctgctggaagaacgcgacattaacaaatttgaagtgcagaaagaatttatcaaccgcaacctggttgatacccgttatgcaacccgtgaactgaccaattatctgaaagcatatttcagcgccaacaacatgaacgtgaaagtgaaaacgattaacggcagctttaccgattatctgcgtaaagtgtggaaattcaaaaaagaacgcaaccacggctataaacatcatgccgaagatgccctgattattgcaaatgcagatttcctgtttaaagaaaacaaaaaactgaaagccgtcaacagcgtgctggaaaaaccggaaattgagacaaatcagaatgacattcaggttgatagcgaagataattacagcgaaatgtttatcatcccgaaacaggtgcaggatatcaaagattttcgcaacttcaaatatagccaccgcgttgacaaaaaacctaatcgtcagctgattaacgataccctgtatagcacccgcaaaaaagataacagcacctatattgtgcagaccattaaagacatctacgccaaagataataccaccctgaaaaaacagttcgacaaaagcccagaaaaatttctgatgtatcagcatgatccgcgtaccttcgaaaaactggaagttattatgaaacagtatgccaacgagaaaaatccgctggccaaatatcacgaagaaaccggtgaatatctgaccaaatattccaagaagaacaacggtccgatcgttaaatccctgaaatatatcggtaataaactgggcagccatctggatgttacccatcagtttaaaagctccacaaagaagctggttaaactgtccatcaaaccgtatcgctttgatgtgtatctgaccgacaaaggctataaattcattaccatcagctatctggacgtgctgaaaaaagacaactattattatatcccggaacagaaatatgataaactgaaactgggtaaagccatcgataaaaacgccaaatttatcgccagcttctacaaaaacgacctgattaaactggatggcgagatctataaaatcatcggtgttaatagcgacacccgcaatatgattgagctggatctgccggatattcgctataaagaatattgcgaactgaacaacattaaaggcgaaccgcgtatcaaaaagaccatcggcaaaaaagtgaatagcatcgagaaactgaccaccgatgttctgggtaatgtgtttaccaatacccagtataccaaacctcagctgctgttcaaacgcggtaat

SEQ ID NO: 26 - DNA, 3159 bp, Synthetic construct
  source 1..3159: mol_type = other DNA
atgaaccagaaatttatcctgggtctggatattggtattaccagcgttggttatggcctgattgattacgaaaccaaaaacattattgatgccggtgttcgtctgtttccggaagcaaatgttgaaaataatgaaggtcgtcgtagcaaacgtggtagccgtcgtctgaaacgtcgtcgtattcatcgtctggaacgtgttaaaagcctgctgagcgaatataagattattagcggtctggcaccgaccaataatcagccgtataacattcgtgttaaaggtctgaccgaacagctgaccaaagatgaactggcagttgcactgctgcatattgccaaacgccgtggcattcataaaatcgatgtgattgatagcaatgacgatgtgggtaatgaactgagcaccaaagaacagctgaacaaaaatagcaaactgctgaaagacaaattcgtgtgtcagattcagctggaacgtatgaatgaaggccaggttcgtggtgaaaagaatcgctttaaaaccgcagacatcatcaaagaaattatccagctgctgaacgtgcagaaaaacttccatcagctggatgaaaacttcatcaacaaatacatcgagctggttgaaatgcgtcgcgaatattttgaaggtcctggtcagggtagcccgtttggttggaatggtgatctgaaaaaatggtacgaaatgctgatgggtcactgtacctattttccgcaagaactgcgtagcgttaaatatgcctatagcgcagacctgtttaatgcactgaatgatctgaacaacctgattattcagcgcgataatagcgagaaactggaataccatgagaagtatcacatcatcgagaacgtgttcaagcagaaaaaaaagccgacgctgaaacaaatcgcaaaagagattggcgttaacccggaagatattaaaggttatcgtattaccaaaagcggtacaccggaattcaccgaatttaaactgtatcacgatctgaaaagcgtgctgtttgatcagagcattctggaaaatgaagatgtgctggaccagattgcagaaattctgaccatttatcaggacaaagacagcatcaaaagcaaactgaccgaactggatattctgctgaatgaagaagataaagagaacattgcacagctgaccggttataacggcacacatcgcctgagcctgaaatgtattcgtctggtactggaagaacagtggtatagcagccgtaatcagatggaaatctttacccatctgaacattaaaccgaagaaaatcaatctgaccgcagccaacaaaattccgaaagccatgattgatgagtttattctgagtccggttgtgaaacgcacctttattcagagcatcaacgtgatcaacaaagtgatcgagaaatatggcatccccgaagatatcattatcgaactggcacgtgaaaataactccgatgatcgcaaaaagttcatcaacaacctgcagaaaaagaatgaagcaacccgcaaacgcattaacgaaattattggtcagaccggtaatcagaatgccaaacgtattgtggaaaaaatccgtctgcatgatcagcaagaggggaaatgtctgtatagcctggaaagcattgccctgatggatctgctgaataacccgcagaattatgaagtggatcatattattccgcgtagcgtggcatttgataattccattcataacaaagtgctggtgaagcagatcgagaatagcaaaaaaggtaatcgtacgccgtatcagtatctgaatagcagtgatgcaaaactgagctacaaccagtttaaacagcatattctgaatctgagcaaaagcaaagatcgcatcagcaaaaaaaagaaggactacctgctggaagaacgcgatatcaacaaatttgaagtccagaaagagtttatcaaccgcaatctggttgatacccgttatgcaacccgtgaactgaccagctatctgaaagcatatttcagcgccaataacatggacgtgaaagtgaaaacaattaacggcagctttaccaaccatctgcgtaaagtttggcgctttgataaatatcgcaaccacggctataaacatcatgcagaagatgccctgattattgcaaatgcagatttcctgtttaaagaaaacaaaaaactgaaagccgtcaacagcgtgctggaaaaaccggaaattgagacaaaacagctggacattcaggttgatagcgaagataattacagcgaaatgtttatcatcccgaaacaggtgcaggatatcaaagattttcgcaacttcaaatatagccaccgcgttgacaaaaaacctaatcgtcagctgattaacgataccctgtatagcacccgcaaaaaagataacagcacctatattgtgcagaccattaaagacatctacgccaaagataataccaccctgaaaaaacagttcgacaaaagcccagaaaaatttctgatgtatcagcatgatccgcgtaccttcgaaaaactggaagttattatgaaacagtatgccaacgagaaaaatccgctggccaaatatcacgaagaaaccggtgaatatctgaccaaatattccaagaagaacaacggtccgatcgttaaatccctgaaatatatcggtaataaactgggcagccatctggatgttacccatcagtttaaaagctccacaaagaagctggttaaactgtccatcaaaccgtatcgctttgatgtgtatctgaccgacaaaggctataaattcattaccatcagctatctggacgtgctgaaaaaagacaactattattatatcccggaacagaaatatgataaactgaaactgggtaaagccatcgataaaaacgccaaatttatcgccagcttctacaaaaacgacctgattaaactggatggcgagatctataaaatcatcggtgttaatagcgacacccgcaatatgattgagctggatctgccggatattcgctataaagaatattgcgaactgaacaacattaaaggcgaaccgcgtatcaaaaagaccatcggcaaaaaagtgaatagcatcgagaaactgaccaccgatgttctgggtaatgtgtttaccaatacccagtataccaaacctcagctgctgttcaaacgcggtaat

SEQ ID NO: 27 - DNA, 3159 bp, Synthetic construct
  source 1..3159: mol_type = other DNA
atgaaccaaaagttcattctggggctcgatatcggcatcacctccgtgggatatggtctgatcgactacgagactaagaacatcatcgacgctggagtgcgactgttcccggaagcgaacgtggagaacaacgaaggccgcagatccaagcgcgggtccagaaggctcaagaggcggaggatccatagactcgagagagtgaagtcgctcctttcggaatacaagattatcagcggtcttgcccccaccaacaaccaaccgtacaacatcagagtgaagggcctgaccgaacagctgaccaaagatgaactggccgtcgccctgctgcatattgccaaacggcgcggaatccataagatcgacgtgattgacagcaacgatgacgtgggaaacgagctgtcaaccaaggaacagcttaacaagaacagcaaattgctgaaggacaagtttgtctgccaaattcaactggaacggatgaacgagggacaagtcaggggagagaaaaaccggttcaagaccgccgacatcatcaaggagatcatccaactgctgaacgtgcagaagaacttccaccaactggatgaaaacttcattaacaagtacattgaactggtggaaatgcggagggagtacttcgagggacctggacagggatcccctttcggctggaatggggaccttaagaagtggtacgaaatgttgatgggccattgcacttactttccgcaagaactccggtccgtgaagtacgcatactctgccgacctgttcaatgcactcaacgaccttaacaacttgatcatccagcgcgataactcggaaaagttggaataccacgaaaagtatcacatcatcgagaacgtgttcaagcagaaaaagaagccaactctgaagcagattgccaaggaaattggcgtgaatccggaggatatcaagggataccggatcactaagtccggcacgccagagttcaccgagttcaagctgtaccacgatctgaagtcggtgctctttgaccagtccatcctggaaaacgaagatgtgctggaccagattgctgagatcctgaccatctaccaggacaaggactcgattaagtccaagctcaccgagctggacattctgctgaacgaagaagataaggagaacatcgcgcagctcaccggttacaatggtacccaccgcttgtcccttaagtgcatccgcctggtgctggaggaacagtggtactcgagccggaaccagatggagatcttcactcacttgaacatcaagccgaaaaagattaacctgactgccgccaacaagatacccaaggccatgatcgacgagtttatcctctcaccggtggtcaagcgcaccttcattcaatctatcaacgtgatcaacaaggtcatcgagaagtacggcattcctgaggatatcatcatcgagctggctcgggagaacaactcagacgataggaagaagttcattaacaacctccagaaaaagaacgaggccactcgcaagcggattaatgagatcatcggtcagaccgggaaccagaacgccaagcggatcgtggaaaagattcggctccacgaccaacaggagggaaagtgtctgtactcgctggagtcgattgcactgatggacctcctgaacaacccacagaactacgaagtcgatcacataatcccccgcagcgtggcattcgacaactccatccataacaaggtcctcgtgaagcagatcgagaatagcaagaaggggaaccggactccgtaccagtacctgaactcctccgacgccaagctgtcatacaatcagttcaaacagcacattctcaacctgtccaagtcaaaggacaggatctccaagaagaagaaggactaccttctcgaggaacgggatatcaataagttcgaggtgcagaaggagtttatcaatagaaacctggtggacactcgctatgccacccgcgaactgaccagctacctgaaggcgtacttctccgccaacaacatggacgtgaaggtcaaaactattaacggcagcttcaccaaccatctgcgcaaggtctggaggttcgacaagtaccgcaaccacggttacaagcaccacgcggaagatgcgctgattatcgccaacgctgacttcctgttcaaggaaaacaagaagctcaaggccgtgaactcagtgctcgagaagcctgaaatcgagactaagcagctggacatccaggtcgattcggaagataactactccgaaatgttcatcatccctaagcaagtgcaggacatcaaggacttcaggaatttcaagtacagccatcgcgtggacaagaagccaaacagacagctgatcaacgatacactgtattccacccggaagaaggacaactccacctacatcgtccaaaccattaaggacatctacgcaaaggacaacaccacgcttaagaagcagttcgacaagagccccgaaaagttcctcatgtaccagcacgaccccagaaccttcgagaagcttgaagtgatcatgaagcagtacgccaacgaaaagaacccactggctaagtaccacgaggaaaccggcgaatacctgaccaagtactccaaaaagaacaacggaccgatcgtcaagtccctgaagtacattgggaacaagctcggctcgcacctcgatgtgacccaccagttcaagtcctcgaccaaaaagctcgtgaagctgtccatcaagccgtaccggttcgacgtgtacctgactgacaagggatataagttcatcaccatttcctacctcgacgtgttgaagaaggataactactactacattccggaacagaagtacgacaagctcaagctcggaaaggccatcgacaaaaatgcgaagttcatcgcgagcttctacaagaatgacttgatcaagctggatggcgaaatctacaagatcatcggggtcaactccgatacccgcaacatgattgagctggatctgcccgacattcggtacaaggaatactgcgagctgaacaacatcaagggagaaccgcggatcaagaaaaccatcggaaagaaagtgaacagcatcgagaaactgactactgacgtcctgggaaacgtgttcaccaacacacaatacaccaaaccccagctgctgtttaagcgcgggaac

SEQ ID NO: 28 - DNA, 3159 bp, Synthetic construct
  source 1..3159: mol_type = other DNA
atgaaccagaaatttatcctgggtctggatattggtattaccagcgttggttatggcctgattgattacgaaaccaaaaacattattgatgccggtgttcgtctgtttccggaagcaaatgttgaaaataatgaaggtcgtcgtagcaaacgtggtagccgtcgtctgaaacgtcgtcgtattcatcgtctggaacgtgttaaaagcctgctgagcgaatataagattattagcggtctggcaccgaccaataatcagccgtataacattcgtgttaaaggtctgaccgaacagctgaccaaagatgaactggcagttgcactgctgcatattgccaaacgccgtggcattcataaaatcgatgtgattgatagcaatgacgatgtgggtaatgaactgagcaccaaagaacagctgaacaaaaatagcaaactgctgaaagacaaattcgtgtgtcagattcagctggaacgtatgaatgaaggccaggttcgtggtgaaaagaatcgctttaaaaccgcagacatcatcaaagaaattatccagctgctgaacgtgcagaaaaacttccatcagctggatgaaaacttcatcaacaaatacatcgagctggttgaaatgcgtcgcgaatattttgaaggtcctggtcagggtagcccgtttggttggaatggtgatctgaaaaaatggtacgaaatgctgatgggtcactgtacctattttccgcaagaactgcgtagcgttaaatatgcctatagcgcagacctgtttaatgcactgaatgatctgaacaacctgattattcagcgcgataatagcgagaaactggaataccatgagaagtatcacatcatcgagaacgtgttcaagcagaaaaaaaagccgacgctgaaacaaatcgcaaaagagattggcgttaacccggaagatattaaaggttatcgtattaccaaaagcggtacaccggaattcaccgaatttaaactgtatcacgatctgaaaagcgtgctgtttgatcagagcattctggaaaatgaagatgtgctggaccagattgcagaaattctgaccatttatcaggacaaagacagcatcaaaagcaaactgaccgaactggatattctgctgaatgaagaagataaagagaacattgcacagctgaccggttataacggcacacatcgcctgagcctgaaatgtattcgtctggtactggaagaacagtggtatagcagccgtaatcagatggaaatctttacccatctgaacattaaaccgaagaaaatcaatctgaccgcagccaacaaaattccgaaagccatgattgatgagtttattctgagtccggttgtgaaacgcacctttattcagagcatcaacgtgatcaacaaagtgatcgagaaatatggcatccccgaagatatcattatcgaactggcacgtgaaaataactccgatgatcgcaaaaagttcatcaacaacctgcagaaaaagaatgaagcaacccgcaaacgcattaacgaaattattggtcagaccggtaatcagaatgccaaacgtattgtggaaaaaatccgtctgcatgatcagcaagaggggaaatgtctgtatagcctggaaagcattgccctgatggatctgctgaataacccgcagaattatgaagtggatcatattattccgcgtagcgtggcatttgataattccattcataacaaagtgctggtgaagcagatcgagaatagcaaaaaaggtaatcgtacgccgtatcagtatctgaatagcagtgatgcaaaactgagctacaaccagtttaaacagcatattctgaatctgagcaaaagcaaagatcgcatcagcaaaaaaaagaaggactacctgctggaagaacgcgatatcaacaaatttgaagtccagaaagagtttatcaaccgcaatctggttgatacccgttatgcaacccgtgaactgaccagctatctgaaagcatatttcagcgccaataacatggacgtgaaagtgaaaacaattaacggcagctttaccaaccatctgcgtaaagtttggcgctttgataaatatcgcaaccacggctataaacatcatgcagaagatgccctgattattgcaaatgcagatttcctgtttaaagaaaacaaaaaactgaaagccgtcaacagcgtgctggaaaaaccggaaattgagacagatcagaaagacattcaggttgatagcgaagataattacagcgaaatgtttatcatcccgaaacaggtgcaggatatcaaagattttcgcaacttcaaatatagccaccgcgttgacaaaaaacctaatcgtcagctgattaacgataccctgtatagcacccgcaaaaaagataacagcacctatattgtgcagaccattaaagacatctacgccaaagataataccaccctgaaaaaacagttcgacaaaagcccagaaaaatttctgatgtatcagcatgatccgcgtaccttcgaaaaactggaagttattatgaaacagtatgccaacgagaaaaatccgctggccaaatatcacgaagaaaccggtgaatatctgaccaaatattccaagaagaacaacggtccgatcgttaaatccctgaaatatatcggtaataaactgggcagccatctggatgttacccatcagtttaaaagctccacaaagaagctggttaaactgtccatcaaaccgtatcgctttgatgtgtatctgaccgacaaaggctataaattcattaccatcagctatctggacgtgctgaaaaaagacaactattattatatcccggaacagaaatatgataaactgaaactgggtaaagccatcgataaaaacgccaaatttatcgccagcttctacaaaaacgacctgattaaactggatggcgagatctataaaatcatcggtgttaatagcgacacccgcaatatgattgagctggatctgccggatattcgctataaagaatattgcgaactgaacaacattaaaggcgaaccgcgtatcaaaaagaccatcggcaaaaaagtgaatagcatcgagaaactgaccaccgatgttctgggtaatgtgtttaccaatacccagtataccaaacctcagctgctgttcaaacgcggtaat

SEQ ID NO: 29 - DNA, 3159 bp, Synthetic construct
  source 1..3159: mol_type = other DNA
atgaaccagaaatttatcctgggtctggatattggtattaccagcgttggttatggcctgattgattacgaaaccaaaaacattattgatgccggtgttcgtctgtttccggaagcaaatgttgaaaataatgaaggtcgtcgtagcaaacgtggtagccgtcgtctgaaacgtcgtcgtattcatcgtctggaacgtgttaaaagcctgctgagcgaatataagattattagcggtctggcaccgaccaataatcagccgtataacattcgtgttaaaggtctgaccgaacagctgaccaaagatgaactggcagttgcactgctgcatattgccaaacgccgtggcattcataaaatcgatgtgattgatagcaatgacgatgtgggtaatgaactgagcaccaaagaacagctgaacaaaaatagcaaactgctgaaagacaaattcgtgtgtcagattcagctggaacgtatgaatgaaggccaggttcgtggtgaaaagaatcgctttaaaaccgcagacatcatcaaagaaattatccagctgctgaacgtgcagaaaaacttccatcagctggatgaaaacttcatcaacaaatacatcgagctggttgaaatgcgtcgcgaatattttgaaggtcctggtcagggtagcccgtttggttggaatggtgatctgaaaaaatggtacgaaatgctgatgggtcactgtacctattttccgcaagaactgcgtagcgttaaatatgcctatagcgcagacctgtttaatgcactgaatgatctgaacaacctgattattcagcgcgataatagcgagaaactggaataccatgagaagtatcacatcatcgagaacgtgttcaagcagaaaaaaaagccgacgctgaaacaaatcgcaaaagagattggcgttaacccggaagatattaaaggttatcgtattaccaaaagcggtacaccggaattcaccgaatttaaactgtatcacgatctgaaaagcgtgctgtttgatcagagcattctggaaaatgaagatgtgctggaccagattgcagaaattctgaccatttatcaggacaaagacagcatcaaaagcaaactgaccgaactggatattctgctgaatgaagaagataaagagaacattgcacagctgaccggttataacggcacacatcgcctgagcctgaaatgtattcgtctggtactggaagaacagtggtatagcagccgtaatcagatggaaatctttacccatctgaacattaaaccgaagaaaatcaatctgaccgcagccaacaaaattccgaaagccatgattgatgagtttattctgagtccggttgtgaaacgcacctttattcagagcatcaacgtgatcaacaaagtgatcgagaaatatggcatccccgaagatatcattatcgaactggcacgtgaaaataactccgatgatcgcaaaaagttcatcaacaacctgcagaaaaagaatgaagcaacccgcaaacgcattaacgaaattattggtcagaccggtaatcagaatgccaaacgtattgtggaaaaaatccgtctgcatgatcagcaagaggggaaatgtctgtatagcctggaaagcattgccctgatggatctgctgaataacccgcagaattatgaagtggatcatattattccgcgtagcgtggcatttgataattccattcataacaaagtgctggtgaagcagatcgagaatagcaaaaaaggtaatcgtacgccgtatcagtatctgaatagcagtgatgcaaaactgagctacaaccagtttaaacagcatattctgaatctgagcaaaagcaaagatcgcatcagcaaaaaaaagaaggactacctgctggaagaacgcgatatcaacaaatttgaagtccagaaagagtttatcaaccgcaatctggttgatacccgttatgcaacccgtgaactgaccagctatctgaaagcatatttcagcgccaataacatggacgtgaaagtgaaaacaattaacggcagctttaccaaccatctgcgtaaagtttggcgctttgataaatatcgcaaccacggctataaacatcatgcagaagatgccctgattattgcaaatgcagatttcctgtttaaagaaaacaaaaaactgaaagccgtcaacagcgtgctggaaaaaccggaaattgagacaaatcagaatgacattcaggttgatagcgaagataattacagcgaaatgtttatcatcccgaaacaggtgcaggatatcaaagattttcgcaacttcaaatatagccaccgcgttgacaaaaaacctaatcgtcagctgattaacgataccctgtatagcacccgcaaaaaagataacagcacctatattgtgcagaccattaaagacatctacgccaaagataataccaccctgaaaaaacagttcgacaaaagcccagaaaaatttctgatgtatcagcatgatccgcgtaccttcgaaaaactggaagttattatgaaacagtatgccaacgagaaaaatccgctggccaaatatcacgaagaaaccggtgaatatctgaccaaatattccaagaagaacaacggtccgatcgttaaatccctgaaatatatcggtaataaactgggcagccatctggatgttacccatcagtttaaaagctccacaaagaagctggttaaactgtccatcaaaccgtatcgctttgatgtgtatctgaccgacaaaggctataaattcattaccatcagctatctggacgtgctgaaaaaagacaactattattatatcccggaacagaaatatgataaactgaaactgggtaaagccatcgataaaaacgccaaatttatcgccagcttctacaaaaacgacctgattaaactggatggcgagatctataaaatcatcggtgttaatagcgacacccgcaatatgattgagctggatctgccggatattcgctataaagaatattgcgaactgaacaacattaaaggcgaaccgcgtatcaaaaagaccatcggcaaaaaagtgaatagcatcgagaaactgaccaccgatgttctgggtaatgtgtttaccaatacccagtataccaaacctcagctgctgttcaaacgcggtaat

SEQ ID NO: 30 - protein, 1054 aa, Synthetic construct
MNQKFILGLDIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGKPQFTEFKLYHDLKSVLFDQSILENEDVLDQIAEILTIYQDKDSIKSKLTELDILLNEEDKENIAQLTGYTGTHRLSLKCIRLVLEEQWYSSRNQMEIFTHLNIKPKKINLTAANKIPKAMIDEFILSPVVKRTFGQAINLINKIIEKYGVPEDIIIELARENNSKDKQKFINEMQKKNENTRKRINEIIGKYGNQNAKRLVEKIRLHDEQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 31 - protein, 1054 aa, Synthetic construct
MKEKYILGLDLGITSVGYGIINFETKKIIDAGVRLFPEANVDNNEGRRSKRGSRRLKRRRIHRLERVKLLLTEYDLINKEQIPTSNNPYQIRVKGLSEILSKDELAIALLHLAKRRGIHNINVSSEDEDASNELSTKEQINRNNKLLKDKYVCEVQLQRLKEGQIRGEKNRFKTTDILKEIDQLLKVQKDYHNLDIDFINQYKEIVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPQFTEFKLYHDLKSIVFDKSILENEAILDQIAEILTIYQDEQSIKEELNKLPEILNEQDKAEIAKLIGYNGTHRLSLKCIHLINEELWQTSRNQMEIFNYLNIKPNKVDLSEQNKIPKDMVNDFILSPVVKRTFIQSINVINKVIEKYGIPEDIIIELARENNSDDRKKFINNLQKKNEATRKRINEIIGQTGNQNAKRIVEKIRLHDQQEGKCLYSLESIALMDLLNNPQNYEVDHIIPRSVAFDNSIHNKVLVKQIENSKKGNRTPYQYLNSSDAKLSYNQFKQHILNLSKSKDRISKKKKDYLLEERDINKFEVQKEFINRNLVDTRYATRELTSYLKAYFSANNMDVKVKTINGSFTNHLRKVWRFDKYRNHGYKHHAEDALIIANADFLFKENKKLQNTNKILEKPTIENNTKKVTVEKEEDYNNVFETPKLVEDIKQYRDYKFSHRVDKKPNRQLINDTLYSTRMKDEHDYIVQTITDIYGKDNTNLKKQFNKNPEKFLMYQNDPKTFEKLSIIMKQYSDEKNPLAKYYEETGEYLTKYSKKNNGPIVKKIKLLGNKVGNHLDVTNKYENSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 32 - protein, 1051 aa, Synthetic construct
MNNYILGLDIGITSVGYGIVDSDTREIKDAGVRLFPEANVDNNEGRRSKRGARRLKRRRIHRLDRVKHLLAEYDLLDLTNIPKSTNPYQTRVKGLNEKLSKDELVIALLHIAKRRGIHNVNVMMDDNDSGNELSTKDQLKKNAKALSDKYVCELQLERFEQDYKVRGEKNRFKTEDFVREARKLLETQSKFFEIDQTFIMRYIELIETRREYFEGPGKGSPFGWEGNIKKWFEQMMGHCTYFPEELRSVKYSYSAELFNALNDLNNLVITRDEDAKLNYGEKFQIIENVFKQKKTPNLKQIAIEIGVHETEIKGYRVNKSGKPEFTQFKLYHDLKNIFKDPKYLNDIQLMDNIAEIITIYQDAESIIKELNQLPELLSEREKEKISALSGYSGTHRLSLKCINLLLDDLWESSLNQMELFTKLNLKPKKIDLSQQHKIPSKLVDDFILSPVVKRAFIQSIQVVNAIIDKYGLPEDIIIELARENNSDDRRKFLNQLQKQNEETRKQVEKVLREYGNDNAKRIVQKIKLHNMQEGKCLYSLKDIPLEDLLRNPHHYEVDHIIPRSVAFDNSMHNKVLVRADENSKKGNRTPYQYLNSSESSLSYNEFKQHILNLSKTKDRITKKKREYLLEERDINKFDVQKEFINRNLVDTRYATRELTSLLKAYFSANNLDVKVKTINGSFTNYLRKVWKFDKDRNKGYKHHAEDALIIANADFLFKHNKKLRNINKVLDAPSKEVDKKRVTVQSEDEYNQIFEDTQKAQAIKKFEIRKFSHRVDKKPNRQLINDTLYSTRNIDGIEYVVESIKDIYSVNNDKVKTKFKKDPHRLLMYRNDPQTFEKFEKVFKQYESEKNPFAKYYEETGEKIRKFSKTGQGPYINKIKYLRERLGRHCDVTNKYINSRNKIVQLKIYSYRFDIYQYGNNYKMITISYIDLEQKSNYYYISREKYEQKKKDKQIDDSYKFIGSFYKNDIINYNGEMYRVIGVNDSEKNKIQLDMIDISIKDYMELNNIKKTGVIYKTIGKSTTHIEKYTTDILGNLYKAAPPKKPQLIFK

SEQ ID NO: 33 - protein, 1052 aa, Synthetic construct
MEKDYILGLDIGIGSVGYGLIDYDTKSIIDAGVRLFPEANADNNLGRRAKRGARRLKRRRIHRLERVKSLLSEYKIISGLAPTNNQPYNIRVKGLTEQLTKDELAVALLHIAKRRGIHNVDVAADKEETASDSLSTKDQINKNAKFLESRYVCELQKERLENEGHVRGVENRFLTKDIVREAKKIIDTQMQYYPEIDETFKEKYISLVETRREYYEGPGKGSPYGWDADVKKWYQLMMGHCTYFPVEFRSVKYAYTADLYNALNDLNNLTIARDDNPKLEYHEKYHIIENVFKQKRNPTLKQIAKEIGVNDINISGYRVTKSGKPQFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRSFKQAIGVVNAIIKKYGLPKDIIIELARESNSAEKSRYLRAIQKKNEKTRERIEAIIKEYGNENAKGLVQKIKLHDAQEGKCLYSLKDIPLEDLLRNPNNYDIDHIIPRSVSFDDSMHNKVLVRREQNAKKNNQTPYQYLTSGYADIKYSVFKQHVLNLAENKDRMTKKKREYLLEERNINKYDVQKEFINRNLVDTRYTTRELTTLLKTYFTINNLDVKVKTINGSFTDFLRKRWGFKKNRDEGYKHHAEDALIIANADYLFKEHKLLKEIKDVSDLAGDERNSNVKDEDQYEEVFGGYFKIEDIKKYKIKKFSHRVDKKPNRQLINDTIYSTRVKDDKRYLINTLKNLYDKSNGDLKERMQKDPESLLMYHHDPQTFEKLKIVMSQYENEKNPLAKYFEETGQYLTKYAKHDNGPAIHKIKYYGNKLVEHLDITKNYHNPQNKVVQLSQKSFRFDVYQTDKGYKFISIAYLTLKNEKNYYAISQEKYDQLKSEKKISNNAVFIGSFYTSDIIEINNEKFRVIGVNSDKNNLIEVDRIDIRQKEFIELEEEKKNNRIKVTIGRKTTNIEKFHTDILGNMYKSKRPKAPQLVFKKG

SEQ ID NO: 34 - protein, 67 aa, Synthetic construct
MNQKFILGLDIGITSVGYGLIDYETKNIIDAGVRLFPEANVENNEGRRSKRGSRRLKRRRIHRLERV

SEQ ID NO: 35 - protein, 67 aa, Synthetic construct
MKEKYILGLDLGITSVGYGIINFETKKIIDAGVRLFPEANVDNNEGRRSKRGSRRLKRRRIHRLERV

SEQ ID NO: 36 - protein, 66 aa, Synthetic construct
MNNYILGLDIGITSVGYGIVDSDTREIKDAGVRLFPEANVDNNEGRRSKRGARRLKRRRIHRLDRV

SEQ ID NO: 37 - protein, 67 aa, Synthetic construct
MEKDYILGLDIGIGSVGYGLIDYDTKSIIDAGVRLFPEANADNNLGRRAKRGARRLKRRRIHRLERV

SEQ ID NO: 38 - protein, 52 aa, Synthetic construct
KKLLEDYNLLDQSQIPQSTNPYAIRVKGLSEALSKDELVIALLHIAKRRGIH

SEQ ID NO: 39 - protein, 52 aa, Synthetic construct
KLLLTEYDLINKEQIPTSNNPYQIRVKGLSEILSKDELAIALLHLAKRRGIH

SEQ ID NO: 40 - protein, 52 aa, Synthetic construct
KHLLAEYDLLDLTNIPKSTNPYQTRVKGLNEKLSKDELVIALLHIAKRRGIH

SEQ ID NO: 41 - protein, 51 aa, Synthetic construct
KSLLSEYKIISGLAPTNNQPYNIRVKGLTEQLTKDELAVALLHIAKRRGIH

SEQ ID NO: 42 - protein, 92 aa, Synthetic construct
KIDVIDSNDDVGNELSTKEQLNKNSKLLKDKFVCQIQLERMNEGQVRGEKNRFKTADIIKEIIQLLNVQKNFHQLDENFINKYIELVEMRRE

SEQ ID NO: 43 - protein, 92 aa, Synthetic construct
NINVSSEDEDASNELSTKEQINRNNKLLKDKYVCEVQLQRLKEGQIRGEKNRFKTTDILKEIDQLLKVQKDYHNLDIDFINQYKEIVETRRE

SEQ ID NO: 44 - protein, 93 aa, Synthetic construct
NVNVMMDDNDSGNELSTKDQLKKNAKALSDKYVCELQLERFEQDYKVRGEKNRFKTEDFVREARKLLETQSKFFEIDQTFIMRYIELIETRRE

SEQ ID NO: 45 - protein, 95 aa, Synthetic construct
NVDVAADKEETASDSLSTKDQINKNAKFLESRYVCELQKERLENEGHVRGVENRFLTKDIVREAKKIIDTQMQYYPEIDETFKEKYISLVETRRE

SEQ ID NO: 46 - protein, 111 aa, Synthetic construct
YFEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGK

SEQ ID NO: 47 - protein, 111 aa, Synthetic construct
YFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGT

SEQ ID NO: 48 - protein, 111 aa, Synthetic construct
YFEGPGKGSPFGWEGNIKKWFEQMMGHCTYFPEELRSVKYSYSAELFNALNDLNNLVITRDEDAKLNYGEKFQIIENVFKQKKTPNLKQIAIEIGVHETEIKGYRVNKSGK

SEQ ID NO: 49 - protein, 111 aa, Synthetic construct
YYEGPGKGSPYGWDADVKKWYQLMMGHCTYFPVEFRSVKYAYTADLYNALNDLNNLTIARDDNPKLEYHEKYHIIENVFKQKRNPTLKQIAKEIGVNDINISGYRVTKSGK

SEQ ID NO: 50 - protein, 131 aa, Synthetic construct
PQFTEFKLYHDLKSVLFDQSILENEDVLDQIAEILTIYQDKDSIKSKLTELDILLNEEDKENIAQLTGYTGTHRLSLKCIRLVLEEQWYSSRNQMEIFTHLNIKPKKINLTAANKIPKAMIDEFILSPVVK

SEQ ID NO: 51 - protein, 131 aa, Synthetic construct
PQFTEFKLYHDLKSIVFDKSILENEAILDQIAEILTIYQDEQSIKEELNKLPEILNEQDKAEIAKLIGYNGTHRLSLKCIHLINEELWQTSRNQMEIFNYLNIKPNKVDLSEQNKIPKDMVNDFILSPVVK

SEQ ID NO: 52 - protein, 131 aa, Synthetic construct
PEFTQFKLYHDLKNIFKDPKYLNDIQLMDNIAEIITIYQDAESIIKELNQLPELLSEREKEKISALSGYSGTHRLSLKCINLLLDDLWESSLNQMELFTKLNLKPKKIDLSQQHKIPSKLVDDFILSPVVK

SEQ ID NO: 53 - protein, 131 aa, Synthetic construct
PQFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVK

SEQ ID NO: 54 - protein, 89 aa, Synthetic construct
RTFGQAINLINKIIEKYGVPEDIIIELARENNSKDKQKFINEMQKKNENTRKRINEIIGKYGNQNAKRLVEKIRLHDEQEGKCLYSLES

SEQ ID NO: 55 - protein, 89 aa, Synthetic construct
RTFIQSINVINKVIEKYGIPEDIIIELARENNSDDRKKFINNLQKKNEATRKRINEIIGQTGNQNAKRIVEKIRLHDQQEGKCLYSLES

SEQ ID NO: 56 - protein, 89 aa, Synthetic construct
RAFIQSIQVVNAIIDKYGLPEDIIIELARENNSDDRRKFLNQLQKQNEETRKQVEKVLREYGNDNAKRIVQKIKLHNMQEGKCLYSLKD

SEQ ID NO: 57 - protein, 89 aa, Synthetic construct
RSFKQAIGVVNAIIKKYGLPKDIIIELARESNSAEKSRYLRAIQKKNEKTRERIEAIIKEYGNENAKGLVQKIKLHDAQEGKCLYSLKD

SEQ ID NO: 58 - protein, 116 aa, Synthetic construct
IPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATREL

SEQ ID NO: 59 - protein, 116 aa, Synthetic construct
IALMDLLNNPQNYEVDHIIPRSVAFDNSIHNKVLVKQIENSKKGNRTPYQYLNSSDAKLSYNQFKQHILNLSKSKDRISKKKKDYLLEERDINKFEVQKEFINRNLVDTRYATREL

SEQ ID NO: 60 - protein, 116 aa, Synthetic construct
IPLEDLLRNPHHYEVDHIIPRSVAFDNSMHNKVLVRADENSKKGNRTPYQYLNSSESSLSYNEFKQHILNLSKTKDRITKKKREYLLEERDINKFDVQKEFINRNLVDTRYATREL

SEQ ID NO: 61 - protein, 116 aa, Synthetic construct
IPLEDLLRNPNNYDIDHIIPRSVSFDDSMHNKVLVRREQNAKKNNQTPYQYLTSGYADIKYSVFKQHVLNLAENKDRMTKKKREYLLEERNINKYDVQKEFINRNLVDTRYTTREL

SEQ ID NO: 62 - protein, 396 aa, Synthetic construct
TNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 63 - protein, 396 aa, Synthetic construct
TSYLKAYFSANNMDVKVKTINGSFTNHLRKVWRFDKYRNHGYKHHAEDALIIANADFLFKENKKLQNTNKILEKPTIENNTKKVTVEKEEDYNNVFETPKLVEDIKQYRDYKFSHRVDKKPNRQLINDTLYSTRMKDEHDYIVQTITDIYGKDNTNLKKQFNKNPEKFLMYQNDPKTFEKLSIIMKQYSDEKNPLAKYYEETGEYLTKYSKKNNGPIVKKIKLLGNKVGNHLDVTNKYENSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 64 - protein, 393 aa, Synthetic construct
TSLLKAYFSANNLDVKVKTINGSFTNYLRKVWKFDKDRNKGYKHHAEDALIIANADFLFKHNKKLRNINKVLDAPSKEVDKKRVTVQSEDEYNQIFEDTQKAQAIKKFEIRKFSHRVDKKPNRQLINDTLYSTRNIDGIEYVVESIKDIYSVNNDKVKTKFKKDPHRLLMYRNDPQTFEKFEKVFKQYESEKNPFAKYYEETGEKIRKFSKTGQGPYINKIKYLRERLGRHCDVTNKYINSRNKIVQLKIYSYRFDIYQYGNNYKMITISYIDLEQKSNYYYISREKYEQKKKDKQIDDSYKFIGSFYKNDIINYNGEMYRVIGVNDSEKNKIQLDMIDISIKDYMELNNIKKTGVIYKTIGKSTTHIEKYTTDILGNLYKAAPPKKPQLIFK

SEQ ID NO: 65 - protein, 392 aa, Synthetic construct
TTLLKTYFTINNLDVKVKTINGSFTDFLRKRWGFKKNRDEGYKHHAEDALIIANADYLFKEHKLLKEIKDVSDLAGDERNSNVKDEDQYEEVFGGYFKIEDIKKYKIKKFSHRVDKKPNRQLINDTIYSTRVKDDKRYLINTLKNLYDKSNGDLKERMQKDPESLLMYHHDPQTFEKLKIVMSQYENEKNPLAKYFEETGQYLTKYAKHDNGPAIHKIKYYGNKLVEHLDITKNYHNPQNKVVQLSQKSFRFDVYQTDKGYKFISIAYLTLKNEKNYYAISQEKYDQLKSEKKISNNAVFIGSFYTSDIIEINNEKFRVIGVNSDKNNLIEVDRIDIRQKEFIELEEEKKNNRIKVTIGRKTTNIEKFHTDILGNMYKSKRPKAPQLVFKKG

SEQ ID NO: 66 - protein, 41 aa, Synthetic construct
MNQKFILGLDIGITSVGYGLIDYETKNIIDAGVRLFPEANV

SEQ ID NO: 67 - protein, 41 aa, Synthetic construct
MKEKYILGLDLGITSVGYGIINFETKKIIDAGVRLFPEANV

SEQ ID NO: 68 - protein, 40 aa, Synthetic construct
MNNYILGLDIGITSVGYGIVDSDTREIKDAGVRLFPEANV

SEQ ID NO: 69 - protein, 41 aa, Synthetic construct
MEKDYILGLDIGIGSVGYGLIDYDTKSIIDAGVRLFPEANA

SEQ ID NO: 70 - protein, 23 aa, Synthetic construct
ENNEGRRSKRGSRRLKRRRIHRL

SEQ ID NO: 71 - protein, 23 aa, Synthetic construct
DNNEGRRSKRGSRRLKRRRIHRL

SEQ ID NO: 72 - protein, 23 aa, Synthetic construct
DNNEGRRSKRGARRLKRRRIHRL

SEQ ID NO: 73 - protein, 23 aa, Synthetic construct
DNNLGRRAKRGARRLKRRRIHRL

SEQ ID NO: 74 - protein, 55 aa, Synthetic construct
ERVKKLLEDYNLLDQSQIPQSTNPYAIRVKGLSEALSKDELVIALLHIAKRRGIH

SEQ ID NO: 75 - protein, 55 aa, Synthetic construct
ERVKLLLTEYDLINKEQIPTSNNPYQIRVKGLSEILSKDELAIALLHLAKRRGIH

SEQ ID NO: 76 - protein, 55 aa, Synthetic construct
DRVKHLLAEYDLLDLTNIPKSTNPYQTRVKGLNEKLSKDELVIALLHIAKRRGIH

SEQ ID NO: 77 - protein, 54 aa, Synthetic construct
ERVKSLLSEYKIISGLAPTNNQPYNIRVKGLTEQLTKDELAVALLHIAKRRGIH

SEQ ID NO: 78 - protein, 93 aa, Synthetic construct
KIDVIDSNDDVGNELSTKEQLNKNSKLLKDKFVCQIQLERMNEGQVRGEKNRFKTADIIKEIIQLLNVQKNFHQLDENFINKYIELVEMRREY

SEQ ID NO: 79 - protein, 93 aa, Synthetic construct
NINVSSEDEDASNELSTKEQINRNNKLLKDKYVCEVQLQRLKEGQIRGEKNRFKTTDILKEIDQLLKVQKDYHNLDIDFINQYKEIVETRREY

SEQ ID NO: 80 - protein, 94 aa, Synthetic construct
NVNVMMDDNDSGNELSTKDQLKKNAKALSDKYVCELQLERFEQDYKVRGEKNRFKTEDFVREARKLLETQSKFFEIDQTFIMRYIELIETRREY

SEQ ID NO: 81 - protein, 96 aa, Synthetic construct
NVDVAADKEETASDSLSTKDQINKNAKFLESRYVCELQKERLENEGHVRGVENRFLTKDIVREAKKIIDTQMQYYPEIDETFKEKYISLVETRREY

SEQ ID NO: 82 - protein, 114 aa, Synthetic construct
FEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGKPQFT

SEQ ID NO: 83 - protein, 114 aa, Synthetic construct
FEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPQFT

SEQ ID NO: 84 - protein, 114 aa, Synthetic construct
FEGPGKGSPFGWEGNIKKWFEQMMGHCTYFPEELRSVKYSYSAELFNALNDLNNLVITRDEDAKLNYGEKFQIIENVFKQKKTPNLKQIAIEIGVHETEIKGYRVNKSGKPEFT

SEQ ID NO: 85 - protein, 114 aa, Synthetic construct
YEGPGKGSPYGWDADVKKWYQLMMGHCTYFPVEFRSVKYAYTADLYNALNDLNNLTIARDDNPKLEYHEKYHIIENVFKQKRNPTLKQIAKEIGVNDINISGYRVTKSGKPQFT

SEQ ID NO: 86 - protein, 128 aa, Synthetic construct
EFKLYHDLKSVLFDQSILENEDVLDQIAEILTIYQDKDSIKSKLTELDILLNEEDKENIAQLTGYTGTHRLSLKCIRLVLEEQWYSSRNQMEIFTHLNIKPKKINLTAANKIPKAMIDEFILSPVVKR

SEQ ID NO: 87 - protein, 128 aa, Synthetic construct
EFKLYHDLKSIVFDKSILENEAILDQIAEILTIYQDEQSIKEELNKLPEILNEQDKAEIAKLIGYNGTHRLSLKCIHLINEELWQTSRNQMEIFNYLNIKPNKVDLSEQNKIPKDMVNDFILSPVVKR

SEQ ID NO: 88 - protein, 128 aa, Synthetic construct
QFKLYHDLKNIFKDPKYLNDIQLMDNIAEIITIYQDAESIIKELNQLPELLSEREKEKISALSGYSGTHRLSLKCINLLLDDLWESSLNQMELFTKLNLKPKKIDLSQQHKIPSKLVDDFILSPVVKR

SEQ ID NO: 89 - protein, 128 aa, Synthetic construct
SFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKR

SEQ ID NO: 90 - protein, 86 aa, Synthetic construct
TFGQAINLINKIIEKYGVPEDIIIELARENNSKDKQKFINEMQKKNENTRKRINEIIGKYGNQNAKRLVEKIRLHDEQEGKCLYSL

SEQ ID NO: 91 - protein, 86 aa, Synthetic construct
TFIQSINVINKVIEKYGIPEDIIIELARENNSDDRKKFINNLQKKNEATRKRINEIIGQTGNQNAKRIVEKIRLHDQQEGKCLYSL

SEQ ID NO: 92 - protein, 86 aa, Synthetic construct
AFIQSIQVVNAIIDKYGLPEDIIIELARENNSDDRRKFLNQLQKQNEETRKQVEKVLREYGNDNAKRIVQKIKLHNMQEGKCLYSL

SEQ ID NO: 93 - protein, 86 aa, Synthetic construct
SFKQAIGVVNAIIKKYGLPKDIIIELARESNSAEKSRYLRAIQKKNEKTRERIEAIIKEYGNENAKGLVQKIKLHDAQEGKCLYSL

SEQ ID NO: 94 - protein, 92 aa, Synthetic construct
ESIPLEDLLNNPNHYEVDHIIPRSVSFDNSYHNKVLVKQSENSKKSNLTPYQYFNSGKSKLSYNQFKQHILNLSKSQDRISKKKKEYLLEER

SEQ ID NO: 95 - protein, 92 aa, Synthetic construct
ESIALMDLLNNPQNYEVDHIIPRSVAFDNSIHNKVLVKQIENSKKGNRTPYQYLNSSDAKLSYNQFKQHILNLSKSKDRISKKKKDYLLEER

SEQ ID NO: 96 - protein, 92 aa, Synthetic construct
KDIPLEDLLRNPHHYEVDHIIPRSVAFDNSMHNKVLVRADENSKKGNRTPYQYLNSSESSLSYNEFKQHILNLSKTKDRITKKKREYLLEER

SEQ ID NO: 97 - protein, 92 aa, Synthetic construct
KDIPLEDLLRNPNNYDIDHIIPRSVSFDDSMHNKVLVRREQNAKKNNQTPYQYLTSGYADIKYSVFKQHVLNLAENKDRMTKKKREYLLEER

SEQ ID NO: 98 - protein, 27 aa, Synthetic construct
DINKFEVQKEFINRNLVDTRYATRELT

SEQ ID NO: 99 - protein, 27 aa, Synthetic construct
DINKFEVQKEFINRNLVDTRYATRELT

SEQ ID NO: 100 - protein, 27 aa, Synthetic construct
DINKFDVQKEFINRNLVDTRYATRELT

SEQ ID NO: 101 - protein, 27 aa, Synthetic construct
NINKYDVQKEFINRNLVDTRYTTRELT

SEQ ID NO: 102 - protein, 64 aa, Synthetic construct
NYLKAYFSANNMNVKVKTINGSFTDYLRKVWKFKKERNHGYKHHAEDALIIANADFLFKENKKL

SEQ ID NO: 103 - protein, 64 aa, Synthetic construct
SYLKAYFSANNMDVKVKTINGSFTNHLRKVWRFDKYRNHGYKHHAEDALIIANADFLFKENKKL

SEQ ID NO: 104 - protein, 64 aa, Synthetic construct
SLLKAYFSANNLDVKVKTINGSFTNYLRKVWKFDKDRNKGYKHHAEDALIIANADFLFKHNKKL

SEQ ID NO: 105 - protein, 64 aa, Synthetic construct
TLLKTYFTINNLDVKVKTINGSFTDFLRKRWGFKKNRDEGYKHHAEDALIIANADYLFKEHKLL

SEQ ID NO: 106 - protein, 186 aa, Synthetic construct
KAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIK

SEQ ID NO: 107 - protein, 186 aa, Synthetic construct
QNTNKILEKPTIENNTKKVTVEKEEDYNNVFETPKLVEDIKQYRDYKFSHRVDKKPNRQLINDTLYSTRMKDEHDYIVQTITDIYGKDNTNLKKQFNKNPEKFLMYQNDPKTFEKLSIIMKQYSDEKNPLAKYYEETGEYLTKYSKKNNGPIVKKIKLLGNKVGNHLDVTNKYENSTKKLVKLSIK

SEQ ID NO: 108 - protein, 184 aa, Synthetic construct
RNINKVLDAPSKEVDKKRVTVQSEDEYNQIFEDTQKAQAIKKFEIRKFSHRVDKKPNRQLINDTLYSTRNIDGIEYVVESIKDIYSVNNDKVKTKFKKDPHRLLMYRNDPQTFEKFEKVFKQYESEKNPFAKYYEETGEKIRKFSKTGQGPYINKIKYLRERLGRHCDVTNKYINSRNKIVQLK

SEQ ID NO: 109 - protein, 183 aa, Synthetic construct
KEIKDVSDLAGDERNSNVKDEDQYEEVFGGYFKIEDIKKYKIKKFSHRVDKKPNRQLINDTIYSTRVKDDKRYLINTLKNLYDKSNGDLKERMQKDPESLLMYHHDPQTFEKLKIVMSQYENEKNPLAKYFEETGQYLTKYAKHDNGPAIHKIKYYGNKLVEHLDITKNYHNPQNKVVQLSQK

SEQ ID NO: 110 - protein, 145 aa, Synthetic construct
PYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 111 - protein, 145 aa, Synthetic construct
NYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 112 - protein, 144 aa, Synthetic construct
IYSYRFDIYQYGNNYKMITISYIDLEQKSNYYYISREKYEQKKKDKQIDDSYKFIGSFYKNDIINYNGEMYRVIGVNDSEKNKIQLDMIDISIKDYMELNNIKKTGVIYKTIGKSTTHIEKYTTDILGNLYKAAPPKKPQLIFK

SEQ ID NO: 113 - protein, 144 aa, Synthetic construct
SFRFDVYQTDKGYKFISIAYLTLKNEKNYYAISQEKYDQLKSEKKISNNAVFIGSFYTSDIIEINNEKFRVIGVNSDKNNLIEVDRIDIRQKEFIELEEEKKNNRIKVTIGRKTTNIEKFHTDILGNMYKSKRPKAPQLVFKKG

SEQ ID NO: 114 - RNA, 104 nt, Synthetic construct
  source 1..104: mol_type = other RNA
ggaccccctccaccccgcctcgttttagtactctggaaacagaatctactgaaacaagacaatatgtcgtgtttatcccatcaatttattggtgggattttttt

SEQ ID NO: 115 - DNA, 27 bp, Synthetic construct
  source 1..27: mol_type = other DNA
accccctccaccccgcctccgggcgcg

SEQ ID NO: 116 - DNA, 33 bp, Synthetic construct
  source 1..33: mol_type = other DNA
gcgcgcgcccggaggcggggtggagggggtcgg

SEQ ID NO: 117 - RNA, 20 nt, Synthetic construct
  source 1..20: mol_type = other RNA
gggtcagggtggtcacgagg

SEQ ID NO: 118 - DNA, 2188 bp, Synthetic construct
  source 1..2188: mol_type = other DNA
ccactgtagaagagcaaatgccacctgacgtctaagaaattcgcgttaaatttttgttaaatcagctcattttttaaccaataggccgaaatcggcaaaatcccttataaatcaaaagaatagaccgagatagggttgagtggccgctacactagggcgcgtctaatacgactcactataggaccccctccaccccgcctcgttttagtactctggaaacagaatctactgaaacaagacaatatgtcgtgtttatcccatcaatttattggtgggatttttttctagcataaccccttggggcctctaaacgggtcttgaggggttttttgtcactgcccgctttccagtcgggaaacctgtcgtgccagctgcattaatgaatcgttcgtcagaactgcttacgcggtttgtcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctcaatctaaagtatatatgagtaaacttggtctgacagttattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtg

SEQ ID NO: 119 - DNA, 9489 bp, Synthetic construct
  source 1..9489: mol_type = other DNA
ccggtgataccacgatactatgactgagagtcaacgccatgagcggcctcatttcttattctgagttacaacagtccgcaccgctgccggtagctattgactatccggctgcactagccctgcgtcagatggctctgatccaaggcaaactgccaaaatatctgctggcaccggaagtcagcgccctgcaccattatgttccggatctgcatcgcaggatgctgctggctaccctgtggaacacctacatctgtattaacgaagcgctggcattgaccctgagtgatttttctctggtgccgccctatccctttgtgcagcttgccacgctcaaaggggtttgaggtccaaccgtacgaaaacgtacggtaagaggaaaattatcgtctgaaaaatcgattagtagacaagaaagtccgttaagtgccaattttcgattaaaaagacaccgttttgatggcgttttccaatgtacattatgtttcgatatatcagacagttacttcactaacgtacgttttcgttctattggccttcagaccccatatccttaatgtcctttatttgctggggttatcagatccccccgacacgtttaattaatgctttctccgccggagatcgacgcacagcgttctgtgctctatgatgttatttcttaataatcatccaggtattctctttatcaccatacgtagtgcgagtgtccaccttaacgcagggctttccgtcacagcgcgatatgtcagccagcggggctttcttttgccagaccgcttccatcctctgcatttcagcaatctggctatacccgtcattcataaaccacgtaaatgccgtcacgcaggaagccaggacgaagaatatcgtcagtacaagataaatcgcggatttccacgtatagcgtgacatctcacgacgcatttcatggatcatcgctttcgccgtatcggcagcctgattcagcgcttctgtcgccggtttctgctgtgctaatccggcttgtttcagttctttctcaacctgagtgagcgcggaactcaccgatttcctgacggtgtcagtcatattaccggacgcgctgtccagctcacgaatgaccctgctcagcgtttcactttgctgctgtaattgtgatgaggcggcctgaaactgttctgtcagagaagtaacacgcttttccagcgcctgatgatgcccgataagggcggcaatttgtttaatttcgtcgctcatacaaaatcctgcctatcgtgagaatgaccagcctttatccggcttctgtcgtatctgttcggcgagtcgctgtcgttctttctcctgctgacgctgtttttccgccagacgttcgcgctctctctgcctttccatctcctgatgtatcccctggaactccgccatcgcatcgttaacaagggactgaagatcgatttcttcctgtatatccttcatggcatcactgaccagtgcgttcagcttgtcaggctctttttcaaaatcaaacgttctgccggaatgggattcctgctcaggctctgacttcagctcctgttttagcgtcagagtatccctctcgctgagggcttcccgtaacgaggtagtcacgtcaattacgctgtcacgttcatcacgggactgctgcacctgcctttcagcctccctgcgctcaagaatggcctgtagctgctcagtatcgaatcgctgaacctgacccgcgcccagatgccgctcaggctcacggtcaatgccctgcgccttcagggaacgggaatcaacccggtcagcgtgctgataccgttcaaggtgcttattctggaggtcagcccagcgttccctctgggcaacaaggtattctttgcgttcggtcggtgtttccccgaaacgtgccttttttgcgccaccgcgctccggctctttggtgttagcccgtttaaaatactgctcagggtcacggtgaataccgtcattaatgcgttcagagaacatgatatgggcgtggggctgctcgccaccggctatcgctgctttcggattatggatagcgaactgataggcatggcggtcgccaatttcctgttggacaaaatcgcggacaagctcaagacgttgttcgggttttaactcacgcggcagggcaatctcgatttcacggtaggtacagccgttggcacgttcagacgtgtcagcggctttccagaactcggacggtttatgcgctgcccacgccggcatattgccggactccttgtgctcaaggtcggagtctttttcacgggcatactttccctcacgcgcaatataatcggcatgaggagaggcactgccttttccgccggtttttacgctgagatgataggatgccatcgtgttttatcccgctgaaggcgcgcaccgtttctgaacgaagtgaagaaacgtctaagtgcgccctgataaataaaagagttatcagggattgtagtgggatttgacctcctctgccatcactgagcataatcattccgttagcattcaggaggtaaacagcatgaataaaagcgaaaaacaggaacaatgggcagcagaaagagtgcagtatattcgcggcttaaagtcgccgaatgagcaacagaaacttatgctgatactgacggataaagcagataaaacagcacaggatatcaaaacgctgtccctgctgatgaaggctgaacaggcagcagagaaagcgcaggaagccagagcgaaagtcatgaacctgatacaggcagaaaagcgagccgaagccagagccgcccgtaaagcccgtgaccatgctctgtaccagtctgccggattgcttatcctggcgggtctggttgacagtaagacgggtaagcctgttgatgataccgctgccttactgggtgcattagccagtctgaatgacctgtcacgggataatccgaagtggtcagactggaaaatcagagggcaggaactgctgaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacataacggactaagaaaaacactacacgatgctttaactgcaaaaattcagctcaccagttttgaggcaaaatttttgagtgacatgcaaagtaagtatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttacatatcaaagggaaaactgtccatatgcacagatgaaaacggtgtaaaaaagatagatacatcagagcttttacgagtttttggtgcattcaaagctgttcaccatgaacagatcgacaatgtaacagatgaacagcatgtaacacctaatagaacaggtgaaaccagtaaaacaaagcaactagaacatgaaattgaacacctgagacaacttgttacagctcaacagtcacacatagacagcctgaaacaggcgatgctgcttatcgaatcaaagctgccgacaacacgggagccagtgacgcctcccgtggggaaaaaatcatggcaattctggaagaaatagcgctttcagccggcaaaccggctgaagccggatctgcgattctgataacaaactagcaacaccagaacagcccgtttgcgggcagcaaaacccgtacttttggacgttccggcggttttttgtggcgagtggtgttcgggcggtgcgcgcaagatccattatgttaaacgggcgagtttacatctcaaaaccgcccgcttaacaccatcagaaatcctcagcgcgattttaagcaccaacccccccccgtaacacccaaatccatactgaaagtggctttgttgaataaatcgaacttttgctgagttgaaggatcagatcacgcatcctcccgacaacacagaccattccgtggcaaagcaaaagttcagaatcaccaactggtccacctacaacaaagctctcatcaaccgtggctccctcactttctggctggatgatgaggcgattcaggcctggtatgagtcggcaacaccttcatcacgaggaaggccccagcgctattctgatctcgccatcaccaccgttctggtgattaaacgcgtattccggctgaccctgcgggctgcgcagggttttattgattccatttttgccctgatgaacgttccgttgcgctgcccggattacaccagtgtcagtaagcgggcaaagtcggttaatgtcagtttcaaaacgtccacccggggtgaaatcgcacacctggtgattgattccaccgggctgaaggtctttggtgaaggcgaatggaaagtcagaaagcacggcaaagagcgccgtcgtatctggcgaaagttgcatcttgctgttgacagcaacacacatgaagttgtctgtgcagacctgtcgctgaataacgtcacggactcagaagccttcccgggccttatccggcagactcacagaaaaatcagggcagccgcggcagacggggcttacgatacccggctctgtcacgatgaactgcgccgcaaaaaaatcagcgcgcttattcctccccgaaaaggtgcgggttactggcccggtgaatatgcagaccgtaaccgtgcagtggctaatcagcgaatgaccgggagtaatgcgcggtggaaatggacaacagattacaaccgtcgctcgatagcggaaacggcgatgtaccgggtaaaacagctgttcgggggttcactgacgctgcgtgactacgatggtcaggttgcggaggctatggccctggtacgagcgctgaacaaaatgacgaaagcaggtatgcctgaaagcgtgcgtattgcctgaaaacacaacccgctacgggggagacttacccgaaatctgatttattcaacaaagccgggtgtggtgaactacaaagcagacccgttgaggttatcagttcgatgcacaatcagcagcgcataaaatatgcacaagaacaggagcacccttcgcattaagctgtggtggtaacaagtagtgccgggctaccatcagcgagcatgatgcgctcccacagcattcgccttggcagtatggaagttcctcgctccagttcgggccggtatccacctcgagctttctacggggtctgacgctcagtggaacgaaaactcacgttaagggattttggtcatgagattatcaaaaaggatcttcacctagatccttttcgaccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccttgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggattattgactaccggaagcagtgtgaccgtgtgcttctcaaatgcctgaggccagtttgctcaggctctccccgtggaggtaataattgacgatatgatcctttttttctgatcaaaagtgctccatggaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggaaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtcgtgcgcaacgcgtgacatcgactaggaggcctttctatgcagttcaaagtgtatacctataaacgcgaaagccgttatcgtctgtttgttgatgttcagagcgatattattgatacaccgggtcgtcgtatggttattccgctggcaagcgcacgtctgctgagcgataaagttagccgtgaactgtatccggttgttcatattggtgatgaaagctggcgtatgatgaccaccgatatggcaagcgttccggttagcgttattggtgaagaagttgcagatctgagccatcgtgaaaacgatatcaaaaatgccatcaacctgatgttttggggcatcttataggaccccctccaccccgcctcgcgggcagtttgcctggcggcagtagcgcggtggtcccacctgaccccatgccgaactcagaagtgaaacgccgtagcgccgatggtagtgtgggctctccccatgcgagagtagggaactgccaggcatcaaataaaacgaaaggctcagtcgaaagactgggcctta

SEQ ID NO: 120 - DNA, 6740 bp, Synthetic construct
  source 1..6740: mol_type = other DNA
ggtggaagcggaggcagcgggggatcaggccatcatcatcaccatcattaatgaacgggtcttgaggggttttttggacttttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccactcctcttgctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagatggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagatagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaatattctgaaatgagctgttgacaattaatcatccggctcgtataatgtgtggaattgtgagcggataacaattttcacacaggaaacagaattctagcattgtgagcggataacaattcccctctagaaataattttgtttaactttaagaaggagatataccatgaaacgtccggcagcaaccaaaaaagcaggtcaggccaagaaaaaaaaaggtggtggttcaggtaaccagaaatttatcctgggtctggatattggtattaccagcgttggttatggcctgattgattacgaaaccaaaaacattattgatgccggtgttcgtctgtttccggaagcaaatgttgaaaataatgaaggtcgtcgtagcaaacgtggtagccgtcgtctgaaacgtcgtcgtattcatcgtctggaacgtgttaaaaaactgctggaagattataacctgctggatcagagccagattccgcagagcaccaatccgtatgcaattcgtgttaaaggtctgagcgaagcactgagcaaagatgaactggttattgcactgctgcatattgcaaaacgccgtggcattcataaaatcgatgtgattgatagcaatgacgatgtgggtaatgaactgagcaccaaagaacagctgaacaaaaatagcaaactgctgaaagacaaattcgtgtgtcagattcagctggaacgtatgaatgaaggccaggttcgtggtgaaaagaatcgctttaaaaccgcagacatcatcaaagaaattatccagctgctgaacgtgcagaaaaacttccatcagctggatgaaaacttcatcaacaaatacatcgagctggttgaaatgcgtcgcgaatattttgaaggtccgggtaaaggtagcccgtatggttgggaaggtgatccgaaagcatggtatgaaaccctgatgggtcattgtacctattttccggatgaactgcgtagcgttaaatatgcctatagcgcagacctgtttaatgcactgaatgatctgaataacctggtgattcagcgtgatggtctgagcaaactggaatatcatgagaaatatcacatcatcgaaaacgtgttcaaacagaagaagaaaccgaccctgaaacaaatcgccaacgaaattaatgtgaacccggaagatattaaaggctaccgtattaccaaaagcggtaaaccgcagttcaccgaatttaaactgtatcacgatctgaaaagcgtgctgtttgatcagagcattctggaaaatgaagatgtgctggaccagattgcagaaattctgaccatttatcaggacaaagacagcatcaaaagcaaactgaccgaactggatattctgctgaatgaagaagataaagagaacattgcacagctgaccggttataccggcacccatcgtctgagcctgaaatgtattcgtctggtactggaagaacagtggtatagcagccgtaatcagatggaaatctttacccatctgaacattaaaccgaagaaaatcaatctgaccgcagccaacaaaattccgaaagccatgattgatgagtttattctgagtccggttgtgaaacgtacctttggtcaggcaattaacctgatcaacaaaatcattgaaaaatatggcgtgcctgaggatatcattattgaactggcacgtgaaaacaacagcaaagataaacagaaattcatcaacgagatgcagaagaagaacgaaaatacccgcaaacggattaacgagatcattggcaaatatggtaatcagaatgccaaacgcctggtggaaaaaattcgtctgcatgatgaacaagagggcaaatgtctgtatagcctggaaagcattcctctggaagatctgctgaacaatccgaatcattatgaagtggatcacattattccgcgtagcgtgagctttgataattcctatcataataaagtgctggtgaaacagagcgaaaactccaaaaaatccaacctgacaccgtatcagtatttcaatagcggcaaatccaaactgagctacaaccagtttaaacagcatattctgaacctgagcaaaagccaggatcgcatcagcaagaagaagaaggagtacctgctggaagaacgcgacatcaacaaatttgaagtgcagaaagaatttatcaaccgcaacctggttgatacccgttatgcaacccgtgaactgaccaattatctgaaagcatatttcagcgccaacaacatgaacgtgaaagtgaaaacgattaacggcagctttaccgattatctgcgtaaagtgtggaaattcaaaaaagaacgcaaccacggctataaacatcatgccgaagatgccctgattattgcaaatgcagatttcctgtttaaagaaaacaaaaaactgaaagccgtcaacagcgtgctggaaaaaccggaaattgagacaaaacagctggacattcaggttgatagcgaagataattacagcgaaatgtttatcatcccgaaacaggtgcaggatatcaaagattttcgcaacttcaaatatagccaccgcgttgacaaaaaacctaatcgtcagctgattaacgataccctgtatagcacccgcaaaaaagataacagcacctatattgtgcagaccattaaagacatctacgccaaagataataccaccctgaaaaaacagttcgacaaaagcccagaaaaatttctgatgtatcagcatgatccgcgtaccttcgaaaaactggaagttattatgaaacagtatgccaacgagaaaaatccgctggccaaatatcacgaagaaaccggtgaatatctgaccaaatattccaagaagaacaacggtccgatcgttaaatccctgaaatatatcggtaataaactgggcagccatctggatgttacccatcagtttaaaagctccacaaagaagctggttaaactgtccatcaaaccgtatcgctttgatgtgtatctgaccgacaaaggctataaattcattaccatcagctatctggacgtgctgaaaaaagacaactattattatatcccggaacagaaatatgataaactgaaactgggtaaagccatcgataaaaacgccaaatttatcgccagcttctacaaaaacgacctgattaaactggatggcgagatctataaaatcatcggtgttaatagcgacacccgcaatatgattgagctggatctgccggatattcgctataaagaatattgcgaactgaacaacattaaaggcgaaccgcgtatcaaaaagaccatcggcaaaaaagtgaatagcatcgagaaactgaccaccgatgttctgggtaatgtgtttaccaatacccagtataccaaacctcagctgctgttcaaacgcggtaatggcggaggatctggcccccctaagaaaaagcggaaggtg

SEQ ID NO: 121 - DNA, 6740 bp, Synthetic construct
  source 1..6740: mol_type = other DNA
tgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagatggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagatagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaatattctgaaatgagctgttgacaattaatcatccggctcgtataatgtgtggaattgtgagcggataacaattttcacacaggaaacagaattctagcattgtgagcggataacaattcccctctagaaataattttgtttaactttaagaaggagatataccatgaaacgtccggcagcaaccaaaaaagcaggtcaggccaagaaaaaaaaaggtggtggttcaggcaacaactatattctgggtctggatattggtattaccagcgttggttatggtattgttgatagcgatacccgtgaaattaaagatgccggtgttcgtctgtttccggaagcaaatgttgataataatgaaggtcgtcgtagcaaacgtggtgcacgtcgtctgaaacgtcgtcgtattcatcgtctggatcgtgttaaacatctgctggcagaatatgatctgctggatctgaccaatattccgaaaagcaccaatccgtatcagacccgtgttaaaggtctgaatgaaaagctgagcaaagatgaactggttattgcactgctgcatattgcaaaacgccgtggcattcataatgtgaatgttatgatggatgataacgatagcggtaatgaactgagcaccaaagatcagctgaagaagaatgcaaaagcactgagcgataaatatgtttgtgaactgcagctggaacgctttgagcaggattataaagttcgtggtgaaaagaaccgcttcaaaaccgaagattttgttcgtgaagcacgtaaactgctggaaacccagagcaaatttttcgaaattgatcagacgttcatcatgcgctatatcgaactgattgaaacccgtcgcgaatattttgaaggtccgggtaaaggtagcccgtttggttgggaaggtaatatcaagaaatggtttgagcagatgatgggccactgtacctattttccagaagaactgcgtagcgtcaaatatagctattcagccgaactgtttaacgccctgaatgatctgaataatctggtgattacccgtgatgaagatgccaaactgaattatggtgagaaattccagatcatcgaaaacgtgttcaaacagaagaaaacaccgaacctgaaacaaatcgccattgaaattggtgtgcatgaaaccgaaatcaaaggttatcgtgtgaacaaaagcggcaaaccggaatttacccagttcaaactgtatcacgatctgaagaacatcttcaaagacccgaaatacctgaacgatatccagctgatggataacattgcagaaatcatcaccatttatcaggatgccgaaagcatcatcaaagaactgaatcagctgccggaactgctgagcgaacgtgaaaaggagaagatcagcgcactgagcggttatagcggcacccatcgtctgagcctgaaatgtattaatctgctgctggatgatctgtgggaaagcagcctgaatcagatggaactgttcacaaaactgaatctgaaaccgaagaaaatcgatctgagccagcagcataaaattccgagcaaactggtggatgactttattctgagtccggttgttaaacgtgcatttattcagagtatccaggtggtgaatgccatcattgataaatacggtctgccggaagatatcatcattgaactggcacgtgaaaacaatagtgatgatcgtcgcaaatttctgaaccagctgcaaaagcagaatgaggaaacccgtaaacaggttgaaaaggtgctgcgtgaatatggcaatgataatgccaaacgtatcgtgcagaaaatcaaactgcataatatgcaagaggggaaatgtctgtatagcctgaaagatattccgctggaagatctgctgcgtaatccgcatcattatgaagtggatcatattattccgcgtagcgtggcatttgataatagcatgcataataaagttctggtgcgtgccgatgagaatagcaagaaaggtaatcgtaccccgtatcagtatctgaatagcagcgaaagcagtctgagctataacgaatttaaacagcatatcctgaacctgagcaaaaccaaagaccgcatcaccaagaagaagcgtgaatacctgctggaagaacgcgacatcaacaaatttgatgtgcagaaagaattcatcaaccgcaatctggttgatacacgttatgcaacccgtgaactgaccagcctgctgaaagcatatttcagcgcaaataacctggacgtgaaagtgaaaacgattaatggcagctttaccaactatctgcgtaaagtgtggaaattcgataaagatcgcaacaaaggctataaacatcatgcagaagatgccctgattattgccaatgcagattttctgttcaaacacaacaagaaactgcgcaacattaacaaagtgctggatgcaccgagcaaagaagttgataagaaacgcgtgacagttcagagcgaagatgaatataaccagatttttgaggatacccagaaagcccaggcaatcaagaaattcgaaatccgcaaatttagccaccgcgtggacaagaaaccgaatcgtcagctgattaatgataccctgtatagcacccgtaatatcgatggtattgaatatgtggtcgagagcatcaaagatatctatagcgtgaacaacgataaagttaaaaccaaattcaagaaagacccgcaccgtctgctgatgtatcgtaatgatccgcagacctttgagaaattcgagaaagtctttaaacagtatgagagcgagaagaacccgttcgccaaatattacgaagaaaccggtgagaaaattcgcaaattcagcaaaaccggtcagggtccgtatatcaacaaaatcaaatatctgcgtgaacgcctgggtcgtcattgtgatgttaccaacaaatatatcaatagccgcaacaaaattgtgcaactgtccatcaaaccgtatcgctttgatgtgtatctgaccgacaaaggctataaattcattaccatcagctatctggacgtgctgaaaaaagacaactattattatatcccggaacagaaatatgataaactgaaactgggtaaagccatcgataaaaacgccaaatttatcgccagcttctacaaaaacgacctgattaaactggatggcgagatctataaaatcatcggtgttaatagcgacacccgcaatatgattgagctggatctgccggatattcgctataaagaatattgcgaactgaacaacattaaaggcgaaccgcgtatcaaaaagaccatcggcaaaaaagtgaatagcatcgagaaactgaccaccgatgttctgggtaatgtgtttaccaatacccagtataccaaacctcagctgctgttcaaacgcggtaatggcggaggatctggcccccctaagaaaaagcggaaggtgggtggaagcggaggcagcgggggatcaggccatcatcatcaccatcattaatgaacgggtcttgaggggttttttggacttttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccactcctcttgctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgt

SEQ ID NO: 122 - DNA, 1431 bp, Synthetic construct
  source 1..1431: mol_type = other DNA
cggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagatagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaatattctgaaatgagctgttgacaattaatcatccggctcgtataatgtgtggaattgtgagcggataacaattttcacacaggaaacagaattctagcattgtgagcggataacaattcccctctagaaataattttgtttaactttaagaaggagatataccatgaaacgtccggcagcaaccaaaaaagcaggtcaggccaagaaaaaaaaaggtggtggttcaggcaacaactatattctgggtctggatattggtattaccagcgttggttatggtattgttgatagcgatacccgtgaaattaaagatgccggtgttcgtctgtttccggaagcaaatgttgataataatgaaggtcgtcgtagcaaacgtggtgcacgtcgtctgaaacgtcgtcgtattcatcgtctggatcgtgttaaacatctgctggcagaatatgatctgctggatctgaccaatattccgaaaagcaccaatccgtatcagacccgtgttaaaggtctgaatgaaaagctgagcaaagatgaactggttattgcactgctgcatattgcaaaacgccgtggcattcataatgtgaatgttatgatggatgataacgatagcggtaatgaactgagcaccaaagatcagctgaagaagaatgcaaaagcactgagcgataaatatgtttgtgaactgcagctggaacgctttgagcaggattataaagttcgtggtgaaaagaaccgcttcaaaaccgaagattttgttcgtgaagcacgtaaactgctggaaacccagagcaaatttttcgaaattgatcagacgttcatcatgcgctatatcgaactgattgaaacccgtcgcgaatattttgaaggtccgggtaaaggtagcccgtttggttgggaaggtaatatcaagaaatggtttgagcagatgatgggccactgtacctattttccagaagaactgcgtagcgtcaaatatagc

SEQ ID NO: 123 - DNA, 156 bp, Synthetic construct
  source 1..156: mol_type = other DNA
cttgtacttatacctaaaattacagaatctactgaaacaagacaatatgtcgtgtttatcccatcaatttattggtgggatttttttatgtttttagcaaaaagtaataccatactttatatttttaaattataataaagatataaataaaggtgg

SEQ ID NO: 124 - DNA, 3315 bp, Synthetic construct
  source 1..3315: mol_type = other DNA
atgaaacgtccggcagcaaccaaaaaagcaggtcaggccaagaaaaaaaaaggtggtggttcaggtaaccagaaatttatcctgggtctggatattggtattaccagcgttggttatggcctgattgattacgaaaccaaaaacattattgatgccggtgttcgtctgtttccggaagcaaatgttgaaaataatgaaggtcgtcgtagcaaacgtggtagccgtcgtctgaaacgtcgtcgtattcatcgtctggaacgtgttaaaaaactgctggaagattataacctgctggatcagagccagattccgcagagcaccaatccgtatgcaattcgtgttaaaggtctgagcgaagcactgagcaaagatgaactggttattgcactgctgcatattgcaaaacgccgtggcattcataaaatcgatgtgattgatagcaatgacgatgtgggtaatgaactgagcaccaaagaacagctgaacaaaaatagcaaactgctgaaagacaaattcgtgtgtcagattcagctggaacgtatgaatgaaggccaggttcgtggtgaaaagaatcgctttaaaaccgcagacatcatcaaagaaattatccagctgctgaacgtgcagaaaaacttccatcagctggatgaaaacttcatcaacaaatacatcgagctggttgaaatgcgtcgcgaatattttgaaggtccgggtaaaggtagcccgtatggttgggaaggtgatccgaaagcatggtatgaaaccctgatgggtcattgtacctattttccggatgaactgcgtagcgttaaatatgcctatagcgcagacctgtttaatgcactgaatgatctgaataacctggtgattcagcgtgatggtctgagcaaactggaatatcatgagaaatatcacatcatcgaaaacgtgttcaaacagaagaagaaaccgaccctgaaacaaatcgccaacgaaattaatgtgaacccggaagatattaaaggctaccgtattaccaaaagcggtaaaccgcagttcaccgaatttaaactgtatcacgatctgaaaagcgtgctgtttgatcagagcattctggaaaatgaagatgtgctggaccagattgcagaaattctgaccatttatcaggacaaagacagcatcaaaagcaaactgaccgaactggatattctgctgaatgaagaagataaagagaacattgcacagctgaccggttataccggcacccatcgtctgagcctgaaatgtattcgtctggtactggaagaacagtggtatagcagccgtaatcagatggaaatctttacccatctgaacattaaaccgaagaaaatcaatctgaccgcagccaacaaaattccgaaagccatgattgatgagtttattctgagtccggttgtgaaacgtacctttggtcaggcaattaacctgatcaacaaaatcattgaaaaatatggcgtgcctgaggatatcattattgaactggcacgtgaaaacaacagcaaagataaacagaaattcatcaacgagatgcagaagaagaacgaaaatacccgcaaacggattaacgagatcattggcaaatatggtaatcagaatgccaaacgcctggtggaaaaaattcgtctgcatgatgaacaagagggcaaatgtctgtatagcctggaaagcattcctctggaagatctgctgaacaatccgaatcattatgaagtggatcacattattccgcgtagcgtgagctttgataattcctatcataataaagtgctggtgaaacagagcgaaaactccaaaaaatccaacctgacaccgtatcagtatttcaatagcggcaaatccaaactgagctacaaccagtttaaacagcatattctgaacctgagcaaaagccaggatcgcatcagcaagaagaagaaggagtacctgctggaagaacgcgacatcaacaaatttgaagtgcagaaagaatttatcaaccgcaacctggttgatacccgttatgcaacccgtgaactgaccaattatctgaaagcatatttcagcgccaacaacatgaacgtgaaagtgaaaacgattaacggcagctttaccgattatctgcgtaaagtgtggaaattcaaaaaagaacgcaaccacggctataaacatcatgccgaagatgccctgattattgcaaatgcagatttcctgtttaaagaaaacaaaaaactgaaagccgtcaacagcgtgctggaaaaaccggaaattgagacaaaacagctggacattcaggttgatagcgaagataattacagcgaaatgtttatcatcccgaaacaggtgcaggatatcaaagattttcgcaacttcaaatatagccaccgcgttgacaaaaaacctaatcgtcagctgattaacgataccctgtatagcacccgcaaaaaagataacagcacctatattgtgcagaccattaaagacatctacgccaaagataataccaccctgaaaaaacagttcgacaaaagcccagaaaaatttctgatgtatcagcatgatccgcgtaccttcgaaaaactggaagttattatgaaacagtatgccaacgagaaaaatccgctggccaaatatcacgaagaaaccggtgaatatctgaccaaatattccaagaagaacaacggtccgatcgttaaatccctgaaatatatcggtaataaactgggcagccatctggatgttacccatcagtttaaaagctccacaaagaagctggttaaactgtccatcaaaccgtatcgctttgatgtgtatctgaccgacaaaggctataaattcattaccatcagctatctggacgtgctgaaaaaagacaactattattatatcccggaacagaaatatgataaactgaaactgggtaaagccatcgataaaaacgccaaatttatcgccagcttctacaaaaacgacctgattaaactggatggcgagatctataaaatcatcggtgttaatagcgacacccgcaatatgattgagctggatctgccggatattcgctataaagaatattgcgaactgaacaacattaaaggcgaaccgcgtatcaaaaagaccatcggcaaaaaagtgaatagcatcgagaaactgaccaccgatgttctgggtaatgtgtttaccaatacccagtataccaaacctcagctgctgttcaaacgcggtaatggcggaggatctggcccccctaagaaaaagcggaaggtgggtggaagcggaggcagcgggggatcaggccatcatcatcaccatcattaa

SEQ ID NO: 125 - protein, 1056 aa, Synthetic construct
MNQKFILGLDIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGKPQFTEFKLYHDLKSVLFDQSILENEDVLDQIAEILTIYQDKDSIKSKLTELDILLNEEDKENIAQLTGYTGTHRLSLKCIRLVLEEQWYSSRNQMEIFTHLNIKPKKINLTAANKIPKAMIDEFILSPVVKRTFGQAINLINKIIEKYGVPEDIIIELARENNSKDKQKFINEMQKKNENTRKRINEIIGKYGNQNAKRLVEKIRLHDEQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGNGG

SEQ ID NO: 126 - protein, 11 aa, Synthetic construct
YGRKKRRQRRR

SEQ ID NO: 127 - protein, 16 aa, Synthetic construct
RQIKIWFQNRRMKWKK

SEQ ID NO: 128 - RNA, 150 nt, Synthetic construct
  source 1..150: mol_type = other RNA
ggtgaacgtggatgaagttggttttagtactctggaaacagaatctactgaaacaagacaatatgtcgtgtttatcccatcaatttattggtgggatttttttctagcataaccccttggggcctctaaacgggtcttgaggggtttttt

SEQ ID NO: 129 - DNA, 28 bp, Synthetic construct
  source 1..28: mol_type = other DNA
ggtgaacgtggatgaagttgtggggtac

SEQ ID NO: 130 - DNA, 33 bp, Synthetic construct
  source 1..33: mol_type = other DNA
gcggtaccccacaacttcatccacgttcaccgg

SEQ ID NO: 131 - DNA, 28 bp, Synthetic construct
  source 1..28: mol_type = other DNA
agtgaacgtggatgaagttgtggggtac

SEQ ID NO: 132 - DNA, 33 bp, Synthetic construct
  source 1..33: mol_type = other DNA
gcggtaccccacaacttcatccacgttcactgg

SEQ ID NO: 133 - DNA, 28 bp, Synthetic construct
  source 1..28: mol_type = other DNA
cgtgaacgtggatgaagttgtggggtac

SEQ ID NO: 134 - DNA, 33 bp, Synthetic construct
  source 1..33: mol_type = other DNA
gcggtaccccacaacttcatccacgttcacggg

SEQ ID NO: 135 - DNA, 28 bp, Synthetic construct
  source 1..28: mol_type = other DNA
tgtgaacgtggatgaagttgtggggtac

SEQ ID NO: 136 - DNA, 33 bp, Synthetic construct
  source 1..33: mol_type = other DNA
gcggtaccccacaacttcatccacgttcacagg

SEQ ID NO: 137 - DNA, 28 bp, Synthetic construct
  source 1..28: mol_type = other DNA
gatgaacgtggatgaagttgtggggtac

SEQ ID NO: 138 - DNA, 33 bp, Synthetic construct
  source 1..33: mol_type = other DNA
gcggtaccccacaacttcatccacgttcatcgg

SEQ ID NO: 139 - DNA, 28 bp, Synthetic construct
  source 1..28: mol_type = other DNA
gctgaacgtggatgaagttgtggggtac

SEQ ID NO: 140 - DNA, 33 bp, Synthetic construct
  source 1..33: mol_type = other DNA
gcggtaccccacaacttcatccacgttcagcgg

SEQ ID NO: 141 - DNA, 28 bp, Synthetic construct
  source 1..28: mol_type = other DNA
gttgaacgtggatgaagttgtggggtac

SEQ ID NO: 142 - DNA, 33 bp, Synthetic construct
  source 1..33: mol_type = other DNA
gcggtaccccacaacttcatccacgttcaacgg

SEQ ID NO: 143 - DNA, 28 bp, Synthetic construct
  source 1..28: mol_type = other DNA
ggagaacgtggatgaagttgtggggtac

SEQ ID NO: 144 - DNA, 33 bp, Synthetic construct
  source 1..33: mol_type = other DNA
gcggtaccccacaacttcatccacgttctccgg

SEQ ID NO: 145 - DNA, 28 bp, Synthetic construct
  source 1..28: mol_type = other DNA
ggcgaacgtggatgaagttgtggggtac

SEQ ID NO: 146 - DNA, 33 bp, Synthetic construct
  source 1..33: mol_type = other DNA
gcggtaccccacaacttcatccacgttcgccgg

SEQ ID NO: 147 - DNA, 28 bp, Synthetic construct
  source 1..28: mol_type = other DNA
ggggaacgtggatgaagttgtggggtac

SEQ ID NO: 148 - DNA, 33 bp, Synthetic construct
  source 1..33: mol_type = other DNA
gcggtaccccacaacttcatccacgttccccgg

SEQ ID NO: 149 - DNA, 28 bp, Synthetic construct
  source 1..28: mol_type = other DNA
ggtaaacgtggatgaagttgtggggtac

SEQ ID NO: 150 - DNA, 33 bp, Synthetic construct
  source 1..33: mol_type = other DNA
gcggtaccccacaacttcatccacgtttaccgg

SEQ ID NO: 151 - DNA, 28 bp, Synthetic construct
  source 1..28: mol_type = other DNA
ggtcaacgtggatgaagttgtggggtac

SEQ ID NO: 152 - DNA, 33 bp, Synthetic construct
  source 1..33: mol_type = other DNA
gcggtaccccacaacttcatccacgttgaccgg

SEQ ID NO: 153 - DNA, 28 bp, Synthetic construct
  source 1..28: mol_type = other DNA
ggttaacgtggatgaagttgtggggtac

SEQ ID NO: 154 - DNA, 33 bp, Synthetic construct
  source 1..33: mol_type = other DNA
gcggtaccccacaacttcatccacgttaaccgg

SEQ ID NO: 155 - DNA, 28 bp, Synthetic construct
  source 1..28: mol_type = other DNA
ggtgcacgtggatgaagttgtggggtac

SEQ ID NO: 156 - DNA, 33 bp, Synthetic construct
  source 1..33: mol_type = other DNA
gcggtaccccacaacttcatccacgtgcaccgg

SEQ ID NO: 157 - DNA, 28 bp, Synthetic construct
  source 1..28: mol_type = other DNA
ggtgtacgtggatgaagttgtggggtac

SEQ ID NO: 158 - DNA, 33 bp, Synthetic construct
  source 1..33: mol_type = other DNA
gcggtaccccacaacttcatccacgtacaccgg

SEQ ID NO: 159 - DNA, 28 bp, Synthetic construct
  source 1..28: mol_type = other DNA
ggtggacgtggatgaagttgtggggtac

SEQ ID NO: 160 - DNA, 33 bp, Synthetic construct
  source 1..33: mol_type = other DNA
gcggtaccccacaacttcatccacgtccaccgg

SEQ ID NO: 161 - DNA, 28 bp, Synthetic construct
  source 1..28: mol_type = other DNA
ggtgaccgtggatgaagttgtggggtac

SEQ ID NO: 162 - DNA, 33 bp, Synthetic construct
  source 1..33: mol_type = other DNA
gcggtaccccacaacttcatccacggtcaccgg

SEQ ID NO: 163 - DNA, 28 bp, Synthetic construct
  source 1..28: mol_type = other DNA
ggtgatcgtggatgaagttgtggggtac

SEQ ID NO: 164 - DNA, 33 bp, Synthetic construct
  source 1..33: mol_type = other DNA
gcggtaccccacaacttcatccacgatcaccgg

SEQ ID NO: 165 - DNA, 28 bp, Synthetic construct
  source 1..28: mol_type = other DNA
ggtgagcgtggatgaagttgtggggtac

SEQ ID NO: 166 - DNA, 33 bp, Synthetic construct
  source 1..33: mol_type = other DNA
gcggtaccccacaacttcatccacgctcaccgg

SEQ ID NO: 167 - DNA, 28 bp, Synthetic construct
  source 1..28: mol_type = other DNA
ggtgaaagtggatgaagttgtggggtac

SEQ ID NO: 168 - DNA, 33 bp, Synthetic construct
  source 1..33: mol_type = other DNA
gcggtaccccacaacttcatccactttcaccgg

SEQ ID NO: 169 - DNA, 28 bp, Synthetic construct
  source 1..28: mol_type = other DNA
ggtgaatgtggatgaagttgtggggtac

SEQ ID NO: 170 - DNA, 33 bp, Synthetic construct
  source 1..33: mol_type = other DNA
gcggtaccccacaacttcatccacattcaccgg

SEQ ID NO: 171 - DNA, 28 bp, Synthetic construct
  source 1..28: mol_type = other DNA
ggtgaaggtggatgaagttgtggggtac

SEQ ID NO: 172 - DNA, 33 bp, Synthetic construct
  source 1..33: mol_type = other DNA
gcggtaccccacaacttcatccaccttcaccgg

SEQ ID NO: 173 - DNA, 28 bp, Synthetic construct
  source 1..28: mol_type = other DNA
ggtgaacatggatgaagttgtggggtac

SEQ ID NO: 174 - DNA, 33 bp, Synthetic construct
  source 1..33: mol_type = other DNA
gcggtaccccacaacttcatccatgttcaccgg

SEQ ID NO: 175 - DNA, 28 bp, Synthetic construct
  source 1..28: mol_type = other DNA
ggtgaacctggatgaagttgtggggtac

SEQ ID NO: 176 - DNA, 33 bp, Synthetic construct
  source 1..33: mol_type = other DNA
gcggtaccccacaacttcatccaggttcaccgg

SEQ ID NO: 177 - DNA, 28 bp, Synthetic construct
  source 1..28: mol_type = other DNA
ggtgaacttggatgaagttgtggggtac

SEQ ID NO: 178 - DNA, 33 bp, Synthetic construct
  source 1..33: mol_type = other DNA
gcggtaccccacaacttcatccaagttcaccgg

SEQ ID NO: 179 - DNA, 28 bp, Synthetic construct
  source 1..28: mol_type = other DNA
ggtgaacgaggatgaagttgtggggtac

SEQ ID NO: 180 - DNA, 33 bp, Synthetic construct
  source 1..33: mol_type = other DNA
gcggtaccccacaacttcatcctcgttcaccgg

SEQ ID NO: 181 - DNA, 28 bp, Synthetic construct
  source 1..28: mol_type = other DNA
ggtgaacgcggatgaagttgtggggtac

SEQ ID NO: 182 - DNA, 33 bp, Synthetic construct
  source 1..33: mol_type = other DNA
gcggtaccccacaacttcatccgcgttcaccgg

SEQ ID NO: 183 - DNA, 28 bp, Synthetic construct
  source 1..28: mol_type = other DNA
ggtgaacggggatgaagttgtggggtac

SEQ ID NO: 184 - DNA, 33 bp, Synthetic construct
  source 1..33: mol_type = other DNA
gcggtaccccacaacttcatccccgttcaccgg

SEQ ID NO: 185 - DNA, 28 bp, Synthetic construct
  source 1..28: mol_type = other DNA
ggtgaacgtagatgaagttgtggggtac

SEQ ID NO: 186 - DNA, 33 bp, Synthetic construct
  source 1..33: mol_type = other DNA
gcggtaccccacaacttcatctacgttcaccgg

SEQ ID NO: 187 - DNA, 28 bp, Synthetic construct
  source 1..28: mol_type = other DNA
ggtgaacgtcgatgaagttgtggggtac

SEQ ID NO: 188 - DNA, 33 bp, Synthetic construct
  source 1..33: mol_type = other DNA
gcggtaccccacaacttcatcgacgttcaccgg

SEQ ID NO: 189 - DNA, 28 bp, Synthetic construct
  source 1..28: mol_type = other DNA
ggtgaacgttgatgaagttgtggggtac

SEQ ID NO: 190 - DNA, 33 bp, Synthetic construct
  source 1..33: mol_type = other DNA
gcggtaccccacaacttcatcaacgttcaccgg

SEQ ID NO: 191 - DNA, 28 bp, Synthetic construct
  source 1..28: mol_type = other DNA
ggtgaacgtgaatgaagttgtggggtac

SEQ ID NO: 192 - DNA, 33 bp, Synthetic construct
  source 1..33: mol_type = other DNA
gcggtaccccacaacttcattcacgttcaccgg

SEQ ID NO: 193 - DNA, 28 bp, Synthetic construct
  source 1..28: mol_type = other DNA
ggtgaacgtgcatgaagttgtggggtac

SEQ ID NO: 194 - DNA, 33 bp, Synthetic construct
  source 1..33: mol_type = other DNA
gcggtaccccacaacttcatgcacgttcaccgg

SEQ ID NO: 195 - DNA, 28 bp, Synthetic construct
  source 1..28: mol_type = other DNA
ggtgaacgtgtatgaagttgtggggtac

SEQ ID NO: 196 - DNA, 33 bp, Synthetic construct
  source 1..33: mol_type = other DNA
gcggtaccccacaacttcatacacgttcaccgg

SEQ ID NO: 197 - DNA, 28 bp, Synthetic construct
  source 1..28: mol_type = other DNA
ggtgaacgtggctgaagttgtggggtac

SEQ ID NO: 198 - DNA, 33 bp, Synthetic construct
  source 1..33: mol_type = other DNA
gcggtaccccacaacttcagccacgttcaccgg

SEQ ID NO: 199 - DNA, 28 bp, Synthetic construct
  source 1..28: mol_type = other DNA
ggtgaacgtggttgaagttgtggggtac

SEQ ID NO: 200 - DNA, 33 bp, Synthetic construct
  source 1..33: mol_type = other DNA
gcggtaccccacaacttcaaccacgttcaccgg

SEQ ID NO: 201 - DNA, 28 bp, Synthetic construct
  source 1..28: mol_type = other DNA
ggtgaacgtgggtgaagttgtggggtac

SEQ ID NO: 202 - DNA, 33 bp, Synthetic construct
  source 1..33: mol_type = other DNA
gcggtaccccacaacttcacccacgttcaccgg

SEQ ID NO: 203 - DNA, 28 bp, Synthetic construct
  source 1..28: mol_type = other DNA
ggtgaacgtggaagaagttgtggggtac

SEQ ID NO: 204 - DNA, 33 bp, Synthetic construct
  source 1..33: mol_type = other DNA
gcggtaccccacaacttcttccacgttcaccgg

SEQ ID NO: 205 - DNA, 28 bp, Synthetic construct
  source 1..28: mol_type = other DNA
ggtgaacgtggacgaagttgtggggtac

SEQ ID NO: 206 - DNA, 33 bp, Synthetic construct
  source 1..33: mol_type = other DNA
gcggtaccccacaacttcgtccacgttcaccgg

SEQ ID NO: 207 - DNA, 28 bp, Synthetic construct
  source 1..28: mol_type = other DNA
ggtgaacgtggaggaagttgtggggtac

SEQ ID NO: 208 - DNA, 33 bp, Synthetic construct
  source 1..33: mol_type = other DNA
gcggtaccccacaacttcctccacgttcaccgg

SEQ ID NO: 209 - DNA, 28 bp, Synthetic construct
  source 1..28: mol_type = other DNA
ggtgaacgtggataaagttgtggggtac

SEQ ID NO: 210 - DNA, 33 bp, Synthetic construct
  source 1..33: mol_type = other DNA
gcggtaccccacaactttatccacgttcaccgg

SEQ ID NO: 211 - DNA, 28 bp, Synthetic construct
  source 1..28: mol_type = other DNA
ggtgaacgtggatcaagttgtggggtac

SEQ ID NO: 212 - DNA, 33 bp, Synthetic construct
  source 1..33: mol_type = other DNA
gcggtaccccacaacttgatccacgttcaccgg

SEQ ID NO: 213 - DNA, 28 bp, Synthetic construct
  source 1..28: mol_type = other DNA
ggtgaacgtggattaagttgtggggtac

SEQ ID NO: 214 - DNA, 33 bp, Synthetic construct
  source 1..33: mol_type = other DNA
gcggtaccccacaacttaatccacgttcaccgg

SEQ ID NO: 215 - DNA, 28 bp, Synthetic construct
  source 1..28: mol_type = other DNA
ggtgaacgtggatgcagttgtggggtac

SEQ ID NO: 216 - DNA, 33 bp, Synthetic construct
  source 1..33: mol_type = other DNA
gcggtaccccacaactgcatccacgttcaccgg

SEQ ID NO: 217 - DNA, 28 bp, Synthetic construct
  source 1..28: mol_type = other DNA
ggtgaacgtggatgtagttgtggggtac

SEQ ID NO: 218 - DNA, 33 bp, Synthetic construct
  source 1..33: mol_type = other DNA
gcggtaccccacaactacatccacgttcaccgg

SEQ ID NO: 219 - DNA, 28 bp, Synthetic construct
  source 1..28: mol_type = other DNA
ggtgaacgtggatggagttgtggggtac

SEQ ID NO: 220 - DNA, 33 bp, Synthetic construct
  source 1..33: mol_type = other DNA
gcggtaccccacaactccatccacgttcaccgg

SEQ ID NO: 221 - DNA, 28 bp, Synthetic construct
  source 1..28: mol_type = other DNA
ggtgaacgtggatgacgttgtggggtac

SEQ ID NO: 222 - DNA, 33 bp, Synthetic construct
  source 1..33: mol_type = other DNA
gcggtaccccacaacgtcatccacgttcaccgg

SEQ ID NO: 223 - DNA, 28 bp, Synthetic construct
  source 1..28: mol_type = other DNA
ggtgaacgtggatgatgttgtggggtac

SEQ ID NO: 224 - DNA, 33 bp, Synthetic construct
  source 1..33: mol_type = other DNA
gcggtaccccacaacatcatccacgttcaccgg

SEQ ID NO: 225 - DNA, 28 bp, Synthetic construct
  source 1..28: mol_type = other DNA
ggtgaacgtggatgaggttgtggggtac

SEQ ID NO: 226 - DNA, 33 bp, Synthetic construct
  source 1..33: mol_type = other DNA
gcggtaccccacaacctcatccacgttcaccgg

SEQ ID NO: 227 - DNA, 27 bp, Synthetic construct
  source 1..27: mol_type = other DNA
ggtgaacgtggatgaaattgtggggta

SEQ ID NO: 228 - DNA, 33 bp, Synthetic construct
  source 1..33: mol_type = other DNA
gcggtaccccacaatttcatccacgttcaccgg

SEQ ID NO: 229 - DNA, 28 bp, Synthetic construct
  source 1..28: mol_type = other DNA
ggtgaacgtggatgaacttgtggggtac

SEQ ID NO: 230 - DNA, 33 bp, Synthetic construct
  source 1..33: mol_type = other DNA
gcggtaccccacaagttcatccacgttcaccgg

SEQ ID NO: 231 - DNA, 28 bp, Synthetic construct
  source 1..28: mol_type = other DNA
ggtgaacgtggatgaatttgtggggtac

SEQ ID NO: 232 - DNA, 33 bp, Synthetic construct
  source 1..33: mol_type = other DNA
gcggtaccccacaaattcatccacgttcaccgg

SEQ ID NO: 233 - DNA, 28 bp, Synthetic construct
  source 1..28: mol_type = other DNA
ggtgaacgtggatgaagatgtggggtac

SEQ ID NO: 234 - DNA, 33 bp, Synthetic construct
  source 1..33: mol_type = other DNA
gcggtaccccacatcttcatccacgttcaccgg

SEQ ID NO: 235 - DNA, 28 bp, Synthetic construct
  source 1..28: mol_type = other DNA
ggtgaacgtggatgaagctgtggggtac

SEQ ID NO: 236 - DNA, 33 bp, Synthetic construct
  source 1..33: mol_type = other DNA
gcggtaccccacagcttcatccacgttcaccgg

SEQ ID NO: 237 - DNA, 28 bp, Synthetic construct
  source 1..28: mol_type = other DNA
ggtgaacgtggatgaaggtgtggggtac

SEQ ID NO: 238 - DNA, 33 bp, Synthetic construct
  source 1..33: mol_type = other DNA
gcggtaccccacaccttcatccacgttcaccgg

SEQ ID NO: 239 - DNA, 28 bp, Synthetic construct
  source 1..28: mol_type = other DNA
ggtgaacgtggatgaagtagtggggtac

SEQ ID NO: 240 - DNA, 33 bp, Synthetic construct
  source 1..33: mol_type = other DNA
gcggtaccccactacttcatccacgttcaccgg

SEQ ID NO: 241 - DNA, 28 bp, Synthetic construct
  source 1..28: mol_type = other DNA
ggtgaacgtggatgaagtcgtggggtac

SEQ ID NO: 242 - DNA, 33 bp, Synthetic construct
  source 1..33: mol_type = other DNA
gcggtaccccacgacttcatccacgttcaccgg

SEQ ID NO: 243 - DNA, 28 bp, Synthetic construct
  source 1..28: mol_type = other DNA
ggtgaacgtggatgaagtggtggggtac

SEQ ID NO: 244 - DNA, 33 bp, Synthetic construct
  source 1..33: mol_type = other DNA
gcggtaccccaccacttcatccacgttcaccgg

SEQ ID NO: 245 - DNA, 28 bp, Synthetic construct
  source 1..28: mol_type = other DNA
ggtgaacgtggatgaagttatggggtac

SEQ ID NO: 246 - DNA, 33 bp, Synthetic construct
  source 1..33: mol_type = other DNA
gcggtaccccataacttcatccacgttcaccgg

SEQ ID NO: 247 - DNA, 28 bp, Synthetic construct
  source 1..28: mol_type = other DNA
ggtgaacgtggatgaagttctggggtac

SEQ ID NO: 248 - DNA, 33 bp, Synthetic construct
  source 1..33: mol_type = other DNA
gcggtaccccagaacttcatccacgttcaccgg

SEQ ID NO: 249 - DNA, 28 bp, Synthetic construct
  source 1..28: mol_type = other DNA
ggtgaacgtggatgaagttttggggtac

SEQ ID NO: 250 - DNA, 33 bp, Synthetic construct
  source 1..33: mol_type = other DNA
gcggtaccccaaaacttcatccacgttcaccgg

SEQ ID NO: 251 - DNA, 21 bp, Synthetic construct
  source 1..21: mol_type = other DNA
ggagtccgagcagaagaagaa

SEQ ID NO: 252 - DNA, 21 bp, Synthetic construct
  source 1..21: mol_type = other DNA
ggacatcgatgtcctccccat

SEQ ID NO: 253 - DNA, 21 bp, Synthetic construct
  source 1..21: mol_type = other DNA
ggtcacctccaatgactaggg

SEQ ID NO: 254 - DNA, 22 bp, Synthetic construct
  source 1..22: mol_type = other DNA
ggctccccattggcctgcttcg

SEQ ID NO: 255 - DNA, 22 bp, Synthetic construct
  source 1..22: mol_type = other DNA
ggtgcgccaccggttgatgtga

SEQ ID NO: 256 - DNA, 21 bp, Synthetic construct
  source 1..21: mol_type = other DNA
ggcgccaccggttgatgtgat

SEQ ID NO: 257 - DNA, 21 bp, Synthetic construct
  source 1..21: mol_type = other DNA
ggccgtttgtactttgtcctc

SEQ ID NO: 258 - DNA, 21 bp, Synthetic construct
  source 1..21: mol_type = other DNA
ggcacagatgagaaactcagg

SEQ ID NO: 259 - DNA, 22 bp, Synthetic construct
  source 1..22: mol_type = other DNA
ggattgggtgttcagggcagag

SEQ ID NO: 260 - DNA, 21 bp, Synthetic construct
  source 1..21: mol_type = other DNA
ggtggcgagaggggccgagat

SEQ ID NO: 261 - DNA, 20 bp, Synthetic construct
  source 1..20: mol_type = other DNA
ggggccgagattgggtgttc

SEQ ID NO: 262 - DNA, 21 bp, Synthetic construct
  source 1..21: mol_type = other DNA
ggtgccattagctaaatgcat

SEQ ID NO: 263 - DNA, 21 bp, Synthetic construct
  source 1..21: mol_type = other DNA
ggtaccacccacaggtgccag

SEQ ID NO: 264 - DNA, 21 bp, Synthetic construct
  source 1..21: mol_type = other DNA
ggaaagcctctgggccaggaa

SEQ ID NO: 265 - DNA, 22 bp, Synthetic construct
  source 1..22: mol_type = other DNA
ggacacccacggagagcgctgt

SEQ ID NO: 266 - DNA, 21 bp, Synthetic construct
  source 1..21: mol_type = other DNA
ggtccagttggtgacaaatac

SEQ ID NO: 267 - DNA, 22 bp, Synthetic construct
  source 1..22: mol_type = other DNA
ggttttaacacttccctagcca

SEQ ID NO: 268 - DNA, 22 bp, Synthetic construct
  source 1..22: mol_type = other DNA
ggcttaattagctcctttggct

SEQ ID NO: 269 - DNA, 21 bp, Synthetic construct
  source 1..21: mol_type = other DNA
ggcttaattagctcctttggc

SEQ ID NO: 270 - DNA, 22 bp, Synthetic construct
  source 1..22: mol_type = other DNA
ggtgcatatgatgatagtatta

SEQ ID NO: 271 - DNA, 22 bp, Synthetic construct
  source 1..22: mol_type = other DNA
ggaatgtatgctggcttttaag

SEQ ID NO: 272 - DNA, 22 bp, Synthetic construct
  source 1..22: mol_type = other DNA
ggaaatgtatgctggcttttaa

SEQ ID NO: 273 - DNA, 22 bp, Synthetic construct
  source 1..22: mol_type = other DNA
ggaaaatgtatgctggctttta

SEQ ID NO: 274 - DNA, 22 bp, Synthetic construct
  source 1..22: mol_type = other DNA
ggtaaaatgtatgctggctttt

SEQ ID NO: 275 - DNA, 22 bp, Synthetic construct
  source 1..22: mol_type = other DNA
ggcttaaagatgatctcttacc

SEQ ID NO: 276 - DNA, 22 bp, Synthetic construct
  source 1..22: mol_type = other DNA
ggttaaagatgatctcttacct

SEQ ID NO: 277 - DNA, 22 bp, Synthetic construct
  source 1..22: mol_type = other DNA
ggtaataaaattcaaacatcct

SEQ ID NO: 278 - DNA, 22 bp, Synthetic construct
  source 1..22: mol_type = other DNA
ggtaaagcatagtgcaatggat

SEQ ID NO: 279 - DNA, 22 bp, Synthetic construct
  source 1..22: mol_type = other DNA
ggttaaataaagcatagtgcaa

SEQ ID NO: 280 - DNA, 22 bp, Synthetic construct
  source 1..22: mol_type = other DNA
ggatttatgagatcaacagcac

SEQ ID NO: 281 - DNA, 22 bp, Synthetic construct
  source 1..22: mol_type = other DNA
ggcccatgctctgaccgctcga

SEQ ID NO: 282 - DNA, 22 bp, Synthetic construct
  source 1..22: mol_type = other DNA
ggccatcgagcggtcagagcat

SEQ ID NO: 283 - DNA, 20 bp, Synthetic construct
  source 1..20: mol_type = other DNA
gggcaaaagctcatgtgata

SEQ ID NO: 284 - DNA, 22 bp, Synthetic construct
  source 1..22: mol_type = other DNA
ggactgaggcttatgttccatg

SEQ ID NO: 285 - DNA, 21 bp, Synthetic construct
  source 1..21: mol_type = other DNA
ggagagtgtacaaactcacaa

SEQ ID NO: 286 - DNA, 22 bp, Synthetic construct
  source 1..22: mol_type = other DNA
ggactagcattataatgcacca

SEQ ID NO: 287 - DNA, 22 bp, Synthetic construct
  source 1..22: mol_type = other DNA
ggtcagaagagattagttagta

SEQ ID NO: 288 - DNA, 22 bp, Synthetic construct
  source 1..22: mol_type = other DNA
ggtgagagtgccatctcttcct

SEQ ID NO: 289 - DNA, 22 bp, Synthetic construct
  source 1..22: mol_type = other DNA
ggtggaccacatggctttgctc

SEQ ID NO: 290 - DNA, 22 bp, Synthetic construct
  source 1..22: mol_type = other DNA
ggtgagcaaagccatgtggtcc

SEQ ID NO: 291 - DNA, 21 bp, Synthetic construct
  source 1..21: mol_type = other DNA
ggattaatgacatacgcattt

SEQ ID NO: 292 - DNA, 22 bp, Synthetic construct
  source 1..22: mol_type = other DNA
ggagattaatgacatacgcatt

SEQ ID NO: 293 - DNA, 22 bp, Synthetic construct
  source 1..22: mol_type = other DNA
ggttcaatcctcttgtcacctg

SEQ ID NO: 294 - DNA, 21 bp, Synthetic construct
  source 1..21: mol_type = other DNA
ggacatctctatgtcggccac

SEQ ID NO: 295 - DNA, 22 bp, Synthetic construct
  source 1..22: mol_type = other DNA
ggaaaacctaccgcaagttgcc

SEQ ID NO: 296 - DNA, 22 bp, Synthetic construct
  source 1..22: mol_type = other DNA
ggtggactacatagttgtgtga

SEQ ID NO: 297 - DNA, 22 bp, Synthetic construct
  source 1..22: mol_type = other DNA
ggctgccagactctctgaaccc

SEQ ID NO: 298 - DNA, 20 bp, Synthetic construct
  source 1..20: mol_type = other DNA
ggatccctcgtataacaata

SEQ ID NO: 299 - DNA, 22 bp, Synthetic construct
  source 1..22: mol_type = other DNA
ggtatcgcatagtgcaaagaat

SEQ ID NO: 300 - DNA, 22 bp, Synthetic construct
  source 1..22: mol_type = other DNA
ggctcggtcctctcgtcacctg

SEQ ID NO: 301 - DNA, 22 bp, Synthetic construct
  source 1..22: mol_type = other DNA
ggacgctcccggagagcgctgg

SEQ ID NO: 302 - DNA, 22 bp, Synthetic construct
  source 1..22: mol_type = other DNA
ggccatggcgcggtcagagggt

SEQ ID NO: 303 - DNA, 22 bp, Synthetic construct
  source 1..22: mol_type = other DNA
ggctcggctccaatgacgaggg

SEQ ID NO: 304 - DNA, 22 bp, Synthetic construct
  source 1..22: mol_type = other DNA
ggcacttcgatgacctccccgt

SEQ ID NO: 305 - DNA, 22 bp, Synthetic construct
  source 1..22: mol_type = other DNA
ggagtgggtgttcaggaccgcg

SEQ ID NO: 306 - DNA, 22 bp, Synthetic construct
  source 1..22: mol_type = other DNA
ggctgctacccacaggtgcaag

SEQ ID NO: 307 - DNA, 22 bp, Synthetic construct
  source 1..22: mol_type = other DNA
ggaagatgtatcctggaattta

SEQ ID NO: 308 - DNA, 22 bp, Synthetic construct
  source 1..22: mol_type = other DNA
ggttttatcactacccaagaca

SEQ ID NO: 309 - DNA, 22 bp, Synthetic construct
  source 1..22: mol_type = other DNA
ggcggccaggctccctgaaacc

SEQ ID NO: 310 - DNA, 22 bp, Synthetic construct
  source 1..22: mol_type = other DNA
ggactggtattataatacaaca

SEQ ID NO: 311 - DNA, 22 bp, Synthetic construct
  source 1..22: mol_type = other DNA
ggtgagagtgacatttcgtact

SEQ ID NO: 312 - DNA, 22 bp, Synthetic construct
  source 1..22: mol_type = other DNA
ggtgcgcccccgggtgaggtgc

SEQ ID NO: 313 - DNA, 22 bp, Synthetic construct
  source 1..22: mol_type = other DNA
ggcagtttgagcagaaaaagaa

SEQ ID NO: 314 - DNA, 22 bp, Synthetic construct
  source 1..22: mol_type = other DNA
ggtctgtagagataagtgagta

SEQ ID NO: 315 - DNA, 22 bp, Synthetic construct
  source 1..22: mol_type = other DNA
ggaagtgtgtacaaactcaaga

SEQ ID NO: 316 - DNA, 22 bp, Synthetic construct
  source 1..22: mol_type = other DNA
ggtatcatggatgctgactttt

SEQ ID NO: 317 - DNA, 21 bp, Synthetic construct
  source 1..21: mol_type = other DNA
ggctttactagcccctgtggc

SEQ ID NO: 318 - DNA, 21 bp, Synthetic construct
  source 1..21: mol_type = other DNA
ggcacggaggagaaaagcagg

SEQ ID NO: 319 - DNA, 22 bp, Synthetic construct
  source 1..22: mol_type = other DNA
ggatttgtgagatctaaagaac

SEQ ID NO: 320 - DNA, 22 bp, Synthetic construct
  source 1..22: mol_type = other DNA
ggcgttgatgacattcgcattt

SEQ ID NO: 321 - DNA, 22 bp, Synthetic construct
  source 1..22: mol_type = other DNA
ggcttatagatcatctaatacc

SEQ ID NO: 322 - DNA, 22 bp, Synthetic construct
  source 1..22: mol_type = other DNA
ggtggcctccatagttgggtgg

SEQ ID NO: 323 - DNA, 22 bp, Synthetic construct
  source 1..22: mol_type = other DNA
ggtactagagttcaaacatact

SEQ ID NO: 324 - DNA, 22 bp, Synthetic construct
  source 1..22: mol_type = other DNA
ggcccctgctctgagcactcgg

SEQ ID NO: 325 - DNA, 22 bp, Synthetic construct
  source 1..22: mol_type = other DNA
ggaggaccacatggcctgcctc

SEQ ID NO: 326 - DNA, 22 bp, Synthetic construct
  source 1..22: mol_type = other DNA
ggaacagctgccgcaaattgcc

SEQ ID NO: 327 - DNA, 22 bp, Synthetic construct
  source 1..22: mol_type = other DNA
ggctccgtattggctggcttcg

SEQ ID NO: 328 - DNA, 20 bp, Synthetic construct
  source 1..20: mol_type = other DNA
ggccagccgatgacaaatac

SEQ ID NO: 329 - DNA, 21 bp, Synthetic construct
  source 1..21: mol_type = other DNA
ggacgtctctacgtcggcggc

SEQ ID NO: 330 - DNA, 22 bp, Synthetic construct
  source 1..22: mol_type = other DNA
ggccggcgcgaggggccgagag

SEQ ID NO: 331 - DNA, 22 bp, Synthetic construct
  source 1..22: mol_type = other DNA
ggactgtttgtactttttcatc

SEQ ID NO: 332 - DNA, 22 bp, Synthetic construct
  source 1..22: mol_type = other DNA
ggtgcttatgatgatgacatta

SEQ ID NO: 333 - DNA, 22 bp, Synthetic construct
  source 1..22: mol_type = other DNA
ggccgcgtccgattgatgtgat

SEQ ID NO: 334 - DNA, 22 bp, Synthetic construct
  source 1..22: mol_type = other DNA
ggctttatgagcaccttaggct

SEQ ID NO: 335 - DNA, 20 bp, Synthetic construct
  source 1..20: mol_type = other DNA
ggatccctcctatgaccatg

SEQ ID NO: 336 - DNA, 22 bp, Synthetic construct
  source 1..22: mol_type = other DNA
ggataaaggtgacctcttacgt

SEQ ID NO: 337 - DNA, 22 bp, Synthetic construct
  source 1..22: mol_type = other DNA
ggactgtggcttagggtccagg

SEQ ID NO: 338 - DNA, 22 bp, Synthetic construct
  source 1..22: mol_type = other DNA
ggtgaggaaacccaggcggtcc

SEQ ID NO: 339 - DNA, 22 bp, Synthetic construct
  source 1..22: mol_type = other DNA
ggaaatgtatacaggcaattaa

SEQ ID NO: 340 - DNA, 22 bp, Synthetic construct
  source 1..22: mol_type = other DNA
ggttgaatcaagcatagtccga

SEQ ID NO: 341 - DNA, 20 bp, Synthetic construct
  source 1..20: mol_type = other DNA
gggctacggctcatgagata

SEQ ID NO: 342 - DNA, 22 bp, Synthetic construct
  source 1..22: mol_type = other DNA
ggcgggccgcgattggcagttc

SEQ ID NO: 343 - DNA, 22 bp, Synthetic construct
  source 1..22: mol_type = other DNA
ggctgcgattatctaaatccat

SEQ ID NO: 344 - DNA, 22 bp, Synthetic construct
  source 1..22: mol_type = other DNA
ggagcttgattacatacgcatg

SEQ ID NO: 345 - DNA, 22 bp, Synthetic construct
  source 1..22: mol_type = other DNA
ggaatgtttgctggcctctagg

SEQ ID NO: 346 - DNA, 21 bp, Synthetic construct
  source 1..21: mol_type = other DNA
ggaaagcctctgcgccggggg

SEQ ID NO: 347 - DNA, 31 bp, Synthetic construct
  source 1..31: mol_type = other DNA
cctgagtccgagcagaagaagaatggggtac

SEQ ID NO: 348 - DNA, 36 bp, Synthetic construct
  source 1..36: mol_type = other DNA
gcggtaccccattcttcttctgctcggactcagggg

SEQ ID NO: 349 - DNA, 31 bp, Synthetic construct
  source 1..31: mol_type = other DNA
ggtgacatcgatgtcctccccattggggtac

SEQ ID NO: 350 - DNA, 36 bp, Synthetic construct
  source 1..36: mol_type = other DNA
gcggtaccccaatggggaggacatcgatgtcaccgg

SEQ ID NO: 351 - DNA, 31 bp, Synthetic construct
  source 1..31: mol_type = other DNA
gatgtcacctccaatgactagggtggggtac

SEQ ID NO: 352 - DNA, 36 bp, Synthetic construct
  source 1..36: mol_type = other DNA
gcggtaccccaccctagtcattggaggtgacatcgg

SEQ ID NO: 353 - DNA, 31 bp, Synthetic construct
  source 1..31: mol_type = other DNA
gtcctccccattggcctgcttcgtggggtac

SEQ ID NO: 354 - DNA, 36 bp, Synthetic construct
  source 1..36: mol_type = other DNA
gcggtaccccacgaagcaggccaatggggaggacgg

SEQ ID NO: 355 - DNA, 31 bp, Synthetic construct
  source 1..31: mol_type = other DNA
caatgcgccaccggttgatgtgatggggtac

SEQ ID NO: 356 - DNA, 36 bp, Synthetic construct
  source 1..36: mol_type = other DNA
gcggtaccccatcacatcaaccggtggcgcattggg

SEQ ID NO: 357 - DNA, 31 bp, Synthetic construct
  source 1..31: mol_type = other DNA
aatgcgccaccggttgatgtgattggggtac

SEQ ID NO: 358 - DNA, 36 bp, Synthetic construct
  source 1..36: mol_type = other DNA
gcggtaccccaatcacatcaaccggtggcgcattgg

SEQ ID NO: 359 - DNA, 31 bp, Synthetic construct
  source 1..31: mol_type = other DNA
tctgccgtttgtactttgtcctctggggtac

SEQ ID NO: 360 - DNA, 36 bp, Synthetic construct
  source 1..36: mol_type = other DNA
gcggtaccccagaggacaaagtacaaacggcagagg

SEQ ID NO: 361 - DNA, 31 bp, Synthetic construct
  source 1..31: mol_type = other DNA
ggggcacagatgagaaactcaggtggggtac

SEQ ID NO: 362 - DNA, 36 bp, Synthetic construct
  source 1..36: mol_type = other DNA
gcggtaccccacctgagtttctcatctgtgccccgg

SEQ ID NO: 363 - DNA, 31 bp, Synthetic construct
  source 1..31: mol_type = other DNA
gagattgggtgttcagggcagagtggggtac

SEQ ID NO: 364 - DNA, 36 bp, Synthetic construct
  source 1..36: mol_type = other DNA
gcggtaccccactctgccctgaacacccaatctcgg

SEQ ID NO: 365 - DNA, 31 bp, Synthetic construct
  source 1..31: mol_type = other DNA
agggtggcgagaggggccgagattggggtac

SEQ ID NO: 366 - DNA, 36 bp, Synthetic construct
  source 1..36: mol_type = other DNA
gcggtaccccaatctcggcccctctcgccaccctgg

SEQ ID NO: 367 - DNA, 31 bp, Synthetic construct
  source 1..31: mol_type = other DNA
agaggggccgagattgggtgttctggggtac

SEQ ID NO: 368 - DNA, 36 bp, Synthetic construct
  source 1..36: mol_type = other DNA
gcggtaccccagaacacccaatctcggcccctctgg

SEQ ID NO: 369 - DNA, 31 bp, Synthetic construct
  source 1..31: mol_type = other DNA
tgagtgccattagctaaatgcattggggtac

SEQ ID NO: 370 - DNA, 36 bp, Synthetic construct
  source 1..36: mol_type = other DNA
gcggtaccccaatgcatttagctaatggcactcagg

SEQ ID NO: 371 - DNA, 31 bp, Synthetic construct
  source 1..31: mol_type = other DNA
agggtaccacccacaggtgccagtggggtac

SEQ ID NO: 372 - DNA, 36 bp, Synthetic construct
  source 1..36: mol_type = other DNA
gcggtaccccactggcacctgtgggtggtaccctgg

SEQ ID NO: 373 - DNA, 31 bp, Synthetic construct
  source 1..31: mol_type = other DNA
tgggaaagcctctgggccaggaatggggtac

SEQ ID NO: 374 - DNA, 36 bp, Synthetic construct
  source 1..36: mol_type = other DNA
gcggtaccccattcctggcccagaggctttcccagg

SEQ ID NO: 375 - DNA, 31 bp, Synthetic construct
  source 1..31: mol_type = other DNA
aggacacccacggagagcgctgttggggtac

SEQ ID NO: 376 - DNA, 36 bp, Synthetic construct
  source 1..36: mol_type = other DNA
gcggtaccccaacagcgctctccgtgggtgtcctgg

SEQ ID NO: 377 - DNA, 31 bp, Synthetic construct
  source 1..31: mol_type = other DNA
agggtccagttggtgacaaatactggggtac

SEQ ID NO: 378 - DNA, 36 bp, Synthetic construct
  source 1..36: mol_type = other DNA
gcggtaccccagtatttgtcaccaactggaccctgg

SEQ ID NO: 379 - DNA, 31 bp, Synthetic construct
  source 1..31: mol_type = other DNA
aggttttaacacttccctagccatggggtac

SEQ ID NO: 380 - DNA, 36 bp, Synthetic construct
  source 1..36: mol_type = other DNA
gcggtaccccatggctagggaagtgttaaaacctgg

SEQ ID NO: 381 - DNA, 31 bp, Synthetic construct
  source 1..31: mol_type = other DNA
aggcttaattagctcctttggcttggggtac

SEQ ID NO: 382 - DNA, 36 bp, Synthetic construct
  source 1..36: mol_type = other DNA
gcggtaccccaagccaaaggagctaattaagcctgg

SEQ ID NO: 383 - DNA, 31 bp, Synthetic construct
  source 1..31: mol_type = other DNA
agggcttaattagctcctttggctggggtac

SEQ ID NO: 384 - DNA, 36 bp, Synthetic construct
  source 1..36: mol_type = other DNA
gcggtaccccagccaaaggagctaattaagccctgg

SEQ ID NO: 385 - DNA, 31 bp, Synthetic construct
  source 1..31: mol_type = other DNA
aggtgcatatgatgatagtattatggggtac

SEQ ID NO: 386 - DNA, 36 bp, Synthetic construct
  source 1..36: mol_type = other DNA
gcggtaccccataatactatcatcatatgcacctgg

SEQ ID NO: 387 - DNA, 31 bp, Synthetic construct
  source 1..31: mol_type = other DNA
aggaatgtatgctggcttttaagtggggtac

SEQ ID NO: 388 - DNA, 36 bp, Synthetic construct
  source 1..36: mol_type = other DNA
gcggtaccccacttaaaagccagcatacattcctgg

SEQ ID NO: 389 - DNA, 31 bp, Synthetic construct
  source 1..31: mol_type = other DNA
aggaaatgtatgctggcttttaatggggtac

SEQ ID NO: 390 - DNA, 36 bp, Synthetic construct
  source 1..36: mol_type = other DNA
gcggtaccccattaaaagccagcatacatttcctgg

SEQ ID NO: 391 - DNA, 31 bp, Synthetic construct
  source 1..31: mol_type = other DNA
aggaaaatgtatgctggcttttatggggtac

SEQ ID NO: 392 - DNA, 36 bp, Synthetic construct
  source 1..36: mol_type = other DNA
gcggtaccccataaaagccagcatacattttcctgg

SEQ ID NO: 393 - DNA, 31 bp, Synthetic construct
  source 1..31: mol_type = other DNA
aggtaaaatgtatgctggctttttggggtac

SEQ ID NO: 394 - DNA, 36 bp, Synthetic construct
  source 1..36: mol_type = other DNA
gcggtaccccaaaaagccagcatacattttacctgg

SEQ ID NO: 395 - DNA, 31 bp, Synthetic construct
  source 1..31: mol_type = other DNA
aggcttaaagatgatctcttacctggggtac

SEQ ID NO: 396 - DNA, 36 bp, Synthetic construct
  source 1..36: mol_type = other DNA
gcggtaccccaggtaagagatcatctttaagcctgg

SEQ ID NO: 397 - DNA, 31 bp, Synthetic construct
  source 1..31: mol_type = other DNA
aggttaaagatgatctcttaccttggggtac

SEQ ID NO: 398 - DNA, 36 bp, Synthetic construct
  source 1..36: mol_type = other DNA
gcggtaccccaaggtaagagatcatctttaacctgg

SEQ ID NO: 399 - DNA, 31 bp, Synthetic construct
  source 1..31: mol_type = other DNA
aggtaataaaattcaaacatccttggggtac

SEQ ID NO: 400 - DNA, 36 bp, Synthetic construct
  source 1..36: mol_type = other DNA
gcggtaccccaaggatgtttgaattttattacctgg

SEQ ID NO: 401 - DNA, 31 bp, Synthetic construct
  source 1..31: mol_type = other DNA
aggtaaagcatagtgcaatggattggggtac

SEQ ID NO: 402 - DNA, 36 bp, Synthetic construct
  source 1..36: mol_type = other DNA
gcggtaccccaatccattgcactatgctttacctgg

SEQ ID NO: 403 - DNA, 31 bp, Synthetic construct
  source 1..31: mol_type = other DNA
aggttaaataaagcatagtgcaatggggtac

SEQ ID NO: 404 - DNA, 36 bp, Synthetic construct
  source 1..36: mol_type = other DNA
gcggtaccccattgcactatgctttatttaacctgg

SEQ ID NO: 405 - DNA, 31 bp, Synthetic construct
  source 1..31: mol_type = other DNA
aggatttatgagatcaacagcactggggtac

SEQ ID NO: 406 - DNA, 36 bp, Synthetic construct
  source 1..36: mol_type = other DNA
gcggtaccccagtgctgttgatctcataaatcctgg

SEQ ID NO: 407 - DNA, 31 bp, Synthetic construct
  source 1..31: mol_type = other DNA
aggcccatgctctgaccgctcgatggggtac

SEQ ID NO: 408 - DNA, 36 bp, Synthetic construct
  source 1..36: mol_type = other DNA
gcggtaccccatcgagcggtcagagcatgggcctgg

SEQ ID NO: 409 - DNA, 31 bp, Synthetic construct
  source 1..31: mol_type = other DNA
aggccatcgagcggtcagagcattggggtac

SEQ ID NO: 410 - DNA, 36 bp, Synthetic construct
  source 1..36: mol_type = other DNA
gcggtaccccaatgctctgaccgctcgatggcctgg

SEQ ID NO: 411 - DNA, 31 bp, Synthetic construct
  source 1..31: mol_type = other DNA
agggggcaaaagctcatgtgatatggggtac

SEQ ID NO: 412 - DNA, 36 bp, Synthetic construct
  source 1..36: mol_type = other DNA
gcggtaccccatatcacatgagcttttgccccctgg

SEQ ID NO: 413 - DNA, 31 bp, Synthetic construct
  source 1..31: mol_type = other DNA
aggactgaggcttatgttccatgtggggtac

SEQ ID NO: 414 - DNA, 36 bp, Synthetic construct
  source 1..36: mol_type = other DNA
gcggtaccccacatggaacataagcctcagtcctgg

SEQ ID NO: 415 - DNA, 31 bp, Synthetic construct
  source 1..31: mol_type = other DNA
agggagagtgtacaaactcacaatggggtac

SEQ ID NO: 416 - DNA, 36 bp, Synthetic construct
  source 1..36: mol_type = other DNA
gcggtaccccattgtgagtttgtacactctccctgg

SEQ ID NO: 417 - DNA, 31 bp, Synthetic construct
  source 1..31: mol_type = other DNA
aggactagcattataatgcaccatggggtac

SEQ ID NO: 418 - DNA, 36 bp, Synthetic construct
  source 1..36: mol_type = other DNA
gcggtaccccatggtgcattataatgctagtcctgg

SEQ ID NO: 419 - DNA, 31 bp, Synthetic construct
  source 1..31: mol_type = other DNA
aggtcagaagagattagttagtatggggtac

SEQ ID NO: 420 - DNA, 36 bp, Synthetic construct
  source 1..36: mol_type = other DNA
gcggtaccccatactaactaatctcttctgacctgg

SEQ ID NO: 421 - DNA, 31 bp, Synthetic construct
  source 1..31: mol_type = other DNA
aggtgagagtgccatctcttccttggggtac

SEQ ID NO: 422 - DNA, 36 bp, Synthetic construct
  source 1..36: mol_type = other DNA
gcggtaccccaaggaagagatggcactctcacctgg

SEQ ID NO: 423 - DNA, 31 bp, Synthetic construct
  source 1..31: mol_type = other DNA
aggtggaccacatggctttgctctggggtac

SEQ ID NO: 424 - DNA, 36 bp, Synthetic construct
  source 1..36: mol_type = other DNA
gcggtaccccagagcaaagccatgtggtccacctgg

SEQ ID NO: 425 - DNA, 31 bp, Synthetic construct
  source 1..31: mol_type = other DNA
aggtgagcaaagccatgtggtcctggggtac

SEQ ID NO: 426 - DNA, 36 bp, Synthetic construct
  source 1..36: mol_type = other DNA
gcggtaccccaggaccacatggctttgctcacctgg

SEQ ID NO: 427 - DNA, 31 bp, Synthetic construct
  source 1..31: mol_type = other DNA
agggattaatgacatacgcattttggggtac

SEQ ID NO: 428 - DNA, 36 bp, Synthetic construct
  source 1..36: mol_type = other DNA
gcggtaccccaaaatgcgtatgtcattaatccctgg

SEQ ID NO: 429 - DNA, 31 bp, Synthetic construct
  source 1..31: mol_type = other DNA
aggagattaatgacatacgcatttggggtac

SEQ ID NO: 430 - DNA, 36 bp, Synthetic construct
  source 1..36: mol_type = other DNA
gcggtaccccaaatgcgtatgtcattaatctcctgg

SEQ ID NO: 431 - DNA, 31 bp, Synthetic construct
  source 1..31: mol_type = other DNA
aggttcaatcctcttgtcacctgtggggtac

SEQ ID NO: 432 - DNA, 36 bp, Synthetic construct
  source 1..36: mol_type = other DNA
gcggtaccccacaggtgacaagaggattgaacctgg

SEQ ID NO: 433 - DNA, 31 bp, Synthetic construct
  source 1..31: mol_type = other DNA
agggacatctctatgtcggccactggggtac

SEQ ID NO: 434 - DNA, 36 bp, Synthetic construct
  source 1..36: mol_type = other DNA
gcggtaccccagtggccgacatagagatgtccctgg

SEQ ID NO: 435 - DNA, 31 bp, Synthetic construct
  source 1..31: mol_type = other DNA
aggaaaacctaccgcaagttgcctggggtac

SEQ ID NO: 436 - DNA, 36 bp, Synthetic construct
  source 1..36: mol_type = other DNA
gcggtaccccaggcaacttgcggtaggttttcctgg

SEQ ID NO: 437 - DNA, 31 bp, Synthetic construct
  source 1..31: mol_type = other DNA
aggtggactacatagttgtgtgatggggtac

SEQ ID NO: 438 - DNA, 36 bp, Synthetic construct
  source 1..36: mol_type = other DNA
gcggtaccccatcacacaactatgtagtccacctgg

SEQ ID NO: 439 - DNA, 31 bp, Synthetic construct
  source 1..31: mol_type = other DNA
aggctgccagactctctgaaccctggggtac

SEQ ID NO: 440 - DNA, 36 bp, Synthetic construct
  source 1..36: mol_type = other DNA
gcggtaccccagggttcagagagtctggcagcctgg

SEQ ID NO: 441 - DNA, 31 bp, Synthetic construct
  source 1..31: mol_type = other DNA
aggggatccctcgtataacaatatggggtac

SEQ ID NO: 442 - DNA, 36 bp, Synthetic construct
  source 1..36: mol_type = other DNA
gcggtaccccatattgttatacgagggatcccctgg

SEQ ID NO: 443 - DNA, 31 bp, Synthetic construct
  source 1..31: mol_type = other DNA
aggtatcgcatagtgcaaagaattggggtac

SEQ ID NO: 444 - DNA, 36 bp, Synthetic construct
  source 1..36: mol_type = other DNA
gcggtaccccaattctttgcactatgcgatacctgg

SEQ ID NO: 445 - DNA, 31 bp, Synthetic construct
  source 1..31: mol_type = other DNA
aggctcggtcctctcgtcacctgtggggtac

SEQ ID NO: 446 - DNA, 36 bp, Synthetic construct
  source 1..36: mol_type = other DNA
gcggtaccccacaggtgacgagaggaccgagcctgg

SEQ ID NO: 447 - DNA, 31 bp, Synthetic construct
  source 1..31: mol_type = other DNA
aggacgctcccggagagcgctggtggggtac

SEQ ID NO: 448 - DNA, 36 bp, Synthetic construct
  source 1..36: mol_type = other DNA
gcggtaccccaccagcgctctccgggagcgtcctgg

SEQ ID NO: 449 - DNA, 31 bp, Synthetic construct
  source 1..31: mol_type = other DNA
aggccatggcgcggtcagagggttggggtac

SEQ ID NO: 450 - DNA, 36 bp, Synthetic construct
  source 1..36: mol_type = other DNA
gcggtaccccaaccctctgaccgcgccatggcctgg

SEQ ID NO: 451 - DNA, 31 bp, Synthetic construct
  source 1..31: mol_type = other DNA
aggctcggctccaatgacgagggtggggtac

SEQ ID NO: 452 - DNA, 36 bp, Synthetic construct
  source 1..36: mol_type = other DNA
gcggtaccccaccctcgtcattggagccgagcctgg

SEQ ID NO: 453 - DNA, 31 bp, Synthetic construct
  source 1..31: mol_type = other DNA
aggcacttcgatgacctccccgttggggtac

SEQ ID NO: 454 - DNA, 36 bp, Synthetic construct
  source 1..36: mol_type = other DNA
gcggtaccccaacggggaggtcatcgaagtgcctgg

SEQ ID NO: 455 - DNA, 31 bp, Synthetic construct
  source 1..31: mol_type = other DNA
aggagtgggtgttcaggaccgcgtggggtac

SEQ ID NO: 456 - DNA, 36 bp, Synthetic construct
  source 1..36: mol_type = other DNA
gcggtaccccacgcggtcctgaacacccactcctgg

SEQ ID NO: 457 - DNA, 31 bp, Synthetic construct
  source 1..31: mol_type = other DNA
aggctgctacccacaggtgcaagtggggtac

SEQ ID NO: 458 - DNA, 36 bp, Synthetic construct
  source 1..36: mol_type = other DNA
gcggtaccccacttgcacctgtgggtagcagcctgg

SEQ ID NO: 459 - DNA, 31 bp, Synthetic construct
  source 1..31: mol_type = other DNA
aggaagatgtatcctggaatttatggggtac

SEQ ID NO: 460 - DNA, 36 bp, Synthetic construct
  source 1..36: mol_type = other DNA
gcggtaccccataaattccaggatacatcttcctgg

SEQ ID NO: 461 - DNA, 31 bp, Synthetic construct
  source 1..31: mol_type = other DNA
aggttttatcactacccaagacatggggtac

SEQ ID NO: 462 - DNA, 36 bp, Synthetic construct
  source 1..36: mol_type = other DNA
gcggtaccccatgtcttgggtagtgataaaacctgg

SEQ ID NO: 463 - DNA, 31 bp, Synthetic construct
  source 1..31: mol_type = other DNA
aggcggccaggctccctgaaacctggggtac

SEQ ID NO: 464 - DNA, 36 bp, Synthetic construct
  source 1..36: mol_type = other DNA
gcggtaccccaggtttcagggagcctggccgcctgg

SEQ ID NO: 465 - DNA, 31 bp, Synthetic construct
  source 1..31: mol_type = other DNA
aggactggtattataatacaacatggggtac

SEQ ID NO: 466 - DNA, 36 bp, Synthetic construct
  source 1..36: mol_type = other DNA
gcggtaccccatgttgtattataataccagtcctgg

SEQ ID NO: 467 - DNA, 31 bp, Synthetic construct
  source 1..31: mol_type = other DNA
aggtgagagtgacatttcgtacttggggtac

SEQ ID NO: 468 - DNA, 36 bp, Synthetic construct
  source 1..36: mol_type = other DNA
gcggtaccccaagtacgaaatgtcactctcacctgg

SEQ ID NO: 469 - DNA, 31 bp, Synthetic construct
  source 1..31: mol_type = other DNA
aggtgcgcccccgggtgaggtgctggggtac

SEQ ID NO: 470 - DNA, 36 bp, Synthetic construct
  source 1..36: mol_type = other DNA
gcggtaccccagcacctcacccgggggcgcacctgg

SEQ ID NO: 471 - DNA, 31 bp, Synthetic construct
  source 1..31: mol_type = other DNA
aggcagtttgagcagaaaaagaatggggtac

SEQ ID NO: 472 - DNA, 36 bp, Synthetic construct
  source 1..36: mol_type = other DNA
gcggtaccccattctttttctgctcaaactgcctgg

SEQ ID NO: 473 - DNA, 31 bp, Synthetic construct
  source 1..31: mol_type = other DNA
aggtctgtagagataagtgagtatggggtac

SEQ ID NO: 474 - DNA, 36 bp, Synthetic construct
  source 1..36: mol_type = other DNA
gcggtaccccatactcacttatctctacagacctgg

SEQ ID NO: 475 - DNA, 31 bp, Synthetic construct
  source 1..31: mol_type = other DNA
aggaagtgtgtacaaactcaagatggggtac

SEQ ID NO: 476 - DNA, 36 bp, Synthetic construct
  source 1..36: mol_type = other DNA
gcggtaccccatcttgagtttgtacacacttcctgg

SEQ ID NO: 477 - DNA, 31 bp, Synthetic construct
  source 1..31: mol_type = other DNA
aggtatcatggatgctgactttttggggtac

SEQ ID NO: 478 - DNA, 36 bp, Synthetic construct
  source 1..36: mol_type = other DNA
gcggtaccccaaaaagtcagcatccatgatacctgg

SEQ ID NO: 479 - DNA, 31 bp, Synthetic construct
  source 1..31: mol_type = other DNA
agggctttactagcccctgtggctggggtac

SEQ ID NO: 480 - DNA, 36 bp, Synthetic construct
  source 1..36: mol_type = other DNA
gcggtaccccagccacaggggctagtaaagccctgg

SEQ ID NO: 481 - DNA, 31 bp, Synthetic construct
  source 1..31: mol_type = other DNA
agggcacggaggagaaaagcaggtggggtac

SEQ ID NO: 482 - DNA, 36 bp, Synthetic construct
  source 1..36: mol_type = other DNA
gcggtaccccacctgcttttctcctccgtgccctgg

SEQ ID NO: 483 - DNA, 31 bp, Synthetic construct
  source 1..31: mol_type = other DNA
aggatttgtgagatctaaagaactggggtac

SEQ ID NO: 484 - DNA, 36 bp, Synthetic construct
  source 1..36: mol_type = other DNA
gcggtaccccagttctttagatctcacaaatcctgg

SEQ ID NO: 485 - DNA, 31 bp, Synthetic construct
  source 1..31: mol_type = other DNA
aggcgttgatgacattcgcattttggggtac

SEQ ID NO: 486 - DNA, 36 bp, Synthetic construct
  source 1..36: mol_type = other DNA
gcggtaccccaaaatgcgaatgtcatcaacgcctgg

SEQ ID NO: 487 - DNA, 31 bp, Synthetic construct
  source 1..31: mol_type = other DNA
aggcttatagatcatctaatacctggggtac

SEQ ID NO: 488 - DNA, 36 bp, Synthetic construct
  source 1..36: mol_type = other DNA
gcggtaccccaggtattagatgatctataagcctgg

SEQ ID NO: 489 - DNA, 31 bp, Synthetic construct
  source 1..31: mol_type = other DNA
aggtggcctccatagttgggtggtggggtac

SEQ ID NO: 490 - DNA, 36 bp, Synthetic construct
  source 1..36: mol_type = other DNA
gcggtaccccaccacccaactatggaggccacctgg

SEQ ID NO: 491 - DNA, 31 bp, Synthetic construct
  source 1..31: mol_type = other DNA
aggtactagagttcaaacatacttggggtac

SEQ ID NO: 492 - DNA, 36 bp, Synthetic construct
  source 1..36: mol_type = other DNA
gcggtaccccaagtatgtttgaactctagtacctgg

SEQ ID NO: 493 - DNA, 31 bp, Synthetic construct
  source 1..31: mol_type = other DNA
aggcccctgctctgagcactcggtggggtac

SEQ ID NO: 494 - DNA, 36 bp, Synthetic construct
  source 1..36: mol_type = other DNA
gcggtaccccaccgagtgctcagagcaggggcctgg

SEQ ID NO: 495 - DNA, 31 bp, Synthetic construct
  source 1..31: mol_type = other DNA
aggaggaccacatggcctgcctctggggtac

SEQ ID NO: 496 - DNA, 36 bp, Synthetic construct
  source 1..36: mol_type = other DNA
gcggtaccccagaggcaggccatgtggtcctcctgg

SEQ ID NO: 497 - DNA, 31 bp, Synthetic construct
  source 1..31: mol_type = other DNA
aggaacagctgccgcaaattgcctggggtac

SEQ ID NO: 498 - DNA, 36 bp, Synthetic construct
  source 1..36: mol_type = other DNA
gcggtaccccaggcaatttgcggcagctgttcctgg

SEQ ID NO: 499 - DNA, 31 bp, Synthetic construct
  source 1..31: mol_type = other DNA
aggctccgtattggctggcttcgtggggtac

SEQ ID NO: 500 - DNA, 36 bp, Synthetic construct
  source 1..36: mol_type = other DNA
gcggtaccccacgaagccagccaatacggagcctgg

SEQ ID NO: 501 - DNA, 31 bp, Synthetic construct
  source 1..31: mol_type = other DNA
aggggccagccgatgacaaatactggggtac

SEQ ID NO: 502 - DNA, 36 bp, Synthetic construct
  source 1..36: mol_type = other DNA
gcggtaccccagtatttgtcatcggctggcccctgg

SEQ ID NO: 503 - DNA, 31 bp, Synthetic construct
  source 1..31: mol_type = other DNA
agggacgtctctacgtcggcggctggggtac

SEQ ID NO: 504 - DNA, 36 bp, Synthetic construct
  source 1..36: mol_type = other DNA
gcggtaccccagccgccgacgtagagacgtccctgg

SEQ ID NO: 505 - DNA, 31 bp, Synthetic construct
  source 1..31: mol_type = other DNA
aggccggcgcgaggggccgagagtggggtac

SEQ ID NO: 506 - DNA, 36 bp, Synthetic construct
  source 1..36: mol_type = other DNA
gcggtaccccactctcggcccctcgcgccggcctgg

SEQ ID NO: 507 - DNA, 31 bp, Synthetic construct
  source 1..31: mol_type = other DNA
aggactgtttgtactttttcatctggggtac

SEQ ID NO: 508 - DNA, 36 bp, Synthetic construct
  source 1..36: mol_type = other DNA
gcggtaccccagatgaaaaagtacaaacagtcctgg

SEQ ID NO: 509 - DNA, 31 bp, Synthetic construct
  source 1..31: mol_type = other DNA
aggtgcttatgatgatgacattatggggtac

SEQ ID NO: 510 - DNA, 36 bp, Synthetic construct
  source 1..36: mol_type = other DNA
gcggtaccccataatgtcatcatcataagcacctgg

SEQ ID NO: 511 - DNA, 31 bp, Synthetic construct
  source 1..31: mol_type = other DNA
aggccgcgtccgattgatgtgattggggtac

SEQ ID NO: 512 - DNA, 36 bp, Synthetic construct
  source 1..36: mol_type = other DNA
gcggtaccccaatcacatcaatcggacgcggcctgg

SEQ ID NO: 513 - DNA, 31 bp, Synthetic construct
  source 1..31: mol_type = other DNA
aggctttatgagcaccttaggcttggggtac

SEQ ID NO: 514 - DNA, 36 bp, Synthetic construct
  source 1..36: mol_type = other DNA
gcggtaccccaagcctaaggtgctcataaagcctgg

SEQ ID NO: 515 - DNA, 31 bp, Synthetic construct
  source 1..31: mol_type = other DNA
aggggatccctcctatgaccatgtggggtac

SEQ ID NO: 516 - DNA, 36 bp, Synthetic construct
  source 1..36: mol_type = other DNA
gcggtaccccacatggtcataggagggatcccctgg

SEQ ID NO: 517 - DNA, 31 bp, Synthetic construct
  source 1..31: mol_type = other DNA
aggataaaggtgacctcttacgttggggtac

SEQ ID NO: 518 - DNA, 36 bp, Synthetic construct
  source 1..36: mol_type = other DNA
gcggtaccccaacgtaagaggtcacctttatcctgg

SEQ ID NO: 519 - DNA, 31 bp, Synthetic construct
  source 1..31: mol_type = other DNA
aggactgtggcttagggtccaggtggggtac

SEQ ID NO: 520 - DNA, 36 bp, Synthetic construct
  source 1..36: mol_type = other DNA
gcggtaccccacctggaccctaagccacagtcctgg

SEQ ID NO: 521 - DNA, 31 bp, Synthetic construct
  source 1..31: mol_type = other DNA
aggtgaggaaacccaggcggtcctggggtac

SEQ ID NO: 522 - DNA, 36 bp, Synthetic construct
  source 1..36: mol_type = other DNA
gcggtaccccaggaccgcctgggtttcctcacctgg

SEQ ID NO: 523 - DNA, 31 bp, Synthetic construct
  source 1..31: mol_type = other DNA
aggaaatgtatacaggcaattaatggggtac

SEQ ID NO: 524 - DNA, 36 bp, Synthetic construct
  source 1..36: mol_type = other DNA
gcggtaccccattaattgcctgtatacatttcctgg

SEQ ID NO: 525 - DNA, 31 bp, Synthetic construct
  source 1..31: mol_type = other DNA
aggttgaatcaagcatagtccgatggggtac

SEQ ID NO: 526 - DNA, 36 bp, Synthetic construct
  source 1..36: mol_type = other DNA
gcggtaccccatcggactatgcttgattcaacctgg

SEQ ID NO: 527 - DNA, 31 bp, Synthetic construct
  source 1..31: mol_type = other DNA
agggggctacggctcatgagatatggggtac

SEQ ID NO: 528 - DNA, 36 bp, Synthetic construct
  source 1..36: mol_type = other DNA
gcggtaccccatatctcatgagccgtagccccctgg

SEQ ID NO: 529 - DNA, 31 bp, Synthetic construct
  source 1..31: mol_type = other DNA
aggcgggccgcgattggcagttctggggtac

SEQ ID NO: 530 - DNA, 36 bp, Synthetic construct
  source 1..36: mol_type = other DNA
gcggtaccccagaactgccaatcgcggcccgcctgg

SEQ ID NO: 531 - DNA, 31 bp, Synthetic construct
  source 1..31: mol_type = other DNA
aggctgcgattatctaaatccattggggtac

SEQ ID NO: 532 - DNA, 36 bp, Synthetic construct
  source 1..36: mol_type = other DNA
gcggtaccccaatggatttagataatcgcagcctgg

SEQ ID NO: 533 - DNA, 31 bp, Synthetic construct
  source 1..31: mol_type = other DNA
aggagcttgattacatacgcatgtggggtac

SEQ ID NO: 534 - DNA, 36 bp, Synthetic construct
  source 1..36: mol_type = other DNA
gcggtaccccacatgcgtatgtaatcaagctcctgg

SEQ ID NO: 535 - DNA, 31 bp, Synthetic construct
  source 1..31: mol_type = other DNA
aggaatgtttgctggcctctaggtggggtac

SEQ ID NO: 536 - DNA, 36 bp, Synthetic construct
  source 1..36: mol_type = other DNA
gcggtaccccacctagaggccagcaaacattcctgg

SEQ ID NO: 537 - DNA, 31 bp, Synthetic construct
  source 1..31: mol_type = other DNA
agggaaagcctctgcgccgggggtggggtac

SEQ ID NO: 538 - DNA, 36 bp, Synthetic construct
  source 1..36: mol_type = other DNA
gcggtaccccacccccggcgcagaggctttccctgg

(19) United States
(12) Patent Application Publication
(10) Pub. No.: US 2025/0320477 A1
(43) Pub. Date: Oct. 16, 2025

(54) NOVEL RNA-PROGRAMMABLE ENDONUCLEASE SYSTEMS AND USES THEREOF

(71) Applicants: CRISPR Therapeutics AG, Zug (CH); BAYER HEALTHCARE LLC, Whippany, NJ (US)

(72) Inventors: Andre COHNEN, Titz-Hasselsweiler (DE); Moritz SCHMIDT, Koln (DE); Wayne COCO, Pulheim (DE); Michael Biag GAMALINDA, Leverkusen (DE); Ashish GUPTA, Leverkusen (DE); Christian PITZLER, Leverkusen (DE); Florian RICHTER, Leverkusen (DE); Jan TEBBE, Leverkusen (DE); Christopher CHENG, Cambridge, MA (US); Ryo TAKEUCHI, Cambridge, MA (US); Caroline W. REISS, Cambridge, MA (US)

(21) Appl. No.: 19/016,469

(22) Filed: Jan. 10, 2025

Related U.S. Application Data
(63) Continuation of application No. 16/982,433, filed on Sep. 18, 2020, now Pat. No. 12,203,110.
(60) Provisional application No. 62/745,239, filed on Oct. 12, 2018.
(60) Provisional application No. 62/745,238, filed on Oct. 12, 2018.
(60) Provisional application No. 62/745,246, filed on Oct. 12, 2018.
(60) Provisional application No. 62/745,240, filed on Oct. 12, 2018.

(30) Foreign Application Priority Data
Mar. 19, 2018 (EP) 18162681.3
Mar. 19, 2018 (EP) 18162683.9
May 16, 2018 (EP) 18172625.8
May 29, 2018 (EP) 18174707.2
Jul. 4, 2018 (EP) 18181680.2

(51) Int. Cl. C12N 9/22; C12N 15/11; C12N 15/113; C12N 15/90

(52) U.S. Cl. CPC C12N 9/22; C12N 15/11; C12N 15/113; C12N 15/907; C12N 2310/20; C12N 2800/80

(57) ABSTRACT

Aspects of this invention, inter alia, relate to novel systems for targeting, editing or manipulating DNA in a cell, using novel synthetic RNA-guided nucleases (sRGNs). The sRGNs are derived from wildtype or parental small type II CRISPR Cas9 endonucleases.

### CROSS-REFERENCE TO RELATED APPLICATIONS

The present application is a continuation of U.S. patent application Ser. No. 16/982,433 filed Sep. 18, 2020, allowed as U.S. Pat. No. 12,203,110, which is a 371 national stage filing of International Application No. PCT/US2019/023044 filed Mar. 19, 2019, which claims priority to European Patent Application No. 18162681.3, filed on Mar. 19, 2018, European Patent Application No. 18162683.9, filed on Mar. 19, 2018, European Patent Application No. 18172625.8, filed on May 16, 2018, European Patent Application No. 18174707.2, filed on May 16, 2018, European Patent Application No. 18181680.2, filed on Jul. 4, 2018, U.S. Patent Application No. 62/745,238, filed on Oct. 12, 2018, U.S. Patent Application No. 62/745,239, filed on Oct. 12, 2018, U.S. Patent Application No. 62/745,240, filed on Oct. 12, 2018, and U.S. Patent Application No. 62/745,246, filed on Oct. 12, 2018, the contents of each of which is herein expressly incorporated by reference in its entirety, including any drawings.

### SEQUENCE LISTING

The text of the computer readable sequence listing filed herewith, titled “CRISP_42656_306_SequenceListing.xml”, created Jan. 9, 2025, having a file size of 586,952 bytes, is hereby incorporated by reference in its entirety.

### FIELD

The present disclosure generally relates to the field of molecular biology, including compositions and methods relating to novel systems including RNA-programmable endonucleases, associated guide RNAs and/or target sequences, and methods for producing and using the same in various applications, including methods for modulating transcription, as well as methods for targeting, editing, and/or manipulating DNA in a cell.

### BACKGROUND

Endonucleases such as Zinc-finger endonucleases (ZFNs), transcription-activator like effector nucleases (TALENs), and ribonucleases have been harnessed as site-specific nucleases for genome targeting, genome editing, gene silencing, transcription modulation, promoting recombination and other molecular biological techniques. Clustered Regularly Interspaced Short Palindromic Repeats (CRISPR)-CRISPR associated proteins (Cas) systems provide a source of novel nucleases and endonucleases, including CRISPR-Cas9.

CRISPR systems are prokaryotic systems that provides the prokaryote with resistance to foreign genetic elements such as phage. As demonstrated in Streptococcus pyogenes, Cas9 guided by the duplex formed between mature activating tracr RNA and targeting crRNA introduces site-specific double-stranded DNA (dsDNA) breaks in the invading cognate DNA. Cas9 is a multi-domain enzyme that uses an HNH nuclease domain to cleave the target strand (defined as complementary to the spacer sequence of crRNA) and a RuvC-like domain to cleave the non-target strand, enabling the conversion of the dsDNA cleaving Cas9 into a nickase by selective motif inactivation. DNA cleavage specificity is determined by two parameters: the variable, spacer-derived sequence of crRNA targeting the protospacer sequence (the sequence on the DNA target that is non-complementary to the spacer of crRNA) and a short sequence, the Protospacer Adjacent Motif (PAM), located immediately 3′ (downstream) of the protospacer on the non-target DNA strand.

Editing genomes using the RNA-guided DNA targeting principle of CRISPR-Cas, as described in WO2013/176722, has been exploited widely over the past few years. Studies have demonstrated that RNA-guided Cas9 can be employed as a genome editing tool in human cells, mice, zebrafish, drosophila, worms, plants, yeast and bacteria, as well as various other species. The system is versatile, enabling multiplex genome engineering by programming Cas9 to edit several sites in a genome simultaneously by using multiple guide RNAs. The conversion of Cas9 into a nickase was shown to facilitate homology-directed repair in mammalian genomes with reduced mutagenic activity. In addition, the DNA-binding activity of a Cas9 catalytic inactive mutant has, for example, been exploited to engineer RNA-programmable transcriptional silencing and activating devices or epigenetic modifiers.

Despite the promise of CRISPR-Cas 9 systems for gene editing, a number of problems are associated with the use of the system. For example, they have one or more of the following disadvantages:
a) Their size is too large to be carried inside the genome of established therapeutically-suitable viral transfection systems such as adeno-associated viruses (AAVs).
b) Their activity in heterologous environments is generally too low for use in these environments, for example, too low for efficient use in eukaryotic, and in particular in mammalian environments.
c) Their nuclease activity lacks fidelity, leading to unwanted off-target effects that would, for example, make them unsuitable for gene therapeutic uses or other applications requiring high precision.
d) They may trigger an immune response that can limit their use for in vivo applications in mammals.
e) They require complex and/or long PAMs that restrict target selection for the DNA-targeting segments.
f) They exhibit poor expression from plasmid or viral vectors.

### SUMMARY

This section provides a general summary of the disclosure, and is not comprehensive of its full scope or all of its features.

In some embodiments, provided herein are compositions and methods relating to novel RNA-programmable endonucleases, and systems for using such nucleases. The invention also relates to other components including guide RNAs and/or target sequences, and methods for producing and using the same in various applications. Examples of such applications include methods for modulating transcription, as well as methods for targeting, editing, and/or manipulating DNA using the novel nucleases and other components such as nucleic acids and/or polypeptides. Some embodiments of the disclosure also relate to recombinant cells and kits comprising one or more of the system elements disclosed herein.

In one aspect, provided herein is a synthetic RNA-guided nuclease (sRGN) polypeptide comprising, from N-terminus to C-terminus, 1) mini-domain 1 comprising the amino acid sequence of any one of SEQ ID NOs: 34-37 or a variant thereof having at least about 90% sequence identity to any one of SEQ ID NOs: 34-37, 2) mini-domain 2 comprising the amino acid sequence of any one of SEQ ID NOs: 38-41 or a variant thereof having at least about 90% sequence identity to any one of SEQ ID NOs: 38-41, 3) mini-domain 3 comprising the amino acid sequence of any one of SEQ ID NOs: 42-45 or a variant thereof having at least about 90% sequence identity to any one of SEQ ID NOs: 42-45, 4) mini-domain 4 comprising the amino acid sequence of any one of SEQ ID NOs: 46-49 or a variant thereof having at least about 90% sequence identity to any one of SEQ ID NOs: 46-49, 5) mini-domain 5 comprising the amino acid sequence of any one of SEQ ID NOs: 50-53 or a variant thereof having at least about 90% sequence identity to any one of SEQ ID NOs: 50-53, 6) mini-domain 6 comprising the amino acid sequence of any one of SEQ ID NOs: 54-57 or a variant thereof having at least about 90% sequence identity to any one of SEQ ID NOs: 54-57, 7) mini-domain 7 comprising the amino acid sequence of any one of SEQ ID NOs: 58-61 or a variant thereof having at least about 90% sequence identity to any one of SEQ ID NOs: 58-61, and 8) mini-domain 8 comprising the amino acid sequence of any one of SEQ ID NOs: 62-65 or a variant thereof having at least about 90% sequence identity to any one of SEQ ID NOs: 62-65, wherein at least 2 of the mini-domains are derived from different parental Cas9 endonucleases selected from the group consisting of Staphylococcus lugdunensis Cas9 (SEQ ID NO: 30), Staphylococcus pasteuri Cas9 (SEQ ID NO: 31), Staphylococcus microti Cas9 (SEQ ID NO: 33), and Staphylococcus hyicus Cas9 (SEQ ID NO: 32). In some embodiments, at least 3 of the mini-domains are derived from different parental Cas9 endonucleases selected from the group consisting of Staphylococcus lugdunensis Cas9 (SEQ ID NO: 30), Staphylococcus pasteuri Cas9 (SEQ ID NO: 31), Staphylococcus microti Cas9 (SEQ ID NO: 33), and Staphylococcus hyicus Cas9 (SEQ ID NO: 32). In some embodiments, mini-domain 8 comprises the amino acid sequence of SEQ ID NO: 62 or a variant thereof having at least about 90% sequence identity to SEQ ID NO: 62. In some embodiments, mini-domain 8 comprises the amino acid sequence of SEQ ID NO: 63 or a variant thereof having at least about 90% sequence identity to SEQ ID NO: 63. In some embodiments, mini-domain 1 comprises the amino acid sequence of SEQ ID NO: 34 or a variant thereof having at least about 90% sequence identity to SEQ ID NO: 34.

In another aspect, provided herein is an sRGN polypeptide comprising, from N-terminus to C-terminus, 1) mini-domain 1 comprising the amino acid sequence of any one of SEQ ID NOs: 66-69 or a variant thereof having at least about 90% sequence identity to any one of SEQ ID NOs: 66-69, 2) mini-domain 2 comprising the amino acid sequence of any one of SEQ ID NOs: 70-73 or a variant thereof having at least about 90% sequence identity to any one of SEQ ID NOs: 70-73, 3) mini-domain 3 comprising the amino acid sequence of any one of SEQ ID NOs: 74-77 or a variant thereof having at least about 90% sequence identity to any one of SEQ ID NOs: 74-77, 4) mini-domain 4 comprising the amino acid sequence of any one of SEQ ID NOs: 78-81 or a variant thereof having at least about 90% sequence identity to any one of SEQ ID NOs: 78-81, 5) mini-domain 5 comprising the amino acid sequence of any one of SEQ ID NOs: 82-85 or a variant thereof having at least about 90% sequence identity to any one of SEQ ID NOs: 82-85, 6) mini-domain 6 comprising the amino acid sequence of any one of SEQ ID NOs: 86-89 or a variant thereof having at least about 90% sequence identity to any one of SEQ ID NOs: 86-89, 7) mini-domain 7 comprising the amino acid sequence of any one of SEQ ID NOs: 90-93 or a variant thereof having at least about 90% sequence identity to any one of SEQ ID NOs: 90-93, 8) mini-domain 8 comprising the amino acid sequence of any one of SEQ ID NOs: 94-97 or a variant thereof having at least about 90% sequence identity to any one of SEQ ID NOs: 94-97, 9) mini-domain 9 comprising the amino acid sequence of any one of SEQ ID NOs: 98-101 or a variant thereof having at least about 90% sequence identity to any one of SEQ ID NOs: 98-101, 10) mini-domain 10 comprising the amino acid sequence of any one of SEQ ID NOs: 102-105 or a variant thereof having at least about 90% sequence identity to any one of SEQ ID NOs: 102-105, 11) mini-domain 11 comprising the amino acid sequence of any one of SEQ ID NOs: 106-109 or a variant thereof having at least about 90% sequence identity to any one of SEQ ID NOs: 106-109, and 12) mini-domain 12 comprising the amino acid sequence of any one of SEQ ID NOs: 110-113 or a variant thereof having at least about 90% sequence identity to any one of SEQ ID NOs: 110-113, wherein at least 2 of the mini-domains are derived from different parental Cas9 endonucleases selected from the group consisting of Staphylococcus lugdunensis Cas9 (SEQ ID NO: 30), Staphylococcus pasteuri Cas9 (SEQ ID NO: 31), Staphylococcus microti Cas9 (SEQ ID NO: 33), and Staphylococcus hyicus Cas9 (SEQ ID NO: 32). In some embodiments, at least 3 of the mini-domains are derived from different parental Cas9 endonucleases selected from the group consisting of Staphylococcus lugdunensis Cas9 (SEQ ID NO: 30), Staphylococcus pasteuri Cas9 (SEQ ID NO: 31), Staphylococcus microti Cas9 (SEQ ID NO: 33), and Staphylococcus hyicus Cas9 (SEQ ID NO: 32). In some embodiments, mini-domain 12 comprises the amino acid sequence of SEQ ID NO: 110 or a variant thereof having at least about 90% sequence identity to SEQ ID NO: 110. In some embodiments, mini-domain 12 comprises the amino acid sequence of SEQ ID NO: 111 or a variant thereof having at least about 90% sequence identity to SEQ ID NO: 111. In some embodiments, (i) mini-domain 1 comprises the amino acid sequence of SEQ ID NO: 66 or a variant thereof having at least about 90% sequence identity to SEQ ID NO: 66, and (ii) mini-domain 2 comprises the amino acid sequence of SEQ ID NO: 70 or a variant thereof having at least about 90% sequence identity to SEQ ID NO: 70.

In another aspect, provided herein is an sRGN polypeptide selected from the group consisting of: a) a Gib11 polypeptide comprising the amino acid sequence of SEQ ID NO: 1 or a variant thereof having at least about 90% sequence identity to SEQ ID NO: 1, b) a Gib11Spa-1 polypeptide comprising the amino acid sequence of SEQ ID NO: 2 or a variant thereof having at least about 90% sequence identity to SEQ ID NO: 2, c) a Gib11Spa-2 polypeptide comprising the amino acid sequence of SEQ ID NO: 3 or a variant thereof having at least about 90% sequence identity to SEQ ID NO: 3, d) a Gib11Spa-3 polypeptide comprising the amino acid sequence of SEQ ID NO: 4 or a variant thereof having at least about 90% sequence identity to SEQ ID NO: 4, e) a P2H12 polypeptide comprising the amino acid sequence of SEQ ID NO: 5 or a variant thereof having at least about 90% sequence identity to SEQ ID NO: 5, f) an E2 polypeptide comprising the amino acid sequence of SEQ ID NO: 6 or a variant thereof having at least about 90% sequence identity to SEQ ID NO: 6, g) an E2+K741D+L743K polypeptide comprising the amino acid sequence of SEQ ID NO: 7 or a variant thereof having at least about 90% sequence identity to SEQ ID NO: 7, h) an E2+S670T+N675D polypeptide comprising the amino acid sequence of SEQ ID NO: 8 or a variant thereof having at least about 90% sequence identity to SEQ ID NO: 8, i) an E2+K741N+L743N polypeptide comprising the amino acid sequence of SEQ ID NO: 9 or a variant thereof having at least about 90% sequence identity to SEQ ID NO: 9, j) an F8 polypeptide comprising the amino acid sequence of SEQ ID NO: 10 or a variant thereof having at least about 90% sequence identity to SEQ ID NO: 10; k) an F8+K737D+L739K polypeptide comprising the amino acid sequence of SEQ ID NO: 11 or a variant thereof having at least about 90% sequence identity to SEQ ID NO: 11; and 1) an F8+K737N+L739N polypeptide comprising the amino acid sequence of SEQ ID NO: 12 or a variant thereof having at least about 90% sequence identity to SEQ ID NO: 12.

In some embodiments, the sRGN polypeptide is selected from the group consisting of: a) a Gib11 polypeptide comprising the amino acid sequence of SEQ ID NO: 1 or a variant thereof having at least about 90% sequence identity to SEQ ID NO: 1, b) a Gib11Spa-1 polypeptide comprising the amino acid sequence of SEQ ID NO: 2 or a variant thereof having at least about 90% sequence identity to SEQ ID NO: 2, and c) a Gib11Spa-3 polypeptide comprising the amino acid sequence of SEQ ID NO: 4 or a variant thereof having at least about 90% sequence identity to SEQ ID NO: 4. In some embodiments, the sRGN polypeptide is selected from the group consisting of: a) a Gib11 polypeptide comprising the amino acid sequence of SEQ ID NO: 1, b) a Gib11Spa-1 polypeptide comprising the amino acid sequence of SEQ ID NO: 2, and c) a Gib11Spa-3 polypeptide comprising the amino acid sequence of SEQ ID NO: 4.

In another aspect, provided herein is a nucleic acid encoding an sRGN polypeptide according to any of the embodiments described above. In some embodiments, the nucleic acid is codon-optimized for expression in a host cell. In some embodiments, the nucleic acid comprises the nucleotide sequence of any one of SEQ ID NOs: 13-29 or a variant thereof having at least about 90% sequence identity to any one of SEQ ID NOs: 13-29.

In another aspect, provided herein is a system comprising: (a) an sRGN polypeptide referenced above or a nucleic acid encoding the sRGN polypeptide; and (b) a guide RNA (gRNA) or nucleic acid encoding the gRNA, wherein the gRNA is capable of guiding the sRGN polypeptide or variant thereof to a target polynucleotide sequence. In some embodiments, the system further comprises a donor template comprising a heterologous polynucleotide sequence, wherein the heterologous polynucleotide sequence is capable of being inserted into the target polynucleotide sequence.

In some embodiments, according to any of the systems described above, the nucleic acid encoding the sRGN polypeptide is codon-optimized for expression in a host cell and/or the heterologous polynucleotide sequence is codon-optimized for expression in a host cell.

In some embodiments, according to any of the systems described above, the nucleic acid encoding the sRGN polypeptide is a deoxyribonucleic acid (DNA).

In some embodiments, according to any of the systems described above, the nucleic acid encoding the sRGN polypeptide is a ribonucleic acid (RNA). In some embodiments, the RNA encoding the sRGN polypeptide is an mRNA.

In some embodiments, according to any of the systems described above, the donor template is encoded in an Adeno Associated Virus (AAV) vector.

In some embodiments, according to any of the systems described above, the sRGN polypeptide or nucleic acid encoding the sRGN polypeptide is formulated in a liposome or lipid nanoparticle. In some embodiments, the liposome or lipid nanoparticle also comprises the gRNA or nucleic acid encoding the gRNA.

In some embodiments, according to any of the systems described above, the system comprises the sRGN polypeptide pre-complexed with the gRNA, forming a ribonucleoprotein (RNP) complex.

In another aspect, provided herein is a method of targeting, editing, modifying, or manipulating a target DNA at a target locus, the method comprising providing the following to the target DNA: (a) an sRGN polypeptide according to any of the embodiments described above or a nucleic acid encoding the sRGN polypeptide; and (b) a guide RNA (gRNA) or nucleic acid encoding the gRNA, wherein the gRNA is capable of guiding the sRGN polypeptide to the target locus. In some embodiments, the method further comprises providing to the target DNA a donor template comprising a heterologous polynucleotide sequence, wherein the heterologous polynucleotide sequence is capable of being inserted into the target locus.

In some embodiments, according to any of the methods described above, the nucleic acid encoding the sRGN polypeptide is codon-optimized for expression in a host cell and/or the heterologous polynucleotide sequence is codon-optimized for expression in a host cell.

In some embodiments, according to any of the methods described above, the nucleic acid encoding the sRGN polypeptide is a deoxyribonucleic acid (DNA).

In some embodiments, according to any of the methods described above, the nucleic acid encoding the sRGN polypeptide is a ribonucleic acid (RNA). In some embodiments, the RNA encoding the sRGN polypeptide is an mRNA.

In some embodiments, according to any of the methods described above, the donor template is encoded in an Adeno Associated Virus (AAV) vector.

In some embodiments, according to any of the methods described above, the sRGN polypeptide or nucleic acid encoding the sRGN polypeptide is formulated in a liposome or lipid nanoparticle. In some embodiments, the liposome or lipid nanoparticle also comprises the gRNA or nucleic acid encoding the gRNA. In some embodiments, the method comprises providing to the target DNA the sRGN polypeptide pre-complexed with the gRNA as an RNP complex.

In another aspect, provided herein is a modified cell comprising: (a) an sRGN polypeptide according to any of the embodiments described above or a nucleic acid encoding the sRGN polypeptide; and (b) a guide RNA (gRNA) or nucleic acid encoding the gRNA, wherein the gRNA is capable of guiding the sRGN polypeptide or variant thereof to a target polynucleotide sequence. In some embodiments, the modified cell further comprises a donor template comprising a heterologous polynucleotide sequence, wherein the heterologous polynucleotide sequence is capable of being inserted into the target polynucleotide sequence.

In another aspect, provided herein is a genetically modified cell in which the genome of the cell is edited by a method according to any of the embodiments described above.

In another aspect, provided herein is a kit comprising: (a) an sRGN polypeptide referenced above or a nucleic acid encoding the sRGN polypeptide; and (b) a guide RNA (gRNA) or nucleic acid encoding the gRNA, wherein the gRNA is capable of guiding the sRGN polypeptide or variant thereof to a target polynucleotide sequence. In some embodiments, the kit further comprises a donor template comprising a heterologous polynucleotide sequence, wherein the heterologous polynucleotide sequence is capable of being inserted into the target polynucleotide sequence.

### BRIEF DESCRIPTION OF THE DRAWINGS

FIG. 1 shows a graphic representation of the gene family shuffling technique used to generate Gib11, P2H12, E2, and F8.

FIG. 2A shows the results of a fluorescence polarization-based biochemical activity in the disruption assay (in some cases, activity exceeding that of a wild type Cas9 such as a SluCas9), Fluorescence polarization assay for Gib11. Diamonds depict the condition with sgRNA and boxes depict the control condition without sgRNA.

FIG. 2B shows the results of a fluorescence polarization-based biochemical cleavage assay for SluCas9. Diamonds depict the condition with sgRNA and boxes depict the control condition without sgRNA.

FIG. 2C shows the relative activity of Gib11 and SluCas9 in the cleavage assay, as determined by the initial slope of the curves for the conditions with sgRNA shown in FIGS. 2A and 2B, respectively.

FIG. 3 shows results for recognition of the “NNGG” PAM motif by Gib11, as determined by a “live-dead” assay in liquid culture.

FIG. 4 shows the results of a fluorescence polarization-based biochemical cleavage assay for P2H12. Diamonds depict the condition with sgRNA and boxes depict the control condition without sgRNA.

FIG. 5 shows the results of a BFP disruption-based cleavage assay for E2.

FIG. 6 shows the predicted domain structure of Gib11Spa-1, Gib11Spa-2, and Gib11Spa-3 based on the domain organization of the Staphylococcus aureus protein The bracket named PI denotes the PI domain of the underlying Gib11 sequence. The corresponding cRGN-A portion is shown as a hatched box (in this case for Gib11) and the cRGN-B Staphylococcus pasteuri portion is shown as a white box. Amino acid positions are in reference to the Gib11 protein (SEQ ID NO: 1).

FIG. 7 shows the results of a BFP disruption-based cleavage assay for Gib11Spa-1, Gib11Spa-2, and Gib11Spa-3.

FIG. 8 shows the results of a BFP disruption-based cleavage assay for F8.

FIG. 9 shows specificity profiles of Gib11, Gib11Spa-1, Gib11Spa-3, E2, and F8, as assessed using a biochemical cleavage assay.

FIG. 10 shows the overall specificity of Gib11, Gib11Spa-1, Gib11Spa-3, E2, and F8 based on their specificity profiles.

FIG. 11 shows the distribution of activity profiles for Gib11, Gib11Spa-1, Gib11Spa-3, E2, and F8, as assessed using a biochemical cleavage assay. On the x-axis, going from left to right, are the target DNA substrates sorted by highest to lowest activity for each indicated nuclease.

### DETAILED DESCRIPTION OF THE INVENTION

Most existing type II CRISPR Cas systems are based on the enzyme from Streptococcus pyogenes, which has the particular disadvantage of being too large for packaging into viral vectors as AAV (1638 amino acids). There is an alternative type II CRISPR Cas system based on the nuclease from Staphylococcus aureus (EP 2 898 075) which is significantly smaller in size. However, this nuclease requires a complex PAM which greatly restricts its use for gene editing applications. Furthermore, small nucleases can be more useful for gene editing methods, however, a limited number of such small nucleases have been identified and their optimal features for gene editing are, in at least most cases, not sufficiently defined for use in gene editing.

Provided herein are novel synthetic CRISPR-Cas polypeptides (also referred to herein as “sRGN polypeptides”) derived from artificially defined mini-domains of CRISPR-Cas endonucleases of four different Staphylococcus species (Staphylococcus lugdunensis, Staphylococcus pasteuri, Staphylococcus microti, and Staphylococcus hyicus), and variants thereof having different and advantageous characteristics and functionalities. These sRGNs provides further opportunities for genome editing that previously did not exist. Cas nucleases can have differing activities depending on the system in which they are used. One feature of the present invention is providing additional engineered nucleases that increase the toolbox of nucleases available for gene editing and related methods.

In many cases, naturally occurring, known Cas nucleases target a longer PAM, such as NNAAAA, which may restrict the utility of those nucleases. The present invention provides synthetic nucleases (e.g., small nucleases) that are derived from naturally-occurring small nucleases, but can recognize an NNGG PAM.

The present invention further provides suitable PAM sequences and suitable guides, including single-guide-RNAs (sgRNAs), for use in prokaryotic, eukaryotic, and in vitro environments.

The sRGN polypeptides provided herein can exhibit advantageous characteristics over the already reported CRISPR-Cas endonucleases, such as, for example, a higher activity in prokaryotic, eukaryotic, and/or in vitro environments, and/or greater expression of the sRGN polypeptide from a nucleic acid in eukaryotic environments, such as, e.g., a human host cell. In some cases, an sRGN combines one or more of a small size, a high editing activity, and the requirement of only a short PAM sequence. A small size in reference to an RNA-guided nuclease means a nuclease that is no greater than about 1100 amino acids in length.

Also provided are systems for targeting, editing or manipulating DNA in a cell, comprising an sRGN polypeptide or nucleic acid encoding the sRGN polypeptide, and one or more guide RNAs (gRNAs), e.g., one or more single guide RNAs (sgRNAs), or nucleic acid encoding the one or more gRNAs.

In the following detailed description, reference is made to the accompanying drawings, which form a part hereof. In the drawings, similar symbols typically identify similar components, unless context dictates otherwise. The illustrative alternatives described in the detailed description, drawings, and claims are not meant to be limiting. Other alternatives may be used, and other changes may be made, without departing from the spirit or scope of the subject matter presented here. It will be readily understood that the aspects, as generally described herein, and illustrated in the Figures, can be arranged, substituted, combined, and designed in a wide variety of different configurations, all of which are explicitly contemplated and make part of this application.

Unless otherwise defined, all terms of art, notations and other scientific terms or terminology used herein are intended to have the meanings commonly understood by those of skill in the art to which this application pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

### Definitions

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids/triple helices, or a polymer comprising purine and pyrimidine bases or other natural, chemically or biochemically modified, non-natural, or derivatized nucleotide bases.

“Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” and “nucleic acid” should be understood to include, as applicable to the embodiments being described, single-stranded (such as sense or antisense) and double-stranded polynucleotides.

“Genomic DNA” refers to the DNA of a genome of an organism including, but not limited to, the DNA of the genome of a bacterium, fungus, archea, protists, viral, plant or animal.

“Manipulating” DNA encompasses binding, nicking one strand, or cleaving (i.e., cutting) both strands of the DNA, or encompasses modifying or editing the DNA or a polypeptide associated with the DNA. Manipulating DNA can silence, activate, or modulate (either increase or decrease) the expression of an RNA or polypeptide encoded by the DNA, or prevent or enhance the binding of a polypeptide to DNA.

A “stem-loop structure” refers to a nucleic acid having a secondary structure that includes a region of nucleotides which are known or predicted to form a double-strand (stem portion) that is linked on one side by a region of predominantly single-stranded nucleotides (loop portion). The terms “hairpin” and “fold-back” structures are also used herein to refer to stem-loop structures. Such structures are well known in the art and these terms are used consistently with their known meanings in the art. As is known in the art, a stem-loop structure does not require exact base-pairing. Thus, the stem may include one or more base mismatches. Alternatively, the base-pairing may be exact, i.e., not include any mismatches.

By “hybridizable” or “complementary” or “substantially complementary” it is meant that a nucleic acid (e.g., RNA) comprises a sequence of nucleotides that enables it to non-covalently bind, i.e., form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine (A) pairing with thymidine (T), adenine (A) pairing with uracil (U), and guanine (G) pairing with cytosine (C) [DNA, RNA]. In addition, it is also known in the art that for hybridization between two RNA molecules (e.g., dsRNA), guanine (G) base pairs with uracil (U). For example, G/U base-pairing is partially responsible for the degeneracy (i.e., redundancy) of the genetic code in the context of tRNA anti-codon base-pairing with codons in mRNA. In the context of this disclosure, a guanine (G) of a protein-binding segment (dsRNA duplex) of a guide RNA molecule is considered complementary to a uracil (U), and vice versa. As such, when a G/U base-pair can be made at a given nucleotide position a protein-binding segment (dsRNA duplex) of a guide RNA molecule, the position is not considered to be non-complementary, but is instead considered to be complementary.

Hybridization and washing conditions are well known and exemplified in Sambrook, J. and Russell, W., Molecular Cloning: A Laboratory Manual, Third Edition, Cold Spring Harbor Laboratory Press, Cold Spring Harbor (2001); and Green, M. R., and Sambrook, J., Molecular cloning: A Laboratory Manual, Fourth Edition, Cold Spring Harbor Laboratory Press, Cold Spring Harbor (2012). The conditions of temperature and ionic strength determine the “stringency” of the hybridization.

Hybridization requires that the two nucleic acids contain complementary sequences, although mismatches between bases are possible. The conditions appropriate for hybridization between two nucleic acids depend on the length of the nucleic acids and the degree of complementation, variables well known in the art. The greater the degree of complementation between two nucleotide sequences, the greater the value of the melting temperature (Tm) for hybrids of nucleic acids having those sequences. For hybridizations between nucleic acids with short stretches of complementarity (e.g., complementarity over 35 or less, 30 or less, 25 or less, 22 or less, 20 or less, or 18 or less nucleotides) the position of mismatches becomes important (see Sambrook et al., supra, 11.7-11.8). Generally, the length for a hybridizable nucleic acid is at least 10 nucleotides. Illustrative minimum lengths for a hybridizable nucleic acid are: at least 15 nucleotides; at least 20 nucleotides; at least 22 nucleotides; at least 25 nucleotides; and at least 30 nucleotides). Furthermore, the skilled artisan will recognize that the temperature and wash solution salt concentration may be adjusted as necessary according to factors such as length of the region of complementation and the degree of complementation.

It is understood in the art that the sequence of polynucleotide need not be 100% complementary to that of its target nucleic acid to be specifically hybridizable. Moreover, a polynucleotide may hybridize over one or more segments such that intervening or adjacent segments are not involved in the hybridization event (e.g., a loop structure or hairpin structure). A polynucleotide can comprise at least 70%, at least 80%, at least 90%, at least 95%, at least 99%, or 100% sequence complementarity to a target region within the target nucleic acid sequence to which they are targeted. For example, an antisense nucleic acid in which 18 of 20 nucleotides of the antisense compound are complementary to a target region, and would therefore specifically hybridize, would represent 90 percent complementarity. In this example, the remaining non complementary nucleotides may be clustered or interspersed with complementary nucleotides and need not be contiguous to each other or to complementary nucleotides. Percent complementarity between particular stretches of nucleic acid sequences within nucleic acids can be determined routinely using BLAST programs (basic local alignment search tools) and PowerBLAST programs known in the art (Altschul et al., J. Mol. Biol. 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math. 1981 (2) 482-489).

The terms “peptide”, “polypeptide”, and “protein” are used interchangeably herein, and refer to a polymeric form of amino acids of any length, which can include coded and non-coded amino acids, chemically or biochemically modified or derivatized amino acids, and polypeptides having modified peptide backbones.

“Binding” as used herein (e.g., with reference to an RNA-binding domain of a polypeptide) refers to a non-covalent interaction between macromolecules (e.g., between a protein and a nucleic acid). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence-specific. Binding interactions are generally characterized by a dissociation constant (Kd) of less than 10−6 M, less than 10−7 M, less than 10−8 M, less than 10−9 M, less than 10−10 M, less than 10−11 M, less than 10−12 M, less than 10−13 M, less than 10−14 M, or less than 10−15 M. “Affinity” refers to the strength of binding, increased binding affinity being correlated with a lower Kd.

By “binding domain” it is meant a protein domain that is able to bind non-covalently to another molecule. A binding domain can bind to, for example, a DNA molecule (a DNA-binding protein), an RNA molecule (an RNA-binding protein) and/or a protein molecule (a protein-binding protein). In the case of a protein domain-binding protein, it can bind to itself (to form homo-dimers, homo-trimers, etc.) and/or it can bind to one or more molecules of a different protein or proteins.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine, and asparagine-glutamine.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different manners. To determine sequence identity, sequences can be aligned using various methods and computer programs (e.g., BLAST, T-COFFEE, MUSCLE, MAFFT, etc.), available over the world wide web at sites including ncbi.nlm.nili.gov/BLAST, ebi.ac.uk/Tools/msa/tcoffee, ebi.Ac.Uk!Tools/msa/muscle, mafft.cbrc/alignment/software. See, e.g., Altschul et al. (1990), J. Mol. Biol. 215:403-10. Sequence alignments standard in the art are used according to the invention to determine amino acid residues in one sRGN variant that “correspond to” amino acid residues in another sRGN variant. The amino acid residues of sRGN variants that correspond to amino acid residues of other sRGN variants appear at the same position in alignments of the sequences.

A DNA sequence that “encodes” a particular RNA is a DNA nucleic acid sequence that is transcribed into RNA. A DNA polynucleotide may encode an RNA (mRNA) that is translated into protein, or a DNA polynucleotide may encode an RNA that is not translated into protein (e.g., tRNA, rRNA, or a guide RNA; also called “non-coding” RNA or “ncRNA”). A “protein coding sequence” or a sequence that encodes a particular protein or polypeptide, is a nucleic acid sequence that is transcribed into mRNA (in the case of DNA) and is translated (in the case of mRNA) into a polypeptide in vitro or in vivo when placed under the control of appropriate regulatory sequences. The boundaries of the coding sequence are determined by a start codon at the 5′ terminus (N-terminus) and a translation stop nonsense codon at the 3′ terminus (C-terminus). A coding sequence can include, but is not limited to, cDNA from prokaryotic or eukaryotic mRNA, genomic DNA sequences from prokaryotic or eukaryotic DNA, and synthetic nucleic acids. A transcription termination sequence will usually be located at 3′ of the coding sequence.

As used herein, a “promoter sequence” or “promoter” is a DNA regulatory region capable of binding RNA polymerase and initiating transcription of a downstream (3′ direction) coding or non-coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains responsible for the binding of RNA polymerase. Eukaryotic promoters will often, but not always, contain “TATA” boxes and “CAAT” boxes. Various promoters, including inducible promoters, may be used to drive the various vectors of the present invention. A promoter can be a constitutively active promoter (i.e., a promoter that is constitutively in an active “ON” state), it may be an inducible promoter (i.e., a promoter whose state, active/“ON” or inactive/“OFF”, is controlled by an external stimulus, e.g., the presence of a particular temperature, compound, or protein), it may be a spatially restricted promoter (i.e., transcriptional control element, enhancer, etc.) (e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., hair follicle cycle in mice). Suitable promoters can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6) (Miyagishi et al., Nature Biotechnology 20, 497-500 (2002)), an enhanced U6 promoter (e.g., Xia et al., Nucleic Acids Res. 2003 Sep. 1; 31 (17)), a human H1 promoter (H1), and the like. Examples of inducible promoters include, but are not limited to T7 RNA polymerase promoter, T3 RNA polymerase promoter, isopropyl-beta-D-thiogalactopyranoside (IPTG)-regulated promoter, lactose induced promoter, heat shock promoter, Tetracycline-regulated promoter, Steroid-regulated promoter, Metal-regulated promoter, estrogen receptor-regulated promoter, etc. Inducible promoters can therefore be regulated by molecules including, but not limited to, doxycycline; RNA polymerase, e.g., T7 RNA polymerase; an estrogen receptor; an estrogen receptor fusion; etc.

In some embodiments, the promoter is a spatially restricted promoter (i.e., cell type specific promoter, tissue specific promoter, etc.) such that in a multi-cellular organism, the promoter is active (i.e., “ON”) in a subset of specific cells. Spatially restricted promoters may also be referred to as enhancers, transcriptional control elements, control sequences, etc. Any convenient spatially restricted promoter may be used and the choice of suitable promoter (e.g., a brain specific promoter, a promoter that drives expression in a subset of neurons, a promoter that drives expression in the germline, a promoter that drives expression in the lungs, a promoter that drives expression in muscles, a promoter that drives expression in islet cells of the pancreas, etc.) will depend on the organism. For example, various spatially restricted promoters are known for plants, flies, worms, mammals, mice, etc. Thus, a spatially restricted promoter can be used to regulate the expression of a nucleic acid encoding an sRGN polypeptide in a wide variety of different tissues and cell types, depending on the organism. Some spatially restricted promoters are also temporally restricted such that the promoter is in the “ON” state or “OFF” state during specific stages of embryonic development or during specific stages of a biological process (e.g., hair follicle cycle in mice). For illustration purposes, examples of spatially restricted promoters include, but are not limited to, neuron-specific promoters, adipocyte-specific promoters, cardiomyocyte-specific promoters, smooth muscle-specific promoters, photoreceptor-specific promoters, etc. Neuron-specific spatially restricted promoters include, but are not limited to, a neuron-specific enolase (NSE) promoter (e.g., EMBL HSENO2, X51956); an aromatic amino acid decarboxylase (AADC) promoter; a neurofilament promoter (e.g., GenBank HUMNFL, L04147); a synapsin promoter (e.g., GenBank HUMSYNIB, M55301); a thy-1 promoter (e.g., Chen et al. (1987) Cell 51:7-19; and Llewellyn, et al. (2010) Nat. Med. 16 (10): 1161-1166); a serotonin receptor promoter (e.g., GenBank S62283); a tyrosine hydroxylase promoter (TH) (e.g., Oh et al. (2009) Gene Ther. 16:437; Sasaoka et al. (1992) Mol. Brain Res. 16:274; Boundy et al. (1998) J. Neurosci. 18:9989; and Kaneda et al. (1991) Neuron 6:583-594); a GnRH promoter (e.g., Radovick et al. (1991) Proc. Natl. Acad. Sci. USA 88:3402-3406); an L7 promoter (e.g., Oberdick et al. (1990) Science 248:223-226); a DNMT promoter (e.g., Bartge et al. (1988) Proc. Natl. Acad. Sci. USA 85:3648-3652); an enkephalin promoter (e.g., Comb et al. (1988) EMBO J. 17:3793-3805); a myelin basic protein (MBP) promoter; a Ca2+-calmodulin-dependent protein kinase 11-alpha (CamKIM) promoter (e.g., Mayford et al. (1996) Proc. Natl. Acad. Sci. USA 93:13250; and Casanova et al. (2001) Genesis 31:37); a CMV enhancer/platelet-derived growth factor-p promoter (e.g., Liu et al. (2004) Gene Therapy 11:52-60); and the like.

Adipocyte-specific spatially restricted promoters include, but are not limited to aP2 gene promoter/enhancer, e.g., a region from −5.4 kb to +21 bp of a human aP2 gene (e.g., Tozzo et al. (1997) Endocrinol. 138:1604; Ross et al. (1990) Proc. Natl. Acad. Sci. USA 87:9590; and Pavjani et al. (2005) Nat. Med. 11:797); a glucose transporter-4 (GLUT4) promoter (e.g., Knight et al. (2003) Proc. Natl. Acad. Sci. USA 100:14725); a fatty acid translocase (FAT/CD36) promoter (e.g., Kuriki et al. (2002) Biol. Pharm. Bull. 25:1476; and Sato et al. (2002) J. Biol. Chem. 277:15703); a stearoyi-CoA desaturase-1 (SCD1) promoter (Tabor et. al.. (1999) J. Biol. Chem. 274:20603); a leptin promoter (e.g., Mason et al. (1998) Endocrinol. 139:1013; and Chen et al. (1999) Biochem. Biophys. Res. Comm. 262:187); an adiponectin promoter (e.g., Kita et al. (2005) Biochem. Biophys. Res. Comm. 331:484; and Chakrabarti (2010) Endocrinol. 151:2408); an adipsin promoter (e.g., Platt et al. (1989) Proc. Natl. Acad. Sci. USA 86:7490); a resistin promoter (e.g., Seo et al. (2003) Molec. Endocrinol. 17:1522); and the like.

Cardiomyocyte-specific spatially restricted promoters include, but are not limited to control sequences derived from the following genes: myosin light chain-2, a-myosin heavy chain, AE3, cardiac troponin C, cardiac actin, and the like. Franz et al. (1997) Cardiovasc. Res. 35:560-566; Robbins et al. (1995) Ann. N.Y. Acad. Sci. 752:492-505; Linn et al. (1995) Circ. Res. 76:584591; Parmacek et al. (1994) Mol. Cell. Biol. 14:1870-1885; Hunter et. al.. (1993) Hypertension 22:608-617; and Sartorelli et al. (1992) Proc. Natl. Acad. Sci. USA 89:4047-4051.

Smooth muscle-specific spatially restricted promoters include, but are not limited to an SM22a promoter (e.g., Akyiirek et al. (2000) Mol. Med. 6:983; and U.S. Pat. No. 7,169,874); a smoothelin promoter (e.g., WO 2001/018048); an a-smooth muscle actin promoter; and the like. For example, a 0.4 kb region of the SM22a promoter, within which lie two CArG elements, has been shown to mediate vascular smooth muscle cell-specific expression (e.g., Kim, et al. (1997) Mol. Cell. Biol. 17, 2266-2278; Li, et al., (1996) J. Cell Biol. 132, 849-859; and Moessler, et al. (1996) Development 122, 2415-2425).

Photoreceptor-specific spatially restricted promoters include, but are not limited to, a rhodopsin promoter; a rhodopsin kinase promoter (Young et al. (2003) Ophthalmol. Vis. Sci. 44:4076); a beta phosphodiesterase gene promoter (Nicoud et al. (2007) J. Gene Med. 9:1015); a retinitis pigmentosa gene promoter (Nicoud et al. (2007) supra); an interphotoreceptor retinoid-binding protein (IRBP) gene enhancer (Nicoud et al. (2007) supra); an IRBP gene promoter (Yokoyama et al. (1992) Exp Eye Res. 55:225); and the like.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence (e.g., guide RNA) or a coding sequence (e.g., sRGN polypeptide) and/or regulate translation of an encoded polypeptide.

The term “naturally-occurring” or “unmodified” as used herein as applied to a nucleic acid, a polypeptide, a cell, or an organism, refers to a nucleic acid, polypeptide, cell, or organism that is found in nature. For example, a polypeptide or polynucleotide sequence that is present in an organism (including viruses) that can be isolated from a source in nature and which has not been intentionally modified by a human in the laboratory is naturally occurring.

The term “chimeric” as used herein as applied to a nucleic acid or polypeptide refers to one entity that is composed of structures derived from different sources. For example, where “chimeric” is used in the context of a chimeric polypeptide (e.g., a chimeric sRGN polypeptide), the chimeric polypeptide includes amino acid sequences that are derived from different polypeptides, such as polypeptides that have a function other than a Cas nuclease, including, for example, polypeptides that have DNA-modifying activity, transcription factor activity, and/or DNA-associated polypeptide-modifying activity. A chimeric polypeptide may comprise either modified or naturally-occurring polypeptide sequences (e.g., a first amino acid sequence from a modified or unmodified sRGN protein; and a second amino acid sequence other than the sRGN protein). Similarly, “chimeric” in the context of a nucleic acid encoding a chimeric polypeptide includes nucleotide sequences derived from different coding regions (e.g., a first nucleotide sequence encoding a modified or unmodified sRGN protein; and a second nucleotide sequence encoding a polypeptide other than an sRGN protein).

The term “chimeric polypeptide” refers to a polypeptide which is not naturally occurring, e.g., is made by the artificial combination (i.e., “fusion”) of two or more otherwise separated segments of amino sequence through human intervention. A polypeptide that comprises a chimeric amino acid sequence is a chimeric polypeptide. Some chimeric polypeptides can be referred to as “fusion variants.”

“Heterologous,” as used herein, means a nucleotide or peptide that is not found in the native nucleic acid or protein, respectively. For example, in a chimeric sRGN protein, the RNA-binding domain of a naturally-occurring bacterial sRGN polypeptide (or a variant thereof) may be fused to a heterologous polypeptide sequence (i.e., a polypeptide sequence from a protein other than sRGN or a polypeptide sequence from another organism). The heterologous polypeptide may exhibit an activity (e.g., enzymatic activity) that will also be exhibited by the chimeric sRGN protein (e.g., methyltransferase activity, acetyltransferase activity, kinase activity, ubiquitinating activity, etc.). A heterologous nucleic acid may be linked to a naturally-occurring nucleic acid (or a variant thereof) (e.g., by genetic engineering) to generate a chimeric nucleic acid encoding a chimeric polypeptide. As another example, in a fusion variant sRGN site-directed polypeptide, a variant sRGN site-directed polypeptide may be fused to a heterologous polypeptide (i.e., a polypeptide other than sRGN), which exhibits an activity that will also be exhibited by the fusion variant sRGN site-directed polypeptide. A heterologous nucleic acid may be linked to a variant sRGN site-directed polypeptide (e.g., by genetic engineering) to generate a nucleic acid encoding a fusion variant sRGN site-directed polypeptide. “Heterologous,” as used herein, additionally means a nucleotide or polypeptide in a cell that is not its native cell.

The term “cognate” refers to two biomolecules that normally interact or co-exist in nature.

“Recombinant,” as used herein, means that a particular nucleic acid (DNA or RNA) or vector is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “DNA regulatory sequences”, below). Alternatively, DNA sequences encoding RNA (e.g., guide RNA) that is not translated may also be considered recombinant. Thus, e.g., the term “recombinant” nucleic acid refers to one which is not naturally occurring, e.g., is made by the artificial combination of two otherwise separated segments of sequence through human intervention. This artificial combination can be accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, it is performed to join together nuclei acid segments of desired functions to generate a desired combination of functions. This artificial combination can be accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence. Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.). Thus, a “recombinant” polypeptide is the result of human intervention, but may be a naturally occurring amino acid sequence. The term “non-naturally occurring” includes molecules that are markedly different from their naturally occurring counterparts, including chemically modified or mutated molecules.

A “vector” or “expression vector” is a replicon, such as plasmid, phage, virus, or cosmid, to which another DNA segment, i.e., an “insert”, may be attached so as to bring about the replication of the attached segment in a cell.

An “expression cassette” comprises a DNA coding sequence operably linked to a promoter. “Operably linked” refers to a juxtaposition wherein the components so described are in a relationship permitting them to function in their intended manner. For instance, a promoter is operably linked to a coding sequence if the promoter affects its transcription or expression. The terms “recombinant expression vector,” or “DNA construct” are used interchangeably herein to refer to a DNA molecule comprising a vector and at least one insert. Recombinant expression vectors are usually generated for the purpose of expressing and/or propagating the insert(s), or for the construction of other recombinant nucleotide sequences. The nucleic acid(s) may or may not be operably linked to a promoter sequence and may or may not be operably linked to DNA regulatory sequences.

A cell has been “genetically modified” or “transformed” or “transfected” by exogenous DNA, e.g., a recombinant expression vector, when such DNA has been introduced inside the cell. The presence of the exogenous DNA results in permanent or transient genetic change. The transforming DNA may or may not be integrated (covalently linked) into the genome of the cell.

In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

Suitable methods of genetic modification (also referred to as “transformation”) include but are not limited to, e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (e.g., Panyam et al., Adv Drug Deliv Rev. 2012 Sep. 13. pp: S0169-409X(12) 00283-9. doi: 10.1016/j.addr.2012.09.023), and the like.

The choice of method of genetic modification is generally dependent on the type of cell being transformed and the circumstances under which the transformation is taking place (e.g., in vitro, ex vivo, or in vivo). A general discussion of these methods can be found in Ausubel, et al., Short Protocols in Molecular Biology, 3rd ed., Wiley & Sons, 1995.

A “host cell,” as used herein, denotes an in vivo or in vitro eukaryotic cell, a prokaryotic cell (e.g., bacterial or archaeal cell), or a cell from a multicellular organism (e.g., a cell line) cultured as a unicellular entity, which eukaryotic or prokaryotic cells can be, or have been, used as recipients for a nucleic acid, and include the progeny of the original cell which has been transformed by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a bacterial host cell is a genetically modified bacterial host cell by virtue of introduction into a suitable bacterial host cell of an exogenous nucleic acid (e.g., a plasmid or recombinant expression vector) and a eukaryotic host cell is a genetically modified eukaryotic host cell (e.g., a mammalian germ cell), by virtue of introduction into a suitable eukaryotic host cell of an exogenous nucleic acid.

A “target DNA” as used herein is a DNA polynucleotide that comprises a “target site” or “target sequence.” The terms “target site,” “target sequence,” “target protospacer DNA,” or “protospacer-like sequence” are used interchangeably herein to refer to a nucleic acid sequence present in a target DNA to which a DNA-targeting segment of a guide RNA will bind, provided sufficient conditions for binding exist. For example, the target site (or target sequence) 5′-GAGCATATC-3′ within a target DNA is targeted by (or is bound by, or hybridizes with, or is complementary to) the RNA sequence 5′-GAUAUGCUC-3′. Suitable DNA/RNA binding conditions include physiological conditions normally present in a cell. Other suitable DNA/RNA binding conditions (e.g., conditions in a cell-free system) are known in the art; e.g., Sambrook, supra. The strand of the target DNA that is complementary to and hybridizes with the guide RNA is referred to as the “complementary strand” and the strand of the target DNA that is complementary to the “complementary strand” (and is therefore not complementary to the guide RNA) is referred to as the “non-complementary strand” or “non-complementary strand.” By “site-directed modifying polypeptide” or “RNA-binding site-directed polypeptide” or “RNA-binding site-directed modifying polypeptide” or “site-directed polypeptide” it is meant a polypeptide that binds RNA and is targeted to a specific DNA sequence. A site-directed modifying polypeptide as described herein is targeted to a specific DNA sequence by the RNA molecule to which it is bound. The RNA molecule comprises a sequence that binds, hybridizes to, or is complementary to a target sequence within the target DNA, thus targeting the bound polypeptide to a specific location within the target DNA (the target sequence). By “cleavage” it is meant the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, a complex comprising a guide RNA and a site-directed modifying polypeptide is used for targeted double-stranded DNA cleavage.

“Nuclease” and “endonuclease” are used interchangeably herein to mean an enzyme which possesses endonucleolytic catalytic activity for polynucleotide cleavage.

By “cleavage domain” or “active domain” or “nuclease domain” of a nuclease it is meant the polypeptide sequence or domain within the nuclease which possesses the catalytic activity for DNA cleavage. A cleavage domain can be contained in a single polypeptide chain or cleavage activity can result from the association of two (or more) polypeptides. A single nuclease domain may consist of more than one isolated stretch of amino acids within a given polypeptide.

By “site-directed polypeptide” or “RNA-binding site-directed polypeptide” or “RNA-binding site-directed polypeptide” it is meant a polypeptide that binds RNA and is targeted to a specific DNA sequence. A site-directed polypeptide as described herein is targeted to a specific DNA sequence by the RNA molecule to which it is bound. The RNA molecule comprises a sequence that is complementary to a target sequence within the target DNA, thus targeting the bound polypeptide to a specific location within the target DNA (the target sequence).

The “guide sequence” or DNA-targeting segment (or “DNA-targeting sequence”) comprises a nucleotide sequence that is complementary to a specific sequence within a target DNA (the complementary strand of the target DNA) designated the “protospacer-like” sequence herein. The protein-binding segment (or “protein-binding sequence”) interacts with a site-directed modifying polypeptide. When the site-directed modifying polypeptide is an sRGN or sRGN related polypeptide (described in more detail below), site-specific cleavage of the target DNA occurs at locations determined by both (i) base-pairing complementarity between the guide RNA and the target DNA; and (ii) a short motif (referred to as the protospacer adjacent motif (PAM)) in the target DNA.

The protein-binding segment of a guide RNA comprises, in part, two complementary stretches of nucleotides that hybridize to one another to form a double-stranded RNA duplex (dsRNA duplex).

In some embodiments, a nucleic acid (e.g., a guide RNA, a nucleic acid comprising a nucleotide sequence encoding a guide RNA; a nucleic acid encoding a site-directed polypeptide; etc.) comprises a modification or sequence that provides for an additional desirable feature (e.g., modified or regulated stability; subcellular targeting; tracking, e.g., a fluorescent label; a binding site for a protein or protein complex; etc.). Non-limiting examples include: a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and/or protein complexes); a stability control sequence; a sequence that forms a dsRNA duplex (i.e., a hairpin)); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, etc.); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like); and combinations thereof.

In some embodiments, a guide RNA comprises an additional segment at either the 5′ or 3′ end that provides for any of the features described above. For example, a suitable third segment can comprise a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and protein complexes); a stability control sequence; a sequence that forms a dsRNA duplex (i.e., a hairpin)); a sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, etc.); a modification or sequence that provides a binding site for proteins (e.g., proteins that act on DNA. including transcriptional activators, transcriptional repressors, DNA methyltransferases, DNA demethylases, histone acetyltransferases, histone deacetylases, and the like); and combinations thereof.

A guide RNA and an sRGN polypeptide form a complex (i.e., bind via non-covalent interactions). The guide RNA provides target specificity to the complex by comprising a nucleotide sequence that is complementary to a sequence of a target DNA. The sRGN polypeptide of the complex provides the site-specific activity. In other words, the sRGN polypeptide is guided to a target DNA sequence (e.g., a target sequence in a chromosomal nucleic acid; a target sequence in an extrachromosomal nucleic acid, e.g., an episomal nucleic acid, a minicircle, etc.; a target sequence in a mitochondrial nucleic acid; a target sequence in a chloroplast nucleic acid; a target sequence in a plasmid; etc.) by virtue of its association with the protein-binding segment of the guide RNA. RNA aptamers are known in the art and are generally a synthetic version of a riboswitch. The terms “RNA aptamer” and “riboswitch” are used interchangeably herein to encompass both synthetic and natural nucleic acid sequences that provide for inducible regulation of the structure (and therefore the availability of specific sequences) of the RNA molecule of which they are part. RNA aptamers usually comprise a sequence that folds into a particular structure (e.g., a hairpin), which specifically binds a particular drug (e.g., a small molecule). Binding of the drug causes a structural change in the folding of the RNA, which changes a feature of the nucleic acid of which the aptamer is a part. As non-limiting examples: (i) an activator-RNA with an aptamer may not be able to bind to the cognate targeter-RNA unless the aptamer is bound by the appropriate drug; (ii) a targeter-RNA with an aptamer may not be able to bind to the cognate activator-RNA unless the aptamer is bound by the appropriate drug; and (iii) a targeter-RNA and an activator-RNA, each comprising a different aptamer that binds a different drug, may not be able to bind to each other unless both drugs are present. As illustrated by these examples, a two-molecule guide RNA can be designed to be inducible.

Examples of aptamers and riboswitches can be found, for example, in: Nakamura et al., Genes Cells. 2012 May; 17 (5): 344-64; Vavalle et al., Future Cardiol. 2012 May; 8 (3): 371-82; Citartan et al., Biosens Bioelectron. 2012 Apr. 15; 34 (1): 1-11; and Liberman et al., Wiley Interdiscip Rev RNA. 2012 May-June; 3 (3): 369-84; all of which are herein incorporated by reference in their entirety.

The term “stem cell” is used herein to refer to a cell (e.g., plant stem cell, vertebrate stem cell) that has the ability both to self-renew and to generate a differentiated cell type (see Morrison et al. (1997) Cell 88:287-298). In the context of cell ontogeny, the adjective “differentiated”, or “differentiating” is a relative term. A “differentiated cell” is a cell that has progressed further down the developmental pathway than the cell it is being compared with. Thus, pluripotent stem cells (described below) can differentiate into lineage-restricted progenitor cells (e.g., mesodermal stem cells), which in turn can differentiate into cells that are further restricted (e.g., neuron progenitors), which can differentiate into end-stage cells (i.e., terminally differentiated cells, e.g., neurons. cardiomyocytes, etc.), which play a characteristic role in a certain tissue type, and may or may not retain the capacity to proliferate further. Stem cells may be characterized by both the presence of specific markers (e.g., proteins, RNAs, etc.) and the absence of specific markers. Stem cells may also be identified by functional assays both in vitro and in vivo, particularly assays relating to the ability of stem cells to give rise to multiple differentiated progeny.

Stem cells of interest include pluripotent stem cells (PSCs). The term “pluripotent stem cell” or “PSC” is used herein to mean a stem cell capable of producing all cell types of the organism. Therefore, a PSC can give rise to cells of all germ layers of the organism (e.g., the endoderm, mesoderm, and ectoderm of a vertebrate). Pluripotent cells are capable of forming teratomas and of contributing to ectoderm, mesoderm, or endoderm tissues in a living organism. Pluripotent stem cells of plants are capable of giving rise to all cell types of the plant (e.g., cells of the root, stem, leaves, etc.).

PSCs of animals can be derived in a number of different ways. For example, embryonic stem cells (ESCs) are derived from the inner cell mass of an embryo (Thomson et. al, Science. 1998 Nov. 6; 282 (5391): 1145-7) whereas induced pluripotent stem cells (iPSCs) are derived from somatic cells (Takahashi et. al, Cell. 2007 Nov. 30; 131 (5): 861-72; Takahashi et. al, Nat Protoc. 2007; 2 (12): 3081-9; Yu et. al, Science. 2007 Dec. 21; 318 (5858): 1917-20. Epub 2007 Nov. 20).

Because the term PSC refers to pluripotent stem cells regardless of their derivation, the term PSC encompasses the terms ESC and iPSC, as well as the term embryonic germ stem cells (EGSC), which are another example of a PSC. PSCs may be in the form of an established cell line, they may be obtained directly from primary embryonic tissue, or they may be derived from a somatic cell. PSCs can be target cells of the methods described herein.

By “embryonic stem cell” (ESC) is meant a PSC that was isolated from an embryo, generally from the inner cell mass of the blastocyst. ESC lines are listed in the NIH Human Embryonic Stem Cell Registry, e.g., hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, HSF-6 (University of California at San Francisco); and H1, H7, H9, H13, H14 (Wisconsin Alumni Research Foundation (WiCell Research Institute)). Stem cells of interest also include embryonic stem cells from other primates, such as Rhesus stem cells and marmoset stem cells. The stem cells may be obtained from any mammalian species, e.g., human, equine, bovine, porcine, canine, feline, rodent. e.g., mice, rats. hamster, primate, etc. (Thomson et al. (1998) Science 282:1145; Thomson et al. (1995) Proc. Natl. Acad. Sci. USA 92:7844; Thomson et al. (1996) Biol. Reprod. 55:254; Shamblott et al., Proc. Natl. Acad. Sci. USA 95:13726, 1998). In culture, ESCs generally grow as flat colonies with large nucleo-cytoplasmic ratios, defined borders and prominent nucleoli. In addition, ESCs express SSEA-3, SSEA-4, TRA-1-60, TRA-1-81, and alkaline phosphatase, but not SSEA-1. Examples of methods of generating and characterizing ESCs may be found in, for example, U.S. Pat. Nos. 7,029,913, 5,843,780, and 6,200,806, the disclosures of which are incorporated herein by reference. Methods for proliferating hESCs in the undifferentiated form are described in WO 99/20741, WO 01/51616, and WO 03/020920. By “embryonic germ stem cell” (EGSC) or “embryonic germ cell” or “EG cell” is meant a PSC that is derived from germ cells and/or germ cell progenitors, e.g., primordial germ cells, i.e., those that would become sperm and eggs. Embryonic germ cells (EG cells) are thought to have properties similar to embryonic stem cells as described above. Examples of methods of generating and characterizing EG cells may be found in, for example, U.S. Pat. No. 7,153,684; Matsui, Y., et al., (1992) Cell 70:841; Shamblott, M., et al. (2001) Proc. Natl. Acad. Sci. USA 98:113; Shamblott, M., et al. (1998) Proc. Natl. Acad. Sci. USA, 95:13726; and Koshimizu, U., et al. (1996) Development, 122:1235, the disclosures of which are incorporated herein by reference.

By “induced pluripotent stem cell” or “iPSC” it is meant a PSC that is derived from a cell that is not a PSC (i.e., from a cell this is differentiated relative to a PSC). iPSCs can be derived from multiple different cell types, including terminally differentiated cells. iPSCs have an ES cell-like morphology, growing as flat colonies with large nucleo-cytoplasmic ratios, defined borders and prominent nuclei. In addition, iPSCs express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, Fox03, GDF3, Cyp26a1, TERT, and zfp42.

Examples of methods of generating and characterizing iPSCs may be found in, for example, US Patent Publication Nos. US20090047263, US20090068742, US20090191159, US20090227032, US20090246875, and US20090304646, the disclosures of which are incorporated herein by reference. Generally, to generate iPSCs, somatic cells are provided with reprogramming factors (e.g., Oct4, SOX2, KLF4, MYC, Nanog, Lin28, etc.) known in the art to reprogram the somatic cells to become pluripotent stem cells.

By “somatic cell” it is meant any cell in an organism that, in the absence of experimental manipulation, does not ordinarily give rise to all types of cells in an organism. In other words, somatic cells are cells that have differentiated sufficiently that they will not naturally generate cells of all three germ layers of the body, i.e., ectoderm, mesoderm and endoderm. For example, somatic cells would include both neurons and neural progenitors, the latter of which may be able to naturally give rise to all or some cell types of the central nervous system but cannot give rise to cells of the mesoderm or endoderm lineages.

By “mitotic cell” it is meant a cell undergoing mitosis. Mitosis is the process by which a eukaryotic cell separates the chromosomes in its nucleus into two identical sets in two separate nuclei. It is generally followed immediately by cytokinesis, which divides the nuclei, cytoplasm, organelles and cell membrane into two cells containing roughly equal shares of these cellular components.

By “post-mitotic cell” it is meant a cell that has exited from mitosis, i.e., it is “quiescent”, i.e., it is no longer undergoing divisions. This quiescent state may be temporary, i.e., reversible, or it may be permanent.

By “meiotic cell” it is meant a cell that is undergoing meiosis. Meiosis is the process by which a cell divides its nuclear material for the purpose of producing gametes or spores. Unlike mitosis, in meiosis, the chromosomes undergo a recombination step which shuffles genetic material between chromosomes. Additionally, the outcome of meiosis is four (genetically unique) haploid cells, as compared with the two (genetically identical) diploid cells produced from mitosis.

By “recombination” it is meant a process of exchange of genetic information between two polynucleotides. As used herein, “homology-directed repair (HDR)” refers to the specialized form DNA repair that takes place, for example, during repair of double-strand breaks in cells. This process requires nucleotide sequence homology, uses a “donor” molecule to template repair of a “target” molecule (i.e., the one that experienced the double-strand break), and leads to the transfer of genetic information from the donor to the target. Homology-directed repair may result in an alteration of the sequence of the target molecule (e.g., insertion, deletion, mutation), if the donor polynucleotide differs from the target molecule and part or all of the sequence of the donor polynucleotide is incorporated into the target DNA In some embodiments, the donor polynucleotide, a portion of the donor polynucleotide, a copy of the donor polynucleotide, or a portion of a copy of the donor polynucleotide integrates into the target DNA.

By “non-homologous end joining (NHEJ) it is meant the repair of double-strand breaks in DNA by direct ligation of the break ends to one another without the need for a homologous template (in contrast to homology-directed repair, which requires a homologous sequence to guide repair). NHEJ often results in the loss (deletion) of nucleotide sequence near the site of the double-strand break.

The terms “treatment”, ‘treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may be predisposed to acquiring the disease or symptom but has not yet been diagnosed as having it; (b) inhibiting the disease or symptom, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The therapy will desirably be administered during the symptomatic stage of the disease, and in some cases after the symptomatic stage of the disease.

The terms “individual,” “subject,” “host,” and “patient,” are used interchangeably herein and refer to any mammalian subject for whom diagnosis, treatment, or therapy is desired, particularly humans.

General methods in molecular and cellular biochemistry can be found in such standard textbooks as Molecular Cloning: A Laboratory Manual, 3rd Ed. (Sambrook et al., Harboor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (1. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

The phrase “consisting essentially of” is meant herein to exclude anything that is not the specified active component or components of a system, or that is not the specified active portion or portions of a molecule.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Synthetic RNA-Guided Nuclease (sRGN)

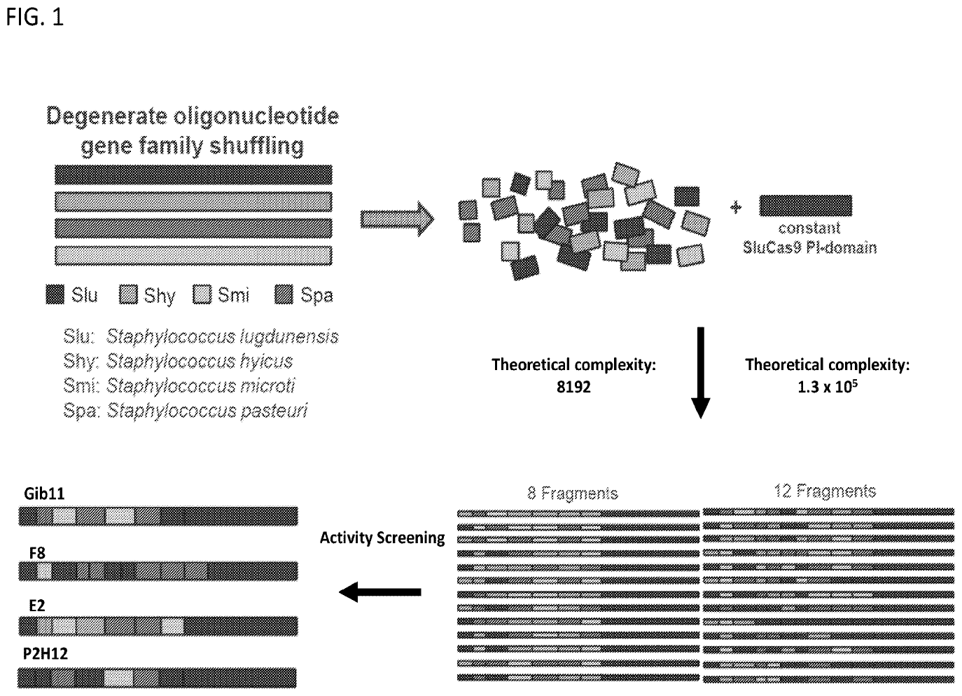

Synthetic RNA-guided nucleases (sRGNs) can be generated, for example, using homology-based gene family shuffling of artificially defined mini-domains of CRISPR-Cas9 endonucleases of different species, e.g., four different Staphylococcus species (Staphylococcus lugdunensis, Staphylococcus pasteuri, Staphylococcus microti, and Staphylococcus hyicus). One such gene family shuffling protocol is schematically depicted in FIG. 1. Briefly, the sequences of different Cas9 endonucleases are compared to identify regions with high homology to serve as anchor points, breaking each of the Cas9 endonucleases up into a plurality (such as 5, 6, 7, 8, 9, 10, 11, 12, 13, 14, 15, or more) corresponding mini-domains. Libraries of gene family shuffled sRGNs are prepared by randomly assigning each mini-domain to be derived from one of the original Cas9 endonucleases. In some instances, it may be desirable to keep the PAM-interacting (PI) domain fixed so that a single PAM sequence is recognized by each of the library constructs. For example, a library can be prepared by breaking each of the Cas9 endonucleases into 8 mini-domains, holding the C-terminal mini-domain fixed, resulting in a theoretical complexity of 8192. In another example, a library can be prepared by breaking each of the Cas9 endonucleases into 12 mini-domains, holding the C-terminal mini-domain fixed, resulting in a theoretical complexity of 1.3×10⁵. The libraries can be screened using known assays for RNA-guided endonuclease activity, such as bacterial live/dead assays and reporter gene disruption assays, to identify sRGNs having high activity.

In one aspect, provided herein are candidate sRGNs designed according to a homology-based gene family shuffling protocol. The candidate sRGNs have not yet been shown to have any activity. Also provided are sRGNs that have been demonstrated to have at least one activity. In some embodiments, the at least one activity includes, for example, activity in a cleavage assay, such as a fluorescence polarization-based biochemical cleavage assay, a gene-editing assay such as a GFP-to-BFP conversion (Glaser et al., 2016, infra), a gene insertion assay, or assay of other suitable parameters. In some embodiments, on-target cutting is assayed, off-target cutting is assayed, and/or on-target editing (for example, correction of a mutation or insertion of a gene or portion of a gene) is assayed, which may be compared to off-target editing. In some cases, activity is compared to a reference or a control nuclease such as a wild type Cas9. Assays are described in the art and are available from commercial resources. A suitable assay may be carried out in an organism, for example, an animal having a deleterious mutation, and efficacy of an sRGN is assayed by improvement of one or more symptoms associated with the mutation in an animal to which an sRGN system was administered. In some embodiments, the at least one activity is greater than the corresponding activity in one or more of the parental Cas9 endonucleases from which the sRGN is derived. In some embodiments, the at least one activity is greater than the corresponding activity in another wild-type Cas9, such as SluCas9.

In some embodiments, provided herein is a candidate sRGN comprising 8 mini-domains, wherein the mini-domains, from N-terminus to C-terminus, are 1) mini-domain 1 comprising the amino acid sequence of any one of SEQ ID NOs: 34-37 or a variant thereof having at least about 90% (such as at least about any of 91%, 92%, 93%, 94%, 95%, 96%, 97%, 98%, 99%, or more) sequence identity to any one of SEQ ID NOs: 34-37, 2) mini-domain 2 comprising the amino acid sequence of any one of SEQ ID NOs: 38-41 or a variant thereof having at least about 90% (such as at least about any of 91%, 92%, 93%, 94%, 95%, 96%, 97%, 98%, 99%, or more) sequence identity to any one of SEQ ID NOs: 38-41, 3) mini-domain 3 comprising the amino acid sequence of any one of SEQ ID NOs: 42-45 or a variant thereof having at least about 90% (such as at least about any of 91%, 92%, 93%, 94%, 95%, 96%, 97%, 98%, 99%, or more) sequence identity to any one of SEQ ID NOs: 42-45, 4) mini-domain 4 comprising the amino acid sequence of any one of SEQ ID NOs: 46-49 or a variant thereof having at least about 90% (such as at least about any of 91%, 92%, 93%, 94%, 95%, 96%, 97%, 98%, 99%, or more) sequence identity to any one of SEQ ID NOs: 46-49, 5) mini-domain 5 comprising the amino acid sequence of any one of SEQ ID NOs: 50-53 or a variant thereof having at least about 90% (such as at least about any of 91%, 92%, 93%, 94%, 95%, 96%, 97%, 98%, 99%, or more) sequence identity to any one of SEQ ID NOs: 50-53, 6) mini-domain 6 comprising the amino acid sequence of any one of SEQ ID NOs: 54-57 or a variant thereof having at least about 90% (such as at least about any of 91%, 92%, 93%, 94%, 95%, 96%, 97%, 98%, 99%, or more) sequence identity to any one of SEQ ID NOs: 54-57, 7) mini-domain 7 comprising the amino acid sequence of any one of SEQ ID NOs: 58-61 or a variant thereof having at least about 90% (such as at least about any of 91%, 92%, 93%, 94%, 95%, 96%, 97%, 98%, 99%, or more) sequence identity to any one of SEQ ID NOs: 58-61, and 8) mini-domain 8 comprising the amino acid sequence of any one of SEQ ID NOs: 62-65 or a variant thereof having at least about 90% (such as at least about any of 91%, 92%, 93%, 94%, 95%, 96%, 97%, 98%, 99%, or more) sequence identity to any one of SEQ ID NOs: 62-65. In some embodiments, the candidate sRGN comprises mini-domains from Staphylococcus lugdunensis, Staphylococcus pasteuri, Staphylococcus microti, and Staphylococcus hyicus Cas9s. In some embodiments, the candidate sRGN comprises mini-domains from less than all 4 (such as 2 or 3) Cas9s of Staphylococcus lugdunensis, Staphylococcus pasteuri, Staphylococcus microti, and Staphylococcus hyicus. In some embodiments, mini-domain 8 comprises the amino acid sequence of SEQ ID NO: 62 or a variant thereof having at least about 90% (such as at least about any of 91%, 92%, 93%, 94%, 95%, 96%, 97%, 98%, 99%, or more) sequence identity to SEQ ID NO: 62. In some embodiments, mini-domain 8 comprises the amino acid sequence of SEQ ID NO: 63 or a variant thereof having at least about 90% (such as at least about any of 91%, 92%, 93%, 94%, 95%, 96%, 97%, 98%, 99%, or more) sequence identity to SEQ ID NO: 63. In some embodiments, mini-domain 1 comprises the amino acid sequence of SEQ ID NO: 34 or a variant thereof having at least about 90% (such as at least about any of 91%, 92%, 93%, 94%, 95%, 96%, 97%, 98%, 99%, or more) sequence identity to SEQ ID NO: 34.

In some embodiments, provided herein is a candidate sRGN comprising 12 mini-domains, wherein the mini-domains, from N-terminus to C-terminus, are 1) mini-domain 1 comprising the amino acid sequence of any one of SEQ ID NOs: 66-69 or a variant thereof having at least about 90% (such as at least about any of 91%, 92%, 93%, 94%, 95%, 96%, 97%, 98%, 99%, or more) sequence identity to any one of SEQ ID NOs: 66-69, 2) mini-domain 2 comprising the amino acid sequence of any one of SEQ ID NOs: 70-73 or a variant thereof having at least about 90% (such as at least about any of 91%, 92%, 93%, 94%, 95%, 96%, 97%, 98%, 99%, or more) sequence identity to any one of SEQ ID NOs: 70-73, 3) mini-domain 3 comprising the amino acid sequence of any one of SEQ ID NOs: 74-77 or a variant thereof having at least about 90% (such as at least about any of 91%, 92%, 93%, 94%, 95%, 96%, 97%, 98%, 99%, or more) sequence identity to any one of SEQ ID NOs: 74-77, 4) mini-domain 4 comprising the amino acid sequence of any one of SEQ ID NOs: 78-81 or a variant thereof having at least about 90% (such as at least about any of 91%, 92%, 93%, 94%, 95%, 96%, 97%, 98%, 99%, or more) sequence identity to any one of SEQ ID NOs: 78-81, 5) mini-domain 5 comprising the amino acid sequence of any one of SEQ ID NOs: 82-85 or a variant thereof having at least about 90% (such as at least about any of 91%, 92%, 93%, 94%, 95%, 96%, 97%, 98%, 99%, or more) sequence identity to any one of SEQ ID NOs: 82-85, 6) mini-domain 6 comprising the amino acid sequence of any one of SEQ ID NOs: 86-89 or a variant thereof having at least about 90% (such as at least about any of 91%, 92%, 93%, 94%, 95%, 96%, 97%, 98%, 99%, or more) sequence identity to any one of SEQ ID NOs: 86-89, 7) mini-domain 7 comprising the amino acid sequence of any one of SEQ ID NOs: 90-93 or a variant thereof having at least about 90% (such as at least about any of 91%, 92%, 93%, 94%, 95%, 96%, 97%, 98%, 99%, or more) sequence identity to any one of SEQ ID NOs: 90-93, 8) mini-domain 8 comprising the amino acid sequence of any one of SEQ ID NOs: 94-97 or a variant thereof having at least about 90% (such as at least about any of 91%, 92%, 93%, 94%, 95%, 96%, 97%, 98%, 99%, or more) sequence identity to any one of SEQ ID NOs: 94-97, 9) mini-domain 9 comprising the amino acid sequence of any one of SEQ ID NOs: 98-101 or a variant thereof having at least about 90% (such as at least about any of 91%, 92%, 93%, 94%, 95%, 96%, 97%, 98%, 99%, or more) sequence identity to any one of SEQ ID NOs: 98-101, 10) mini-domain 10 comprising the amino acid sequence of any one of SEQ ID NOs: 102-105 or a variant thereof having at least about 90% (such as at least about any of 91%, 92%, 93%, 94%, 95%, 96%, 97%, 98%, 99%, or more) sequence identity to any one of SEQ ID NOs: 102-105, 11) mini-domain 11 comprising the amino acid sequence of any one of SEQ ID NOs: 106-109 or a variant thereof having at least about 90% (such as at least about any of 91%, 92%, 93%, 94%, 95%, 96%, 97%, 98%, 99%, or more) sequence identity to any one of SEQ ID NOs: 106-109, and 12) mini-domain 12 comprising the amino acid sequence of any one of SEQ ID NOs: 110-113 or a variant thereof having at least about 90% (such as at least about any of 91%, 92%, 93%, 94%, 95%, 96%, 97%, 98%, 99%, or more) sequence identity to any one of SEQ ID NOs: 110-113. In some embodiments, the candidate sRGN comprises mini-domains from Staphylococcus lugdunensis, Staphylococcus pasteuri, Staphylococcus microti, and Staphylococcus hyicus Cas9s. In some embodiments, the candidate sRGN comprises mini-domains from less than all 4 (such as 2 or 3) Cas9s of Staphylococcus lugdunensis, Staphylococcus pasteuri, Staphylococcus microti, and Staphylococcus hyicus. In some embodiments, mini-domain 12 comprises the amino acid sequence of SEQ ID NO: 110 or a variant thereof having at least about 90% (such as at least about any of 91%, 92%, 93%, 94%, 95%, 96%, 97%, 98%, 99%, or more) sequence identity to SEQ ID NO: 110. In some embodiments, mini-domain 12 comprises the amino acid sequence of SEQ ID NO: 111 or a variant thereof having at least about 90% (such as at least about any of 91%, 92%, 93%, 94%, 95%, 96%, 97%, 98%, 99%, or more) sequence identity to SEQ ID NO: 111. In some embodiments, (i) mini-domain 1 comprises the amino acid sequence of SEQ ID NO: 66 or a variant thereof having at least about 90% (such as at least about any of 91%, 92%, 93%, 94%, 95%, 96%, 97%, 98%, 99%, or more) sequence identity to SEQ ID NO: 66 and (ii) mini-domain 2 comprises the amino acid sequence of SEQ ID NO: 70 or a variant thereof having at least about 90% (such as at least about any of 91%, 92%, 93%, 94%, 95%, 96%, 97%, 98%, 99%, or more) sequence identity to SEQ ID NO: 70.

The sRGN polypeptides Gib11 (SEQ ID NO: 1), P2H12 (SEQ ID NO: 5), E2 (SEQ ID NO: 6), and F8 (SEQ ID NO: 10) disclosed herein were identified using such a gene family shuffling protocol, and Gib11Spa variants (SEQ ID NOs: 2-4) were generated by replacing different C-terminal portions of Gib11 with the corresponding portions of Staphylococcus pasteuri Cas9.

The sRGN polypeptides disclosed herein are small compared to Cas9 from Streptococcus pyogenes (also referred to herein as “SpCas9” or “SpyCas9”), and interact with an NNGG PAM sequence.

In one aspect, provided herein is an sRGN polypeptide comprising the amino acid sequence of any one of SEQ ID NOs: 1-12 or a variant thereof having at least about 90% (such as at least about any of 91%, 92%, 93%, 94%, 95%, 96%, 97%, 98%, 99%, or more) sequence identity to any one of SEQ ID NOs: 1-12. In some embodiments, the sRGN polypeptide comprises the amino acid sequence of any one of SEQ ID NOs: 1-12.

In some embodiments, provided herein is an sRGN polypeptide comprising an amino acid sequence having at least about 90% (such as at least about any of 91%, 92%, 93%, 94%, 95%, 96%, 97%, 98%, 99%, or more) sequence identity to a subsequence of any one of SEQ ID NOs: 1-12, wherein the subsequence comprises one or more functional sRGN domains. In some embodiments, the sRGN polypeptide comprises (or consists of) a subsequence of any one of SEQ ID NOs: 1-12. In some embodiments, the one or more sRGN domains include a nuclease domain (e.g., an HNH or RuvC domain). In some embodiments, the one or more sRGN domains include an RNA-binding domain (e.g., a Rec I domain). In some embodiments, the one or more sRGN domains include a PAM-interacting domain.

In some embodiments, provided herein is a Gib11 variant including:
(I) variants of at least 90% (such as at least about any of 91%, 92%, 93%, 94%, 95%, 96%, 97%, 98%, 99%, or more) sequence identity to the amino acid sequence of SEQ ID NO: 1 over its entire length, or over the sequence from position 787 to position 1057 of SEQ ID NO: 1;
(II) variants according to (I) that further comprise components such as, e.g., nuclear localization signals, to obtain appropriate activity of a Gib11 CRISPR system not only in a cell-free reaction or in prokaryotic cells but also in eukaryotic cellular environments such as, for example, live organisms like plants or animals;
(III) codon-optimized variants of the corresponding polynucleotide sequences encoding Gib11 and the variants according to (I) and (II);
(IV) variants according to any of (I) to (III) that further comprise:
(a) one or more modification(s) or mutation(s) that result in an sRGN with a significantly reduced or non-detectable nuclease activity, for example, having an alanine, or any other exchange leading to a significantly reduced nuclease activity, in position 10 (e.g., D10A), position 562 (e.g., H562A), and/or position 585 (e.g., N585A) with respect to SEQ ID NO: 1; and
(b) a C- or N-terminally attached polypeptide with a nucleotide base editing activity or deaminase activity, such as via a linker peptide.

In some embodiments, provided herein is a P2H12 variant including:
(I) variants of at least 90% (such as at least about any of 91%, 92%, 93%, 94%, 95%, 96%, 97%, 98%, 99%, or more) sequence identity to the amino acid sequence of SEQ ID NO: 5 over its entire length, or over the sequence from position 784 to position 1055 of SEQ ID NO: 5;
(II) variants according to (I) that further comprise components such as, e.g., nuclear localization signals, to obtain appropriate activity of a P2H12 CRISPR system not only in a cell-free reaction or in prokaryotic cells but also in eukaryotic cellular environments such as, for example, live organisms like plants or animals;
(III) codon-optimized variants of the corresponding polynucleotide sequences encoding P2H12 and the variants according to (I) and (II);
(IV) variants according to any of (I) to (III) that further comprise:
(a) one or more modification(s) or mutation(s) that result in an sRGN with a significantly reduced or non-detectable nuclease activity, for example, having an alanine, or any other exchange leading to a significantly reduced nuclease activity, in position 10 (e.g., D10A), position 559 (e.g., H559A), and/or position 582 (e.g., N582A) with respect to SEQ ID NO: 5; and
(b) a C- or N-terminally attached polypeptide with a nucleotide base editing activity or deaminase activity, such as via a linker peptide.

In some embodiments, provided herein is an E2 variant including:
(I) variants of at least 90% (such as at least about any of 91%, 92%, 93%, 94%, 95%, 96%, 97%, 98%, 99%, or more) sequence identity to the amino acid sequence of SEQ ID NO: 6 over its entire length, or over the sequence from position 1 to position 789 of SEQ ID NO: 6;
(II) variants according to (I) that further comprise components such as, e.g., a nuclear localization signal, to obtain appropriate activity of a E2 CRISPR system not only in a cell-free reaction or in prokaryotic cells but also in eukaryotic cellular environments such as, for example, live organisms like plants or animals;
(III) codon-optimized variants of the corresponding polynucleotide sequences encoding E2 and the variants according to (I) and (II);
(IV) variants according to any of (I) to (III) that further comprise:
(a) one or more modification(s) or mutation(s) that result in an sRGN with a significantly reduced or non-detectable nuclease activity, for example, having an alanine, or any other exchange leading to a significantly reduced nuclease activity, in position 10 (e.g., D10A), position 562 (e.g., H562A), and/or position 585 (e.g., N585A) with respect to SEQ ID NO: 6; and
(b) a C- or N-terminally attached polypeptide with a nucleotide base editing activity or deaminase activity, such as via a linker peptide.

In some embodiments, provided herein is an F8 variant including:
(I) variants of at least 90% (such as at least about any of 91%, 92%, 93%, 94%, 95%, 96%, 97%, 98%, 99%, or more) sequence identity to the amino acid sequence of SEQ ID NO: 10 over its entire length, or over the sequence from position 1 to position 789 of SEQ ID NO: 10;
(II) variants according to (I) that further comprise components such as, e.g., nuclear localization signals, to obtain appropriate activity of a F8 CRISPR system not only in a cell-free reaction or in prokaryotic cells but also in eukaryotic cellular environments such as, for example, live organisms like plants or animals;
(III) codon-optimized variants of the corresponding polynucleotide sequences encoding F8 and the variants according to (I) and (II);
(IV) variants according to any of (I) to (III) that further comprise:
(a) one or more modification(s) or mutation(s) that result in an sRGN with a significantly reduced or non-detectable nuclease activity, for example, having an alanine, or any other exchange leading to a significantly reduced nuclease activity, in position 10 (e.g., D10A), position 562 (e.g., H562A), and/or position 585 (e.g., N585A) with respect to SEQ ID NO: 10; and
(b) a C- or N-terminally attached polypeptide with a nucleotide base editing activity or deaminase activity, such as via a linker peptide.

In some embodiments, provided herein is a Gib11Spa-1 variant including:
(I) variants of at least about 90% (such as at least about any of 91%, 92%, 93%, 94%, 95%, 96%, 97%, 98%, 99%, or more) sequence identity to the amino acid sequence of SEQ ID NO: 2;
(II) variants according to (I) that further comprise components such as, e.g., nuclear localization signals, to obtain appropriate activity of a Gib11Spa-1 CRISPR system not only in a cell-free reaction or in prokaryotic cells but also in eukaryotic cellular environments such as, for example, live organisms like plants or animals;
(III) codon-optimized variants of the corresponding polynucleotide sequences encoding Gib11Spa and the variants according to (I) and (II); (IV) variants according to any of (I) to (III) that further comprise:
(a) one or more modification(s) or mutation(s) that result in an sRGN with a significantly reduced or non-detectable nuclease activity, for example, having an alanine, or any other exchange leading to a significantly reduced nuclease activity, in position 10 (e.g., D10A), position 562 (e.g., H562A), and/or position 585 (e.g., N585A) with respect to SEQ ID NO: 2; and
(b) a C- or N-terminally attached polypeptide with a nucleotide base editing activity or deaminase activity, such as via a linker peptide.

In some embodiments, provided herein is a Gib11Spa-2 variant including:
(I) variants of at least about 90% (such as at least about any of 91%, 92%, 93%, 94%, 95%, 96%, 97%, 98%, 99%, or more) sequence identity to the amino acid sequence of SEQ ID NO: 3;
(II) variants according to (I) that further comprise components such as, e.g., nuclear localization signals, to obtain appropriate activity of a Gib11Spa-2 CRISPR system not only in a cell-free reaction or in prokaryotic cells but also in eukaryotic cellular environments such as, for example, live organisms like plants or animals;
(III) codon-optimized variants of the corresponding polynucleotide sequences encoding Gib11Spa and the variants according to (I) and (II);
(IV) variants according to any of (I) to (III) that further comprise:
(a) one or more modification(s) or mutation(s) that result in an sRGN with a significantly reduced or non-detectable nuclease activity, for example, having an alanine, or any other exchange leading to a significantly reduced nuclease activity, in position 10 (e.g., D10A), position 562 (e.g., H562A), and/or position 585 (e.g., N585A) with respect to SEQ ID NO: 3; and
(b) a C- or N-terminally attached polypeptide with a nucleotide base editing activity or deaminase activity, such as via a linker peptide.

In some embodiments, provided herein is a Gib11Spa-3 variant including:
(I) variants of at least about 90% (such as at least about any of 91%, 92%, 93%, 94%, 95%, 96%, 97%, 98%, 99%, or more) sequence identity to the amino acid sequence of SEQ ID NO: 4;
(II) variants according to (I) that further comprise components such as, e.g., nuclear localization signals, to obtain appropriate activity of a Gib11Spa-3 CRISPR system not only in a cell-free reaction or in prokaryotic cells but also in eukaryotic cellular environments such as, for example, live organisms like plants or animals;
(III) codon-optimized variants of the corresponding polynucleotide sequences encoding Gib11Spa and the variants according to (I) and (II);
(IV) variants according to any of (I) to (III) that further comprise:
(a) one or more modification(s) or mutation(s) that result in an sRGN with a significantly reduced or non-detectable nuclease activity, for example, having an alanine, or any other exchange leading to a significantly reduced nuclease activity, in position 10 (e.g., D10A), position 562 (e.g., H562A), and/or position 585 (e.g., N585A) with respect to SEQ ID NO: 4; and
(b) a C- or N-terminally attached polypeptide with a nucleotide base editing activity or deaminase activity, such as via a linker peptide.

The term “significantly reduced” for example used in the context of an enzymatic activity means that such enzymatic activity is lower than about 10% of the activity of a reference protein (e.g., any one of SEQ ID NOs: 1-12), for example, lower than 5%, lower than 2%, lower than 1%, or lower than 0.1% of such reference enzymatic activity.

Suitable polypeptides under (IV) (b) and their attachment to the C-terminal domain of an sRGN under (I), (II), and (III) are described for example in WO2017/070632, Gaudelli et al., Nature 551 (23 Nov. 2017), 464-471; Komor et al., Sci. Adv. 2017; 3eaao4774; Kim et al., Nature Biotechnol. 2017 April; 35 (4) 371-376; Komor et al., Nature 533 (7603); 420-424.

Instead of introducing double-strand breaks into the target DNA sequence, sRGN variants under (IV) could allow the conversion of any base pair into any possible other base pair. One non-limiting example is a deaminase activity that could act upon cytosine, guanine or adenine bases and their subsequent replication through the deaminated site and repair within the cell to yield guanine, thymine, and guanine, respectively.

If not otherwise specified, the term sRGN includes the sRGN polypeptide of any one of SEQ ID NOs: 1-12 as well as all of the variants thereof described herein, such as those specified under (I), (II), (III), and (IV).

In some embodiments, provided herein are mutants of E2 that with respect to SEQ ID NO: 6, comprise: a) an aspartate residue in position 741, and a lysine residue in position 743 (E2+K741D+L743K, SEQ ID NO: 7); or b) a threonine residue in position 670, and an aspartate residue in position 675 (E2+S670T+N675D, SEQ ID NO: 8); or c) an asparagine residue in position 741, and an asparagine residue in position 743 (E2+K741N+L743N, SEQ ID NO: 9); or d) any combination of the above (a) to (c).

In some embodiments, provided herein are mutants of E2 comprising i) at least about 90% (such as at least about any of 91%, 92%, 93%, 94%, 95%, 96%, 97%, 98%, 99%, or more) amino acid identity to the sequence according to SEQ ID NO: 6 over its entire length, or to the sequence from position 1 to position 789 of SEQ ID NO: 6; and ii) when aligned to a continuous stretch starting with 5 positions before al and ending 5 positions behind al of Table 1, exhibits the amino acid residue in position al as shown in Table 1; and iii) when aligned to a continuous stretch starting with 5 positions before a2 and ending 5 positions behind a2 of Table 1, exhibits the amino acid residue in position a2 as shown in Table 1; wherein aligned means the alignment of the stretch of 11 amino acid residues to the sequence according to SEQ ID NO: 6 using a standard local alignment tool or algorithm, including for example Protein BLAST (blast.ncbi.nlm.nih.gov, the National Center for Biotechnology [cited on May 17, 2018] Information; Altschul, S. F., Gish, W., Miller, W., Myers, E. W. & Lipman, D. J. (1990) “Basic local alignment search tool.” J. Mol. Biol. 215:403-410.) using standard parameters, and wherein at least 4 of the 5 preceding amino acids of al or a2 and 4 of the 5 amino acids behind a1 or a2 are identical.

TABLE 1

| Positions | Amino acid | Amino acid |
|---|---|---|
| according to | residue in | residue in |
| SEQ ID NO: 6 | position a1 | position a2 |
| a1 = 670, a2 = 675 | threonine | aspartate |
| a1 = 741, a2 = 743 | aspartate | lysine |
| a1 = 741, a2 = 743 | asparagine | asparagine |

If not otherwise specified, the term E2 comprises the E2 mutants, including E2+K741D+L743K; E2+S670T+N675D; and E2+K741N+L743N.

In some embodiments, provided herein are mutants of F8 that with respect to SEQ ID NO: 10 comprise a) an aspartate residue in position 737, and a lysine residue in position 739 (F8+K737D+L739K, SEQ ID NO: 11); or b) an asparagine residue in position 737, and an asparagine residue in position 739 (F8+K737N+L739N, SEQ ID NO: 12).

In some embodiments, provided herein are mutants of F8 comprising i) at least about 90% (such as at least about any of 91%, 92%, 93%, 94%, 95%, 96%, 97%, 98%, 99%, or more) amino acid identity to the sequence according to SEQ ID NO: 10 over its entire length, or to the sequence from position 1 to position 787 of SEQ ID NO: 10; and ii) when aligned to a continuous stretch starting with 5 positions before al and ending 5 positions behind al of Table 2 exhibits the amino acid residue in position al as shown in Table 2; and iii) when aligned to a continuous stretch starting with 5 positions before a2 and ending 5 positions behind a2 of Table 2 exhibits the amino acid residue in position a2 as shown in Table 2; wherein aligned means the alignment of the stretch of 11 amino acid residues to the sequence according to SEQ ID NO: 10 using a standard local alignment tool or algorithm, like for example Protein BLAST (blast.ncbi.nlm.nih.gov, the National Center for Biotechnology [cited on May 17, 2018] Information; Altschul, S. F., Gish, W., Miller, W., Myers, E. W. & Lipman, D. J. (1990) “Basic local alignment search tool.” J. Mol. Biol. 215:403-410.) using standard parameters, and wherein at least 4 of the 5 preceding amino acids of al or a2 and 4 of the 5 amino acids behind a1 or a2 are identical.

TABLE 2

| Positions | Amino acid | Amino acid |
|---|---|---|
| according to | residue in | residue in |
| SEQ ID NO: 10 | position a1 | position a2 |
| a1 = 737, a2 = 739 | aspartate | lysine |
| a1 = 737, a2 = 739 | asparagine | asparagine |

If not otherwise specified, the term F8 comprises the F8 mutants, including F8+K737D+L739K; and F8+K737N+L739N.

Additional sRGNs can be generated by identifying pairwise combinations of mini-domains in highly active sRGNs generated by gene family shuffling using a given library, and including at least one of these pairwise combination in a rationally designed sRGN, where the remaining sRGN mini-domains are selected from the library. The remaining mini-domains may be selected from those originating from the same parental Cas9s as the mini-domains in the pairwise combination. The rationally designed sRGN may further include one or more additional pairwise combinations of mini-domains in highly active sRGNs. For example, the rationally designed sRGN may comprise 2, 3, 4, 5, or more pairwise combinations of mini-domains present in highly active sRGNs.

Chimeric RNA-Guided Nuclease (cRGN)

Also provided herein are chimeric RNA-guided nucleases (cRGNs) comprising: A) an N-terminal part (cRGN-A), taken either from a type II CRISPR RNA-guided endonuclease Cas9 of a Staphylococcus, or a synthetic RNA-guided nuclease generated by DNA shuffling sequence of two or more Staphylococcus type II CRISPR RNA-guided endonuclease Cas proteins, such as any of the sRGNs described herein; and b) a C-terminal part (cRGN-B) taken from the type II CRISPR RNA-guided endonuclease Cas9 from Staphylococcus pasteuri (NCBI Refseq WP_023374365.1); wherein cRGN-B comprises i) the entire PI domain of the type II CRISPR RNA-guided endonuclease Cas9 from Staphylococcus pasteuri (NCBI Refseq WP_023374365.1), which is the sequence starting from amino acid position 905 and ending at amino acid position 1054 of the sequence according to NCBI Refseq WP_023374365.1; or ii) the entire PI domain of the type II CRISPR RNA-guided endonuclease Cas9 from Staphylococcus pasteuri (NCBI Refseq WP_023374365.1) and the entire WED domain, which is located from amino acid position 784 to amino acid position 904 of the sequence according to NCBI Refseq WP_023374365.1; cRGN-A comprises the first 700 amino acids of the respective type II CRISPR RNA-guided endonuclease Cas9 sequence, such as the first 739 amino acids, or the first 783 amino acids, with the exception of a sequence which is more than 99.9% identical, in some cases more than 99.5% identical on amino acid level to the respective part of NCBI Refseq WP_023374365.1 from Staphylococcus pasteuri; and cRGN-A is attached to cRGN-B either directly or by a linker peptide sequence. In some embodiments, cRGN-A comprises more than the first 700 amino acids of the respective type II CRISPR RNA-guided endonuclease Cas9 sequence, such as the first 739 amino acids, or the first 783 amino acids.

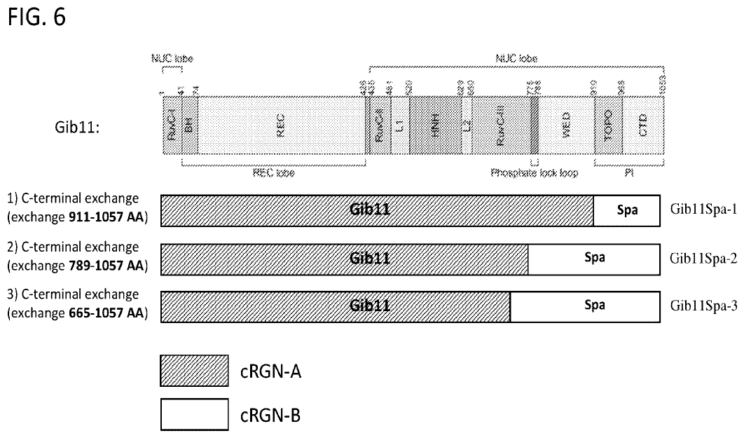

The PI domain and WED domain for the type II CRISPR RNA-guided endonuclease Cas9 of Staphylococcus aureus are described in Nishimasu et al., Cell. 2015 Aug. 27; 162 (5): 1113-1126. The potential localization of both domains is further shown in FIG. 6.

Any reference to the NCBI Refseq database within this patent application refers to the following citation: National Center for Biotechnology Information (NCBI) [Internet]. Bethesda (MD): National Library of Medicine (US), National Center for Biotechnology Information; [1988]-[cited 2018 May 11]. Available from: www.ncbi.nlm.nih.gov/protein. For example, for NCBI Refseq WP_048803085, this is www.ncbi.nlm.nih.gov/protein/WP_048803085.

Any reference to Staphylococcus alone means the genus Staphylococcus.

DNA shuffling in the context of this invention means the exchange of sequence fragments between different (e.g., from different Staphylococcus species) type II CRISPR Cas9 endonuclease DNA sequences to yield chimeric DNA sequences encoding synthetic proteins having an RNA-guided endonuclease activity, such as the activity of type II CRISPR Cas9 endonuclease. Such techniques are inter alia described in Joern J. M. (2003) DNA Shuffling. In: Arnold F. H., Georgiou G. (eds) Directed Evolution Library Creation. Methods in Molecular Biology™, vol 231. Humana Press, as well as in Gibbs et al., Gene. 2001 Jun. 13; 271 (1): 13-20.

In some embodiments, the type II CRISPR RNA-guided endonuclease Cas9 from Staphylococcus is identified by one of the following entries of the NCBI Refseq database: WP_053019794, WP_071859985, WP_049437627, WP_081329738, WP_081330634, WP_088922804, WP_061854099, WP_096792116, WP_107588422, WP_107543406, WP_105966910, WP_083326931, WP_105977729, WP_107578657, WP_107533955, WP_107597066, WP_105980293, WP_105978400, WP_107539784, WP_083326835, WP_096754380, WP_107580550, WP_107530431, WP_107597643, WP_107596301, WP_082732265, WP_083310250, WP_082709447, WP_105994700, WP_107568091, WP_107544007, WP_103361957, WP_001573634, WP_107576310, WP_107571611, WP_107593728, WP_103167028, WP_107532850, WP_002460848, WP_107506206, WP_104681501, WP_107546539, WP_058710220, WP_075777761, WP_050331073, WP_050345681, WP_053017934, WP_049415449, WP_060829977, WP_070855141, WP_107641154, WP_107392933, WP_107389582, WP_107390356, WP_107378401, WP_107378676, WP_060552032, WP_107376447, WP_039643679, WP_101457364, WP_104039168, WP_104052030, WP_085237539, WP_103357343, WP_044361501, WP_107366415, WP_107393309, WP_107633689, WP_107397003, WP_107386954, WP_107371508, WP_107538271, WP_107642914, WP_107536061, WP_107605852, WP_107547877, WP_107634675, WP_107532082, WP_107604007, WP_103356745, WP_107642811, WP_103298901, WP_107611983, WP_107623815, WP_107522281, WP_107560076, WP_107368542, WP_081502240, WP_107604002, WP_107377516, WP_016424980, WP_009382362, WP_001573633, WP_016424981, WP_103298687, WP_103361956, WP_107637979, WP_107612621, WP_107642817, WP_103356723, WP_107593719, WP_070592992, WP_107536245, WP_103209613, WP_063284667, WP_101457463, WP_105503156, WP_096548249, WP_096536567, WP_014613259, WP_063278948, WP_096601671, WP_103863320, WP_096559644, WP_019167918, WP_086428210, WP_096665615, WP_105976295, WP_107576302, WP_105977863, WP_107544006, WP_107538330, WP_070848771, WP_096605716, WP_095105824, WP_105966809, WP_070469119, WP_096589032, WP_105996442, WP_107552556, WP_060803934, WP_107549437, WP_107579080, WP_107547813, WP_107591747, WP_105978348, WP_096598476, WP_107587102, WP_107533825, WP_107588308, WP_107567989, WP_096544347, WP_023015764, WP_103322053, WP_060803559, WP_048803085, WP_107530433, WP_107580731, WP_107368971, WP_107559912, WP_107371387, WP_107571609, WP_107559911, WP_107571610, WP_107530436, WP_107580472, WP_017000597, WP_023374365, WP_062197343, WP_035512507, WP_079708828, WP_101040307, WP_053216997, WP_106588293, WP_107585021, WP_082099322, WP_017185731, WP_101662761, WP_084780162, WP_016837331, WP_084781893, WP_106494556, WP_088825434, WP_088816271, WP_059140148, WP_007547525, WP_084347835, WP_070710475, WP_077907981, WP_035723552, WP_058095017, WP_006446566, WP_074693676, WP_050980543, WP_001271093, WP_001271092, WP_087971021, WP_061668060, WP_016119566, WP_016106885, WP_088031364, WP_088038716, WP_007476473, WP_003343632, WP_088049424, WP_003354196, WP_066148467, WP_082806588, WP_087094968, WP_048723014, WP_033844707, WP_055358891, WP_081209836, WP_033018780, WP_023633350, WP_046580811, WP_081157433, WP_087959824, WP_041267823, WP_018922791, WP_063577905, WP_066227285, WP_044736072, WP_033016936, WP_100664518, WP_064213580, WP_053532223, WP_042224718, WP_084138993, WP_010632729, WP_018130201, WP_088027793, WP_065212529, WP_048539452, WP_001105083, WP_001105082, WP_086397116, WP_086390158, WP_070006567, WP_003739838, WP_070034634, WP_075702521, WP_029090905, WP_038409211, WP_069125601, WP_046323366, WP_069887401, WP_069134523, WP_061665472, WP_070294293, WP_003749665, WP_070784981, WP_031669209, WP_106787163, WP_077287021, WP_085392451, WP_023548323, WP_061108493, WP_101152964, WP_101143843, WP_101143453, WP_101140817, WP_066465432, WP_033920898, WP_069001072, WP_085400884, WP_101057368, WP_088838826, WP_072240946, WP_033838504, WP_010991369, WP_072218760, WP_072238933, WP_070227966, WP_061663015, WP_003733029, WP_101142252, WP_070031693, WP_070785826, WP_003730785, WP_070274575, WP_003727705, WP_069890501, WP_070233243, WP_070039312, WP_031665337, WP_070222802, WP_070264592, WP_103682188, WP_070299153, WP_070307355, WP_061395959, WP_070764199, WP_061128889, WP_070283519, WP_069009724, WP_070228842, WP_014601172, WP_103757671, WP_070293394, WP_060587936, WP_070238603, WP_070215465, WP_003723650, WP_070214481, WP_058876445, WP_060567941, WP_029499861, WP_027129613, WP_003145379, WP_080151624, WP_021752441, WP_069132012, WP_084038511, WP_004632196, WP_066230975, WP_072520207, WP_082729137, WP_080149038, WP_072233091, WP_072240445, WP_072218465, WP_072239624, WP_080151670, WP_077914265, WP_080151712, WP_052025682, WP_077914264, WP_100353964, WP_046517046, WP_008341324,

In some embodiments, a cRGN-A is from Gib11 (SEQ ID NO: 1). In some embodiments, the cRGN is Gib11Spa-1 (SEQ ID NO: 2), Gib11Spa-2 (SEQ ID NO: 3), or Gib11Spa-3 (SEQ ID NO: 4).

### Other Small Cas Nucleases

In one aspect, provided herein is a Cas9 from Staphylococcus pasteuri, Staphylococcus microti, or Staphylococcus hyicus. In some embodiments, the Cas9 is from Staphylococcus pasteuri, and comprises the amino acid sequence of SEQ ID NO: 31 or a variant thereof having at least about 90% (such as at least about any of 91%, 92%, 93%, 94%, 95%, 96%, 97%, 98%, 99%, or more) sequence identity to SEQ ID NO: 31. Staphylococcus pasteuri Cas9, or “SpaCas9,” recognizes the PAM sequence NNGG. In some embodiments, the Cas9 is from Staphylococcus microti, and comprises the amino acid sequence of SEQ ID NO: 33 or a variant thereof having at least about 90% (such as at least about any of 91%, 92%, 93%, 94%, 95%, 96%, 97%, 98%, 99%, or more) sequence identity to SEQ ID NO: 33. Staphylococcus microti Cas9, or “SmiCas9,” recognizes the PAM sequence NNGG. In some embodiments, the Cas9 is from Staphylococcus hyicus, and comprises the amino acid sequence of SEQ ID NO: 32 or a variant thereof having at least about 90% (such as at least about any of 91%, 92%, 93%, 94%, 95%, 96%, 97%, 98%, 99%, or more) sequence identity to SEQ ID NO: 32. Staphylococcus hyicus Cas9, or “ShyCas9,” recognizes the PAM sequence NNAAAA. In some cases, these small Cas9s can be used as an alternative to an sRGN in any of the systems, composition, and methods described herein.

Crispr-Cas System Based on sRGN Polypeptides

In some embodiments, provided herein is a system comprising: (a) an sRGN polypeptide or variant thereof having at least about 90% (such as at least about any of 91%, 92%, 93%, 94%, 95%, 96%, 97%, 98%, 99%, or more) sequence identity to any one of SEQ ID NOs: 1-12, or a nucleic acid encoding the sRGN polypeptide or variant thereof; and (b) a guide RNA (gRNA) or nucleic acid encoding the gRNA, wherein the gRNA is capable of guiding the sRGN polypeptide or variant thereof to a target polynucleotide sequence. In some embodiments, the system comprises the sRGN polypeptide or variant thereof. In some embodiments, the sRGN polypeptide comprises (or consists of) the amino acid sequence of any one of SEQ ID NOs: 1-12. In some embodiments, the system comprises the nucleic acid encoding the sRGN. In some embodiments, the nucleic acid has at least about 90% (such as at least about any of 91%, 92%, 93%, 94%, 95%, 96%, 97%, 98%, 99%, or more) sequence identity to SEQ ID NO: 13-29. In some embodiments, the nucleic acid comprises (or consists of) the sequence of SEQ ID NO: 13-29. In some embodiments, the system comprises the gRNA. In some embodiments, the system comprises nucleic acid encoding the gRNA. In some embodiments, the gRNA is a single guide RNA (sgRNA). In some embodiments, the system further comprises one or more additional gRNAs or nucleic acid encoding the one or more additional gRNAs.

In some embodiments, according to any of the nucleic acids comprising a polynucleotide sequence encoding an sRGN polypeptide or variant thereof described herein, the polynucleotide sequence is operably linked to (i) a suitable promoter for the expression in a cellular or in vitro environment; and/or (ii) a suitable nuclear localization signal.

In some embodiments, according to any of the nucleic acids encoding a gRNA described herein, the nucleic acid comprises a polynucleotide sequence encoding the gRNA that is operably linked to (i) a suitable promoter for the expression in a cellular or in vitro environment; and/or (ii) a suitable nuclear localization signal.

In some embodiments, provided herein is a system comprising: (a) an sRGN polypeptide comprising the amino acid sequence of any one of SEQ ID NOs: 1-12 or a variant thereof having at least 90% sequence identity to any one of SEQ ID NOs: 1-12; and (b) a gRNA, wherein the gRNA is capable of guiding the sRGN polypeptide or variant thereof to a target polynucleotide sequence. In some embodiments, the gRNA is an sgRNA.

In some embodiments, provided herein is a system comprising: (a) an sRGN polypeptide comprising the amino acid sequence of any one of SEQ ID NOs: 1-12 or a variant thereof having at least 90% sequence identity to any one of SEQ ID NOs: 1-12; and (b) a nucleic acid encoding a gRNA, wherein the gRNA is capable of guiding the sRGN polypeptide or variant thereof to a target polynucleotide sequence. In some embodiments, the gRNA is an sgRNA.

In some embodiments, provided herein is a system comprising: (a) a nucleic acid encoding an sRGN polypeptide comprising the amino acid sequence of any one of SEQ ID NOs: 1-12 or a variant thereof having at least 90% sequence identity to any one of SEQ ID NOs: 1-12; and (b) a gRNA, wherein the gRNA is capable of guiding the sRGN polypeptide or variant thereof to a target polynucleotide sequence. In some embodiments, the nucleic acid comprises the polynucleotide sequence of any one of SEQ ID NOs: 13-29 or a variant thereof having at least 90% sequence identity to the polynucleotide sequence of any one of SEQ ID NOs: 13-29. In some embodiments, the gRNA is an sgRNA.

In some embodiments, provided herein is a system comprising: (a) a nucleic acid encoding an sRGN polypeptide comprising the amino acid sequence of any one of SEQ ID NOs: 1-12 or a variant thereof having at least 90% sequence identity to any one of SEQ ID NOs: 1-12; and (b) a nucleic acid encoding a gRNA, wherein the gRNA is capable of guiding the sRGN polypeptide or variant thereof to a target polynucleotide sequence. In some embodiments, the nucleic acid comprises the polynucleotide sequence of any one of SEQ ID NOs: 13-29 or a variant thereof having at least 90% sequence identity to the polynucleotide sequence of any one of SEQ ID NOs: 13-29. In some embodiments, the gRNA is an sgRNA. In some embodiments, the system comprises a nucleic acid comprising (i) the nucleic acid encoding an sRGN polypeptide or variant thereof; and (ii) a nucleic acid encoding the gRNA.

In some embodiments, according to any of the systems described herein, the system further comprises a donor template. In some embodiments, the donor template comprises a donor sequence for insertion into the target polynucleotide sequence. In some embodiments, the donor sequence is an exogenous sequence, such as a transgene for expression in a target. In some embodiments, the donor template comprises a donor sequence for modifying one or more bases in the target polynucleotide sequence. In some embodiments, the system further comprises one or more additional donor templates. In some embodiments, the donor template is physically linked to a gRNA in the system.

One embodiment according to the invention provides compositions comprising:
(a) an sRGN polypeptide or a nucleic acid encoding such sRGN;
(b) a single heterologous guide RNA (sgRNA) or a nucleic acid (e.g., DNA) that allows the generation of such sgRNA in situ (e.g., nucleic acid encoding the sgRNA), wherein the sgRNA comprises:
   i. a DNA-targeting segment comprised of RNA and capable of hybridizing to a target sequence in a polynucleotide locus,
   ii. a tracr mate sequence comprised of RNA, and
   iii. a tracr RNA sequence comprised of RNA, wherein the tracr mate sequence is capable of hybridizing to the tracr sequence, and wherein (i), (ii), and (iii) are arranged in a 5′ to 3′ orientation.

Within a sgRNA, a tracr mate sequence and a tracr sequence are generally connected by a suitable loop sequence and form a stem-loop structure.

Further, in some embodiments the nucleic acid encoding the sRGN polypeptide and/or the sgRNAs contains a suitable promoter for the expression in a cellular or in vitro environment and/or a suitable nuclear localization signal.

Another embodiment according to the invention provides methods of targeting, editing, modifying, or manipulating a target DNA at one or more locations in a cell or in vitro, comprising:
(a) Introducing a heterologous sRGN polypeptide or a nucleic acid encoding the sRGN polypeptide into a cell or into an in vitro environment; and
(b) Introducing a single heterologous guide RNA (sgRNA) or a nucleic acid (e.g., DNA) suitable for the generation of such sgRNA in situ (e.g., nucleic acid encoding the sgRNA), wherein the sgRNA comprises:
   i. a DNA-targeting segment comprised of RNA and capable of hybridizing to a target sequence in a polynucleotide locus,
   ii. a tracr mate sequence comprised of RNA, and
   iii. a tracr RNA sequence comprised of RNA,
wherein the tracr mate sequence is capable of hybridizing to the tracr sequence, and wherein (i), (ii), and (iii) are arranged in a 5′ to 3′ orientation; and
(c) creating one or more cuts, nicks or edits in the target DNA, wherein the sRGN polypeptide is directed to the target DNA by the gRNA in its processed or unprocessed form.

Another embodiment according to the invention is the use of a composition comprising:
(a) an sRGN polypeptide or a nucleic acid encoding such sRGN;
(b) a single heterologous guide RNA (sgRNA) or a nucleic acid (e.g., DNA) suitable for the generation of such sgRNA in situ (e.g., nucleic acid encoding the sgRNA), wherein the sgRNA comprises:
   i. a DNA-targeting segment comprised of RNA and capable of hybridizing to such target sequence in a polynucleotide locus,
   ii. a tracr mate sequence comprised of RNA, and
   iii. a tracr RNA sequence comprised of RNA,
wherein the tracr mate sequence is capable of hybridizing to the tracr sequence, and wherein (i), (ii), and (iii) are arranged in a 5′ to 3′ orientation;
for targeting, editing, modifying, or manipulating a target DNA at one or more locations in a cell (e.g., in vitro or in vivo) or in a cell-free system.

Another embodiment according to the invention is a cell ex vivo or in vitro comprising:
(a) a heterologous sRGN polypeptide or a nucleic acid encoding the same
(b) a single heterologous guide RNA (sgRNA) or a nucleic acid (e.g., DNA) suitable for the generation of such sgRNA in situ (e.g., nucleic acid encoding the sgRNA), wherein the sgRNA comprises:
   i. a DNA-targeting segment comprised of RNA and capable of hybridizing to such target sequence in a polynucleotide locus,
   ii. a tracr mate sequence comprised of RNA, and
   iii. a tracr RNA sequence comprised of RNA,
wherein the tracr mate sequence is capable of hybridizing to the tracr sequence, and wherein (i), (ii), and (iii) are arranged in a 5′ to 3′ orientation;
or such a cell whose genome has been targeted, edited, modified, or manipulated using the above (a) and (b).

Additional embodiments according to the invention include kits comprising:
(a) a nucleic acid encoding an sRGN polypeptide, wherein the nucleic acid is operably linked to a promoter or a ribosome binding site; and
(b) a single heterologous guide RNA (sgRNA) or a nucleic acid (e.g., DNA) suitable for the generation of such sgRNA in situ (e.g., nucleic acid encoding the sgRNA), wherein the sgRNA comprises:
   i. a DNA-targeting segment comprised of RNA and capable of hybridizing to such target sequence in a polynucleotide locus,
   ii. a tracr mate sequence comprised of RNA, and
   iii. a tracr RNA sequence comprised of RNA,
wherein the tracr mate sequence is capable of hybridizing to the tracr sequence, and wherein (i), (ii), and (iii) are arranged in a 5′ to 3′ orientation.
or
(a) an sRGN polypeptide; and
(b) one or more single heterologous guide RNAs (sgRNAs), each of which comprise(s):
   iv. a DNA-targeting segment comprised of RNA and capable of hybridizing to such target sequence in a polynucleotide locus,
   v. a tracr mate sequence comprised of RNA, and
   vi. a tracr RNA sequence comprised of RNA,
      wherein the tracr mate sequence is capable of hybridizing to the tracr sequence, and wherein (iv), (v), and (vi) are arranged in a 5′ to 3′ orientation.

Yet another embodiment according to the invention comprises compositions and methods for targeting, editing, modifying, or manipulating one or more target DNA(s) at one or more locations in a cell (e.g., in vitro or in vivo) or in a cell-free system, comprising:
(a) an sRGN polypeptide or nucleic acid encoding the sRGN polypeptide; and
(b) a guide RNA (gRNA) or a nucleic acid (e.g., DNA) suitable for the generation of such gRNA in situ (e.g., nucleic acid encoding the gRNA), wherein the gRNA comprises:
   i. a DNA-targeting segment comprised of RNA and capable of hybridizing to such target sequence in a polynucleotide locus,
   ii. a tracr RNA sequence comprised of RNA;
      wherein (i) and (ii) are on a single RNA molecule and (iii) is on a separate RNA molecule.

### Mutants

In some embodiments, the sRGN polypeptide is a mutant polypeptide with altered endoribonuclease activity or associated half-life of pre-crRNA, intermediate crRNA, or mature crRNA. In some embodiments, the sRGN polypeptide is a mutant polypeptide with altered or abrogated DNA endonuclease activity without substantially diminished or enhanced endoribonuclease activity or binding affinity to DNA. Such modification can allow for the sequence-specific DNA targeting of the sRGN polypeptide for the purpose of transcriptional modulation, activation, or repression; epigenetic modification or chromatin modification by methylation, demethylation, acetylation or deacetylation, or any other modifications of DNA binding and/or DNA-modifying proteins known in the art.

In some embodiments, the sRGN polypeptide is a mutant polypeptide with no DNA endonuclease activity.

In some embodiments, the cell is a bacterial cell, a fungal cell, an archaeal cell, a protist, a plant cell, or an animal cell.

In some embodiments, the sRGN polypeptide and the one or more guide RNAs (gRNAs), such as single heterologous guide RNA(s) (sgRNA(s)), are introduced into the cell by the same or different recombinant vectors encoding the polypeptide and the nucleic acid.

In some embodiments, the nucleic acid encoding the polypeptide, nucleic acid, or both the polypeptide and nucleic acid is modified.

In some embodiments, the method or system further comprises adding a donor DNA sequence, and wherein the target DNA sequence is edited by homology directed repair. In some embodiments, the polynucleotide donor template is physically linked to a crRNA or guide RNA

In another aspect, provided herein is a method for modifying or editing double-stranded DNA or single stranded target DNA, without having activity against single stranded RNA (ssRNA), double-stranded RNA (dsRNA), or heteroduplexes of RNA and DNA.

### Multiplexing

In another aspect, provided herein is a method for editing or modifying DNA at multiple locations in a cell consisting essentially of: i) introducing an sRGN polypeptide or a nucleic acid encoding an sRGN polypeptide into the cell; and ii) introducing a single heterologous nucleic acid comprising two or more pre-CRISPR RNAs (pre-crRNAs) either as RNA or encoded as DNA and under the control of one promoter into the cell, each pre-crRNA comprising a repeat-spacer array or repeat-spacer, wherein the spacer comprises a nucleic acid sequence that is complementary to a target sequence in the DNA and the repeat comprises a stem-loop structure, wherein the sRGN polypeptide cleaves the two or more pre-crRNAs upstream of the stem-loop structure to generate two or more intermediate crRNAs, wherein the two or more intermediate crRNAs are processed into two or more mature crRNAs, and wherein each two or more mature crRNAs guides the sRGN polypeptide to effect two or more double-strand breaks (DSBs) into the DNA. For example, one advantage of an sRGN polypeptide is that it is possible to introduce only one pre-crRNA which comprises several repeat-spacer units, which upon introduction, is processed by the sRGN polypeptide it into active repeat-spacer units targeting several different sequences on the DNA.

In some embodiments the pre-crRNA sequences in the single heterologous nucleic acid are joined together in specific locations, orientations, sequences or with specific chemical linkages to direct or differentially modulate the endonuclease activity of the sRGN polypeptide at each of the sites specified by the different crRNA sequences.

In another aspect, provided herein is an example of a general method for editing or modifying the structure or function of DNA at multiple locations in a cell consisting essentially of: i) introducing an RNA-guided endonuclease, such as sRGN, as a polypeptide or a nucleic acid encoding the RNA-guided endonuclease into the cell; and ii) introducing a single heterologous nucleic acid comprising or encoding two or more guide RNAs, either as RNA or encoded as DNA and under the control of one or more promoters, wherein the activity or function of the RNA-guided endonuclease is directed by the guide RNA sequences in the single heterologous nucleic acid.

Codon Optimized DNA Sequences for sRGN Polypeptides

In some embodiments of the method, the nucleic acid sequence encoding the sRGN polypeptide is a modified nucleic acid, for example, codon optimized. In some embodiments of the method, the single heterologous nucleic acid is a modified nucleic acid. In some embodiments of the method, the method further comprises introducing into the cell a polynucleotide donor template. In some embodiments, the polynucleotide donor template is physically linked to a crRNA or guide RNA. In some embodiments of the method, the DNA is repaired at DSBs by either homology directed repair, non-homologous end joining, or microhomology-mediated end joining.

In some embodiments of the method, the sRGN polypeptide is more readily complexed with a mature crRNA in the local milieu, and thus more readily available for directing DNA endonuclease activity as a consequence of the crRNA being processed by the same sRGN polypeptide from the pre-crRNA in the local milieu.

In some embodiments of the method, the sRGN polypeptide is used to cleave, isolate or purify one or more mature crRNA sequences from a modified pre-crRNA oligonucleotide sequence in which heterologous sequences are incorporated 5′ or 3′ to one or more crRNA sequences within RNA oligonucleotide or DNA expression construct. The heterologous sequences can be incorporated to modify the stability, half-life, expression level or timing, interaction with the sRGN polypeptide or target DNA sequence, or any other physical or biochemical characteristics known in the art.

In some embodiments of the method, the pre-crRNA sequence is modified to provide for differential regulation of two or more mature crRNA sequences within the pre-crRNA sequence, to differentially modify the stability, half-life, expression level or timing, interaction with the sRGN polypeptide or target DNA sequence, or any other physical or biochemical characteristics known in the art.

In some embodiments, the sRGN polypeptide (or nucleic acid encoded variants thereof) is modified to improve desired characteristics such as function, activity, kinetics, half-life or the like. One such non-limiting example of such a modification is to replace a ‘cleavage domain’ of the sRGN polypeptide with a homologous or heterologous cleavage domain from a different nuclease, such as the RuvC or HNH domain from the Type II CRISPR-associated nuclease Cas9.

In one aspect, provided herein is a method for targeting, editing or manipulating DNA in a cell comprising linking an intact or partially or fully deficient sRGN polypeptide or pre-crRNA or crRNA moiety, to a dimeric FOK1 nuclease to direct endonuclease cleavage, as directed to one or more specific DNA target sites by one or more crRNA molecules. In another embodiment, the FOK1 nuclease system is a nickase or temperature sensitive mutant or any other variant known in the art.

In some embodiments, the sRGN polypeptide linked with a dimeric FOK1 nuclease is introduced into the cell together with a single heterologous nucleic acid comprising two or more pre-CRISPR RNAs (pre-crRNAs) either as RNA or encoded as DNA and under the control of one promoter into the cell, each pre-crRNA comprising a repeat-spacer array, wherein the spacer comprises a nucleic acid sequence that is complementary to a target sequence in the DNA and the repeat comprises a stem-loop structure, wherein the sRGN polypeptide cleaves the two or more pre-crRNAs upstream of the stem-loop structure to generate two or more intermediate crRNAs.

In one aspect, provided herein is a method for targeting, editing or manipulating DNA in a cell comprising linking an intact or partially or fully deficient sRGN polypeptide or pre-crRNA, intermediate crRNA, mature crRNA moiety, or gRNA (collectively referred to as crRNA), to a donor single or double-strand DNA donor template to facilitate homologous recombination of exogenous DNA sequences, as directed to one or more specific DNA target sites by one or more guide RNA or crRNA molecules.

In yet another aspect, provided herein is a method for directing a DNA template, for homologous recombination or homology-directed repair, to the specific site of gene editing. In this regard, a single stranded or double-stranded DNA template is linked chemically or by other means known in the art to a crRNA or guide RNA. In some embodiments the DNA template remains linked to the crRNA or guide RNA; in yet other examples, the sRGN polypeptide cleaves the crRNA or guide RNA, liberating the DNA template to enable or facilitate homologous recombination.

In yet another aspect, provided herein is a method for targeting, editing or manipulating DNA in a cell comprising linking an intact or partially or fully deficient sRGN polypeptide or pre-crRNA or crRNA moiety, to a transcriptional activator or repressor, or epigenetic modifier such as a methylase, demethylase, acetylase, or deacetylase, or signaling or detection, all aspects of which have been previously described for Cas9 systems, as directed to one or more specific DNA target sites by one or more crRNA molecules.

In another aspect, provided herein is a composition comprising a polynucleotide donor template linked to a crRNA or a guide RNA. A method for targeting, editing or manipulating DNA in a cell comprising linking a pre-crRNA or crRNA or guide RNA to a donor single or double-strand polynucleotide donor template such that the donor template is cleaved from the pre-crRNA or crRNA or guide RNA by an sRGN polypeptide, thus facilitating homology directed repair by the donor template, as directed to one or more specific DNA target sites by one or more guide RNA or crRNA molecules.

An sRGN polypeptide can also be used to form a chimeric binding protein in which other domains and activities are introduced. By way of illustration, a Fokl domain can be fused to an sRGN protein, which can contain a catalytically active endonuclease domain, or a Fokl domain can be fused to an sRGN protein, which has been modified to render the sRGN polypeptide endonuclease domain inactive. Other domains that can be fused to make chimeric proteins with an sRGN polypeptide include transcriptional modulators, epigenetic modifiers, tags and other labels or imaging agents, histones, and/or other modalities known in the art that modulate or modify the structure or activity of gene sequences.

Nucleic Acid and/or Amino Acid Modifications

In some embodiments, polynucleotides introduced into cells comprise one or more modifications which can be used, for example, to enhance activity, stability or specificity, alter delivery, reduce innate immune responses in host cells, further reduce the protein size, or for other enhancements, as further described herein and known in the art.

In certain embodiments, modified polynucleotides are used in a CRISPR-Cas system based on an sRGN, in which case the guide RNAs and/or a DNA or an RNA encoding an sRGN polypeptide introduced into a cell can be modified, as described and illustrated below. Such modified polynucleotides can be used in the CRISPR-Cas system to edit any one or more genomic loci.

### Components of CRISPR-Cas Systems

### A. Guide RNAs/sgRNAs

In aspects of the invention the terms “chimeric RNA”, “chimeric guide RNA”, “guide RNA”, “single guide RNA” and “synthetic guide RNA” are used interchangeably and refer to the polynucleotide sequence comprising the DNA-targeting segment, the tracr sequence and the tracr mate sequence. The term “guide sequence” or “DNA-targeting sequence” refers to the about 20 bp sequence within the guide RNA that specifies the target site and may be used interchangeably with the terms “guide” or “spacer’. The term “tracr mate sequence” may also be used interchangeably with the term “direct repeat(s)”.

In some aspects, a single guide RNA (sgRNA) according to the invention comprises:
i. a DNA-targeting segment or DNA-targeting sequence comprised of RNA and capable of hybridizing to a target sequence in a polynucleotide locus,
ii. a tracr mate sequence comprised of RNA, and
iii. a tracr RNA sequence comprised of RNA,
   wherein the tracr mate sequence is capable of hybridizing to the tracr sequence, and wherein (i), (ii), and (iii) are arranged in a 5′ to 3′ orientation. In some embodiments, components (i), (ii), and (iii) are on a single chimeric RNA.

Within a sgRNA, a tracr mate sequence and a tracr sequence can connected by a suitable loop sequence and form a stem-loop structure.

In general, a tracr mate sequence includes any sequence that has sufficient complementarity with a tracr sequence to promote one or more of: (1) excision of a DNA-targeting segment flanked by tracr mate sequences in a cell containing the corresponding tracr sequence; and (2) formation of a CRISPR complex at a target sequence, wherein the CRISPR complex comprises the tracr mate sequence hybridized to the tracr sequence. In general, degree of complementarity is with reference to the optimal alignment of the tracr mate sequence and tracr sequence, along the length of the shorter of the two sequences. Optimal alignment may be determined by any suitable alignment algorithm and may further account for secondary structures, such as self-complementarity within either the tracr sequence or tracr mate sequence. In some embodiments, the degree of complementarity between the tracr sequence and tracr mate sequence along the 30 nucleotides length of the shorter of the two when optimally aligned is about or more than 25%, 30%, 40%, 50%, 60%, 70%, 80%, 90%, 95%, 97.5%, 99%, or higher. In some embodiments, the tracr sequence is about or more than 5, 6, 7, 8, 9, 10, 11, 12, 13, 14, 15, 16, 17, 18, 19, 20, 25, 30, 40, 50, or more nucleotides in length. In some embodiments, the tracr sequence and tracr mate sequence are contained within a single transcript, such that hybridization between the two produces a transcript having a secondary structure, such as a hairpin. In an embodiment of the invention, the transcript or transcribed polynucleotide sequence has at least two or more hairpins.

In some embodiments, the transcript has two, three, four or five hairpins. In a further embodiment of the invention, the transcript has at most five hairpins. In a hairpin structure the portion of the sequence 5′ of the final “N” and upstream of the loop corresponds to the tracr mate sequence, and the portion of the sequence 3′ of the loop corresponds to the tracr sequence. Further non-limiting examples of single polynucleotides comprising a DNA-targeting segment, a tracr mate sequence, and a tracr sequence are as follows (listed 5′ to 3′), where “N” represents a base of a DNA-targeting segment, the first block of lower case letters represent the tracr mate sequence, and the second block of lower case letters represent the tracr sequence, and the final poly-T sequence represents the transcription terminator:

The DNA-targeting segment of a guide RNA comprises a nucleotide sequence that is complementary to a sequence in a target DNA. In other words, the DNA-targeting segment of a guide RNA interacts with a target DNA in a sequence-specific manner via hybridization (i.e., base pairing). As such, the nucleotide sequence of the DNA-targeting segment may vary and determines the location within the target DNA that the guide RNA and the target DNA will interact. The DNA-targeting segment of a guide RNA can be modified (e.g., by genetic engineering) to hybridize to any desired sequence within a target DNA.

The DNA-targeting segment can have a length of from 10 nucleotides to 30 nucleotides.

In some embodiments, the DNA-targeting segment has a length of from 13 nucleotides to 25 nucleotides.

In some embodiments, the DNA-targeting segment has a length of from 15 nucleotides to 23 nucleotides.

In some embodiments, the DNA-targeting segment has a length of from 18 nucleotides to 22 nucleotides, such as from 20 to 22 nucleotides.

The percent complementarity between the DNA-targeting sequence of the DNA-targeting segment and the target sequence of the target DNA can be at least 60% (e.g., at least 65%, at least 70%, at least 75%, at least 80%, at least 85%, at least 90%, at least 95%, at least 97%, at least 98%, at least 99%, or 100%) over the 20-22 nucleotides.

A protospacer sequence complementary to the target sequence in the target DNA can be adjacent to a suitable PAM sequence for an sRGN polypeptide at its 3′ end, or such PAM sequence can be part of the 3′ portion of the DNA-targeting segment.

Using the CRISPR-Cas system for purposes of non-limiting illustrations of such uses, modifications of guide RNAs can be used to enhance the formation or stability of the CRISPR-Cas genome editing complex comprising guide RNAs and a Cas endonuclease such as sRGN. Modifications of guide RNAs can also or alternatively be used to enhance the initiation, stability or kinetics of interactions between the genome editing complex with the target sequence in the genome, which can be used for example to enhance on-target activity. Modifications of guide RNAs can also or alternatively be used to enhance specificity, e.g., the relative rates of genome editing at the on-target site as compared to effects at other (off-target) sites.

Modifications can also or alternatively used to increase the stability of a guide RNA, e.g., by increasing its resistance to degradation by ribonucleases (RNases) present in a cell, thereby causing its half-life in the cell to be increased. Modifications enhancing guide RNA half-life can be particularly useful in embodiments in which a Cas endonuclease such as an sRGN polypeptide is introduced into the cell to be edited via an RNA that needs to be translated in order to generate the sRGN polypeptide, since increasing the half life of guide RNAs introduced at the same time as the RNA encoding the endonuclease can be used to increase the time that the guide RNAs and the encoded Cas endonuclease co-exist in the cell.

### B. Protospacer-Adjacent Motif (PAM or PAM Sequence)

Suitable protospacer adjacent motif (PAM) sequences for an sRGN polypeptide include “ANGG” and “NNGG.”

The site of cleavage generally occurs within one to three base pairs upstream of the PAM sequence, such as within either one or three base pairs upstream of the PAM sequence, within three base pairs of the PAM sequence, or within three base pairs upstream of the PAM sequence “NNGG”. Other PAMs are known in the art and an sRGN can be engineered to target such PAMs.

### C. Use in Eukaryotic Cells

In one embodiment according to the invention the sRGN system herein described can be used in eukaryotic cells, such as mammalian cells. In some embodiments, the cells are not human fetal cells or derived from human fetal cells. In some embodiments, the cells are not human embryonic cells or derived from human embryonic cells. In some embodiments, the compositions and methods disclosed herein do not involve the destruction of a human fetus or a human embryo. In some embodiments, the subject (or individual, whatever you're using in the spec) is not human fetus or embryo.

### Other Features of the Invention

Modifications can also or alternatively be used to decrease the likelihood or degree to which RNAs introduced into cells elicit innate immune responses. Such responses, which have been well characterized in the context of RNA interference (RNAi), including small-interfering RNAs (siRNAs), as described below and in the art, tend to be associated with reduced half-life of the RNA and/or the elicitation of cytokines or other factors associated with immune responses.

One or more types of modifications can also be made to RNAs encoding an endonuclease such as an sRGN polypeptide that are introduced into a cell, including, without limitation, modifications that enhance the stability of the RNA (such as by decreasing its degradation by RNases present in the cell), modifications that enhance translation of the resulting product (i.e., the endonuclease), and/or modifications that decrease the likelihood or degree to which the RNAs introduced into cells elicit innate immune responses. Combinations of modifications, such as the foregoing and others, can likewise be used. In the case of CRISPR-Cas, for example, one or more types of modifications can be made to guide RNAs (including those exemplified above), and/or one or more types of modifications can be made to RNAs encoding CAS endonuclease (including those exemplified above).

By way of illustration, guide RNAs used in the CRISPR-Cas system or other smaller RNAs can be readily synthesized by chemical means, enabling a number of modifications to be readily incorporated, as illustrated below and described in the art. While chemical synthetic procedures are continually expanding, purifications of such RNAs by procedures such as high performance liquid chromatography (HPLC, which avoids the use of gels such as PAGE) tends to become more challenging as polynucleotide lengths increase significantly beyond a hundred or so nucleotides. One approach used for generating chemically-modified RNAs of greater length is to produce two or more molecules that are ligated together. Much longer RNAs, such as those encoding an sRGN polypeptide, are more readily generated enzymatically. While fewer types of modifications are generally available for use in enzymatically produced RNAs, there are still modifications that can be used to, e.g., enhance stability, reduced the likelihood or degree of innate immune response, and/or enhance other attributes, as described further below and in the art; and new types of modifications are regularly being developed. By way of illustration of various types of modifications, especially those used frequently with smaller chemically synthesized RNAs, modifications can comprise one or more nucleotides modified at the 2′ position of the sugar, in some embodiments a 2′-0-alkyl, 2′-0-alkyl-0-alkyl or 2′-fluoro-modified nucleotide. In some embodiments, RNA modifications include 2′-fluoro, 2′-amino and 2′0-methyl modifications on the ribose of pyrimidines, basic residues or an inverted base at the 3′ end of the RNA. Such modifications are routinely incorporated into oligonucleotides and these oligonucleotides have been shown to have a higher Tm (i.e., higher target binding affinity) than; 2′-deoxy oligonucleotides against a given target.

A number of nucleotide and nucleoside modifications have been shown to make the oligonucleotide into which they are incorporated more resistant to nuclease digestion than the native oligonucleotide; these modified oligonucleotides survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. Some oligonucleotides are oligonucleotides with phosphorothioate backbones and those with heteroatom backbones, particularly CH₂—NH—O—CH₂, CH, —N(CH₃)-0-CH₂ (known as a methylene(methylimino) or MMI backbone), CH₂—O—N(CH₃)—CH₂, CH₂—N(CH₃)—N(CH₃)—CH₂ and 0-N(CH₃)—CH₂—CH₂ backbones; amide backbones [see De Mesmaeker et al., Ace. Chem. Res., 28:366-374 (1995)]; morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Morpholino-based oligomeric compounds are described in Braasch and David Corey, Biochemistry, 41 (14): 4503-4510 (2002); Genesis, Volume 30, Issue 3, (2001); Heasman, Dev. Biol., 243:209-214 (2002); Nasevicius et al., Nat. Genet., 26:216-220 (2000); Lacenra et a!., Proc. Nat/. Acad. Sci., 97:9591-9596 (2000); and U.S. Pat. No. 5,034,506, issued Jul. 23, 1991. Cyclohexenyl nucleic acid oligonucleotide mimetics are described in Wang et al., J. Am. Chem. Soc., 122:8595-8602 (2000).

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, Sand CH2 component parts; see U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

One or more substituted sugar moieties can also be included, e.g., one of the following at the 2′ position: OH, SH, SCH₃, F, OCN, OCH₃, OCH₃ O(CH₂)n CH₃, O(CH₂)n NH₂ or O(CH₂)n CH₃ where n is from 1 to 10; C₁ to C₁₀ lower alkyl, alkoxyalkoxy, substituted lower alkyl, alkaryl or aralkyl; Cl; Br; CN; CF₃; OCF₃; O—, S—, or N-alkyl; O—, S—, or N-alkenyl: SOCH₃; SO₂ CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. In some embodiments, a modification includes 2′-methoxyethoxy (2′-O—CH2CH2OCH3, also known as 2′-O-(2-methoxyethyl)) (Martin et al., Helv. Chim. Acta, 1995, 78, 486). Other modifications include 2′-methoxy (2′-O—CH3), 2′-propoxy (2′-OCH2 CH2CH3) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group. In some embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds comprise, but are not limited to, U.S. Pat. Nos. 5,539,082; 5,714,331; and 5,719,262. Further teaching of PNA compounds can be found in Nielsen et al, Science, 254:1497-1500 (1991).

Guide RNAs can also include, additionally or alternatively, nucleobase (alternately referred to as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and can be referred to in the art as 5-Me-C), 5-hydroxymethylcytosine (HMC), glycosyl HMC and gentobiosyl HMC, as well as synthetic nucleobases, e.g., 2-aminoadenine, 2-(methylamino) adenine, 2-(imidazolylalkyl) adenine, 2-(aminoalklyamino) adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl) adenine and 2.6-diaminopurine. Kornberg, A, DNA Replication, W. H. Freeman & Co., San Francisco, pp75-77 (1980); Gebeyehu et al., Nucl. Acids Res. 15:4513 (1997). A “universal” base known in the art, e.g., inosine, can also be included. 5-Me-C substitutions have been shown to increase nucleic acid duplex stability by 0.6-1.2 degrees C. (Sanghvi, Y. S., in Crooke, S. T. and Lebleu, B., eds., Antisense Research and Applications, CRC Press, Boca Raton, 1993, pp. 276-278) and are embodiments of base substitutions.

Modified nucleobases comprise other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other a-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine.

Further, nucleobases comprise those disclosed in U.S. Pat. No. 3,687,808, those disclosed in ‘The Concise Encyclopedia of Polymer Science And Engineering’, pages 858-859, Kroschwitz, J. l., ed. John Wiley & Sons, 1990, those disclosed by Englisch et al., Angewandte Chemie, International Edition’, 1991, 30, page 613, and those disclosed by Sanghvi, Y. S., Chapter 15, Antisense Research and Applications’, pages 289-302, Crooke, S. T. and Lebleu, B. ea., CRC Press, 1993. Certain of these nucleobases are particularly useful for increasing the binding affinity of the oligomeric compounds of the invention. These include 5-substituted pyrimidines, 6-azapyrimidines and N-2, N-6 and —O-6 substituted purines, comprising 2-aminopropyladenine, 5-propynyluracil and 5-propynylcytosine. 5-methylcytosine substitutions have been shown to increase nucleic acid duplex stability by 0.6-1.2 oc (Sanghvi, Y. S., Crooke, S. T. and Lebleu, B., eds, ‘Antisense Research and Applications’, CRC Press, Boca Raton, 1993, pp. 276-278) and are embodiments of base substitutions, even more particularly when combined with 2′-O-methoxyethyl sugar modifications. Modified nucleobases are described in U.S. Pat. No. 3,687,808, as well as U.S. Pat. Nos. 4,845,205; 5,130,302; 5,134,066; 5,175,273; 5, 367,066; 5,432,272; 5,457,187; 5,459,255; 5,484,908; 5,502,177; 5,525,711; 5,552,540; 5,587,469; 5,596,091; 5,614,617; 5,681,941; 5,750,692; 5,763,588; 5,830,653; 6,005,096; and US Patent Application Publication 20030158403.

It is not necessary for all positions in a given oligonucleotide to be uniformly modified, and in fact more than one of the aforementioned modifications may be incorporated in a single oligonucleotide or even at within a single nucleoside within an oligonucleotide.

In some embodiments, the guide RNAs and/or mRNA (or DNA) encoding an endonuclease such as an sRGN polypeptide are chemically linked to one or more moieties or conjugates that enhance the activity, cellular distribution, or cellular uptake of the oligonucleotide. Such moieties comprise but are not limited to, lipid moieties such as a cholesterol moiety [Letsinger et al., Proc. Nat/. Acad. Sci. USA, 86:6553-6556 (1989)]; cholic acid [Manoharan et al., Bioorg. Med. Chem. Let., 4:1053-1060 (1994)]; a thioether, e.g., hexyi-S-tritylthiol [Manoharan et al., Ann. N. Y Acad. Sci., 660:306-309 (1992) and Manoharan et al., Bioorg. Med. Chem. Let., 3 . . . 2765-2770 (1993)); a thiocholesterol [Oberhauser et al., Nucl. Acids Res., 20:533-538 (1992)]; an aliphatic chain, e.g., dodecandiol or undecyl residues [Kabanov et al., FEBS Lett., 259:327-330 (1990) and Svinarchuk et al., Biochimie, 75:49-54 (1993)]; a phospholipid, e.g., di-hexadecyl-rac-glycerol or triethylammonium 1,2-di-O-hexadecyl-rac-glycero-3-H-phosphonate [Manoharan et al., Tetrahedron Lett., 36:3651-3654 (1995) and Shea et al., Nucl. Acids Res., 18:3777-3783 (1990)]; a polyamine or a polyethylene glycol chain [Mancharan et al., Nucfeosides & Nucleotides, 14:969-973 (1995)]; adamantane acetic acid [Manoharan et at., Tetrahedron Lett., 36:3651-3654 (1995)]; a palmityl moiety [(Mishra et al., Biochim. Biophys. Acta, 1264:229-237 (1995)]; or an octadecylamine or hexylamino-carbonyl-t oxycholesterol moiety [Crooke et al., J. Pharmacal. Exp. Ther., 277:923-937 (1996)]. See also U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391, 723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

Sugars and other moieties can be used to target proteins and complexes comprising nucleotides, such as cationic polysomes and liposomes, to particular sites. For example, hepatic cell directed transfer can be mediated via asialoglycoprotein receptors (ASGPRs); e.g., Hu, et al., Protein Pept Lett. 21 (1 0): 1025-30 (2014). Other systems known in the art and regularly developed can be used to target biomolecules of use in the present case and/or complexes thereof to particular target cells of interest.

These targeting moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention. Representative conjugate groups are disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

Longer polynucleotides that are less amenable to chemical synthesis and are generally produced by enzymatic synthesis can be modified by various means. Such modifications can include, for example, the introduction of certain nucleotide analogs, the incorporation of particular sequences or other moieties at the 5′ or 3′ ends of molecules, and other modifications. By way of illustration, the mRNA encoding an sRGN polypeptide is approximately 3 kb to 3.3 kb in length and can be synthesized by in vitro transcription. Modifications to the mRNA can be applied to, e.g., increase its translation or stability (such as by increasing its resistance to degradation with a cell), or to reduce the tendency of the RNA to elicit an innate immune response that may occur following introduction of exogenous RNAs, particularly longer RNAs such as those encoding sRGN.

Numerous such modifications have been described in the art, such as polyA tails, 5′ cap analogs (e.g., Anti Reverse Cap Analog (ARCA) or m7G(5′)ppp(5′)G (mCAP)), modified 5′ or 3′ untranslated regions (UTRs), use of modified bases (such as Pseudo-UTP, 2-Thio-UTP, 5-Methylcytidine-5′-Triphosphate (5-methyl-CTP) or N6-methyl-ATP), or treatment with phosphatase to remove 5′ terminal phosphates. These and other modifications are known in the art, and new modifications of RNAs are being developed.

Modified RNAs can be obtained, e.g., from a commercial supplier, including for example, Trilink Biotech, Axolabs, Bio-Synthesis Inc. Dharmacon and many others. As described by Trilink, for example, 5-methyl-CTP can be used to impart desirable characteristics such as increased nuclease stability, increased translation or reduced interaction of innate immune receptors with in vitro transcribed RNA. 5′-Methylcytidine-5′-triphosphate (5-methyl-CTP), N6-methyl-ATP, as well as Pseudo-UTP and 2-Thio-UTP, have also been shown to reduce innate immune stimulation in culture and in vivo while enhancing translation as illustrated in publications by Konmann et al. and Warren et al. referred to below.

It has been shown that chemically modified mRNA delivered in vivo can be used to achieve improved therapeutic effects; e.g., Kormann et al., Nature Biotechnology 29, 154-157 (2011). Such modifications can be used, for example, to increase the stability of the RNA molecule and/or reduce its immunogenicity. Using chemical modifications such as Pseudo-U, N6-methyl-A, 2-Thio-U and 5-methyl-C, it was found substituting just one quarter of the uridine and cytidine residues with 2-Thio-U and 5-methyl-C respectively, resulted in a significant decrease in toll-like receptor (TLR) mediated recognition of the mRNA in mice. By reducing the activation of the innate immune system, these modifications can therefore be used to effectively increase the stability and longevity of the mRNA in vivo; e.g., Konmann et al., supra.

It has also been shown that repeated administration of synthetic messenger RNAs incorporating modifications designed to bypass innate anti-viral responses can reprogram differentiated human cells to pluripotency. See, e.g., Warren, et al., Cell Stem Cell, 7 (5): 618-30 (2010). Such modified mRNAs that act as primary reprogramming proteins can be an efficient means of reprogramming multiple human cell types. Such cells are referred to as induced pluripotency stem cells (iPSCs). and it was found that enzymatically synthesized RNA incorporating 5-methyl-CTP, Pseudo-UTP and an Anti Reverse Cap Analog (ARCA) could be used to effectively evade the cell's antiviral response; e.g., Warren et al., supra. Other modifications of polynucleotides described in the art include, for example, the use of polyA tails, the addition of 5′ cap analogs (such as m7G (5′) ppp (5′) G (mCAP)), modifications of 5′ or 3′ untranslated regions (UTRs), or treatment with phosphatase to remove 5′ terminal phosphates—and new approaches are regularly being developed.

A number of compositions and techniques applicable to the generation of modified RNAs for use herein have been developed in connection with the modification of RNA interference (RNAi), including small-interfering RNAs (siRNAs). siRNAs present particular challenges in vivo because their effects on gene silencing via mRNA interference are generally transient, which can require repeat administration. In addition, siRNAs are double-stranded RNAs (dsRNA) and mammalian cells have immune responses that have evolved to detect and neutralize dsRNA, which is often a by-product of viral infection. Thus, there are mammalian enzymes such as PKR (dsRNA-responsive kinase), and potentially retinoic acid-inducible gene I (RIG-I), that can mediate cellular responses to dsRNA, as well as Toll-like receptors (such as TLR3, TLR7 and TLR8) that can trigger the induction of cytokines in response to such molecules; e.g., the reviews by Angart et al., Pharmaceuticals (Basel) 6 (4): 440-468 (2013); Kanasty et al., Molecular Therapy 20 (3): 513-524 (2012); Burnett et al., Biotechnol J. 6 (9): 1130-46 (2011); Judge and Maclachlan, Hum Gene Ther 19 (2): 111-24 (2008); and references cited therein.

Modifications described in the art have been developed and applied to enhance RNA stability, reduce innate immune responses, and/or achieve other benefits that can be useful in connection with the introduction of polynucleotides into human cells as described herein; e.g. the reviews by Whitehead K A et al., Annual Review of Chemical and Biomolecular Engineering, 2:77-96 (2011); Gaglione and Messere, Mini Rev Med Chem, 10 (7): 578-95 (2010); Chernolovskaya et al, Curr Opin Mol Ther., 12 (2): 158-67 (2010); Deleavey et al., Curr Protoc Nucleic Acid Chem Chapter 16: Unit 16.3 (2009); Behlke, Oligonucleotides 18 (4): 305-19 (2008): Fucini et al., Nucleic Acid Ther 22 (3): 205-210 (2012); Bremsen et al., Front Genet 3:154 (2012).

As noted above, there are a number of commercial suppliers of modified RNAs, many of which have specialized in modifications designed to improve the effectiveness of siRNAs. A variety of approaches are offered based on various findings reported in the literature. For example, Dharmacon notes that replacement of a non-bridging oxygen with sulfur (phosphorothioate, PS) has been extensively used to improve nuclease resistance of siRNAs, as reported by Kale, Nature Reviews Drug Discovery 11:125-140 (2012). Modifications of the 2′-position of the ribose have been reported to improve nuclease resistance of the internucleotide phosphate bond while increasing duplex stability (Tm), which has also been shown to provide protection from immune activation. A combination of moderate PS backbone modifications with small, well-tolerated 2′-substitutions (2′-O-Methyl, 2′-Fluoro, 2′-Hydro) has been associated with highly stable siRNAs for applications in vivo, as reported by Soutschek et al. Nature 432:173-178 (2004); and 2′-O-Methyl modifications have been reported to be effective in improving stability as reported by Volkov, Oligonucleotides 19:191-202 (2009). With respect to decreasing the induction of innate immune responses, modifying specific sequences with 2′-O-methyl, 2′-fluoro, 2′-hydro has been reported to reduce TLR7/TLR8 interaction while generally preserving silencing activity; e.g., Judge et al., Mol. Ther. 13:494-505 (2006); and Cekaite et al., J Mol Biol. 365:90-108 (2007). Additional modifications, such as 2-thiouracil, pseudouracil, 5-methylcytosine, 5-methyluracil, and N6-methyladenosine have also been shown to minimize the immune effects mediated by TLR3, TLR7, and TLR8; e.g. Kariko, K. et al., Immunity 23:165-175 (2005).

As is also known in the art, and commercially available, a number of conjugates can be applied to polynucleotides such as RNAs for use herein that can enhance their delivery and/or uptake by cells, including for example, cholesterol, tocopherol and folic acid, lipids, peptides, polymers, linkers and aptamers; e.g., the review by Winkler, Ther Deliv. 4:791-809 (2013), and references cited therein.

### Mimetics

A nucleic acid can be a nucleic acid mimetic. The term “mimetic” as it is applied to polynucleotides is intended to include polynucleotides wherein only the furanose ring or both the furanose ring and the internucleotide linkage are replaced with non-furanose groups, replacement of only the furanose ring is also referred to in the art as being a sugar surrogate. The heterocyclic base moiety or a modified heterocyclic base moiety is maintained for hybridization with an appropriate target nucleic acid. One such nucleic acid, a polynucleotide mimetic that has been shown to have excellent hybridization properties, is referred to as a peptide nucleic acid (PNA). In PNA, the sugar-backbone of a polynucleotide is replaced with an amide containing backbone, in particular an aminoethylglycine backbone. The nucleotides are retained and are bound directly or indirectly to aza nitrogen atoms of the amide portion of the backbone.

One polynucleotide mimetic that has been reported to have excellent hybridization properties is a peptide nucleic acid (PNA). The backbone in PNA compounds is two or more linked aminoethylglycine units, which gives PNA an amide containing backbone. The heterocyclic base moieties are bound directly or indirectly to aza nitrogen atoms of the amide portion of the backbone. Representative US patents that describe the preparation of PNA compounds include, but are not limited to: U.S. Pat. Nos. 5,539,082; 5,714,331; and 5,719,262.

Another class of polynucleotide mimetic that has been studied is based on linked morpholino units (morpholino nucleic acid) having heterocyclic bases attached to the morpholino ring. A number of linking groups have been reported that link the morpholino monomeric units in a morpholino nucleic acid. One class of linking groups has been selected to give a non-ionic oligomeric compound. The non-ionic morpholino-based oligomeric compounds are less likely to have undesired interactions with cellular proteins. Morpholino-based polynucleotides are nonionic mimics of oligonucleotides, which are less likely to form undesired interactions with cellular proteins (Braasch, D. A., and Corey, D. R., Biochemistry, 2002, 41 (14), 4503-4510). Morpholino-based polynucleotides are disclosed in U.S. Pat. No. 5,034,506. A variety of compounds within the morpholino class of polynucleotides have been prepared, having a variety of different linking groups joining the monomeric subunits.

A further class of polynucleotide mimetic is referred to as cyclohexenyl nucleic acids (GeNA). The furanose ring normally present in a DNA/RNA molecule is replaced with a cydohexenyl ring. GeNA DMT protected phosphoramidite monomers have been prepared and used for oligomeric compound synthesis following classical phosphoramidite chemistry. Fully modified GeNA oligomeric compounds and oligonucleotides having specific positions modified with GeNA have been prepared and studied (see Wang et al., J. Am. Chem. Soc., 2000, 122, 8595-8602). In general the incorporation of GeNA monomers into a DNA chain increases its stability of a DNA/RNA hybrid. GeNA oligoadenylates formed complexes with RNA and DNA complements with similar stability to the native complexes. The study of incorporating GeNA structures into natural nucleic acid structures was shown by NMR and circular dichroism to proceed with easy conformational adaptation.

A further modification includes Locked Nucleic Acids (LNAs) in which the 2′-hydroxyl group is linked to the 4′ carbon atom of the sugar ring thereby forming a 2′-C,4′-C-oxymethylene linkage thereby forming a bicyclic sugar moiety. The linkage can be a methylene (—CHz-), group bridging the 2′ oxygen atom and the 4′ carbon atom wherein n is 1 or 2 (Singh et al., Chem. Commun., 1998, 4, 455-456). LNA and LNA analogs display very high duplex thermal stabilities with complementary DNA and RNA (Tₘ=+3 to +10° C.), stability towards 3′-exonucleolytic degradation and good solubility properties. Potent and nontoxic antisense oligonucleotides containing LNAs have been described (Wahlestedt et al., Proc. Natl. Acad. Sci. U.S.A, 2000, 97, 5633-5638).

The synthesis and preparation of the LNA monomers adenine, cytosine, guanine, 5-methyl-cytosine. thymine and uracil, along with their oligomerization, and nucleic acid recognition properties have been described (Koshkin et al., Tetrahedron, 1998, 54, 3607-3630). LNAs and preparation thereof are also described in WO 98/39352 and WO 99/14226.

### Modified Sugar Moieties

A nucleic acid can also include one or more substituted sugar moieties. Suitable polynucleotides comprise a sugar substituent group selected from: OH; F; O—, S—, or N-alkyl; O—, S—, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly suitable are O((CH₂)nO)mCH₃, O(CHz)nOCH3, O(CHz)nNH₂, O(CH₂)CH₃, O(CH₂)nONH₂, and O(CH₂)nON((CH₂)nCH₃)₂, where n and m are from 1 to about 10. Other suitable polynucleotides comprise a sugar substituent group selected from: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONOz, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A suitable modification includes 2′-methoxyethoxy 2′-O—CH2 CHzOCH3, also known as -2′-O-(2-methoxyethyl) or 2′-MOE) (Martin et al., Helv. Chim. Acta, 1995, 78, 486-504) i.e., an alkoxyalkoxy group. A further suitable modification includes 2′-dimethylaminooxyethoxy, i.e., a O(CHz)zON(CH3)z group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—CHz-O—CHz-N(CH3)z.

Other suitable sugar substituent groups include methoxy (—O—CH₃), aminopropoxy (—O—CH_{z}CH_{z}CH_{z}NH_{z}), allyl (—CH₂—CH═CH_{z}), —O-allyl (—O—CH_{z}—CH═CH_{z}) and fluoro (F). 2′-sugar substituent groups may be in the arabino (up) position or ribo (down) position. A suitable 2′-arabino modification is 2′-F. Similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimetics such as cyclobutyl moieties in place of the pentofuranosyl sugar.

### Base Modifications and Substitutions

A nucleic acid may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido(5,4-b)(1,4)benzoxazin-2(3H)-one), phenothiazine cytidine(1H-pyrimido (5,4-b)(1,4)benzothiazin-2(3H)-one), G-clamps such as a substituted phenoxazine cytidine (e.g., 9-(2-aminoethoxy)-H-pyrimido(5,4-(b)(1,4)benzoxazin-2(3H)-one), carbazole cytidine(2H-pyrimido(4,5-b)indol-2-one), pyridoindole cytidine(H-pyrido (3′,2′: 4,5)pyrrolo(2,3-d)pyrimidin-2-one).

Heterocyclic base moieties may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. 1., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are useful for increasing the binding affinity of an oligomeric compound. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2 oc. (Sanghvi et al., eds., Antisense Research and Applications, CRC Press, Boca Raton, 1993, pp. 276-278) and are suitable base substitutions, e.g., when combined with 2′-O-methoxyethyl sugar modifications.

“Complementary” refers to the capacity for pairing, through base stacking and specific hydrogen bonding, between two sequences comprising naturally or non-naturally occurring (e.g., modified as described above) bases (nucleosides) or analogs thereof. For example, if a base at one position of a nucleic acid is capable of hydrogen bonding with a base at the corresponding position of a target, then the bases are considered to be complementary to each other at that position. Nucleic acids can comprise universal bases, or inert abasic spacers that provide no positive or negative contribution to hydrogen bonding. Base pairings may include both canonical Watson-Crick base pairing and non-Watson-Crick base pairing (e.g., wobble base pairing and Hoogsteen base pairing).

It is understood that for complementary base pairings, adenosine-type bases (A) are complementary to thymidine-type bases (T) or uracil-type bases (U), that cytosine-type bases (C) are complementary to guanosine-type bases (G), and that universal bases such as such as 3-nitropyrrole or 5-nitroindole can hybridize to and are considered complementary to any A, C, U, or T. Nichols et al., Nature,

1994; 369:492-493 and Loakes et al., Nucleic Acids Res., 1994; 22:4039-4043. Inosine (I) has also been considered in the art to be a universal base and is considered complementary to any A, C, U, or T. See Watkins and Santalucia, Nucl. Acids Research, 2005; 33 (19): 6258-6267.

### Conjugates

Another possible modification of a nucleic acid involves chemically linking to the polynucleotide one or more moieties or conjugates which enhance the activity, cellular distribution or cellular uptake of the oligonucleotide. These moieties or conjugates can include conjugate groups covalently bound to functional groups such as primary or secondary hydroxyl groups. Conjugate groups include, but are not limited to, intercalators, reporter molecules, polyamines, polyamides, polyethylene glycols, polyethers, groups that enhance the pharmacodynamic properties of oligomers, and groups that enhance the pharmacokinetic properties of oligomers. Suitable conjugate groups include, but are not limited to, cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation, and/or strengthen sequence-specific hybridization with the target nucleic acid. Groups that enhance the pharmacokinetic properties include groups that improve uptake, distribution, metabolism or excretion of a nucleic acid.

Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyi-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 36513654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacal. Exp. Ther., 1996, 277, 923-937).

A conjugate may include a “Protein Transduction Domain” or PTD (also known as a CPP-cell penetrating peptide), which may refer to a polypeptide, polynucleotide, carbohydrate, or organic or inorganic compound that facilitates traversing a lipid bilayer, micelle, cell membrane, organelle membrane, or vesicle membrane. A PTD attached to another molecule, which can range from a small polar molecule to a large macromolecule and/or a nanoparticle, facilitates the molecule traversing a membrane, for example going from extracellular space to intracellular space, or cytosol to within an organelle. In some embodiments, a PTD is covalently linked to the amino terminus of an exogenous polypeptide (e.g., an sRGN polypeptide). In some embodiments, a PTD is covalently linked to the C-terminus or the N-terminus of an exogenous polypeptide (e.g., an sRGN polypeptide). In some embodiments, a PTD is covalently linked to a nucleic acid (e.g., a guide RNA, a nucleic acid encoding a guide RNA, a nucleic acid encoding an sRGN polypeptide, etc.). Exemplary PTDs include but are not limited to a minimal undecapeptide protein transduction domain (for example corresponding to residues 47-57 of HIV-1 TAT comprising the YGRKKRRQRRR (SEQ ID NO: 126) motif (for identifying but not for purposes of disclosure within this application); a polyarginine sequence comprising a number of arginines sufficient to direct entry into a cell (e.g., 3, 4, 5, 6, 7, 8, 9, 10, or 10-50 arginines); a VP22 domain (Zender et al. (2002) Cancer Gene Ther. 9 (6): 489-96); an Drosophila Antennapedia protein transduction domain (Noguchi et al. (2003) Diabetes 52 (7): 1732-1737); a truncated human calcitonin peptide (Trehin et al. (2004) Pharm. Research 21:1248-1256); polylysine (Wender et al. (2000) Proc. Natl. Acad. Sci. USA97: 13003-13008); In some embodiments, the PTD is an activatable CPP (ACPP) (Aguilera et al. (2009) Integr Biol (Camb) June; 1 (5-6): 371-381). ACPPs comprise a polycationic CPP (e.g., Arg9 or “R9”) connected via a cleavable linker to a matching polyanion (e.g., Glu9 or “E9”), which reduces the net charge to nearly zero and thereby inhibits adhesion and uptake into cells. Upon cleavage of the linker, the polyanion is released, locally unmasking the polyarginine and its inherent adhesiveness, thus “activating” the ACPP to traverse the membrane. In some embodiments the PTD is chemically modified in order to increase the bioavailability of the PTD. Exemplary modifications are disclosed in Expert Opin Drug Deliv. 2009 6 (11): 1195-205.

Nucleic Acids Encoding a Guide RNA and/or an sRGN Polypeptide

The present disclosure provides a nucleic acid comprising a nucleotide sequence encoding a guide RNA and/or an sRGN polypeptide. In some embodiments, a guide RNA-encoding nucleic acid is an expression vector, e.g., a recombinant expression vector.

In some embodiments, a method involves contacting a target DNA or introducing into a cell (or a population of cells) one or more nucleic acids comprising nucleotide sequences encoding a guide RNA and/or an sRGN polypeptide. In some embodiments a cell comprising a target DNA is in vitro. In some embodiments a cell comprising a target DNA is in vivo. Suitable nucleic acids comprising nucleotide sequences encoding a guide RNA and/or an sRGN polypeptide include expression vectors, where an expression vector comprising a nucleotide sequence encoding a guide RNA and/or an sRGN polypeptide is a “recombinant expression vector.”

In some embodiments, the recombinant expression vector is a viral construct, e.g., a recombinant adeno-associated virus construct (e.g., U.S. Pat. No. 7,078,387), a recombinant adenoviral construct, a recombinant lentiviral construct, a recombinant retroviral construct, etc.

Suitable expression vectors include, but are not limited to, viral vectors (e.g., viral vectors based on vaccinia virus; poliovirus; adenovirus (e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther4: 683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Viral. (1988) 166:154-165; and Flotte et al., PNAS (1993) 90:10613-10617); SV40; herpes simplex virus; human immunodeficiency virus (e.g., Miyoshi et al., PNAS 94:10319 23, 1997; Takahashi et al., J Viral 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like.

Numerous suitable expression vectors are known to those of skill in the art, and many are commercially available. The following vectors are provided by way of example; for eukaryotic host cells: pXT1, pSGS (Stratagene), pSVK3, pBPV, pMSG, and pSVLSV40 (Phanmacia). However, any other vector may be used so long as it is compatible with the host cell. Depending on the host/vector system utilized, any of a number of suitable transcription and translation control elements, including constitutive and inducible promoters, transcription enhancer elements, transcription terminators, etc. may be used in the expression vector (e.g., Bitter et al. (1987) Methods in Enzymology, 153:516-544).

In some embodiments, a nucleotide sequence encoding a guide RNA and/or an sRGN polypeptide is operably linked to a control element, e.g., a transcriptional control element, such as a promoter. The transcriptional control element may be functional in either a eukaryotic cell, e.g., a mammalian cell; or a prokaryotic cell (e.g., bacterial or archaeal cell). In some embodiments, a nucleotide sequence encoding a guide RNA and/or an sRGN polypeptide is operably linked to multiple control elements that allow expression of the nucleotide sequence encoding a guide RNA and/or an sRGN polypeptide in both prokaryotic and eukaryotic cells.

Non-limiting examples of suitable eukaryotic promoters (promoters functional in a eukaryotic cell) include those from cytomegalovirus (CMV) immediate early, herpes simplex virus (HSV) thymidine kinase, early and late SV40, long terminal repeats (LTRs) from retrovirus, and mouse metallothionein-1. Selection of the appropriate vector and promoter is within the level of ordinary skill in the art. The expression vector may also contain a ribosome binding site for translation initiation and a transcription terminator. The expression vector may also include appropriate sequences for amplifying expression. The expression vector may also include nucleotide sequences encoding protein tags (e.g., 6×His tag, hemagglutinin tag, green fluorescent protein, etc.) that are fused to the sRGN polypeptide, thus resulting in a chimeric polypeptide.

In some embodiments, a nucleotide sequence encoding a guide RNA and/or an sRGN polypeptide is operably linked to an inducible promoter. In some embodiments, a nucleotide sequence encoding a guide RNA and/or an sRGN polypeptide is operably linked to a constitutive promoter.

Methods of introducing a nucleic acid into a host cell are known in the art, and such methods can be used to introduce a nucleic acid (e.g., an expression construct) into a cell. Suitable methods include, e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (e.g., Panyam et al., Adv Drug Deliv Rev. 2012 Sep. 13. pii: S0169-409X(12) 00283-9.doi: 10.1016/j.addr.2012.09.023), and the like.

### Chimeric Polypeptides

The present disclosure provides a chimeric site-directed modifying polypeptide comprising a sequence derived from an sRGN polypeptide. A chimeric site-directed modifying polypeptide interacts with (e.g., binds to) a guide RNA (described above). The guide RNA guides the chimeric site-directed modifying polypeptide to a target sequence within target DNA (e.g., a chromosomal sequence or an extrachromosomal sequence, e.g., an episomal sequence, a minicircle sequence, a mitochondrial sequence, a chloroplast sequence, cell-free polynucleotide, etc.). A chimeric site-directed modifying polypeptide modifies target DNA (e.g., cleavage or methylation of target DNA) and/or a polypeptide associated with target DNA (e.g., methylation or acetylation of a histone tail). A chimeric site-directed modifying polypeptide is also referred to herein as a “chimeric site-directed polypeptide” or a “chimeric RNA binding site-directed modifying polypeptide.”

A chimeric site-directed modifying polypeptide comprises two portions, an RNA-binding portion and an activity portion. A chimeric site-directed modifying polypeptide comprises amino acid sequences that are derived from at least two different polypeptides. A chimeric site-directed modifying polypeptide can comprise modified and/or naturally occurring polypeptide sequences (e.g., a first amino acid sequence from a modified or unmodified sRGN protein; and a second amino acid sequence other than the sRGN protein).

### RNA-Binding Portion

In some embodiments, the RNA-binding portion of a chimeric site-directed modifying polypeptide comprises an amino acid sequence having at least 75%, at least 80%, at least 85%, at least 90%, at least 95%, at least 98%, at least 99%, or 100%, amino acid sequence identity to the RNA-binding portion of a polypeptide set forth in any one of SEQ ID NOs: 1-12.

### Activity Portion

In addition to the RNA-binding portion, the chimeric site-directed modifying polypeptide comprises an “activity portion.” In some embodiments, the activity portion of a chimeric site-directed modifying polypeptide comprises the activity portion of a site-directed modifying polypeptide (e.g., a Cas9 endonuclease or an sRGN). In other embodiments, the activity portion of a subject chimeric site-directed modifying polypeptide comprises a modified amino acid sequence (e.g., substitution, deletion, insertion) of a naturally-occurring activity portion of a site-directed modifying polypeptide. Naturally-occurring activity portions of interest are derived from site-directed modifying polypeptides known in the art. The activity portion of a chimeric site-directed modifying polypeptide is variable and may comprise any heterologous polypeptide sequence that may be useful in the methods disclosed herein. In some embodiments, the activity portion of a chimeric site-directed modifying polypeptide comprises a portion of an sRGN polypeptide that is at least 90% identical to the activity portion of any one of SEQ ID NOs: 1-12. In some embodiments, a chimeric site-directed modifying polypeptide comprises: (i) an RNA-binding portion that interacts with a guide RNA, wherein the guide RNA comprises a nucleotide sequence that is complementary to a sequence in a target DNA; (ii) an activity portion that exhibits site-directed enzymatic activity (e.g., activity for RNA cleavage), wherein the site of enzymatic activity is determined by the palindromic hairpin structures formed by the repeats of pre-crRNA and cleaves the pre-crRNA 4 nt upstream of the hairpins generating intermediate forms of crRNAs composed of repeat spacer (5′-3′); and (iii) an activity portion that exhibits site-directed enzymatic activity (e.g., activity for DNA cleavage), wherein the site of enzymatic activity is determined by the guide RNA.

### Exemplary Chimeric Site-Directed Modifying Polypeptides

In some embodiments, the activity portion of the chimeric site-directed modifying polypeptide comprises a modified form of the sRGN protein, including modified forms of any of the sRGN orthologs. In some instances, the modified form of the sRGN protein comprises an amino acid change (e.g., deletion, insertion, or substitution) that reduces or increases the naturally occurring nuclease activity of the sRGN protein. For example, in some instances, the modified form of the sRGN protein has less than 50%, less than 40%, less than 30%, less than 20%, less than 10%, less than 5%, or less than 1% of the nuclease activity of the corresponding wild-type sRGN polypeptide. In some cases, the modified form of the sRGN polypeptide has no substantial nuclease activity. In other cases, it may have 50%, 2-fold, 4-fold or up to an over 10-fold more nuclease activity.

In some cases, the chimeric site-directed modifying polypeptide comprises an amino acid sequence having at least 75%, at least 80%, at least 85%, at least 90%, at least 95%, at least 99% or 100% amino acid sequence identity to any one of SEQ ID NOs: 1-12. In some embodiments, the activity portion of the site-directed modifying polypeptide comprises a heterologous polypeptide that has DNA-modifying activity and/or transcription factor activity and/or DNA-associated polypeptide-modifying activity. In some cases, a heterologous polypeptide replaces a portion of the sRGN polypeptide that provides nuclease activity. In other embodiments, a site-directed modifying polypeptide comprises both a portion of the sRGN polypeptide that normally provides nuclease activity (and that portion can be fully active or can instead be modified to have less than 100% of the corresponding wild-type activity) and a heterologous polypeptide. In other words, in some cases, a chimeric site-directed modifying polypeptide is a fusion polypeptide comprising both the portion of the sRGN polypeptide that normally provides nuclease activity and the heterologous polypeptide. In other cases, a chimeric site-directed modifying polypeptide is a fusion polypeptide comprising a modified variant of the activity portion of the sRGN polypeptide (e.g., amino acid change, deletion, insertion) and a heterologous polypeptide. In yet other cases, a chimeric site-directed modifying polypeptide is a fusion polypeptide comprising a heterologous polypeptide and the RNA-binding portion of a naturally occurring or a modified site-directed modifying polypeptide.

For example, in a chimeric sRGN protein, a naturally occurring (or modified, e.g., mutation, deletion, insertion) sRGN polypeptide may be fused to a heterologous polypeptide sequence (i.e., a polypeptide sequence from a protein other than sRGN or a polypeptide sequence from another organism). The heterologous polypeptide sequence may exhibit an activity (e.g., enzymatic activity) that will also be exhibited by the chimeric sRGN protein (e.g., methyltransferase activity, acetyltransferase activity, kinase activity, ubiquitinating activity, etc.). A heterologous nucleic acid sequence may be linked to another nucleic acid sequence (e.g., by genetic engineering) to generate a chimeric nucleotide sequence encoding a chimeric polypeptide. In some embodiments, a chimeric sRGN polypeptide is generated by fusing an sRGN polypeptide (e.g., wild type sRGN or an sRGN variant, e.g., an sRGN with reduced or inactivated nuclease activity) with a heterologous sequence that provides for subcellular localization (e.g., a nuclear localization signal (NLS) for targeting to the nucleus; a mitochondrial localization signal for targeting to the mitochondria; a chloroplast localization signal for targeting to a chloroplast: an ER retention signal; and the like). In some embodiments, the heterologous sequence can provide a tag for ease of tracking or purification (e.g., a fluorescent protein, e.g., green fluorescent protein (GFP), YFP, RFP, CFP, mCherry, tdTomato, and the like; a HIS tag, e.g., a 6×His tag; a hemagglutinin (HA) tag; a FLAG tag; a Myc tag; and the like). In some embodiments, the heterologous sequence can provide for increased or decreased stability. In some embodiments, the heterologous sequence can provide a binding domain (e.g., to provide the ability of a chimeric sRGN polypeptide to bind to another protein of interest, e.g., a DNA or histone modifying protein, a transcription factor or transcription repressor, a recruiting protein, etc.) or to a nucleotide of interest (e.g., an aptamer or target site of a nucleotide binding protein).

### Nucleic Acid Encoding a Chimeric Site-Directed Modifying Polypeptide

The present disclosure provides a nucleic acid comprising a nucleotide sequence encoding a chimeric site-directed modifying polypeptide. In some embodiments, the nucleic acid comprising a nucleotide sequence encoding a chimeric site-directed modifying polypeptide is an expression vector, e.g., a recombinant expression vector.

In some embodiments, a method involves contacting a target DNA or introducing into a cell (or a population of cells) one or more nucleic acids comprising a chimeric site-directed modifying polypeptide. Suitable nucleic acids comprising nucleotide sequences encoding a chimeric site-directed modifying polypeptide include expression vectors, where an expression vector comprising a nucleotide sequence encoding a chimeric site-directed modifying polypeptide is a “recombinant expression vector.”

In some embodiments, the recombinant expression vector is a viral construct, e.g., a recombinant adeno-associated virus construct (e.g., U.S. Pat. No. 7,078,387), a recombinant adenoviral construct, a recombinant lentiviral construct, etc. Suitable expression vectors include, but are not limited to, viral vectors (e.g., viral vectors based on vaccinia virus; poliovirus; adenovirus (e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769: WO 93/19191; WO 94/28938; WO 95/11984 and WO 95/00655); adeno-associated virus (e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Viral. (1988) 166:154-165; and Flotte et al., PNAS (1993) 90:10613-10617); SV40; herpes simplex virus; human immunodeficiency virus (e.g., Miyoshi et al., PNAS 94:10319 23, 1997; Takahashi et al., J Virol 73: 7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like.

Numerous suitable expression vectors are known to those of skill in the art, and many are commercially available. The following vectors are provided by way of example; for eukaryotic host cells: pXT1, pSGS (Stratagene), pSVK3, pBPV, pMSG, and pSVLSV40 (Pharmacia). However, any other vector may be used so long as it is compatible with the host cell.

Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

In some embodiments, a nucleotide sequence encoding a chimeric site-directed modifying polypeptide is operably linked to a control element, e.g., a transcriptional control element, such as a promoter. The transcriptional control element may be functional in either a eukaryotic cell, e.g., a mammalian cell; or a prokaryotic cell (e.g., bacterial or archaeal cell). In some embodiments, a nucleotide sequence encoding a chimeric site-directed modifying polypeptide is operably linked to multiple control elements that allow expression of the nucleotide sequence encoding a chimeric site-directed modifying polypeptide in both prokaryotic and eukaryotic cells.

Non-limiting examples of suitable eukaryotic promoters (promoters functional in a eukaryotic cell) include those from cytomegalovirus (CMV) immediate early, herpes simplex virus (HSV) thymidine kinase, early and late SV40, long terminal repeats (LTRs) from retrovirus, and mouse metallothionein-1. Selection of the appropriate vector and promoter is well within the level of ordinary skill in the art. The expression vector may also contain a ribosome binding site for translation initiation and a transcription terminator. The expression vector may also include appropriate sequences for amplifying expression. The expression vector may also include nucleotide sequences encoding protein tags (e.g., 6×His tag, hemagglutinin (HA) tag, a fluorescent protein (e.g., a green fluorescent protein; a yellow fluorescent protein, etc.), etc.) that are fused to the chimeric site-directed modifying polypeptide.

In some embodiments, a nucleotide sequence encoding a chimeric site-directed modifying polypeptide is operably linked to an inducible promoter (e.g., heat shock promoter, Tetracycline-regulated promoter, Steroid-regulated promoter, Metal-regulated promoter, estrogen receptor-regulated promoter, etc.). In some embodiments, a nucleotide sequence encoding a chimeric site-directed modifying polypeptide is operably linked to a spatially restricted and/or temporally restricted promoter (e.g., a tissue specific promoter, a cell type specific promoter, etc.). In some embodiments, a nucleotide sequence encoding a chimeric site-directed modifying polypeptide is operably linked to a constitutive promoter.

Methods of introducing a nucleic acid into a host cell are known in the art, and any known method can be used to introduce a nucleic acid (e.g., an expression construct) into a stem cell or progenitor cell. Suitable methods include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (e.g., Panyam et., al Adv Drug Deliv Rev. 2012 Sep. 13. pp: 50169-409X(12) 00283-9. doi: 10.1016/j.addr.2012.09.023), and the like.

### Methods

The present disclosure provides methods for modifying a target DNA and/or a target DNA-associated polypeptide. Generally, a method involves contacting a target DNA with a complex (a “targeting complex”), which complex comprises a guide RNA and an sRGN polypeptide.

As discussed above, a gRNA or sgRNA and an sRGN polypeptide form a complex. The guide RNA provides target specificity to the complex by comprising a nucleotide sequence that is complementary to a sequence of a target DNA. The sRGN polypeptide of the complex provides the site-specific activity. In some embodiments, a complex modifies a target DNA, leading to, for example, DNA cleavage, DNA methylation, DNA damage, DNA repair, etc. In other embodiments, a complex modifies a target polypeptide associated with target DNA (e.g., a histone, a DNA-binding protein, etc.), leading to, for example, histone methylation, histone acetylation, histone ubiquitination, and the like. The target DNA may be, for example, naked (i.e., unbound by DNA associated proteins) DNA in vitro, chromosomal DNA in cells in vitro, chromosomal DNA in cells in vivo, etc.

sRGN proteins from various species may require different PAM sequences in the target DNA. Thus, for a particular sRGN protein of choice, the PAM sequence requirement may be different than the PAM sequence described above.

Exemplary methods are provided that take advantage of characteristics of sRGN orthologs include the following.

The nuclease activity cleaves target DNA to produce double-strand breaks. These breaks are then repaired by the cell in one of two ways: non-homologous end joining, and homology-directed repair. In non-homologous end joining (NHEJ), the double-strand breaks are repaired by direct ligation of the break ends to one another. In the process a few base pairs can be inserted or deleted at the cleavage site. In homology-directed repair, a donor polynucleotide with homology to the cleaved target DNA sequence is used as a template for repair of the cleaved target DNA sequence, resulting in the transfer of genetic information from the donor polynucleotide to the target DNA As such, new nucleic acid material may be inserted/copied into the site. In some cases, a target DNA is contacted with a donor polynucleotide. In some cases, a donor polynucleotide is introduced into a cell. The modifications of the target DNA due to NHEJ and/or homology-directed repair lead to, for example, gene correction, gene replacement, gene tagging, transgene insertion, nucleotide deletion, nucleotide insertion, gene disruption, gene mutation, sequence replacement, etc. Accordingly, cleavage of DNA by an sRGN polypeptide may be used to delete nucleic acid material from a target DNA sequence (e.g., to disrupt a gene that makes cells susceptible to infection (e.g., the CCRS or CXCR4 gene, which makes T cells susceptible to HIV infection, to remove disease-causing trinucleotide repeat sequences in neurons, to create gene knockouts and mutations as disease models in research, etc.) by cleaving the target DNA sequence and allowing the cell to repair the sequence in the absence of an exogenously provided donor polynucleotide. Thus, the methods can be used to knock out a gene (resulting in complete lack of transcription or altered transcription) or to knock in genetic material into a locus of choice in the target DNA

Alternatively, if a guide RNA and an sRGN polypeptide are co-administered to cells with a donor polynucleotide sequence that includes at least a segment with homology to the target DNA sequence, the subject methods may be used to add, i.e., insert or replace, nucleic acid material to a target DNA sequence (e.g., to “knock in” a nucleic acid that encodes for a protein, an siRNA, an miRNA, etc.), to add a tag (e.g., 6×His, a fluorescent protein (e.g., a green fluorescent protein; a yellow fluorescent protein, etc.), hemagglutinin (HA), FLAG, etc.), to add a regulatory sequence to a gene (e.g., promoter, polyadenylation signal, internal ribosome entry sequence (IRES), 2A peptide, start codon, stop codon, splice signal, localization signal, etc.), to modify a nucleic acid sequence (e.g., introduce a mutation), and the like. As such, a complex comprising a guide RNA and an sRGN polypeptide is useful in any in vitro or in vivo application in which it is desirable to modify DNA in a site-specific, i.e., “targeted”, way, for example gene knock-out, gene knock-in, gene editing, gene tagging, sequence replacement, etc., as used in, for example, gene therapy, e.g., to treat a disease or as an antiviral, antipathogenic, or anticancer therapeutic, the production of genetically modified organisms in agriculture, the large scale production of proteins by cells for therapeutic, diagnostic, or research purposes, the induction of iPS cells, biological research, the targeting of genes of pathogens for deletion or replacement, etc.

In some embodiments, the sRGN polypeptide comprises a modified form of the sRGN protein. In some instances, the modified form of the sRGN protein comprises an amino acid change (e.g., deletion, insertion, or substitution) that reduces the naturally occurring nuclease activity of the sRGN protein. For example, in some instances, the modified form of the sRGN protein has less than 50%, less than 40%, less than 30%, less than 20%, less than 10%, less than 5%, or less than 1% of the nuclease activity of the corresponding wild-type sRGN polypeptide. In some cases, the modified form of the sRGN polypeptide has no substantial nuclease activity. When an sRGN polypeptide is a modified form of the sRGN protein that has no substantial nuclease activity, it can be referred to as “dsRGN.”

In some embodiments, the sRGN polypeptide comprises a heterologous sequence (e.g., a fusion). In some embodiments, a heterologous sequence can provide for subcellular localization of the sRGN polypeptide (e.g., a nuclear localization signal (NLS) for targeting to the nucleus; a mitochondrial localization signal for targeting to the mitochondria; a chloroplast localization signal for targeting to a chloroplast; an ER retention signal; and the like). In some embodiments, a heterologous sequence can provide a tag for ease of tracking or purification (e.g., a fluorescent protein, e.g., green fluorescent protein (GFP), YFP, RFP, CFP, mCherry, tdTomato, and the like; a his tag, e.g., a 6×His tag; a hemagglutinin (HA) tag; a FLAG tag; a Myc tag; and the like). In some embodiments, the heterologous sequence can provide for increased or decreased stability.

In certain embodiments, modified polynucleotides are used in a CRISPR-sRGN system described herein, in which the guide RNAs and/or a DNA or an RNA comprising a polynucleotide sequence encoding an sRGN polypeptide or variant thereof can be modified, as described and illustrated below. Such modified polynucleotides can be used in the CRISPR-sRGN system, for example, to edit any one or more genomic loci. In some embodiments, such modifications in the polynucleotides of the disclosure are achieved via codon-optimization, e.g., codon-optimization based on specific host cells in which the encoded polypeptide is to be expressed. It will be appreciated by the skilled artisan that any nucleotide sequence and/or recombinant nucleic acid of the present disclosure can be codon optimized for expression in any species of interest. Codon optimization is well known in the art and involves modification of a nucleotide sequence for codon usage bias using species specific codon usage tables. The codon usage tables are generated based on a sequence analysis of the most highly expressed genes for the species of interest. In a non-limiting example, when the nucleotide sequences are to be translated in a host cell, the codon usage tables are generated based on a sequence analysis of highly expressed nuclear genes for the species of interest. The modifications of the nucleotide sequences are determined by comparing the species specific codon usage table with the codons present in the native polynucleotide sequences. For example, if the intended target cell were a human cell, a human codon-optimized polynucleotide sequence encoding an sRGN (or an sRGN variant, e.g., enzymatically inactive variant) would be suitable. As another non-limiting example, if the intended host cell were a mouse cell, then a mouse codon-optimized polynucleotide sequence encoding an sRGN (or an sRGN variant, e.g., enzymatically inactive variant) would be suitable.

Strategies and methodologies for codon optimization are known in the art and have been described for various systems including, but not limited to yeast (Outchkourov et al., Protein Expr Purif, 24 (1): 18-24 (2002)) and E. coli (Feng et al., Biochemistry, 39 (50): 15399-15409 (2000)). In some embodiments, the codon optimization is performed by using GeneGPS® Expression Optimization Technology (ATUM) and using the manufacturer's recommended expression optimization algorithms. In some embodiments, the polynucleotides of the disclosure are codon-optimized for increased expression in a human cell. In some embodiments, the polynucleotides of the disclosure are codon-optimized for increased expression in an E. coli cell. In some embodiments, the polynucleotides of the disclosure are codon-optimized for increased expression in an insect cell. In some embodiments, the expression optimization algorithms used in a codon optimization procedure are defined to avoid putative poly-A signals (e.g., AATAAA and ATTAAA) as well as long (greater than 4) stretches of A's which can lead to polymerase slippage. Polyadenylation signals can also be chosen to optimize expression in the intended host. Polyadenylation signals can also be chosen to optimize expression in the intended host.

As is well understood in the art, codon optimization of a nucleotide sequence results in a nucleotide sequence having less than 100% identity (e.g., less than 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%) to the native nucleotide sequence but which still encodes a polypeptide having the same function as that encoded by the original, native nucleotide sequence. Thus, in representative embodiments of the disclosure, the nucleotide sequence and/or recombinant nucleic acid of the disclosure can be codon optimized for expression in the particular species of interest.

In some embodiments, a codon-optimized polynucleotide sequence encoding an sRGN has at least 90%, 92%, 93%, 94%, 95%, 96%, 97%, 98%, 99%, 99.2%, 99.5%, 99.8%, 99.9%, or 100% sequence identity to any one of SEQ ID NOs: 15, 18, 20, 22, and 27. In some embodiments, the polynucleotides of the disclosure are codon-optimized for increased expression of the encoded sRGN polypeptide in a target cell. In some embodiments, the polynucleotides of the disclosure are codon-optimized for increased expression in a human cell. In some embodiments, the polynucleotides of the disclosure are codon-optimized for increased expression in an E. coli cell. In some embodiments, the polynucleotides of the disclosure are codon-optimized for increased expression in an insect cell.

In some embodiments, an sRGN polypeptide can be codon-optimized. This type of optimization is known in the art and entails the mutation of foreign-derived DNA to mimic the codon preferences of the intended host organism or cell while encoding the same protein. Thus, the codons are changed, but the encoded protein remains unchanged. For example, if the intended target cell were a human cell, a human codon-optimized sRGN (or variant, e.g., enzymatically inactive variant) would be suitable. Any suitable sRGN polypeptide (e.g., any sRGN such as the sequence set forth in any one of SEQ ID NOs: 1-12) can be codon optimized. As another non-limiting example, if the intended host cell were a mouse cell, than a mouse codon-optimized sRGN (or variant, e.g., enzymatically inactive variant) would be suitable.

In one aspect, provided herein is a nucleic acid comprising a codon-optimized polynucleotide sequence encoding an sRGN polypeptide (e.g., a polypeptide comprising the amino acid sequence of any one of SEQ ID NOs: 1-12) or a variant thereof having at least about 90% (such as at least about any of 91%, 92%, 93%, 94%, 95%, 96%, 97%, 98%, 99%, or more) sequence identity to any one of SEQ ID NOs: 1-12. Such a codon-optimized nucleic acid encoding an sRGN or a variant thereof is also referred to herein as a “codon-optimized sRGN polypeptide” or a “codon-optimized sRGN.” In some embodiments, the codon-optimized sRGN encodes the sRGN polypeptide of any one of SEQ ID NOs: 1-12. In some embodiments, the codon-optimized sRGN is codon-optimized for expression in prokaryotic cells, such as bacterial cells. In some embodiments, the codon-optimized sRGN is codon-optimized for expression in eukaryotic cells, such as insect or mammalian cells. In some embodiments, the codon-optimized sRGN is codon-optimized for expression in human cells. In some embodiments, the codon optimization is performed according to GeneGPS® optimization (ATUM). In some embodiments, the codon optimization is performed according to a GeneOptimizer process (ThermoFisher Scientific). In some embodiments, the codon-optimized sRGN has at least about 90% (such as at least about any of 91%, 92%, 93%, 94%, 95%, 96%, 97%, 98%, 99%, or more) sequence identity to any one of SEQ ID NOs: 15, 18, 20, 22, and 27. In some embodiments, the codon-optimized sRGN comprises (or consists of) the sequence of any one of SEQ ID NOs: 15, 18, 20, 22, and 27.

In some embodiments, according to any of the nucleic acids described herein comprising a codon-optimized polynucleotide sequence encoding an sRGN polypeptide or a variant thereof, the nucleic acid further includes a nucleotide sequence encoding a gRNA. In some embodiments, the nucleic acid is an expression vector, e.g., a recombinant expression vector. In some embodiments, the nucleotide sequence encoding an sRGN polypeptide or a variant thereof comprises the codon-optimized polynucleotide sequence of any one of SEQ ID NOs: 15, 18, 20, 22, and 27 or a variant thereof having at least 90% sequence identity to the codon-optimized polynucleotide sequence of any one of SEQ ID NOs: 15, 18, 20, 22, and 27.

In some embodiments, provided herein is a nucleic acid comprising a polynucleotide sequence having at least 90%, 91%, 92%, 93%, 94%, 95%, 96%, 97%, 98%, or 99% sequence identity to any one of SEQ ID NOs: 15, 18, 20, 22, and 27, wherein the polynucleotide sequence encodes an sRGN polypeptide or variant thereof as described herein. In some embodiments, the polynucleotide sequence comprises (or consists of) the codon-optimized polynucleotide sequence of any one of SEQ ID NOs: 15, 18, 20, 22, and 27.

In some embodiments, provided herein is a nucleic acid comprising a polynucleotide sequence having at least 90%, 91%, 92%, 93%, 94%, 95%, 96%, 97%, 98% or 99% sequence identity to a subsequence of any one of SEQ ID NOs: 15, 18, 20, 22, and 27, wherein the polynucleotide sequence encodes one or more functional sRGN domains. In some embodiments, the polynucleotide sequence comprises (or consists of) a subsequence of any one of SEQ ID NOs: 15, 18, 20, 22, and 27. In some embodiments, the one or more sRGN domains include a nuclease domain (e.g., an HNH or RuvC domain). In some embodiments, the one or more sRGN domains include an RNA-binding domain (e.g., a Rec I domain). In some embodiments, the one or more sRGN domains include a PAM-interacting domain.

In some embodiments, according to any of the nucleic acids described herein comprising a codon-optimized polynucleotide sequence, the expression in a host cell of an sRGN polypeptide or variant thereof from the nucleic acid is increased as compared to the expression in the host cell of the sRGN polypeptide or variant thereof from a corresponding nucleic acid comprising a reference polynucleotide sequence from which the codon-optimized polynucleotide sequence is derived. In some embodiments, the codon-optimized polynucleotide sequence comprises the codon-optimized polynucleotide sequence of any one of SEQ ID NOS: 15, 18, 20, 22, and 27, or a variant thereof having at least 90% sequence identity to the codon-optimized polynucleotide sequence of any one of SEQ ID NOs: 15, 18, 20, 22, and 27. In some embodiments, the reference polynucleotide sequence comprises the sRGN-encoding polynucleotide sequence from any one of SEQ ID NOs: 13, 14, 16, 17, 19, 21, 23-26, 28, and 29, or a variant thereof having at least 90% sequence identity to the sRGN-encoding polynucleotide sequence from any one of SEQ ID NOs: 13, 14, 16, 17, 19, 21, 23-26, 28, and 29. In some embodiments, the expression of the sRGN polypeptide or variant thereof from the codon-optimized polynucleotide sequence in a host cell is increased by at least about 10% (such as by at least about any of 15%, 20%, 25%, 30%, 35%, 40%, 45%, 50%, 55%, 60%, 65%, 70%, 75%, 80%, 85%, 90%, 95%, 100%, or more) as compared to the expression in the host cell of the sRGN polypeptide or variant thereof from the reference polynucleotide sequence.

In some embodiments, a guide RNA and an sRGN polypeptide are used as an inducible system for shutting off gene expression in bacterial cells. In some cases, nucleic acids encoding an appropriate guide RNA and/or an appropriate sRGN polypeptide are incorporated into the chromosome of a target cell and are under control of an inducible promoter. When the guide RNA and/or the sRGN polypeptide are induced, the target DNA is cleaved (or otherwise modified) at the location of interest (e.g., a target gene on a separate plasmid), when both the guide RNA and the sRGN polypeptide are present and form a complex. As such, in some cases, bacterial expression strains are engineered to include nucleic acid sequences encoding an appropriate sRGN polypeptide in the bacterial genome and/or an appropriate guide RNA on a plasmid (e.g., under control of an inducible promoter), allowing experiments in which the expression of any targeted gene (expressed from a separate plasmid introduced into the strain) could be controlled by inducing expression of the guide RNA and the sRGN polypeptide. In some cases, the sRGN polypeptide has enzymatic activity that modifies target DNA in ways other than introducing double-strand breaks. Enzymatic activity of interest that may be used to modify target DNA (e.g., by fusing a heterologous polypeptide with enzymatic activity to an sRGN polypeptide, thereby generating a chimeric sRGN polypeptide) includes, but is not limited methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity). Methylation and demethylation is recognized in the art as an important mode of epigenetic gene regulation while DNA damage and repair activity is essential for cell survival and for proper genome maintenance in response to environmental stresses. As such, the methods herein find use in the epigenetic modification of target DNA and may be employed to control epigenetic modification of target DNA at any location in a target DNA by genetically engineering the desired complementary nucleic acid sequence into the DNA-targeting segment of a guide RNA. The methods herein also find use in the intentional and controlled damage of DNA at any desired location within the target DNA. The methods herein also find use in the sequence-specific and controlled repair of DNA at any desired location within the target DNA. Methods to target DNA-modifying enzymatic activities to specific locations in target DNA find use in both research and clinical applications.

In some cases, the sRGN polypeptide has activity that modulates the transcription of target DNA (e.g., in the case of a chimeric sRGN polypeptide, etc.). In some cases, a chimeric sRGN polypeptides comprising a heterologous polypeptide that exhibits the ability to increase or decrease transcription (e.g., transcriptional activator or transcription repressor polypeptides) is used to increase or decrease the transcription of target DNA at a specific location in a target DNA, which is guided by the DNA-targeting segment of the guide RNA. Examples of source polypeptides for providing a chimeric sRGN polypeptide with transcription modulatory activity include, but are not limited to light-inducible transcription regulators, small molecule/drug-responsive transcription regulators, transcription factors, transcription repressors, etc. In some cases, the method is used to control the expression of a targeted coding-RNA (protein-encoding gene) and/or a targeted non-coding RNA (e.g., tRNA, IRNA, snoRNA, siRNA, miRNA, long ncRNA, etc.). In some cases, the sRGN polypeptide has enzymatic activity that modifies a polypeptide associated with DNA (e.g., histone). In some embodiments, the enzymatic activity is methyltransferase activity, demethylase activity, acetyltransferase activity, deacetylase activity, kinase activity, phosphatase activity, ubiquitin ligase activity (i.e., ubiquitination activity), deubiquitinating activity, adenylation activity, deadenylation activity, SUMOylating activity, deSUMOylating activity, ribosylation activity, deribosylation activity, myristoylation activity, demyristoylation activity glycosylation activity (e.g., from GlcNAc transferase) or deglycosylation activity. The enzymatic activities listed herein catalyze covalent modifications to proteins. Such modifications are known in the art to alter the stability or activity of the target protein (e.g., phosphorylation due to kinase activity can stimulate or silence protein activity depending on the target protein). Of particular interest as protein targets are histones. Histone proteins are known in the art to bind DNA and form complexes known as nucleosomes. Histones can be modified (e.g., by methylation, acetylation, ubiquitination, phosphorylation) to elicit structural changes in the surrounding DNA, thus controlling the accessibility of potentially large portions of DNA to interacting factors such as transcription factors, polymerases and the like. A single histone can be modified in many different ways and in many different combinations (e.g., trimethylation of lysine 27 of histone 3, H3K27, is associated with DNA regions of repressed transcription while trimethylation of lysine 4 of histone 3, H3K4, is associated with DNA regions of active transcription). Thus, a site-directed modifying polypeptide with histone-modifying activity finds use in the site specific control of DNA structure and can be used to alter the histone modification pattern in a selected region of target DNA. Such methods find use in both research and clinical applications.

In some embodiments, multiple guide RNAs are used simultaneously to simultaneously modify different locations on the same target DNA or on different target DNAs. In some embodiments, two or more guide RNAs target the same gene or transcript or locus. In some embodiments, two or more guide RNAs target different unrelated loci. In some embodiments, two or more guide RNAs target different, but related loci.

In some cases, the sRGN polypeptide is provided directly as a protein. As one non-limiting example, fungi (e.g., yeast) can be transformed with exogenous protein and/or nucleic acid using spheroplast transformation (see Kawai et al., Bioeng Bugs. 2010 November-December; 1 (6): 395-403: ‘Transformation of Saccharomyces cerevisiae and other fungi: methods and possible underlying mechanism”; and Tanka et al., Nature. 2004 Mar. 18; 428 (6980): 323-8: “Conformational variations in an infectious protein determine prion strain differences”; both of which are herein incorporated by reference in their entirety). Thus, an sRGN polypeptide can be incorporated into a spheroplast (with or without nucleic acid encoding a guide RNA and with or without a donor polynucleotide) and the spheroplast can be used to introduce the content into a yeast cell. A sRGN polypeptide can be introduced into a cell (provided to the cell) by any convenient method; such methods are known to those of ordinary skill in the art. As another non-limiting example, an sRGN polypeptide can be injected directly into a cell (e.g., with or without nucleic acid encoding a guide RNA and with or without a donor polynucleotide), e.g., a cell of a zebrafish embryo, the pronucleus of a fertilized mouse oocyte, etc.

### Target Cells of Interest

In some of the above applications, the methods may be employed to induce DNA cleavage, DNA modification, and/or transcriptional modulation in mitotic or post-mitotic cells in vivo and/or ex vivo and/or in vitro (e.g., to produce genetically modified cells that can be reintroduced into an individual). Because the guide RNA provide specificity by hybridizing to target DNA, a mitotic and/or post-mitotic cell of interest in the disclosed methods may include a cell from any organism (e.g., a bacterial cell, an archaeal cell, a cell of a single-cell eukaryotic organism, a plant cell, an algal cell, e.g., Botryococcus braunii, Chlamydomonas reinhardtii, Nannochloropsis gaditana, Chlorella pyrenoidosa, Sargassum patens C. Agardh, and the like, a fungal cell (e.g., a yeast cell), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal, a cell from a rodent, a cell from a primate, a cell from a human, etc.).

Any type of cell may be of interest (e.g., a stem cell, e.g., an embryonic stem (ES) cell, an induced pluripotent stem (iPS) cell, a germ cell; a somatic cell, e.g., a fibroblast, a hematopoietic cell, a neuron, a muscle cell, a bone cell, a hepatocyte, a pancreatic cell; an in vitro or in vivo embryonic cell of an embryo at any stage, e.g., a 1-cell, 2-cell, 4-cell, 8-cell, etc. stage zebrafish embryo; etc.). Cells may be from established cell lines or they may be primary cells, where “primary cells”, “primary cell lines”, and “primary cultures” are used interchangeably herein to refer to cells and cells cultures that have been derived from an organism and allowed to grow in vitro for a limited number of passages, i.e., splittings, of the culture. For example, primary cultures are cultures that may have been passaged 0 times, 1 time, 2 times, 4 times, 5 times, 10 times, or 15 times, but not enough times go through the crisis stage. Generally, the primary cell lines of the present invention are maintained for fewer than 10 passages in vitro. Target cells are in many embodiments unicellular organisms, or are grown in culture.

If the cells are primary cells, they may be harvest from an individual by any convenient method. For example, leukocytes may be conveniently harvested by apheresis, leukocytapheresis, density gradient separation, etc., while cells from tissues such as skin, muscle, bone marrow, spleen, liver, pancreas, lung, intestine, stomach, etc. are most conveniently harvested by biopsy. An appropriate solution may be used for dispersion or suspension of the harvested cells. Such solution will generally be a balanced salt solution, e.g., normal saline, phosphate-buffered saline (PBS), Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc. The cells may be used immediately, or they may be stored, frozen, for long periods of time, being thawed and capable of being reused. In such cases, the cells will usually be frozen in 10% DMSO, 50% serum, 40% buffered medium, or some other such solution as is commonly used in the art to preserve cells at such freezing temperatures, and thawed in a manner as commonly known in the art for thawing frozen cultured cells.

Nucleic Acids Encoding a Guide RNA and/or an sRGN Polypeptide

In some embodiments, a method involves contacting a target DNA or introducing into a cell (or a population of cells) one or more nucleic acids comprising nucleotide sequences encoding a guide RNA and/or an sRGN polypeptide and/or a donor polynucleotide. Suitable nucleic acids comprising nucleotide sequences encoding a guide RNA and/or an sRGN polypeptide include expression vectors, where an expression vector comprising a nucleotide sequence encoding a guide RNA and/or an sRGN polypeptide is a “recombinant expression vector.”

In some embodiments, the recombinant expression vector is a viral construct, e.g., a recombinant adeno-associated virus construct (e.g., U.S. Pat. No. 7,078,387), a recombinant adenoviral construct, a recombinant lentiviral construct, etc.

Suitable expression vectors include, but are not limited to, viral vectors (e.g., viral vectors based on vaccinia virus; poliovirus; adenovirus (e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94112649, WO 93/03769; WO 93/19191; WO 94/28938; WO 95/11984 and WO 95/00655); adeno-associated virus (e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Viral. (1988) 166:154-165; and Flotte et al., PNAS (1993) 90:10613-10617); SV40; herpes simplex virus; human immunodeficiency virus (e.g., Miyoshi et al., PNAS 94:10319 23, 1997; Takahashi et al., J Virol 73: 7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like.

Numerous suitable expression vectors are known to those of skill in the art, and many are commercially available. The following vectors are provided by way of example; for eukaryotic host cells: pXT1, pSG5 (Stratagene), pSVK3, pBPV, pMSG, and pSVLSV40 (Pharmacia). However, any other vector may be used so long as it is compatible with the host cell.

In some embodiments, a nucleotide sequence encoding a guide RNA and/or an sRGN polypeptide is operably linked to a control element, e.g., a transcriptional control element, such as a promoter. The transcriptional control element may be functional in either a eukaryotic cell, e.g., a mammalian cell, or a prokaryotic cell (e.g., bacterial or archaeal cell). In some embodiments, a nucleotide sequence encoding a guide RNA and/or an sRGN polypeptide is operably linked to multiple control elements that allow expression of the nucleotide sequence encoding a guide RNA and/or an sRGN polypeptide in both prokaryotic and eukaryotic cells.

Depending on the host/vector system utilized, any of a number of suitable transcription and translation control elements, including constitutive and inducible promoters, transcription enhancer elements, transcription terminators, etc. may be used in the expression vector (e.g., U6 promoter, H1 promoter, etc.; see above) (see e.g., Bitter et al. (1987) Methods in Enzymology, 153:516-544).

In some embodiments, a guide RNA and/or an sRGN polypeptide can be provided as RNA. In such cases, the guide RNA and/or the RNA encoding the sRGN polypeptide can be produced by direct chemical synthesis or may be transcribed in vitro from a DNA encoding the guide RNA. Methods of synthesizing RNA from a DNA template are well known in the art. In some cases, the guide RNA and/or the RNA encoding the sRGN polypeptide will be synthesized in vitro using an RNA polymerase enzyme (e.g., T7 polymerase, T3 polymerase, SP6 polymerase, etc.). Once synthesized, the RNA may directly contact a target DNA or may be introduced into a cell by any of the well-known techniques for introducing nucleic acids into cells (e.g., microinjection, electroporation, transfection, etc.).

Nucleotides encoding a guide RNA (introduced either as DNA or RNA) and/or an sRGN polypeptide (introduced as DNA or RNA) and/or a donor polynucleotide may be provided to the cells using well-developed transfection techniques; e.g., Angel and Yanik (2010) PLOS ONE 5 (7): e 11756, and the commercially available TransMessenger® reagents from Qiagen, Stemfect™ RNA Transfection Kit from Stemgent, and TransiT®-mRNA Transfection Kit from Mims Bio. See also Beumer et al. (2008) Efficient gene targeting in Drosophila by direct embryo injection with zinc-finger nucleases. PNAS 105 (50): 19821-19826. Alternatively, nucleic acids encoding a guide RNA and/or an sRGN polypeptide and/or a chimeric sRGN polypeptide and/or a donor polynucleotide may be provided on DNA vectors. Many vectors, e.g., plasmids, cosmids, minicircles, phage, viruses, etc., useful for transferring nucleic acids into target cells are available. The vectors comprising the nucleic acid(s) may be maintained episomally, e.g., as plasmids, minicircle DNAs, viruses such cytomegalovirus, adenovirus, etc., or they may be integrated into the target cell genome, through homologous recombination or random integration, e.g., retrovirus-derived vectors such as MMLV, HIV-1, ALV, etc.

Vectors may be provided directly to the cells. In other words, the cells are contacted with vectors comprising the nucleic acid encoding guide RNA and/or an sRGN polypeptide and/or a chimeric sRGN polypeptide and/or a donor polynucleotide such that the vectors are taken up by the cells. Methods for contacting cells with nucleic acid vectors that are plasmids, including electroporation, calcium chloride transfection, microinjection, and lipofection are well known in the art. For viral vector delivery, the cells are contacted with viral particles comprising the nucleic acid encoding a guide RNA and/or an sRGN polypeptide and/or a chimeric sRGN polypeptide and/or a donor polynucleotide. Retroviruses, for example, lentiviruses, are particularly suitable to the method of the invention. Commonly used retroviral vectors are “defective”, i.e., unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein (ecotropic, amphotropic or xenotropic) to be incorporated into the capsid, this envelope protein determining the specificity of the viral particle for the cells (ecotropic for murine and rat; amphotropic for most mammalian cell types including human, dog and mouse; and xenotropic for most mammalian cell types except murine cells). The appropriate packaging cell line may be used to ensure that the cells are targeted by the packaged viral particles. Methods of introducing the retroviral vectors comprising the nucleic acid encoding the reprogramming factors into packaging cell lines and of collecting the viral particles that are generated by the packaging lines are well known in the art. Nucleic acids can also be introduced by direct micro-injection (e.g., injection of RNA into a zebrafish embryo).

Vectors used for providing the nucleic acids encoding guide RNA and/or an sRGN polypeptide and/or a chimeric sRGN polypeptide and/or a donor polynucleotide to the cells will generally comprise suitable promoters for driving the expression, that is, transcriptional activation, of the nucleic acid of interest. In other words, the nucleic acid of interest will be operably linked to a promoter. This may include ubiquitously acting promoters, for example, the CMV-13-actin promoter, or inducible promoters, such as promoters that are active in particular cell populations or that respond to the presence of drugs such as tetracycline. By transcriptional activation, it is intended that transcription will be increased above basal levels in the target cell by at least 10 fold, by at least 100 fold, more usually by at least 1000 fold. In addition, vectors used for providing a guide RNA and/or an sRGN polypeptide and/or a chimeric sRGN polypeptide and/or a donor polynucleotide to the cells may include nucleic acid sequences that encode for selectable markers in the target cells, so as to identify cells that have taken up the guide RNA and/or an sRGN polypeptide and/or a chimeric sRGN polypeptide and/or a donor polynucleotide.

A guide RNA and/or an sRGN polypeptide and/or a chimeric sRGN polypeptide may instead be used to contact DNA or introduced into cells as RNA. Methods of introducing RNA into cells are known in the art and may include, for example, direct injection, transfection, or any other method used for the introduction of DNA.

A sRGN polypeptide may be provided to cells as a polypeptide. Such a polypeptide may optionally be fused to a polypeptide domain that increases solubility of the product. The domain may be linked to the polypeptide through a defined protease cleavage site, e.g., a TEV sequence, which is cleaved by TEV protease. The linker may also include one or more flexible sequences, e.g., from 1 to 10 glycine residues. In some embodiments, the cleavage of the fusion protein is performed in a buffer that maintains solubility of the product, e.g., in the presence of from 0.5 to 2 M urea, in the presence of polypeptides and/or polynucleotides that increase solubility, and the like. Domains of interest include endosomolytic domains, e.g., influenza HA domain; and other polypeptides that aid in production, e.g., IF2 domain, GST domain, GRPE domain, and the like. The polypeptide may be formulated for improved stability. For example, the peptides may be PEGylated, where the polyethyleneoxy group provides for enhanced lifetime in the blood stream.

Additionally or alternatively, the sRGN polypeptide may be fused to a polypeptide permeant domain to promote uptake by the cell. A number of permeant domains are known in the art and may be used in the non-integrating polypeptides of the present invention, including peptides, peptidomimetics, and non-peptide carriers. For example, a permeant peptide may be derived from the third alpha helix of Drosophila melanogaster transcription factor Antennapaedia, referred to as penetratin, which comprises the amino acid sequence RQIKIWFQNRRMKWKK (SEQ ID NO: 127) motif (for identifying but not for purposes of disclosure within this application) also disclosed in J. Biol. Chem., 271 (1996), pp. 18188-18193. As another example, the permeant peptide comprises the HIV-1 tat basic region amino acid sequence, which may include, for example, amino acids 49-57 of naturally occurring tat protein.

Other permeant domains include poly-arginine motifs, for example, the region of amino acids 34-56 of HIV-1 rev protein, nona-arginine, acta-arginine, and the like. (See, for example, Futaki et al. (2003) Curr Protein Pept Sci. 2003 April; 4 (2): 87-9 and 446; and Wender et al. (2000) Proc. Natl. Acad. Sci. U.S.A. 2000 Nov. 21; 97 (24): 13003-8; published US Patent Application Publications Nos. 20030220334; 20030083256; 20030032593; and 20030022831, herein specifically incorporated by reference for the teachings of translocation peptides and peptoids). The nona-arginine (R9) sequence is one of the more efficient PTDs that have been characterized (Wender et al. 2000; Uemura et al. 2002). The site at which the fusion is made may be selected in order to optimize the biological activity, secretion or binding characteristics of the polypeptide. The optimal site will be determined by routine experimentation. In some embodiments the polypeptide permeant domain is chemically modified in order to increase the bioavailability of the PTD. Exemplary modifications are disclosed in Expert Opin Drug Deliv. 2009 November; 6 (11): 1195-205.

A sRGN polypeptide may be produced in vitro or by eukaryotic cells, by prokaryotic cells, or by in vitro transcription and translation (IVTT) and it may be further processed by unfolding, e.g., heat denaturation, OTT reduction, etc. and may be further refolded, using methods known in the art.

Modifications of interest that do not alter primary sequence include chemical derivatization of polypeptides, e.g., acylation, acetylation, carboxylation, amidation, etc. Also included are modifications of glycosylation, e.g., those made by modifying the glycosylation patterns of a polypeptide during its synthesis and processing or in further processing steps; e.g., by exposing the polypeptide to enzymes which affect glycosylation, such as mammalian glycosylating or deglycosylating enzymes. Also embraced are sequences that have phosphorylated amino acid residues, e.g., phosphotyrosine, phosphoserine, or phosphothreonine.

Also included in the invention are guide RNAs and sRGN polypeptides that have been modified using ordinary molecular biological techniques and synthetic chemistry so as to improve their resistance to proteolytic degradation, to change the target sequence specificity, to optimize solubility properties, to alter protein activity (e.g., transcription modulatory activity, enzymatic activity, etc.) or to render them more suitable as a therapeutic agent. Analogs of such polypeptides include those containing residues other than naturally occurring L-amino acids, e.g., O-amino acids or non-naturally occurring synthetic amino acids. D-amino acids may be substituted for some or all of the amino acid residues. The sRGN polypeptides may be prepared by in vitro synthesis, using conventional methods as known in the art. Various commercial synthetic apparatuses are available, for example, automated synthesizers by Applied Biosystems, Inc., Beckman, etc. By using synthesizers, naturally occurring amino acids may be substituted with unnatural amino acids. The particular sequence and the manner of preparation will be determined by convenience, economics, purity required, and the like.

If desired, various groups may be introduced into the peptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

An sRGN polypeptide may also be isolated and purified in accordance with known methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, such as at least about 75% by weight or at least about 95% by weight, and for therapeutic purposes, at least 99.5% by weight, in relation to contaminants related to the method of preparation of the product and its purification. Usually, the percentages will be based upon total protein. To induce DNA cleavage and recombination, or any desired modification to a target DNA, or any desired modification to a polypeptide associated with target DNA, the guide RNA and/or the sRGN polypeptide and/or the donor polynucleotide, whether they be introduced as nucleic acids or polypeptides, are provided to the cells for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which may be repeated with a frequency of about every day to about every 4 days, e.g., every 1.5 days, every 2 days, every 3 days, or any other frequency from about every day to about every four days. The agent(s) may be provided to the cells one or more times, e.g., one time, twice, three times, or more than three times, and the cells allowed to incubate with the agent(s) for some amount of time following each contacting event e.g., 16-24 hours, after which time the media is replaced with fresh media and the cells are cultured further. In cases in which two or more different targeting complexes are provided to the cell (e.g., two different guide RNAs that are complementary to different sequences within the same or different target DNA), the complexes may be provided simultaneously (e.g., as two polypeptides and/or nucleic acids), or delivered simultaneously. Alternatively, they may be provided consecutively, e.g., the targeting complex being provided first, followed by the second targeting complex, etc. or vice versa.

Generally, an effective amount of the guide RNA and/or sRGN polypeptide and/or donor polynucleotide is provided to the target DNA or cells to induce target modification. An effective amount of the guide RNA and/or sRGN polypeptide and/or donor polynucleotide is the amount to induce a 2-fold increase or more in the amount of target modification observed between two homologous sequences relative to a negative control, e.g., a cell contacted with an empty vector or irrelevant polypeptide. That is to say, an effective amount or dose of the guide RNA and/or sRGN polypeptide and/or donor polynucleotide will induce a 2-fold increase, a 3-fold increase, a 4-fold increase or more in the amount of target modification observed at a target DNA region, in some instances a 5-fold increase, a 6-fold increase or more, sometimes a 7-fold or 8-fold increase or more in the amount of recombination observed, e.g., an increase of 10-fold, 50-fold, or 100-fold or more, in some instances, an increase of 200-fold, 500-fold, 700-fold, or 1000-fold or more, e.g., a 5000-fold, or 10,000-fold increase in the amount of recombination observed. The amount of target modification may be measured by any convenient method. For example, a silent reporter construct comprising complementary sequence to the targeting segment (targeting sequence) of the guide RNA flanked by repeat sequences that, when recombined, will reconstitute a nucleic acid encoding an active reporter may be co-transfected into the cells, and the amount of reporter protein assessed after contact with the guide RNA and/or sRGN polypeptide and/or donor polynucleotide, e.g., 2 hours, 4 hours, 8 hours, 12 hours, 24 hours, 36 hours, 48 hours, 72 hours or more after contact with the guide RNA and/or sRGN polypeptide and/or donor polynucleotide. As another, more sensitivity assay, for example, the extent of recombination at a genomic DNA region of interest comprising target DNA sequences may be assessed by PCR or Southern hybridization of the region after contact with a guide RNA and/or sRGN polypeptide and/or donor polynucleotide, e.g., 2 hours, 4 hours, 8 hours, 12 hours, 24 hours, 36 hours, 48 hours, 72 hours or more after contact with the guide RNA and/or sRGN polypeptide and/or donor polynucleotide.

Contacting the cells with a guide RNA and/or sRGN polypeptide and/or donor polynucleotide may occur in any culture media and under any culture conditions that promote the survival of the cells. For example, cells may be suspended in any appropriate nutrient medium that is convenient, such as lscove's modified DMEM or RPMI1640, supplemented with fetal calf serum or heat inactivated goat serum (about 5-10%), L-glutamine, a thiol, particularly 2-mercaptoethanol, and antibiotics, e.g., penicillin and streptomycin. The culture may contain growth factors to which the cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors. Conditions that promote the survival of cells are generally permissive of nonhomologous end joining and homology-directed repair. In applications in which it is desirable to insert a polynucleotide sequence into a target DNA sequence, a polynucleotide comprising a donor sequence to be inserted is also provided to the cell. By a “donor sequence” or “donor polynucleotide” it is meant a nucleic acid sequence to be inserted at the cleavage site induced by an sRGN polypeptide. The donor polynucleotide will contain sufficient homology to a genomic sequence at the cleavage site, e.g., 70%, 80%, 85%, 90%, 95%, or 100% homology with the nucleotide sequences flanking the cleavage site, e.g., within about 50 bases or less of the cleavage site, e.g., within about 30 bases, within about 15 bases, within about 10 bases, within about 5 bases, or immediately flanking the cleavage site, to support homology-directed repair between it and the genomic sequence to which it bears homology. Approximately 25, 50, 100, or 200 nucleotides, or more than 200 nucleotides, of sequence homology between a donor and a genomic sequence (or any integral value between 10 and 200 nucleotides, or more) will support homology-directed repair. Donor sequences can be of any length, e.g., 10 nucleotides or more, 50 nucleotides or more, 100 nucleotides or more, 250 nucleotides or more, 500 nucleotides or more, 1000 nucleotides or more, 5000 nucleotides or more, etc.

The donor sequence is generally not identical to the genomic sequence that it replaces. Rather, the donor sequence may contain at least one or more single base changes, insertions, deletions, inversions or rearrangements with respect to the genomic sequence, so long as sufficient homology is present to support homology-directed repair. In some embodiments, the donor sequence comprises a non-homologous sequence flanked by two regions of homology, such that homology-directed repair between the target DNA region and the two flanking sequences results in insertion of the non-homologous sequence at the target region. Donor sequences may also comprise a vector backbone containing sequences that are not homologous to the DNA region of interest and that are not intended for insertion into the DNA region of interest. Generally, the homologous region(s) of a donor sequence will have at least 50% sequence identity to a genomic sequence with which recombination is desired. In certain embodiments, 60%, 70%, 80%, 90%, 95%, 98%, 99%, or 99.9% sequence identity is present. Any value between 1% and 100% sequence identity can be present, depending upon the length of the donor polynucleotide. The donor sequence may comprise certain sequence differences as compared to the genomic sequence, e.g., restriction sites, nucleotide polymorphisms, selectable markers (e.g., drug resistance genes, fluorescent proteins, enzymes etc.), etc., which may be used to assess for successful insertion of the donor sequence at the cleavage site or in some cases may be used for other purposes (e.g., to signify expression at the targeted genomic locus). In some cases, if located in a coding region, such nucleotide sequence differences will not change the amino acid sequence, or will make silent amino acid changes (i.e., changes which do not affect the structure or function of the protein). Alternatively, these sequences differences may include flanking recombination sequences such as FLPs, loxP sequences, or the like, that can be activated at a later time for removal of the marker sequence.

The donor sequence may be provided to the cell as single-stranded DNA, single-stranded RNA, double-stranded DNA, or double-stranded RNA. It may be introduced into a cell in linear or circular form. If introduced in linear form, the ends of the donor sequence may be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al. (1987) Proc. Natl. Acad. Sci. USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. As an alternative to protecting the termini of a linear donor sequence, additional lengths of sequence may be included outside of the regions of homology that can be degraded without impacting recombination. A donor sequence can be introduced into a cell as part of a vector molecule having additional sequences such as, for example, replication origins, promoters and genes encoding antibiotic resistance. Moreover, donor sequences can be introduced as naked (i.e., unmodified) nucleic acid, as nucleic acid complexed with an agent such as a liposome or poloxamer, or can be delivered by viruses (e.g., adenovirus, AAV), as described above for nucleic acids encoding a guide RNA and/or sRGN polypeptide and/or donor polynucleotide.

Following the methods described above, a DNA region of interest may be cleaved and modified, i.e., “genetically modified”, ex vivo. In some embodiments, as when a selectable marker has been inserted into the DNA region of interest, the population of cells may be enriched for those comprising the genetic modification by separating the genetically modified cells from the remaining population. Prior to enriching, the “genetically modified” cells may make up only about 1% or more (e.g., 2% or more, 3% or more, 4% or more, 5% or more, 6% or more, 7% or more, 8% or more, 9% or more, 10% or more, 15% or more, or 20% or more) of the cellular population. Separation of “genetically modified” cells may be achieved by any convenient separation technique appropriate for the selectable marker used. For example, if a fluorescent marker has been inserted, cells may be separated by fluorescence activated cell sorting, whereas if a cell surface marker has been inserted, cells may be separated from the heterogeneous population by affinity separation techniques, e.g., magnetic separation, affinity chromatography, “panning” with an affinity reagent attached to a solid matrix, or other convenient technique. Techniques providing accurate separation include fluorescence activated cell sorters, which can have varying degrees of sophistication. Such as multiple color channels, low angle and obtuse light scattering detecting channels, impedance channels, etc. The cells may be selected against dead cells by employing dyes associated with dead cells (e.g., propidium iodide). Any technique may be employed which is not unduly detrimental to the viability of the genetically modified cells. Cell compositions that are highly enriched for cells comprising modified DNA are achieved in this manner. By “highly enriched”, it is meant that the genetically modified cells will be 70% or more, 75% or more, 80% or more, 85% or more, 90% or more of the cell composition, for example, about 95% or more, or 98% or more of the cell composition. In other words, the composition may be a substantially pure composition of genetically modified cells.

Genetically modified cells produced by the methods described herein may be used immediately. Alternatively, the cells may be frozen at liquid nitrogen temperatures and stored for long periods of time, being thawed and capable of being reused. In such cases, the cells will usually be frozen in 10% dimethylsulfoxide (DMSO), 50% serum, 40% buffered medium, or some other such solution as is commonly used in the art to preserve cells at such freezing temperatures, and thawed in a manner as commonly known in the art for thawing frozen cultured cells.

The genetically modified cells may be cultured in vitro using methods known in the art.

The cells may be expanded in culture, i.e., grown under conditions that promote their proliferation. Culture medium may be liquid or semi-solid, e.g., containing agar, methylcellulose, etc. The cell population may be suspended in an appropriate nutrient medium, such as lscove's modified DMEM or RPMI 1640, normally supplemented with fetal calf serum (about 5-10%), L-glutamine, a thiol, particularly 2-mercaptoethanol, and antibiotics, e.g., penicillin and streptomycin. The culture may contain growth factors to which the respective cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors. Cells that have been genetically modified in this way may be transplanted to a subject for purposes such as gene therapy, e.g., to treat a disease or as an antiviral, antipathogenic, or anticancer therapeutic, for the production of genetically modified organisms in agriculture, or for biological research. In some cases, cells for use in humans are cultured in a Xeno-free medium. The subject may be a neonate, a juvenile, or an adult. Of particular interest are mammalian subjects. Mammalian species that may be treated with the present methods include canines and felines; equines; bovines; ovines; etc. and primates, particularly humans. Animal models, particularly small mammals (e.g., mouse, rat, guinea pig, hamster, lagomorph (e.g., rabbit), etc.) may be used for experimental investigations.

Cells may be provided to the subject alone or with a suitable substrate or matrix, e.g., to support their growth and/or organization in the tissue to which they are being transplanted. Usually, at least 1×10\hat{ }3 cells will be administered, for example 5×10³ cells, 1×10⁴ cells, 5×10⁴ cells, 1×10⁵ cells, 1×10⁶ cells or more. The cells may be introduced to the subject via any of the following routes: parenteral, subcutaneous, intravenous, intracranial, intraspinal, intraocular, or into spinal fluid. The cells may be introduced by injection, catheter, or the like. Examples of methods for local delivery, that is, delivery to the site of injury, include, e.g., through an Ommaya reservoir, e.g., for intrathecal delivery (e.g., U.S. Pat. Nos. 5,222,982 and 5,385,582, incorporated herein by reference); by bolus injection, e.g., by a syringe, e.g., into a joint; by continuous infusion, e.g., by cannulation, e.g., with convection (e.g., U.S. application Ser. No. 20070254842, incorporated herein by reference); or by implanting a device upon which the cells have been reversibly affixed (e.g., U.S. application Nos. 20080081064 and 20090196903, incorporated herein by reference). Cells may also be introduced into an embryo (e.g., a blastocyst) for the purpose of generating a transgenic animal (e.g., a transgenic mouse).

Delivery of sRGN Systems

Guide RNA polynucleotides (RNA or DNA) and/or sRGN polypeptides (RNA or DNA) can be delivered by viral or non-viral delivery vehicles known in the art.

Polynucleotides may be delivered by non-viral delivery vehicles including, but not limited to, nanoparticles, liposomes, ribonucleoproteins, positively charged peptides, small molecule RNA-conjugates, aptamer-RNA chimeras, and RNA-fusion protein complexes. Some exemplary non-viral delivery vehicles are described in Peer and Lieberman, Gene Therapy, 18:1127-1133 (2011) (which focuses on non-viral delivery vehicles for siRNA that are also useful for delivery of other polynucleotides).

A recombinant adeno-associated virus (AAV) vector may be used for delivery. Techniques to produce rAAV particles, in which an AAV genome to be packaged that includes the polynucleotide to be delivered, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs, including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13 and AAV rh.74. Production of pseudotyped rAAV is disclosed in, for example, WO 01/83692.

TABLE 3

| AAV | Genbank Accession |
|---|---|
| Serotype | No. |
| AAV-1 | NC_002077.1 |
| AAV-2 | NC_001401.2 |
| AAV-3 | NC_001729.1 |
| AAV-38 | AF028705.1 |
| AAV-4 | NC_001829.1 |
| AAV-5 | NC_006152.1 |
| AAV-6 | AF028704.1 |
| AAV-7 | NC_006260.1 |
| AAV-8 | NC_006261.1 |
| AAV-9 | AX753250.1 |
| AAV-10 | AY631965.1 |
| AAV-11 | AY631966.1 |
| AAV-12 | 00813647.1 |
| AAV-13 | EU285562.1 |

A method of generating a packaging cell is to create a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Micro Biol. and Immunol., 158:97-129). Various approaches are described in Ratschin et al., Mol. Cell. Bio. 4:2072 (1984); Hermonat et al., Proc. Natl. Acad. Sci. USA, 81:6466 (1984); Tratschin et al., Mo1. Cell. Biol. 5:3251 (1985); Mclaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 96/17947; 95/13392; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595.

AAV vector serotypes can be matched to target cell types. For example, the following exemplary cell types transduced by the indicated AAV serotypes among others.

TABLE 4

| Tissue/Cell Type | Serotype |
|---|---|
| Liver | AAV8, AAV9 |
| Skeletal muscle | AAV1, AAV7, AAV6, AAV8, AAV9 |
| Central nervous system | AAV5, AAV1, AAV4 |
| RPE | AAV5, AAV4 |
| Photoreceptor cells | AAV5 |
| Lung | AAV9 |
| Heart | AAV8 |
| Pancreas | AAV8 |
| Kidney | AAV2 |

The number of administrations of treatment to a subject may vary. Introducing the genetically modified cells into the subject may be a one-time event; but in certain situations, such treatment may elicit improvement for a limited period of time and require an on-going series of repeated treatments. In other situations, multiple administrations of the genetically modified cells may be required before an effect is observed. The exact protocols depend upon the disease or condition, the stage of the disease and parameters of the individual subject being treated.

In other aspects of the disclosure, the guide RNA and/or sRGN polypeptide and/or donor polynucleotide are employed to modify cellular DNA in vivo, for example for purposes such as gene therapy, e.g., to treat a disease or as an antiviral, antipathogenic, or anticancer therapeutic, for the production of genetically modified organisms in agriculture, or for biological research. In these in vivo embodiments, a guide RNA and/or sRGN polypeptide and/or donor polynucleotide are administered directly to the individual. A guide RNA and/or sRGN polypeptide and/or donor polynucleotide may be administered by any of a number of methods known in the art for the administration of peptides, small molecules, and nucleic acids to a subject. A guide RNA and/or sRGN polypeptide and/or donor polynucleotide can be incorporated into a variety of formulations. More particularly, a guide RNA and/or sRGN polypeptide and/or donor polynucleotide of the present invention can be formulated into pharmaceutical compositions by combination with appropriate pharmaceutically acceptable carriers or diluents. In some cases, the guide RNA and sRGN are formulated for ex vivo delivery.

Pharmaceutical preparations are compositions that include one or more a guide RNA and/or sRGN polypeptide and/or donor polynucleotide present in a pharmaceutically acceptable vehicle. “Pharmaceutically acceptable vehicles” may be vehicles approved by a regulatory agency of the Federal or a state government or listed in the US Pharmacopeia or other generally recognized pharmacopeia for use in mammals, such as humans. The term “vehicle” refers to a diluent, adjuvant, excipient, or carrier with which a compound of the invention is formulated for administration to a mammal. Such pharmaceutical vehicles can be lipids, e.g., liposomes, e.g., liposome dendrimers; liquids, such as water and oils, including those of petroleum, animal, vegetable or synthetic origin, such as peanut oil, soybean oil, mineral oil, sesame oil and the like, saline; gum acacia, gelatin, starch paste, talc, keratin, colloidal silica, urea, and the like. In addition, auxiliary, stabilizing, thickening, lubricating and coloring agents may be used. Pharmaceutical compositions may be formulated into preparations in solid, semisolid, liquid or gaseous forms, such as tablets, capsules, powders, granules, ointments, solutions, suppositories, injections, inhalants, gels, microspheres, and aerosols. As such, administration of a guide RNA and/or sRGN polypeptide and/or donor polynucleotide can be achieved in various ways, including oral, buccal, rectal, parenteral, intraperitoneal, intradermal, transdermal, intra-tracheal, intraocular, etc., administration. The active agent may be systemic after administration or may be localized by the use of regional administration, intramural administration, or use of an implant that acts to retain the active dose at the site of implantation. The active agent may be formulated for immediate activity or it may be formulated for sustained release.

For some conditions, particularly central nervous system conditions, it may be necessary to formulate agents to cross the blood-brain barrier (BBB). One strategy for drug delivery through the BBB entails disruption of the BBB, either by osmotic means such as mannitol or leukotrienes, or biochemically by the use of vasoactive substances such as bradykinin. The potential for using BBB opening to target specific agents to brain tumors is also an option. A BBB-disrupting agent can be co-administered with the therapeutic compositions of the invention when the compositions are administered by intravascular injection. Other strategies for delivery through the BBB may entail the use of endogenous transport systems, including Caveolin-1 mediated transcytosis, carrier-mediated transporters such as glucose and amino acid carriers, receptor-mediated transcytosis for insulin or transferrin, and active efflux transporters such asp-glycoprotein. Active transport moieties may also be conjugated to the therapeutic compounds for use in the invention to facilitate transport across the endothelial wall of the blood vessel. Alternatively, drug delivery of therapeutics agents behind the BBB may be by local delivery, for example by intrathecal delivery, e.g., through an Ommaya reservoir (see e.g., U.S. Pat. Nos. 5,222,982 and 5,385,582, incorporated herein by reference); by bolus injection, e.g., by a syringe, e.g., intravitreally or intracranially; by continuous infusion, e.g., by cannulation, e.g., with convection (see e.g., U.S. application No. 20070254842, incorporated here by reference); or by implanting a device upon which the agent has been reversibly affixed (see e.g., U.S. application Nos. 20080081064 and 20090196903, incorporated herein by reference).

Generally, an effective amount of a guide RNA and/or sRGN polypeptide and/or donor polynucleotide are provided. As discussed above with regard to ex vivo methods, an effective amount or effective dose of a guide RNA and/or sRGN polypeptide and/or donor polynucleotide in vivo is the amount to induce a 2 fold increase or more in the amount of recombination observed between two homologous sequences relative to a negative control, e.g., a cell contacted with an empty vector or irrelevant polypeptide. The amount of recombination may be measured by any convenient method, e.g., as described above and known in the art. The calculation of the effective amount or effective dose of a guide RNA and/or sRGN polypeptide and/or donor polynucleotide to be administered is within the skill of one of ordinary skill in the art, and will be routine to those persons skilled in the art. The final amount to be administered will be dependent upon the route of administration and upon the nature of the disorder or condition that is to be treated.

The effective amount given to a particular patient will depend on a variety of factors, several of which will differ from patient to patient. A competent clinician will be able to determine an effective amount of a therapeutic agent to administer to a patient to halt or reverse the progression the disease condition as required. Utilizing LD50 animal data, and other information available for the agent, a clinician can determine the maximum safe dose for an individual, depending on the route of administration. For example, an intravenously administered dose may have a greater amount of therapeutic agent than an intrathecally-administered dose, given the greater body of fluid into which the therapeutic composition is being administered. Similarly, compositions, which are rapidly cleared from the body may be administered at higher doses, or in repeated doses, in order to maintain a therapeutic concentration. Utilizing ordinary skill, the competent clinician will be able to optimize the dosage of a particular therapeutic in the course of routine clinical trials.

For inclusion in a medicament, a guide RNA and/or sRGN polypeptide and/or donor polynucleotide may be obtained from a suitable commercial source. As a general proposition, the total pharmaceutically effective amount of a guide RNA and/or sRGN polypeptide and/or donor polynucleotide administered parenterally per dose will be in a range that can be measured by a dose response curve.

Therapies based on a guide RNA and/or sRGN polypeptide and/or donor polynucleotides, i.e., preparations of a guide RNA and/or sRGN polypeptide and/or donor polynucleotide to be used for therapeutic administration, must be sterile. Sterility is readily accomplished by filtration through sterile filtration membranes (e.g., 0.2 1-1m membranes). Therapeutic compositions generally are placed into a container having a sterile access port, for example, an intravenous solution bag or vial having a stopper pierceable by a hypodermic injection needle. The therapies based on a guide RNA and/or sRGN polypeptide and/or donor polynucleotide may be stored in unit or multi-dose containers, for example, sealed ampules or vials, as an aqueous solution or as a lyophilized formulation for reconstitution. As an example of a lyophilized formulation, 10-ml vials are filled with 5 ml of sterile-filtered 1% (w/v) aqueous solution of compound, and the resulting mixture is lyophilized. The infusion solution is prepared by reconstituting the lyophilized compound using bacteriostatic Water-for-Injection.

Pharmaceutical compositions can include, depending on the formulation desired, pharmaceutically acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The nucleic acids or polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration are known in the art, for example, Remington's Pharmaceutical Sciences, Mace Publishing Company, Philadelphia, Pa., 21st ed. (2006). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Therapies that exhibit large therapeutic indices may be preferred.

The data obtained from cell culture and/or animal studies can be used in formulating a range of dosages for humans. The dosage of the active ingredient generally lies within a range of circulating concentrations that include the ED50 with low toxicity. The dosage can vary within this range depending upon the dosage form employed and the route of administration utilized. The components used to formulate the pharmaceutical compositions are in some embodiments of high purity and substantially free of potentially harmful contaminants (e.g., at least National Food (NF) grade, generally at least analytical grade, including at least pharmaceutical grade). Moreover, compositions intended for in vivo use are usually sterile. To the extent that a given compound must be synthesized prior to use, the resulting product is generally substantially free of any potentially toxic agents, particularly any endotoxins, which may be present during the synthesis or purification process. Compositions for parental administration are also sterile, substantially isotonic and made under GMP conditions.

The effective amount of a therapeutic composition to be given to a particular patient will depend on a variety of factors, several of which will differ from patient to patient. A competent clinician will be able to determine an effective amount of a therapeutic agent to administer to a patient to halt or reverse the progression the disease condition as required. Utilizing LD50 animal data, and other information available for the agent, a clinician can determine the maximum safe dose for an individual, depending on the route of administration. For instance, an intravenously administered dose may be more than an intrathecally administered dose, given the greater body of fluid into which the therapeutic composition is being administered. Similarly, compositions that are rapidly cleared from the body may be administered at higher doses, or in repeated doses, in order to maintain a therapeutic concentration. Utilizing ordinary skill, the competent clinician will be able to optimize the dosage of a particular therapeutic in the course of routine clinical trials.

### Genetically Modified Host Cells

The present disclosure provides genetically modified host cells, including isolated genetically modified host cells, where a genetically modified host cell comprises (has been genetically modified with:

1) an exogenous guide RNA; 2) an exogenous nucleic acid comprising a nucleotide sequence encoding a guide RNA; 3) an exogenous sRGN polypeptide (e.g., a naturally occurring sRGN; a modified, i.e., mutated or variant, sRGN; a chimeric sRGN; etc.); 4) an exogenous nucleic acid comprising a nucleotide sequence encoding an sRGN polypeptide; or 5) any combination of the above. A genetically modified cell is generated by genetically modifying a host cell with, for example: 1) an exogenous guide RNA; 2) an exogenous nucleic acid comprising a nucleotide sequence encoding a guide RNA; 3) an exogenous sRGN polypeptide; 4) an exogenous nucleic acid comprising a nucleotide sequence encoding an sRGN polypeptide; or 5) any combination of the above).

All cells suitable to be a target cell are also suitable to be a genetically modified host cell. For example, a genetically modified host cells of interest can be a cell from any organism (e.g., a bacterial cell, an archaeal cell, a cell of a single-cell eukaryotic organism, a plant cell, an algal cell, e.g., Botryococcus braunii, Chlamydomonas reinhardtii, Nannochloropsis gaditana, Chlorella pyrenoidosa, Sargassum patens C. Agardh, and the like, a fungal cell (e.g., a yeast cell), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent. a rat, a mouse, a non-human primate, a human, etc.), etc.

In some embodiments, a genetically modified host cell has been genetically modified with an exogenous nucleic acid comprising a nucleotide sequence encoding an sRGN polypeptide (e.g., a naturally occurring sRGN; a modified, i.e., mutated or variant, sRGN; a chimeric sRGN; etc.). The DNA of a genetically modified host cell can be targeted for modification by introducing into the cell a guide RNA (or a DNA encoding a guide RNA, which determines the genomic location/sequence to be modified) and optionally a donor nucleic acid. In some embodiments, the nucleotide sequence encoding an sRGN polypeptide is operably linked to an inducible promoter (e.g., heat shock promoter, Tetracycline-regulated promoter, Steroid-regulated promoter, Metal-regulated promoter, estrogen receptor-regulated promoter, etc.). In some embodiments, the nucleotide sequence encoding an sRGN polypeptide is operably linked to a spatially restricted and/or temporally restricted promoter (e.g., a tissue specific promoter, a cell type specific promoter, a cell cycle specific promoter). In some embodiments, the nucleotide sequence encoding an sRGN polypeptide is operably linked to a constitutive promoter.

In some embodiments, a genetically modified host cell is in vitro. In some embodiments, a genetically modified host cell is in vivo. In some embodiments, a genetically modified host cell is a prokaryotic cell or is derived from a prokaryotic cell. In some embodiments, a genetically modified host cell is a bacterial cell or is derived from a bacterial cell. In some embodiments, a genetically modified host cell is an archaeal cell or is derived from an archaeal cell. In some embodiments, a genetically modified host cell is a eukaryotic cell or is derived from a eukaryotic cell. In some embodiments, a genetically modified host cell is a plant cell or is derived from a plant cell. In some embodiments, a genetically modified host cell is an animal cell or is derived from an animal cell. In some embodiments, a genetically modified host cell is an invertebrate cell or is derived from an invertebrate cell. In some embodiments, a genetically modified host cell is a vertebrate cell or is derived from a vertebrate cell. In some embodiments, a genetically modified host cell is a mammalian cell or is derived from a mammalian cell. In some embodiments, a genetically modified host cell is a rodent cell or is derived from a rodent cell. In some embodiments, a genetically modified host cell is a human cell or is derived from a human cell.

The present disclosure further provides progeny of a genetically modified cell, where the progeny can comprise the same exogenous nucleic acid or polypeptide as the genetically modified cell from which it was derived. The present disclosure further provides a composition comprising a genetically modified host cell.

### Genetically Modified Stem Cells and Genetically Modified Progenitor Cells

In some embodiments, a genetically modified host cell is a genetically modified stem cell or progenitor cell. Suitable host cells include, e.g., stem cells (adult stem cells, embryonic stem cells, iPS cells, etc.) and progenitor cells (e.g., cardiac progenitor cells, neural progenitor cells, etc.). Suitable host cells include mammalian stem cells and progenitor cells, including, e.g., rodent stem cells, rodent progenitor cells, human stem cells, human progenitor cells, etc. Suitable host cells include in vitro host cells, e.g., isolated host cells.

In some embodiments, a genetically modified host cell comprises an exogenous guide RNA nucleic acid. In some embodiments, a genetically modified host cell comprises an exogenous nucleic acid comprising a nucleotide sequence encoding a guide RNA. In some embodiments, a genetically modified host cell comprises an exogenous sRGN polypeptide (e.g., a naturally occurring sRGN; a modified, i.e., mutated or variant, sRGN; a chimeric sRGN; etc.).

In some embodiments, a genetically modified host cell comprises an exogenous nucleic acid comprising a nucleotide sequence encoding an sRGN polypeptide. In some embodiments, a genetically modified host cell comprises exogenous nucleic acid comprising a nucleotide sequence encoding 1) a guide RNA and 2) an sRGN polypeptide.

In some cases, the sRGN polypeptide comprises an amino acid sequence having at least 75%, at least 80%, at least 85%, at least 90%, at least 95%, at least 99%, or 100% amino acid sequence identity to any of the sequences in SEQ ID NO: 1-12, or an active portion thereof which is at least 100, 150, 200, 300, 350, 400, or 500 amino acids long. In some embodiments, the active portion comprises an RNase domain. In other embodiments, the active portion comprises a DNase domain.

### Compositions

The present disclosure provides a composition comprising a guide RNA and/or a site-directed modifying polypeptide. In some cases, the sRGN polypeptide is a chimeric polypeptide. A composition is useful for carrying out a method of the present disclosure, e.g., a method for site-specific modification of a target DNA; a method for site-specific modification of a polypeptide associated with a target DNA; etc.

### Compositions Comprising a Guide RNA

The present disclosure provides a composition comprising a guide RNA. The composition can comprise, in addition to the guide RNA, one or more of: a salt, e.g. NaCl, MgCl, KCl, MgS04, etc.; a buffering agent, e.g. a Tris buffer, N-(2-hydroxyethyl) piperazine-N′-(2-ethanesulfonic acid) (HEPES), 2-(N-morpholino) ethanesulfonic acid (MES), MES sodium salt, 3-(N-morpholino) propanesulfonic acid (MOPS), N-tris [hydroxymethyl]methyl-3-aminopropanesulfonic acid (TAPS), etc.; a solubilizing agent; a detergent, e.g. a non-ionic detergent such as Tween®20, etc.; a nuclease inhibitor; and the like. For example, in some cases, a composition comprises a guide RNA and a buffer for stabilizing nucleic acids.

In some embodiments, a guide RNA present in a composition is pure, e.g., at least 75%, at least 80%, at least 85%, at least 90%, at least 95%, at least 98%, at least 99%, or more than 99% pure, where “% purity” means that guide RNA is the recited percent free from other macromolecules, or contaminants that may be present during the production of the guide RNA.

### Compositions Comprising a Chimeric Polypeptide

The present disclosure provides a composition of a chimeric polypeptide. The composition can comprise, in addition to the guide RNA, one or more of: a salt, e.g., NaCl, MgCl, KCl, MgS04, etc.; a buffering agent, e.g., a Tris buffer, HEPES, MES, MES sodium salt, MOPS, TAPS, etc.; a solubilizing agent; a detergent, e.g., a non-ionic detergent such as Tween®20, etc.; a protease inhibitor; a reducing agent (e.g., dithiothreitol); and the like.

In some embodiments, a chimeric polypeptide present in a composition is pure, e.g., at least 75%, at least 80%, at least 85%, at least 90%, at least 95%, at least 98%, at least 99%, or more than 99% pure, where “% purity” means that the site-directed modifying polypeptide is the recited percent free from other proteins, other macromolecules, or contaminants that may be present during the production of the chimeric polypeptide.

Compositions Comprising a Guide RNA and an sRGN Polypeptide

The present disclosure provides a composition comprising: (i) a guide RNA or a DNA nucleic acid encoding the same; and ii) an sRGN polypeptide, or a nucleic acid encoding the same. In some cases, the sRGN polypeptide is a chimeric sRGN polypeptide. In other cases, the sRGN polypeptide is a naturally occurring sRGN polypeptide. In some instances, the sRGN polypeptide exhibits enzymatic activity that modifies a target DNA. In other cases, the sRGN polypeptide exhibits enzymatic activity that modifies a polypeptide that is associated with a target DNA. In still other cases, the sRGN polypeptide modulates transcription of the target DNA.

The present disclosure provides a composition comprising: (i) a guide RNA, as described above, or a DNA nucleic acid encoding the same, the guide RNA comprising: (a) a first segment comprising a nucleotide sequence that is complementary to a sequence in a target DNA; and (b) a second segment that interacts with an sRGN polypeptide; and (ii) the sRGN polypeptide, or a nucleic acid encoding the same, the sRGN polypeptide comprising: (a) an RNA-binding portion that interacts with the guide RNA; and (b) an activity portion that exhibits site-directed enzymatic activity, wherein the site of enzymatic activity is determined by the guide RNA.

In some instances, a composition comprises: (i) a guide RNA, the guide RNA comprising: (a) a first segment comprising a nucleotide sequence that is complementary to a sequence in a target DNA; and (b) a second segment that interacts with an sRGN polypeptide; and (ii) the sRGN polypeptide, the sRGN polypeptide comprising: (a) an RNA-binding portion that interacts with the guide RNA; and (b) an activity portion that exhibits site-directed enzymatic activity, wherein the site of enzymatic activity is determined by the guide RNA.

In other embodiments, a composition comprises: (i) a nucleic acid encoding a guide RNA, the guide RNA comprising: (a) a first segment comprising a nucleotide sequence that is complementary to a sequence in a target DNA; and (b) a second segment that interacts with a site-directed modifying polypeptide; and (ii) a nucleic acid encoding the sRGN polypeptide, the sRGN polypeptide comprising: (a) an RNA-binding portion that interacts with the guide RNA; and (b) an activity portion that exhibits site-directed enzymatic activity, wherein the site of enzymatic activity is determined by the guide RNA.

The present disclosure provides a composition comprising: (i) a guide RNA, or a DNA nucleic acid encoding the same, the guide RNA comprising: (a) a first segment comprising a nucleotide sequence that is complementary to a sequence in a target DNA; and (b) a second segment that interacts with an sRGN polypeptide; and (ii) the sRGN polypeptide, or a nucleic acid encoding the same, the sRGN polypeptide comprising: (a) an RNA-binding portion that interacts with the guide RNA; and (b) an activity portion that modulates transcription within the target DNA, wherein the site of modulated transcription within the target DNA is determined by the guide RNA.

For example, in some cases, a composition comprises: (i) a guide RNA the guide RNA comprising: (a) a first segment comprising a nucleotide sequence that is complementary to a sequence in a target DNA; and (b) a second segment that interacts with an sRGN polypeptide; and (ii) the sRGN polypeptide, the sRGN polypeptide comprising: (a) an RNA-binding portion that interacts with the guide RNA; and (b) an activity portion that modulates transcription within the target DNA, wherein the site of modulated transcription within the target DNA is determined by the guide RNA.

As another example, in some cases, a composition comprises: (i) a DNA nucleic acid encoding a guide RNA, the guide RNA comprising: (a) a first segment comprising a nucleotide sequence that is complementary to a sequence in a target DNA; and (b) a second segment that interacts with an sRGN polypeptide; and (ii) a nucleic acid encoding the site-directed modifying polypeptide, the sRGN polypeptide comprising: (a) an RNA-binding portion that interacts with the guide RNA; and (b) an activity portion that modulates transcription within the target DNA, wherein the site of modulated transcription within the target DNA is determined by the guide RNA. A composition can comprise, in addition to i) a guide RNA, or a DNA nucleic acid encoding the same; and ii) an sRGN polypeptide, or a nucleic acid encoding the same, one or more of: a salt, e.g. NaCl, MgCl, KCl, MgS04, etc.; a buffering agent, e.g. a Tris buffer, HEPES, MES, MES sodium salt, MOPS, TAPS, etc.; a solubilizing agent a detergent, e.g. a non-ionic detergent such as Tween-20, etc.; a protease inhibitor; a reducing agent (e.g. dithiothreitol); and the like.

In some cases, the components of the composition are individually pure, e.g., each of the components is at least 75%, at least 80%, at least 90%, at least 95%, at least 98%, at least 99%, or at least 99%, pure. In some cases, the individual components of a composition are pure before being added to the composition.

For example, in some embodiments, an sRGN polypeptide present in a composition is pure, e.g., at least 75%, at least 80%, at least 85%, at least 90%, at least 95%, at least 98%, at least 99%, or more than 99% pure, where “% purity” means that the sRGN polypeptide is the recited percent free from other proteins (e.g., proteins other than the sRGN polypeptide), other macromolecules, or contaminants that may be present during the production of the sRGN polypeptide.

### Kits

The present disclosure provides kits for carrying out a method. A kit can include one or more of: an sRGN polypeptide; a nucleic acid comprising a nucleotide encoding a site-directed modifying polypeptide; a guide RNA; a nucleic acid comprising a nucleotide sequence encoding a guide RNA. A kit may comprise a complex that comprises two or more of: an sRGN polypeptide; a nucleic acid comprising a nucleotide encoding an sRGN polypeptide; a guide RNA; a nucleic acid comprising a nucleotide sequence encoding a guide RNA. In some embodiments, a kit comprises an sRGN polypeptide, or a nucleic acid encoding the same. In some embodiments, the sRGN polypeptide comprises: (a) an RNA-binding portion that interacts with the guide RNA; and (b) an activity portion that modulates transcription within the target DNA, wherein the guide RNA determines the site of modulated transcription within the target DNA. In some cases, the activity portion of the sRGN polypeptide exhibits reduced or inactivated nuclease activity. In some cases, the sRGN polypeptide is a chimeric sRGN polypeptide.

In some embodiments, a kit comprises: an sRGN polypeptide, or a nucleic acid encoding the same, and a reagent for reconstituting and/or diluting the sRGN polypeptide. In other embodiments, a kit comprises a nucleic acid (e.g., DNA, RNA) comprising a nucleotide encoding an sRGN polypeptide. In some embodiments, a kit comprises: a nucleic acid (e.g., DNA, RNA) comprising a nucleotide encoding an sRGN polypeptide; and a reagent for reconstituting and/or diluting the sRGN polypeptide.

A kit comprising an sRGN polypeptide, or a nucleic acid encoding the same, can further include one or more additional reagents, where such additional reagents can be selected from: a buffer for introducing the sRGN polypeptide into a cell; a wash buffer; a control reagent; a control expression vector or RNA polynucleotide; a reagent for in vitro production of the sRGN polypeptide from DNA, and the like. In some cases, the site-directed modifying polypeptide included in a kit is a chimeric sRGN polypeptide, as described above.

In some embodiments, a kit comprises a guide RNA, or a DNA nucleic acid encoding the same, the guide RNA comprising: (a) a first segment comprising a nucleotide sequence that is complementary to a sequence in a target DNA; and (b) a second segment that interacts with a site-directed modifying polypeptide. In some embodiments, a kit comprises: (i) a guide RNA, or a DNA nucleic acid encoding the same, the guide RNA comprising: (a) a first segment comprising a nucleotide sequence that is complementary to a sequence in a target DNA; and (b) a second segment that interacts with an sRGN polypeptide; and (ii) an sRGN polypeptide, or a nucleic acid encoding the same, the sRGN polypeptide comprising: (a) an RNA-binding portion that interacts with the guide RNA; and (b) an activity portion that exhibits site-directed enzymatic activity, wherein the site of enzymatic activity is determined by the guide RNA. In some embodiments, the activity portion of the sRGN polypeptide does not exhibit enzymatic activity (comprises an inactivated nuclease, e.g., via mutation). In some cases, the kit comprises a guide RNA and an sRGN polypeptide. In other cases, the kit comprises: (i) a nucleic acid comprising a nucleotide sequence encoding a guide RNA; and (ii) a nucleic acid comprising a nucleotide sequence encoding sRGN polypeptide. As another example, a kit can include: (i) a guide RNA, or a DNA nucleic acid encoding the same, comprising:

(a) a first segment comprising a nucleotide sequence that is complementary to a sequence in a target DNA; and (b) a second segment that interacts with an sRGN polypeptide; and (ii) the sRGN polypeptide, or a nucleic acid encoding the same, comprising: (a) an RNA-binding portion that interacts with the guide RNA; and (b) an activity portion that that modulates transcription within the target DNA, wherein the site of modulated transcription within the target DNA is determined by the guide RNA In some cases, the kit comprises: (i) a guide RNA; and an sRGN polypeptide. In other cases, the kit comprises: (i) a nucleic acid comprising a nucleotide sequence encoding a guide RNA; and (ii) a nucleic acid comprising a nucleotide sequence encoding sRGN polypeptide. The present disclosure provides a kit comprising: (1) a recombinant expression vector comprising (i) a nucleotide sequence encoding a guide RNA, wherein the guide RNA comprises: (a) a first segment comprising a nucleotide sequence that is complementary to a sequence in a target DNA; and (b) a second segment that interacts with an sRGN polypeptide; and (ii) a nucleotide sequence encoding the sRGN polypeptide, wherein the sRGN polypeptide comprises: (a) an RNA-binding portion that interacts with the guide RNA; and (b) an activity portion that exhibits site-directed enzymatic activity, wherein the site of enzymatic activity is determined by the guide RNA; and (2) a reagent for reconstitution and/or dilution of the expression vector.

The present disclosure provides a kit comprising: (1) a recombinant expression vector comprising: (i) a nucleotide sequence encoding a guide RNA, wherein the guide RNA comprises: (a) a first segment comprising a nucleotide sequence that is complementary to a sequence in a target DNA; and (b) a second segment that interacts with an sRGN polypeptide; and (ii) a nucleotide sequence encoding the sRGN polypeptide, wherein the sRGN polypeptide comprises: (a) an RNA-binding portion that interacts with the guide RNA; and (b) an activity portion that modulates transcription within the target DNA, wherein the site of modulated transcription within the target DNA is determined by the guide RNA; and (2) a reagent for reconstitution and/or dilution of the recombinant expression vector.

The present disclosure provides a kit comprising: (1) a recombinant expression vector comprising a nucleic acid comprising a nucleotide sequence that encodes a DNA targeting RNA comprising: (i) a first segment comprising a nucleotide sequence that is complementary to a sequence in a target DNA; and (ii) a second segment that interacts with an sRGN polypeptide; and (2) a reagent for reconstitution and/or dilution of the recombinant expression vector. In some embodiments of this kit, the kit comprises: a recombinant expression vector comprising a nucleotide sequence that encodes an sRGN polypeptide, wherein the sRGN polypeptide comprises: (a) an RNA-binding portion that interacts with the guide RNA; and (b) an activity portion that exhibits site-directed enzymatic activity, wherein the site of enzymatic activity is determined by the guide RNA. In other embodiments of this kit, the kit comprises: a recombinant expression vector comprising a nucleotide sequence that encodes an sRGN polypeptide, wherein the sRGN polypeptide comprises: (a) an RNA-binding portion that interacts with the guide RNA; and (b) an activity portion that modulates transcription within the target DNA, wherein the site of modulated transcription within the target DNA is determined by the guide RNA.

In some embodiments of any of the above kits, the kit comprises a single-molecule guide RNA. In some embodiments of any of the above kits, the kit comprises two or more single-molecule guide RNAs. In some embodiments of any of the above kits, a guide RNA (e.g., including two or more guide RNAs) can be provided as an array (e.g., an array of RNA molecules, an array of DNA molecules encoding the guide RNA(s), etc.). Such kits can be useful, for example, for use in conjunction with the above described genetically modified host cells that comprise an sRGN polypeptide. In some embodiments of any of the above kits, the kit further comprises a donor polynucleotide to effect the desired genetic modification. Components of a kit can be in separate containers; or can be combined in a single container.

In some cases, a kit further comprises one or more variant sRGN site-directed polypeptides that exhibit reduced endodeoxyribonuclease activity relative to wild-type sRGN.

In some cases, a kit further comprises one or more nucleic acids comprising a nucleotide sequence encoding a variant sRGN site-directed polypeptide that exhibits reduced endodeoxyribonuclease activity relative to wild-type sRGN.

Any of the above-described kits can further include one or more additional reagents, where such additional reagents can be selected from: a dilution buffer; a reconstitution solution; a wash buffer; a control reagent; a control expression vector or RNA polynucleotide; a reagent for in vitro production of the sRGN polypeptide from DNA, and the like.

In addition to above-mentioned components, a kit can further include instructions for using the components of the kit to practice the methods. The instructions for practicing the methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g., CD-ROM, diskette, flash drive, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

### Non-Human Genetically Modified Organisms

In some embodiments, a genetically modified host cell has been genetically modified with an exogenous nucleic acid comprising a nucleotide sequence encoding an sRGN polypeptide (e.g., a naturally occurring sRGN; a modified, i.e., mutated or variant, sRGN; a chimeric sRGN; etc.). If such a cell is a eukaryotic single-cell organism, then the modified cell can be considered a genetically modified organism. In some embodiments, the non-human genetically modified organism is an sRGN transgenic multicellular organism.

In some embodiments, a genetically modified non-human host cell (e.g., a cell that has been genetically modified with an exogenous nucleic acid comprising a nucleotide sequence encoding a site-directed modifying polypeptide, e.g., a naturally occurring sRGN; a modified, i.e., mutated or variant, sRGN; a chimeric sRGN; etc.) can generate a genetically modified nonhuman organism (e.g., a mouse, a fish, a frog, a fly, a worm, etc.). For example, if the genetically modified host cell is a pluripotent stem cell (i.e., PSC) or a germ cell (e.g., sperm, oocyte, etc.), an entire genetically modified organism can be derived from the genetically modified host cell. In some embodiments, the genetically modified host cell is a pluripotent stem cell (e.g., ESC, iPSC, pluripotent plant stem cell, etc.) or a germ cell (e.g., sperm cell, oocyte, etc.), either in vivo or in vitro that can give rise to a genetically modified organism. In some embodiments the genetically modified host cell is a vertebrate PSC (e.g., ESC, iPSC, etc.) and is used to generate a genetically modified organism (e.g., by injecting a PSC into a blastocyst to produce a chimeric/mosaic animal, which could then be mated to generate non-chimeric/non-mosaic genetically modified organisms; grafting in the case of plants; etc.). Any convenient method/protocol for producing a genetically modified organism, including the methods described herein, is suitable for producing a genetically modified host cell comprising an exogenous nucleic acid comprising a nucleotide sequence encoding an sRGN polypeptide (e.g., a naturally occurring sRGN; a modified, i.e., mutated or variant, sRGN; a chimeric sRGN; etc.). Methods of producing genetically modified organisms are known in the art. For example, see Cho et al., Curr Protoc Cell Biol. 2009 March; Chapter 19: Unit 19.11: Generation of transgenic mice; Gama et al., Brain Struct Funct. 2010 March; 214 (2-3): 91-109. Epub 2009 Nov. 25: Animal transgenesis: an overview; Husaini et al., GM Crops. 2011 June-December; 2 (3): 150-62. Epub 2011 Jun. 1: Approaches for gene targeting and targeted gene expression in plants.

In some embodiments, a genetically modified organism comprises a target cell for methods of the invention, and thus can be considered a source for target cells. For example, if a genetically modified cell comprising an exogenous nucleic acid comprising a nucleotide sequence encoding a site-directed modifying polypeptide (e.g., a naturally occurring sRGN; a modified, i.e., mutated or variant, sRGN; a chimeric sRGN; etc.) is used to generate a genetically modified organism, then the cells of the genetically modified organism comprise the exogenous nucleic acid comprising a nucleotide sequence encoding an sRGN polypeptide (e.g., a naturally occurring sRGN; a modified, i.e., mutated or variant, sRGN; a chimeric sRGN; etc.). In some such embodiments, the DNA of a cell or cells of the genetically modified organism can be targeted for modification by introducing into the cell or cells a guide RNA (or a DNA encoding a guide RNA) and optionally a donor nucleic acid. For example, the introduction of a guide RNA (or a DNA encoding a guide RNA) into a subset of cells (e.g., brain cells, intestinal cells, kidney cells, lung cells, blood cells, etc.) of the genetically modified organism can target the DNA of such cells for modification, the genomic location of which will depend on the DNA-targeting sequence of the introduced guide RNA.

In some embodiments, a genetically modified organism is a source of target cells for methods of the invention. For example, a genetically modified organism comprising cells that are genetically modified with an exogenous nucleic acid comprising a nucleotide sequence encoding an sRGN polypeptide (e.g., a naturally occurring sRGN; a modified, i.e., mutated or variant, sRGN; a chimeric sRGN; etc.) can provide a source of genetically modified cells, for example PSCs (e.g., ESCs, iPSCs, sperm, oocytes, etc.), neurons, progenitor cells, cardiomyocytes, etc.

In some embodiments, a genetically modified cell is a PSC comprising an exogenous nucleic acid comprising a nucleotide sequence encoding an sRGN polypeptide (e.g., a naturally occurring sRGN; a modified, i.e., mutated or variant, sRGN; a chimeric sRGN; etc.). As such, the PSC can be a target cell such that the DNA of the PSC can be targeted for modification by introducing into the PSC a guide RNA (or a DNA encoding a guide RNA) and optionally a donor nucleic acid, and the genomic location of the modification will depend on the DNA-targeting sequence of the introduced guide RNA. Thus, in some embodiments, the methods described herein can be used to modify the DNA (e.g., delete and/or replace any desired genomic location) of PSCs derived from a genetically modified organism. Such modified PSCs can then be used to generate organisms having both (i) an exogenous nucleic acid comprising a nucleotide sequence encoding an sRGN polypeptide (e.g., a naturally occurring sRGN; a modified, i.e., mutated or variant, sRGN; a chimeric sRGN; etc.) and (ii) a DNA modification that was introduced into the PSC.

An exogenous nucleic acid comprising a nucleotide sequence encoding an sRGN polypeptide (e.g., a naturally occurring sRGN; a modified, i.e., mutated or variant, sRGN; a chimeric sRGN; etc.) can be under the control of (i.e., operably linked to) an unknown promoter (e.g., when the nucleic acid randomly integrates into a host cell genome) or can be under the control of (i.e., operably linked to) a known promoter. Suitable known promoters can be any known promoter and include constitutively active promoters (e.g., CMV promoter), inducible promoters (e.g., heat shock promoter, Tetracycline-regulated promoter, Steroid-regulated promoter, Metal-regulated promoter, estrogen receptor-regulated promoter, etc.), spatially restricted and/or temporally restricted promoters (e.g., a tissue specific promoter, a cell type specific promoter, etc.), etc.

A genetically modified organism (e.g., an organism whose cells comprise a nucleotide sequence encoding an sRGN polypeptide, e.g., a naturally occurring sRGN; a modified, i.e., mutated or variant, sRGN; a chimeric sRGN; etc.) can be any organism including for example, a plant; algae; an invertebrate (e.g., a cnidarian, an echinoderm, a worm, a fly, etc.); a vertebrate (e.g., a fish (e.g., zebrafish, puffer fish, goldfish, etc.), an amphibian (e.g., salamander, frog, etc.), a reptile, a bird, a mammal, etc.); an ungulate (e.g., a goat, a pig, a sheep, a cow, etc.); a rodent (e.g., a mouse, a rat, a hamster, a guinea pig); a lagomorph (e.g., a rabbit); etc.

In some cases, the sRGN polypeptide comprises an amino acid sequence having at least 75%, at least 80%, at least 85%, at least 90%, at least 95%, at least 99%, or 100% amino acid sequence identity to SEQ ID NO: 1-12.

### Transgenic Non-Human Animals

As described above, in some embodiments, a nucleic acid (e.g., a nucleotide sequence encoding an sRGN polypeptide, e.g., a naturally occurring sRGN; a modified, i.e., mutated or variant, sRGN; a chimeric sRGN; etc.) or a recombinant expression vector is used as a transgene to generate a transgenic animal that produces an sRGN polypeptide. Thus, the present disclosure further provides a transgenic non-human animal, which animal comprises a transgene comprising a nucleic acid comprising a nucleotide sequence encoding a site-directed modifying polypeptide, e.g., a naturally occurring sRGN; a modified, i.e., mutated or variant, sRGN; a chimeric sRGN; etc., as described above. In some embodiments, the genome of the transgenic non-human animal comprises a nucleotide sequence encoding an sRGN polypeptide. In some embodiments, the transgenic non-human animal is homozygous for the genetic modification. In some embodiments, the transgenic non-human animal is heterozygous for the genetic modification. In some embodiments, the transgenic non-human animal is a vertebrate, for example, a fish (e.g., zebrafish, goldfish, puffer fish, cave fish, etc.), an amphibian (frog, salamander, etc.), a bird (e.g., chicken, turkey, etc.), a reptile (e.g., snake, lizard, etc.), a mammal (e.g., an ungulate, e.g., a pig, a cow, a goat, a sheep, etc.; a lagomorph (e.g., a rabbit); a rodent (e.g., a rat, a mouse); a nonhuman primate; etc.), etc.

An exogenous nucleic acid comprising a nucleotide sequence encoding an sRGN polypeptide (e.g., a naturally occurring sRGN; a modified, i.e., mutated or variant, sRGN; a chimeric sRGN; etc.) can be under the control of (i.e., operably linked to) an unknown promoter (e.g., when the nucleic acid randomly integrates into a host cell genome) or can be under the control of (i.e., operably linked to) a known promoter. Suitable known promoters can be any known promoter and include constitutively active promoters (e.g., CMV promoter), inducible promoters (e.g., heat shock promoter, tetracycline-regulated promoter, steroid-regulated promoter, metal-regulated promoter, estrogen receptor-regulated promoter, etc.), spatially restricted and/or temporally restricted promoters (e.g., a tissue specific promoter, a cell type specific promoter, etc.), etc.

### Transgenic Plants

As described above, in some embodiments, a nucleic acid (e.g., a nucleotide sequence encoding an sRGN polypeptide, e.g., a naturally occurring sRGN; a modified, i.e., mutated or variant, sRGN; a chimeric sRGN; etc.) or a recombinant expression vector is used as a transgene to generate a transgenic plant that produces an sRGN polypeptide. Thus, the present disclosure further provides a transgenic plant, which plant comprises a transgene comprising a nucleic acid comprising a nucleotide sequence encoding sRGN polypeptide, e.g., a naturally occurring sRGN; a modified, i.e., mutated or variant, sRGN; a chimeric sRGN; etc., as described above. In some embodiments, the genome of the transgenic plant comprises a nucleic acid. In some embodiments, the transgenic plant is homozygous for the genetic modification. In some embodiments, the transgenic plant is heterozygous for the genetic modification.

Methods of introducing exogenous nucleic acids into plant cells are well known in the art. Such plant cells are considered “transformed,” as defined above. Suitable methods include viral infection (such as double-stranded DNA viruses), transfection, conjugation, protoplast fusion, electroporation, particle gun technology, calcium phosphate precipitation, direct microinjection, silicon carbide whiskers technology, Agrobacterium-mediated transformation and the like. The choice of method is generally dependent on the type of cell being transformed and the circumstances under which the transformation is taking place (i.e., in vitro, ex vivo, or in vivo). Transformation methods based upon the soil bacterium Agrobacterium tumefaciens are particularly useful for introducing an exogenous nucleic acid molecule into a vascular plant. The wild type form of Agrobacterium contains a Ti (tumor-inducing) plasmid that directs production of tumorigenic crown gall growth on host plants. Transfer of the tumor-inducing T-DNA region of the Ti plasmid to a plant genome requires the Ti plasmid-encoded virulence genes as well as T-DNA borders, which are a set of direct DNA repeats that delineate the region to be transferred. An Agrobacterium-based vector is a modified form of a Ti plasmid, in which the tumor inducing functions are replaced by the nucleic acid sequence of interest to be introduced into the plant host.

Agrobacterium-mediated transformation generally employs cointegrate vectors or binary vector systems, in which the components of the Ti plasmid are divided between a helper vector, which resides permanently in the Agrobacterium host and carries the virulence genes, and a shuttle vector, which contains the gene of interest bounded by T-DNA sequences. A variety of binary vectors are well known in the art and are commercially available, for example, from Clontech (Palo Alto, Calif.). Methods of coculturing Agrobacterium with cultured plant cells or wounded tissue such as leaf tissue, root explants, hypocotyledons, stem pieces or tubers, for example, also are well known in the art. See., e.g., Glick and Thompson, (eds.), Methods in Plant Molecular Biology and Biotechnology, Boca Raton, Fla.: CRC Press (1993).

Microprojectile-mediated transformation also can be used to produce a transgenic plant. This method, first described by Klein et al. (Nature 327:70-73 (1987)), relies on microprojectiles such as gold or tungsten that are coated with the desired nucleic acid molecule by precipitation with calcium chloride, spermidine or polyethylene glycol. The microprojectile particles are accelerated at high speed into an angiosperm tissue using a device such as the BIOLISTIC PD-1000 (Biorad; Hercules Calif.).

A nucleic acid may be introduced into a plant in a manner such that the nucleic acid is able to enter a plant cell(s), e.g., via an in vivo or ex vivo protocol. By “in vivo,” it is meant in the nucleic acid is administered to a living body of a plant e.g., infiltration. By “ex vivo” it is meant that cells or explants are modified outside of the plant, and then such cells or organs are regenerated to a plant. A number of vectors suitable for stable transformation of plant cells or for the establishment of transgenic plants have been described, including those described in Weissbach and Weissbach, (1989) Methods for Plant Molecular Biology Academic Press, and Gelvin et al., (1990) Plant Molecular Biology Manual, Kluwer Academic Publishers. Specific examples include those derived from a Ti plasmid of Agrobacterium tumefaciens, as well as those disclosed by Herrera-Estrella et al. (1983) Nature 303:209, Bevan (1984) Nucl Acid Res. 12:8711-8721, Klee (1985) Bio/Technolo 3:637-642. Alternatively, non-Ti vectors can be used to transfer the DNA into plants and cells by using free DNA delivery techniques. By using these methods transgenic plants such as wheat, rice (Christou (1991) Bio/Technology 9:957-9 and 4462) and corn (Gordon-Kamm (1990) Plant Cell 2:603-618) can be produced. An immature embryo can also be a good target tissue for monocots for direct DNA delivery techniques by using the particle gun (Weeks et al. (1993) Plant Physiol 102:1077-1084; Vasil (1993) Bio/Technolo 10:667-674; Wan and Lemeaux (1994) Plant Physiol104: 37-48 and for

Agrobacterium-mediated DNA transfer (Ishida et al. (1996) Nature Biotech 14:745-750). Exemplary methods for introduction of DNA into chloroplasts are biolistic bombardment, polyethylene glycol transformation of protoplasts, and microinjection (Daniell et al. Nat. Biotechnol 16:345-348, 1998; Staub et al. Nat. Biotechnol 18:333-338, 2000; O'Neill et al. Plant J. 3:729-738, 1993; Knoblauch et al. Nat. Biotechnol 17: 906-909; U.S. Pat. Nos. 5,451,513, 5,545,817, 5,545,818, and 5,576,198; in Inti. Application No. WO 95/16783; and in Boynton et al., Methods in Enzymology 217:510-536 (1993), Svab et al., Proc. Natl. Acad. Sci. USA 90:913-917 (1993), and McBride et al., Proc. Natl. Acad. Sci. USA 91:7301-7305 (1994)). Any vector suitable for the methods of biolistic bombardment, polyethylene glycol transformation of protoplasts and microinjection will be suitable as a targeting vector for chloroplast transformation. Any double-stranded DNA vector may be used as a transformation vector, especially when the method of introduction does not utilize Agrobacterium.

Plants, which can be genetically modified, include grains, forage crops, fruits, vegetables, oil seed crops, palms, forestry, and vines. Specific examples of plants which can be modified follow: maize, banana, peanut, field peas, sunflower, tomato, canola, tobacco, wheat, barley, oats, potato, soybeans, cotton, carnations, sorghum, lupin and rice.

Also provided by the disclosure are transformed plant cells, tissues, plants and products that contain the transformed plant cells. A feature of the transformed cells, and tissues and products that include the same is the presence of a nucleic acid integrated into the genome, and production by plant cells of an sRGN polypeptide, e.g., a naturally occurring sRGN; a modified, i.e., mutated or variant, sRGN; a chimeric sRGN; etc. Recombinant plant cells of the present invention are useful as populations of recombinant cells, or as a tissue, seed, whole plant, stem, fruit, leaf, root, flower, stem, tuber, grain, animal feed, a field of plants, and the like.

A nucleic acid comprising a nucleotide sequence encoding an sRGN polypeptide (e.g., a naturally occurring sRGN; a modified, i.e., mutated or variant, sRGN; a chimeric sRGN; etc.) can be under the control of (i.e., operably linked to) an unknown promoter (e.g., when the nucleic acid randomly integrates into a host cell genome) or can be under the control of (i.e., operably linked to) a known promoter. Suitable known promoters can be any known promoter and include constitutively active promoters, inducible promoters, spatially restricted and/or temporally restricted promoters, etc.

The present disclosure provides methods of modulating transcription of a target nucleic acid in a host cell. The methods generally involve contacting the target nucleic acid with an enzymatically inactive sRGN polypeptide and a guide RNA. The methods are useful in a variety of applications, which are also provided.

A transcriptional modulation method of the present disclosure overcomes some of the drawbacks of methods involving RNAi. A transcriptional modulation method of the present disclosure finds use in a wide variety of applications, including research applications, drug discovery (e.g., high throughput screening), target validation, industrial applications (e.g., crop engineering; microbial engineering, etc.), diagnostic applications, therapeutic applications, and imaging techniques.

### Methods of Modulating Transcription

The present disclosure provides a method of selectively modulating transcription of a target DNA in a host cell. The method generally involves: a) introducing into the host cell: i) a guide RNA, or a nucleic acid comprising a nucleotide sequence encoding the guide RNA; and ii) a variant sRGN site-directed polypeptide (“variant sRGN polypeptide”), or a nucleic acid comprising a nucleotide sequence encoding the variant sRGN polypeptide, where the variant sRGN polypeptide exhibits reduced endodeoxyribonuclease activity.

The guide RNA (also referred to herein as “guide RNA”; or “gRNA”) comprises: i) a first segment comprising a nucleotide sequence that is complementary to a target sequence in a target DNA; ii) a second segment that interacts with an sRGN polypeptide; and iii) a transcriptional terminator. The first segment, comprising a nucleotide sequence that is complementary to a target sequence in a target DNA, is referred to herein as a “targeting segment”. The second segment, which interacts with an sRGN polypeptide, is also referred to herein as a “protein-binding sequence” or “dsRGN-binding hairpin,” or “dsRGN handle.” By “segment” it is meant a segment/section/region of a molecule, e.g., a contiguous stretch of nucleotides in an RNA. The definition of “segment,” unless otherwise specifically defined in a particular context, is not limited to a specific number of total base pairs, and may include regions of RNA molecules that are of any total length and may or may not include regions with complementarity to other molecules. The variant sRGN site-directed polypeptide comprises: i) an RNA-binding portion that interacts with the guide RNA; and an activity portion that exhibits reduced endodeoxyribonuclease activity.

The guide RNA and the variant sRGN polypeptide form a complex in the host cell; the complex selectively modulates transcription of a target DNA in the host cell.

In some cases, a transcription modulation method of the present disclosure provides for selective modulation (e.g., reduction or increase) of a target nucleic acid in a host cell. For example, “selective” reduction of transcription of a target nucleic acid reduces transcription of the target nucleic acid by at least 10%, at least 20%, at least 30%, at least 40%, at least 50%, at least 60%, at least 70%, at least 80%, at least 90%, or greater than 90%, compared to the level of transcription of the target nucleic acid in the absence of a guide RNA/variant sRGN polypeptide complex. Selective reduction of transcription of a target nucleic acid reduces transcription of the target nucleic acid, but does not substantially reduce transcription of a non-target nucleic acid, e.g., transcription of a non-target nucleic acid is reduced, if at all, by less than 10% compared to the level of transcription of the non-target nucleic acid in the absence of the guide RNA/variant sRGN polypeptide complex.

### Increased Transcription

“Selective” increased transcription of a target DNA can increase transcription of the target DNA by at least 1.1 fold (e.g., at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.5 fold, at least 3 fold, at least 3.5 fold, at least 4 fold, at least 4.5 fold, at least 5 fold, at least 6 fold, at least 7 fold, at least 8 fold, at least 9 fold, at least 10 fold, at least 12 fold, at least 15 fold, or at least 20-fold) compared to the level of transcription of the target DNA in the absence of a guide RNA/variant sRGN polypeptide complex. Selective increase of transcription of a target DNA increases transcription of the target DNA, but does not substantially increase transcription of a non-target DNA, e.g., transcription of a non-target DNA is increased, if at all, by less than about 5-fold (e.g., less than about 4-fold, less than about 3-fold, less than about 2-fold, less than about 1.8-fold, less than about 1.6-fold, less than about 1.4-fold, less than about 1.2-fold, or less than about 1.1-fold) compared to the level of transcription of the non-targeted DNA in the absence of the guide RNA/variant sRGN polypeptide complex.

As a non-limiting example, increased transcription can be achieved by fusing dsRGN to a heterologous sequence. Suitable fusion partners include, but are not limited to, a polypeptide that provides an activity that indirectly increases transcription by acting directly on the target DNA or on a polypeptide (e.g., a histone or other DNA-binding protein) associated with the target DNA. Suitable fusion partners include, but are not limited to, a polypeptide that provides for methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, crotonylation, decrotonylation, propionylation, depropionylation, myristoylation activity, or demyristoylation activity.

Additional suitable fusion partners include, but are not limited to, a polypeptide that directly provides for increased transcription of the target nucleic acid (e.g., a transcription activator or a fragment thereof, a protein or fragment thereof that recruits a transcription activator, a small molecule/drug-responsive transcription regulator, etc.).

A non-limiting example of a method using a dsRGN fusion protein to increase transcription in a prokaryote includes a modification of the bacterial one-hybrid (B1H) or two-hybrid (B2H) system. In the B1H system, a DNA binding domain (BD) is fused to a bacterial transcription activation domain (AD, e.g., the alpha subunit of the Escherichia coli RNA polymerase (RNAPa)). Thus, a dsRGN can be fused to a heterologous sequence comprising an AD. When the dsRGN fusion protein arrives at the upstream region of a promoter (targeted there by the guide RNA) the AD (e.g., RNAPa) of the dsRGN fusion protein recruits the RNAP holoenzyme, leading to transcription activation. In the B2H system, the BD is not directly fused to the AD; instead, their interaction is mediated by a protein-protein interaction (e.g., GAL11P-GAL4 interaction). To modify such a system for use in the methods, dsRGN can be fused to a first protein sequence that provides for protein-protein interaction (e.g., the yeast GAL11P and/or GAL4 protein) and RNAa can be fused to a second protein sequence that completes the protein-protein interaction (e.g., GAL4 if GAL11P is fused to dsRGN, GAL11P if GAL4 is fused to dsRGN, etc.). The binding affinity between GAL11P and GAL4 increases the efficiency of binding and transcription firing rate.

A non-limiting example of a method using a dsRGN fusion protein to increase transcription in eukaryotes includes fusion of dsRGN to an activation domain (AD) (e.g., GAL4, herpesvirus activation protein VP16 or VP64, human nuclear factor NF-KB p65 subunit, etc.). To render the system inducible, expression of the dsRGN fusion protein can be controlled by an inducible promoter (e.g., Tet-ON, Tet-OFF, etc.). The guide RNA can be design to target known transcription response elements (e.g., promoters, enhancers, etc.), known upstream activating sequences (UAS), sequences of unknown or known function that are suspected of being able to control expression of the target DNA, etc.

### Additional Fusion Partners

Non-limiting examples of fusion partners to accomplish increased or decreased transcription include, but are not limited to, transcription activator and transcription repressor domains (e.g., the Krüppel associated box (KRAB or SKD); the Mad mSIN3 interaction domain (SID); the ERF repressor domain (ERD), etc.). In some such cases, the dsRGN fusion protein is targeted by the guide RNA to a specific location (i.e., sequence) in the target DNA and exerts locus-specific regulation such as blocking RNA polymerase binding to a promoter (which selectively inhibits transcription activator function), and/or modifying the local chromatin status (e.g., when a fusion sequence is used that modifies the target DNA or modifies a polypeptide associated with the target DNA). In some cases, the changes are transient (e.g., transcription repression or activation). In some cases, the changes are inheritable (e.g., when epigenetic modifications are made to the target DNA or to proteins associated with the target DNA, e.g., nucleosomal histones). In some embodiments, the heterologous sequence can be fused to the C-terminus of the dsRGN polypeptide. In some embodiments, the heterologous sequence can be fused to the N-terminus of the dsRGN polypeptide. In some embodiments, the heterologous sequence can be fused to an internal portion (i.e., a portion other than the N- or C-terminus) of the dsRGN polypeptide. The biological effects of a method using a dsRGN fusion protein can be detected by any convenient method (e.g., gene expression assays; chromatin-based assays, e.g., Chromatin ImmunoPrecipitation (ChIP), Chromatin in vivo Assay (CiA), etc.).

In some cases, a method involves use of two or more different guide RNAs. For example, two different guide RNAs can be used in a single host cell, where the two different guide RNAs target two different target sequences in the same target nucleic acid. Thus, for example, a transcriptional modulation method can further comprise introducing into the host cell a second guide RNA, or a nucleic acid comprising a nucleotide sequence encoding the second guide RNA, where the second guide RNA comprises: i) a first segment comprising a nucleotide sequence that is complementary to a second target sequence in the target DNA; ii) a second segment that interacts with the site-directed polypeptide; and iii) a transcriptional terminator. In some cases, use of two different guide RNAs targeting two different targeting sequences in the same target nucleic acid provides for increased modulation (e.g., reduction or increase) in transcription of the target nucleic acid.

As another example, two different guide RNAs can be used in a single host cell, where the two different guide RNAs target two different target nucleic acids. Thus, for example, a transcriptional modulation method can further comprise introducing into the host cell a second guide RNA, or a nucleic acid comprising a nucleotide sequence encoding the second guide RNA, where the second guide RNA comprises: i) a first segment comprising a nucleotide sequence that is complementary to a target sequence in at least a second target DNA; ii) a second segment that interacts with the site-directed polypeptide; and iii) a transcriptional terminator.

In some embodiments, a nucleic acid (e.g., a guide RNA, e.g., a single-molecule guide RNA; a donor polynucleotide; a nucleic acid encoding an sRGN polypeptide; etc.) comprises a modification or sequence that provides for an additional desirable feature (e.g., modified or regulated stability; subcellular targeting; tracking, e.g., a fluorescent label; a binding site for a protein or protein complex; etc.). Non-limiting examples include: a 5′ cap (e.g., a 7-methylguanylate cap (m 7G)); a 3′ polyadenylated tail (i.e., a 3′ poly(A) tail); a riboswitch sequence or an aptamer sequence (e.g., to allow for regulated stability and/or regulated accessibility by proteins and/or protein complexes); a terminator sequence; a sequence that forms a dsRNA duplex (i.e., a hairpin)); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, etc.); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like); a modification of RNA that alters the structure of such RNA, consequently the sRGN ribonucleoprotein; and combinations thereof.

### DNA-Targeting Segment

The DNA-targeting segment (or “DNA-targeting sequence”) of a guide RNA comprises a nucleotide sequence that is complementary to a specific sequence within a target DNA (the complementary strand of the target DNA).

In other words, the DNA-targeting segment of a guide RNA interacts with a target DNA in a sequence-specific manner via hybridization (i.e., base pairing). As such, the nucleotide sequence of the DNA-targeting segment may vary and determines the location within the target DNA that the guide RNA and the target DNA will interact. The DNA-targeting segment of a guide RNA can be modified (e.g., by genetic engineering) to hybridize to any desired sequence within a target DNA

### Stability Control Sequence (e.g., Transcriptional Terminator Segment)

A stability control sequence influences the stability of an RNA (e.g., a guide RNA). One example of a suitable stability control sequence is a transcriptional terminator segment (i.e., a transcription termination sequence). A transcriptional terminator segment of a guide RNA can have a total length of from about 10 nucleotides to about 100 nucleotides, e.g., from about 10 nucleotides (nt) to about 20 nt, from about 20 nt to about 30 nt, from about 30 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. For example, the transcriptional terminator segment can have a length of from about 15 nucleotides (nt) to about 80 nt, from about 15 nt to about 50 nt, from about 15 nt to about 40 nt, from about 15 nt to about 30 nt or from about 15 nt to about 25 nt.

In some cases, the transcription termination sequence is one that is functional in a eukaryotic cell. In some cases, the transcription termination sequence is one that is functional in a prokaryotic cell.

Nucleotide sequences that can be included in a stability control sequence (e.g., transcriptional termination segment, or in any segment of the guide RNA to provide for increased stability) include, for example, a Rho-independent trp termination site.

### Additional Sequences

In some embodiments, a guide RNA comprises at least one additional segment at either the 5′ or 3′ end. For example, a suitable additional segment can comprise a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and protein complexes); a sequence that forms a dsRNA duplex (i.e., a hairpin)); a sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, etc.); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like) a modification or sequence that provides for increased, decreased, and/or controllable stability; and combinations thereof.

### Multiple Simultaneous Guide RNAs

In some embodiments, multiple guide RNAs are used simultaneously in the same cell to simultaneously modulate transcription at different locations on the same target DNA or on different target DNAs. In some embodiments, two or more guide RNAs target the same gene or transcript or locus. In some embodiments, two or more guide RNAs target different unrelated loci. In some embodiments, two or more guide RNAs target different, but related loci.

Because the guide RNAs are small and robust they can be simultaneously present on the same expression vector and can even be under the same transcriptional control if so desired. In some embodiments, two or more (e.g., 3 or more, 4 or more, 5 or more, 10 or more, 15 or more, 20 or more, 25 or more, 30 or more, 35 or more, 40 or more, 45 or more, or 50 or more) guide RNAs are simultaneously expressed in a target cell (from the same or different vectors/from the same or different promoters). In some cases, multiple guide RNAs can be encoded in an array mimicking naturally occurring CRISPR arrays of targeter RNAs. The targeting segments are encoded as approximately 30 nucleotide long sequences (can be about 16 to about 100 nt) and are separated by CRISPR repeat sequences. The array may be introduced into a cell by DNAs encoding the RNAs or as RNAs.

To express multiple guide RNAs, an artificial RNA processing system mediated by the Csy4 endoribonuclease can be used. For example, multiple guide RNAs can be concatenated into a tandem array on a precursor transcript (e.g., expressed from a U6 promoter), and separated by Csy4-specific RNA sequence. Co-expressed Csy4 protein cleaves the precursor transcript into multiple guide RNAs. Advantages for using an RNA processing system include: first, there is no need to use multiple promoters; second, since all guide RNAs are processed from a precursor transcript, their concentrations are normalized for similar dsRGN-binding.

Csy4 is a small endoribonuclease (RNase) protein derived from bacteria Pseudomonas aeruginosa. Csy4 specifically recognizes a minimal 17-bp RNA hairpin, and exhibits rapid (<1 min) and highly efficient (>99.9%) RNA cleavage. Unlike most RNases, the cleaved RNA fragment remains stable and functionally active. The Csy4-based RNA cleavage can be repurposed into an artificial RNA processing system. In this system, the 17-bp RNA hairpins are inserted between multiple RNA fragments that are transcribed as a precursor transcript from a single promoter. Co-expression of Csy4 is effective in generating individual RNA fragments.

Variant sRGN Site-Directed Polypeptide

As noted above, a guide RNA and a variant sRGN site-directed polypeptide form a complex. The guide RNA provides target specificity to the complex by comprising a nucleotide sequence that is complementary to a sequence of a target DNA. The variant sRGN site-directed polypeptide has reduced endodeoxyribonuclease activity. For example, a variant sRGN site-directed polypeptide suitable for use in a transcription modulation method of the present disclosure exhibits less than about 20%, less than about 15%, less than about 10%, less than about 5%, less than about 1%, or less than about 0.1%, of the endodeoxyribonuclease activity of a reference sRGN polypeptide, e.g., a reference sRGN polypeptide comprising an amino acid sequence set forth in any one of SEQ ID NOs: 1-12. In some embodiments, the variant sRGN site-directed polypeptide has substantially no detectable endodeoxyribonuclease activity (dsRGN). In some embodiments when a variant sRGN site-directed polypeptide has reduced catalytic activity, the polypeptide can still bind to target DNA in a site-specific manner (because it is still guided to a target DNA sequence by a guide RNA) as long as it retains the ability to interact with the guide RNA. In some cases, a suitable variant sRGN site-directed polypeptide comprises an amino acid sequence having at least 75%, at least 80%, at least 85%, at least 90%, at least 95%, at least 99%, or greater amino acid sequence identity to any one of SEQ ID NOs: 1-12.

In some cases, the variant sRGN site-directed polypeptide is a nickase that can cleave the complementary strand of the target DNA but has reduced ability to cleave the non-complementary strand of the target DNA

In some cases, the variant sRGN site-directed polypeptide in a nickase that can cleave the non-complementary strand of the target DNA but has reduced ability to cleave the complementary strand of the target DNA.

In some cases, the variant sRGN site-directed polypeptide has a reduced ability to cleave both the complementary and the non-complementary strands of the target DNA. For example, alanine substitutions are contemplated.

In some cases, the variant sRGN site-directed polypeptide is a fusion polypeptide (a “variant sRGN fusion polypeptide”), i.e., a fusion polypeptide comprising: i) a variant sRGN site-directed polypeptide; and ii) a covalently linked heterologous polypeptide (also referred to as a “fusion partner”).

The heterologous polypeptide may exhibit an activity (e.g., enzymatic activity) that will also be exhibited by the variant sRGN fusion polypeptide (e.g., methyltransferase activity, acetyltransferase activity, kinase activity, ubiquitinating activity, etc.). A heterologous nucleic acid sequence may be linked to another nucleic acid sequence (e.g., by genetic engineering) to generate a chimeric nucleotide sequence encoding a chimeric polypeptide. In some embodiments, a variant sRGN fusion polypeptide is generated by fusing a variant sRGN polypeptide with a heterologous sequence that provides for subcellular localization (i.e., the heterologous sequence is a subcellular localization sequence, e.g., a nuclear localization signal (NLS) for targeting to the nucleus; a mitochondrial localization signal for targeting to the mitochondria; a chloroplast localization signal for targeting to a chloroplast; an ER retention signal; and the like). In some embodiments, the heterologous sequence can provide a tag (i.e., the heterologous sequence is a detectable label) for ease of tracking and/or purification (e.g., a fluorescent protein, e.g., green fluorescent protein (GFP), YFP, RFP, CFP, mCherry, tdTomato, and the like; a histidine tag, e.g., a 6×His tag; a hemagglutinin (HA) tag; a FLAG tag; a Myc tag; and the like). In some embodiments, the heterologous sequence can provide for increased or decreased stability (i.e., the heterologous sequence is a stability control peptide, e.g., a degron, which in some cases is controllable (e.g., a temperature sensitive or drug controllable degron sequence, see below). In some embodiments, the heterologous sequence can provide for increased or decreased transcription from the target DNA (i.e., the heterologous sequence is a transcription modulation sequence, e.g., a transcription factor/activator or a fragment thereof, a protein or fragment thereof that recruits a transcription factor/activator, a transcription repressor or a fragment thereof, a protein or fragment thereof that recruits a transcription repressor, a small molecule/drug-responsive transcription regulator, etc.). In some embodiments, the heterologous sequence can provide a binding domain (i.e., the heterologous sequence is a protein binding sequence, e.g., to provide the ability of a chimeric dsRGN polypeptide to bind to another protein of interest, e.g., a DNA or histone modifying protein, a transcription factor or transcription repressor, a recruiting protein, etc.).

Suitable fusion partners that provide for increased or decreased stability include, but are not limited to degron sequences. Degrons are understood by one of ordinary skill in the art to be amino acid sequences that control the stability of the protein of which they are part. For example, the stability of a protein comprising a degron sequence is controlled at least in part by the degron sequence. In some cases, a suitable degron is constitutive such that the degron exerts its influence on protein stability independent of experimental control (i.e., the degron is not drug inducible, temperature inducible, etc.) In some cases, the degron provides the variant sRGN polypeptide with controllable stability such that the variant sRGN polypeptide can be turned “on” (i.e., stable) or “off” (i.e., unstable, degraded) depending on the desired conditions. For example, if the degron is a temperature sensitive degron, the variant sRGN polypeptide may be functional (i.e., “on”, stable) below a threshold temperature (e.g., 42° C., 41° C., 40° C., 39° C., 38° C., 37° C., 36° C., 35° C., 34° C., 33° C., 32° C., 31° C., 30° C., etc.) but non-functional (i.e., “off”, degraded) above the threshold temperature. As another example, if the degron is a drug inducible degron, the presence or absence of drug can switch the protein from an “off” (i.e., unstable) state to an “on” (i.e., stable) state or vice versa. An exemplary drug inducible degron is derived from the FKBP12 protein. The stability of the degron is controlled by the presence or absence of a small molecule that binds to the degron.

Examples of suitable degrons include, but are not limited to those degrons controlled by Shield-1, DHFR, auxins, and/or temperature. Non-limiting examples of suitable degrons are known in the art (e.g., Dohmen et al., Science, 1994. 263 (5151): p. 1273-1276: Heat-inducible degron: a method for constructing temperature-sensitive mutants; Schoeber et al., Am J Physiol Renal Physiol. 2009 January; 296 (1): F204-11: Conditional fast expression and function of multimeric TRPV5 channels using Shield-1; Chu et al., Bioorg Med Chem Lett. 2008 Nov. 15; 18 (22): 5941-4: Recent progress with FKBP-derived destabilizing domains; Kanemaki, Pflugers Arch. 2012 Dec. 28: Frontiers of protein expression control with conditional degrons; Yang et al., Mol Cell. 2012 Nov. 30; 48 (4): 487-8: Titivated for destruction: the methyl degron; Barbour et al., Biosci Rep. 2013 Jan. 18; 33 (1): Characterization of the bipartite degron that regulates ubiquitin-independent degradation of thymidylate synthase; and Greussing et al., J Vis Exp. 2012 Nov. 10; (69): Monitoring of ubiquitin-proteasome activity in living cells using a Degron (dgn)-destabilized green fluorescent protein (GFP)-based reporter protein; all of which are hereby incorporated in their entirety by reference).

Exemplary degron sequences have been well characterized and tested in both cells and animals. Thus, fusing sRGN to a degron sequence produces a “tunable” and “inducible” sRGN polypeptide. Any of the fusion partners described herein can be used in any desirable combination. As one non-limiting example to illustrate this point, an sRGN fusion protein can comprise a YFP sequence for detection, a degron sequence for stability, and transcription activator sequence to increase transcription of the target DNA Furthermore, the number of fusion partners that can be used in an sRGN fusion protein is unlimited. In some cases, an sRGN fusion protein comprises one or more (e.g., two or more, three or more, four or more, or five or more) heterologous sequences.

Suitable fusion partners include, but are not limited to, a polypeptide that provides for methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, crotonylation activity, decrotonylation activity, propionylation activity, depropionylationa activity, myristoylation activity, or demyristoylation activity, any of which can be directed at modifying the DNA directly (e.g., methylation of DNA) or at modifying a DNA-associated polypeptide (e.g., a histone or DNA binding protein). Further suitable fusion partners include, but are not limited to boundary elements (e.g., CTCF), proteins and fragments thereof that provide periphery recruitment (e.g., LaminA, Lamin B, etc.), and protein docking elements (e.g., FKBP/FRB, Pil 1/Aby 1, etc.).

In some embodiments, an sRGN polypeptide can be codon-optimized. This type of optimization is known in the art and entails the mutation of foreign-derived DNA to mimic the codon preferences of the intended host organism or cell while encoding the same protein. Thus, the codons are changed, but the encoded protein remains unchanged. For example, if the intended target cell were a human cell, a human codon-optimized dsRGN (or dsRGN variant) would be a suitable site-directed modifying polypeptide. As another non-limiting example, if the intended host cell were a mouse cell, than a mouse codon-optimized sRGN (or variant, e.g., enzymatically inactive variant) would be a suitable sRGN site-directed polypeptide. While codon optimization is not required, it is acceptable and may be preferable in certain cases. Polyadenylation signals can also be chosen to optimize expression in the intended host.

### Host Cells

A method of the present disclosure to modulate transcription may be employed to induce transcriptional modulation in mitotic or post-mitotic cells in vivo and/or ex vivo and/or in vitro.

Because the guide RNA provides specificity by hybridizing to target DNA, a mitotic and/or post-mitotic cell can be any of a variety of host cell, where suitable host cells include, but are not limited to, a bacterial cell; an archaeal cell; a single-celled eukaryotic organism; a plant cell; an algal cell, e.g., Botryococcus braunii, Chlamydomonas reinhardtii, Nannochloropsis gaditana, Chlorella pyrenoidosa, Sargassum patens (C. Agardh), and the like; a fungal cell; an animal cell; a cell from an invertebrate animal (e.g., an insect, a cnidarian, an echinoderm, a nematode, etc.); a eukaryotic parasite (e.g., a malarial parasite, e.g., Plasmodium fakiparum; a helminth; etc.); a cell from a vertebrate animal (e.g., fish, amphibian, reptile, bird, mammal); a mammalian cell, e.g., a rodent cell, a human cell, a non-human primate cell, etc. Suitable host cells include naturally occurring cells; genetically modified cells (e.g., cells genetically modified in a laboratory, e.g., by the “hand of man”); and cells manipulated in vitro in any way. In some cases, a host cell is isolated.

Any type of cell may be of interest (e.g., a stem cell, e.g., an embryonic stem (ES) cell, an induced pluripotent stem (iPS) cell, a germ cell; a somatic cell, e.g., a fibroblast, a hematopoietic cell, a neuron, a muscle cell, a bone cell, a hepatocyte, a pancreatic cell; an in vitro or in vivo embryonic cell of an embryo at any stage, e.g., a 1-cell, 2-cell, 4-cell, 8-cell, etc. stage zebrafish embryo; etc.). Cells may be from established cell lines or they may be primary cells, where “primary cells”, “primary cell lines”, and “primary cultures” are used interchangeably herein to refer to cells and cells cultures that have been derived from a subject and allowed to grow in vitro for a limited number of passages, i.e., splittings, of the culture. For example, primary cultures include cultures that may have been passaged 0 times, 1 time, 2 times, 4 times, 5 times, 10 times, or 15 times, but not enough times go through the crisis stage. Primary cell lines can be are maintained for fewer than 10 passages in vitro. Target cells are in many embodiments unicellular organisms, or are grown in culture.

If the cells are primary cells, such cells may be harvested from an individual by any convenient method. For example, leukocytes may be conveniently harvested by apheresis, leukocytapheresis, density gradient separation, etc., while cells from tissues such as skin, muscle, bone marrow, spleen, liver, pancreas, lung, intestine, stomach, etc. are most conveniently harvested by biopsy. An appropriate solution may be used for dispersion or suspension of the harvested cells. Such solution will generally be a balanced salt solution, e.g., normal saline, phosphate-buffered saline (PBS), Hank's balanced salt solution, etc., conveniently supplemented with fetal calf serum or other naturally occurring factors, in conjunction with an acceptable buffer at low concentration, e.g., from 5-25 mM. Convenient buffers include HEPES, phosphate buffers, lactate buffers, etc. The cells may be used immediately, or they may be stored, frozen, for long periods of time, being thawed and capable of being reused. In such cases, the cells will usually be frozen in 10% dimethyl sulfoxide (DMSO), 50% serum, 40% buffered medium, or some other such solution as is commonly used in the art to preserve cells at such freezing temperatures, and thawed in a manner as commonly known in the art for thawing frozen cultured cells.

Introducing Nucleic Acid into a Host Cell

A guide RNA, or a nucleic acid comprising a nucleotide sequence encoding same, can be introduced into a host cell by any of a variety of well-known methods. Similarly, where a method involves introducing into a host cell a nucleic acid comprising a nucleotide sequence encoding a variant sRGN site-directed polypeptide, such a nucleic acid can be introduced into a host cell by any of a variety of well-known methods.

Methods of introducing a nucleic acid into a host cell are known in the art, and any known method can be used to introduce a nucleic acid (e.g., an expression construct) into a stem cell or progenitor cell. Suitable methods include, e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (e.g., Panyam et., al Adv Drug Deliv Rev. 2012 Sep. 13. pii: 50169-409X(12)00283-9. doi: 10.1016/j.addr.2012.09.023), and the like, including but not limiting to exosome delivery.

### Nucleic Acids

The present disclosure provides an isolated nucleic acid comprising a nucleotide sequence encoding a guide RNA. In some cases, a nucleic acid also comprises a nucleotide sequence encoding a variant sRGN site-directed polypeptide.

In some embodiments, a method involves introducing into a host cell (or a population of host cells) one or more nucleic acids comprising nucleotide sequences encoding a guide RNA and/or a variant sRGN site-directed polypeptide. In some embodiments a cell comprising a target DNA is in vitro. In some embodiments a cell comprising a target DNA is in vivo. Suitable nucleic acids comprising nucleotide sequences encoding a guide RNA and/or a variant sRGN site-directed polypeptide include expression vectors, where an expression vector comprising a nucleotide sequence encoding a guide

### Expression Vectors

In some embodiments, the recombinant expression vector is a viral construct, e.g., a recombinant adeno-associated virus construct (e.g., U.S. Pat. No. 7,078,387), a recombinant adenoviral construct, a recombinant lentiviral construct, a recombinant retroviral construct, etc. Suitable expression vectors include, but are not limited to, viral vectors (e.g., viral vectors based on vaccinia virus; poliovirus; adenovirus (e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:10881097, 1999; WO 94/12649, WO 93/03769; WO 93/19191; WO 94/28938; WO 95/11984 and WO 95/00655); adeno-associated virus (e.g., Ali et al., Hum Gene Ther 9:81 86,1998, Flannery et al., PNAS 94:6916 6921, 1997; Bennett et al., Invest Opthalmol Vis Sci 38:2857 2863, 1997; Jomary et al., Gene Ther 4:683-690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Viral. (1988) 166:154-165; and Flotte et al., PNAS (1993) 90:10613-10617); SV40; herpes simplex virus; human immunodeficiency virus (e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like.

Numerous suitable expression vectors are known to those of skill in the art, and many are commercially available. The following vectors are provided by way of example; for eukaryotic host cells: pXT1, pSG5 (Stratagene), pSVK3, pBPV, pMSG, and pSVLSV40 (Pharmacia). However, any other vector may be used so long as it is compatible with the host cell.

Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

In some embodiments, a nucleotide sequence encoding a guide RNA and/or a variant sRGN site-directed polypeptide is operably linked to a control element, e.g., a transcriptional control element, such as a promoter. The transcriptional control element may be functional in either a eukaryotic cell, e.g., a mammalian cell; or a prokaryotic cell (e.g., bacterial or archaeal cell). In some embodiments, a nucleotide sequence encoding a guide RNA and/or a variant sRGN site-directed polypeptide is operably linked to multiple control elements that allow expression of the nucleotide sequence encoding a guide RNA and/or a variant sRGN site-directed polypeptide in both prokaryotic and eukaryotic cells.

A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein), it may be a spatially restricted promoter (i.e., transcriptional control element, enhancer, etc.) (e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., hair follicle cycle in mice).

Suitable promoters can be derived from viruses and can therefore be referred to as viral promoters, or they can be derived from any organism, including prokaryotic or eukaryotic organisms. Suitable promoters can be used to drive expression by any RNA polymerase (e.g., pol I, pollI, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6) (Miyagishi et al., Nature Biotechnology 20, 497-500 (2002)), an enhanced U6 promoter (e.g., Xia et al., Nucleic Acids Res. 2003 Sep. 1; 31 (17)), a human H1 promoter (H1), and the like.

Examples of inducible promoters include, but are not limited to T7 RNA polymerase promoter, T3 RNA polymerase promoter, isopropyl-beta-D-thiogalactopyranoside (IPTG)-regulated promoter, lactose induced promoter, heat shock promoter, tetracycline-regulated promoter (e.g., Tet-ON, Tet-OFF, etc.), steroid-regulated promoter, metal-regulated promoter, estrogen receptor-regulated promoter, etc. Inducible promoters can therefore be regulated by molecules including, but not limited to, doxycycline; RNA polymerase, e.g., T7 RNA polymerase; an estrogen receptor; an estrogen receptor fusion; etc.

In some embodiments, the promoter is a spatially restricted promoter (i.e., cell type specific promoter, tissue specific promoter, etc.) such that in a multi-cellular organism, the promoter is active (i.e., “ON”) in a subset of specific cells. Spatially restricted promoters may also be referred to as enhancers, transcriptional control elements, control sequences, etc. Any convenient spatially restricted promoter may be used and the choice of suitable promoter (e.g., a brain specific promoter, a promoter that drives expression in a subset of neurons, a promoter that drives expression in the germline, a promoter that drives expression in the lungs, a promoter that drives expression in muscles, a promoter that drives expression in islet cells of the pancreas, etc.) will depend on the organism. For example, various spatially restricted promoters are known for plants, flies, worms, mammals, mice, etc. Thus, a spatially restricted promoter can be used to regulate the expression of a nucleic acid encoding a variant sRGN site-directed polypeptide in a wide variety of different tissues and cell types, depending on the organism. Some spatially restricted promoters are also temporally restricted such that the promoter is in the “ON” state or “OFF” state during specific stages of embryonic development or during specific stages of a biological process (e.g., hair follicle cycle in mice).

For illustration purposes, examples of spatially restricted promoters include, but are not limited to, neuron-specific promoters, adipocyte-specific promoters, cardiomyocyte-specific promoters, smooth muscle-specific promoters, photoreceptor-specific promoters, etc. Neuron-specific spatially restricted promoters include, but are not limited to, a neuron-specific enolase (NSE) promoter (e.g., EMBL HSENO2, X51956); an aromatic amino acid decarboxylase (AADC) promoter; a neurofilament promoter (e.g., GenBank HUMNFL, L04147); a synapsin promoter (e.g., GenBank HUMSYNIB, M55301); a thy-1 promoter (e.g., Chen et al. (1987) Ce/151:7-19; and Llewellyn, et al. (2010) Nat. Med. 16 (10): 1161-1166); a serotonin receptor promoter (e.g., GenBank S62283); a tyrosine hydroxylase promoter (TH) (e.g., Oh et al. (2009) Gene Ther 16:437; Sasaoka et al. (1992) Mol. Brain Res. 16:274; Boundy et al. (1998) J. Neurosci. 18:9989; and Kaneda et al. (1991) Neuron 6:583-594); a GnRH promoter (e.g., Radovick et al. (1991) Proc. Nat!. Acad. Sci. USA 88:3402-3406); an L7 promoter (e.g., Oberdick et al. (1990) Science 248:223-226); a DNMT promoter (e.g., Bartge et al. (1988) Proc. Nat/. Acad. Sci. USA 85:3648-3652); an enkephalin promoter (e.g., Comb et al. (1988) EMBO J. 17:3793-3805); a myelin basic protein (MBP) promoter; a Ca2+-calmodulin-dependent protein kinase 11-alpha (CamKIIa) promoter (e.g., Mayford et al. (1996) Proc. Nat/. Acad. Sci. USA 93:13250; and Casanova et al. (2001) Genesis 31:37); a CMV enhancer/platelet-derived growth factor-0 promoter (e.g., Liu et al. (2004) Gene Therapy 11:52-60); and the like.

Adipocyte-specific spatially restricted promoters include, but are not limited to aP2 gene promoter/enhancer, e.g., a region from −5.4 kb to +21 bp of a human aP2 gene (e.g., Tozzo et al. (1997) Endocrinol. 138:1604; Ross et al. (1990) Proc. Nat!. Acad. Sci. USA 87:9590; and Pavjani et al. (2005) Nat. Med. 11:797); a glucose transporter-4 (GLUT4) promoter (e.g., Knight et al. (2003) Proc. Nat/. Acad. Sci. USA 100:14725); a fatty acid translocase (FAT/CD36) promoter (e.g., Kuriki et al. (2002) Biol. Pharm. Bull. 25:1476; and Sato et al. (2002) J. Biol. Chem. 277:15703); a stearoyi-CoA desaturase-1 (SCD1) promoter (Tabor et. al.. (1999) J. Biol. Chem. 274:20603); a leptin promoter (e.g., Mason et al. (1998) Endocrinol. 139:1013; and Chen et al. (1999) Biochem. Biophys. Res. Comm. 262:187); an adiponectin promoter (e.g., Kita et al. (2005) Biochem. Biophys. Res. Comm. 331:484; and Chakrabarti (2010) Endocrinol. 151:2408); an adipsin promoter (e.g., Platt et al. (1989) Proc. Natl. Acad. Sci. USA 86:7490); a resistin promoter (e.g., Seo et al. (2003) Malec. Endocrinol. 17:1522); and the like.

Cardiomyocyte-specific spatially restricted promoters include, but are not limited to control sequences derived from the following genes: myosin light chain-2, a-myosin heavy chain, AE3, cardiac troponin C, cardiac actin, and the like. Franz et al. (1997) Cardiovasc. Res. 35:560-566; Robbins et al. (1995) Ann. N.Y. Acad. Sci. 752:492-505; Linn et al. (1995) Circ. Res. 76:584591; Parmacek et al. (1994) Mol. Cell. Biol. 14:1870-1885; Hunter et al. (1993) Hypertension 22:608-617; and Sartorelli et al. (1992) Proc. Natl. Acad. Sci. USA 89:4047-4051.

Smooth muscle-specific spatially restricted promoters include, but are not limited to an SM22a promoter (e.g., Akyilrek et al. (2000) Mol. Med. 6:983; and U.S. Pat. No. 7,169,874); a smoothelin promoter (e.g., WO 2001/018048); an a-smooth muscle actin promoter; and the like. For example, a 0.4 kb region of the SM22a promoter, within which lie two CArG elements, has been shown to mediate vascular smooth muscle cell-specific expression (e.g., Kim, et al. (1997) Mol. Cell. Biol. 17, 2266-2278; Li, et al., (1996) J. Cell Biol. 132, 849-859; and Moessler, et al. (1996) Development 122, 2415-2425).

Photoreceptor-specific spatially restricted promoters include, but are not limited to, a rhodopsin promoter; a rhodopsin kinase promoter (Young et al. (2003) Ophthalmol. Vis. Sci. 44:4076); a beta phosphodiesterase gene promoter (Nicoud et al. (2007) J. Gene Med. 9:1015); a retinitis pigmentosa gene promoter (Nicoud et al. (2007) supra); an interphotoreceptor retinoid-binding protein (IRBP) gene enhancer (Nicoud et al. (2007) supra); an IRBP gene promoter (Yokoyama et al. (1992) Exp Eye Res. 55:225); and the like.

### Uses

A method for modulating transcription according to the present disclosure finds use in a variety of applications, which are also provided. Applications include research applications; diagnostic applications; industrial applications; and therapeutic applications.

Research applications include, e.g., determining the effect of reducing or increasing transcription of a target nucleic acid on, e.g., development, metabolism, expression of a downstream gene, and the like. High through-put genomic analysis can be carried out using a transcription modulation method, in which only the DNA-targeting segment of the guide RNA needs to be varied, while the protein-binding segment and the transcription termination segment can (in some cases) be held constant. A library (e.g., a library) comprising a plurality of nucleic acids used in the genomic analysis would include: a promoter operably linked to a guide RNA-encoding nucleotide sequence, where each nucleic acid would include a common protein-binding segment, a different DNA-targeting segment, and a common transcription termination segment. A chip could contain over 5×104 unique guide RNAs. Applications would include large-scale phenotyping, gene-to-function mapping, and meta-genomic analysis.

The methods disclosed herein find use in the field of metabolic engineering. Because transcription levels can be efficiently and predictably controlled by designing an appropriate guide RNA, as disclosed herein, the activity of metabolic pathways (e.g., biosynthetic pathways) can be precisely controlled and tuned by controlling the level of specific enzymes (e.g., via increased or decreased transcription) within a metabolic pathway of interest. Metabolic pathways of interest include those used for chemical (fine chemicals, fuel, antibiotics, toxins, agonists, antagonists, etc.) and/or drug production.

Biosynthetic pathways of interest include but are not limited to (1) the mevalonate pathway (e.g., HMG-COA reductase pathway) (converts acetyl-GoA to dimethylallyl pyrophosphate (DMAPP) and isopentenyl pyrophosphate (IPP), which are used for the biosynthesis of a wide variety of biomolecules including terpenoids/isoprenoids), (2) the non-mevalonate pathway (i.e., the “2-C-methyl-D-erythritol 4-phosphate/1-deoxy-D-xylulose 5-phosphate pathway” or “MEP/DOXP pathway” or “DXP pathway”) (also produces DMAPP and IPP, instead by converting pyruvate and glyceraldehyde 3-phosphate into DMAPP and IPP via an alternative pathway to the mevalonate pathway), (3) the polyketide synthesis pathway (produces a variety of polyketides via a variety of polyketide synthase enzymes. Polyketides include naturally occurring small molecules used for chemotherapy (e.g., tetracyclin, and macrolides) and industrially important polyketides include rapamycin (immunosuppressant), erythromycin (antibiotic), lovastatin (anticholesterol drug), and epothilone B (anticancer drug)), (4) fatty acid synthesis pathways, (5) the DAHP (3-deoxy-D-arabino-heptulosonate 7-phosphate) synthesis pathway, (6) pathways that produce potential biofuels (such as short-chain alcohols and alkane, fatty acid methyl esters and fatty alcohols, isoprenoids, etc.), etc.

### Networks and Cascades

The methods disclosed herein can be used to design integrated networks (i.e., a cascade or cascades) of control. For example, a guide RNA I variant sRGN site-directed polypeptide may be used to control (i.e., modulate, e.g., increase, decrease) the expression of another DNA-targeting RNA or another variant sRGN site-directed polypeptide. For example, a first guide RNA may be designed to target the modulation of transcription of a second chimeric dsRGN polypeptide with a function that is different than the first variant sRGN site-directed polypeptide (e.g., methyltransferase activity, demethylase activity, acetyltansferase activity, deacetylase activity, etc.). In addition, because different dsRGN proteins (e.g., derived from different species) may require a different sRGN handle (i.e., protein binding segment), the second chimeric dsRGN polypeptide can be derived from a different species than the first dsRGN polypeptide above. Thus, in some cases, the second chimeric dsRGN polypeptide can be selected such that it may not interact with the first guide RNA. In other cases, the second chimeric dsRGN polypeptide can be selected such that it does interact with the first guide RNA. In some such cases, the activities of the two (or more) dsRGN proteins may compete (e.g., if the polypeptides have opposing activities) or may synergize (e.g., if the polypeptides have similar or synergistic activities). Likewise, as noted above, any of the complexes (i.e., guide RNA I dsRGN polypeptide) in the network can be designed to control other guide RNAs or dsRGN polypeptides. Because a guide RNA and variant sRGN site-directed polypeptide can be targeted to any desired DNA sequence, the methods described herein can be used to control and regulate the expression of any desired target. The integrated networks (i.e., cascades of interactions) that can be designed range from very simple to very complex, and are without limit.

In a network where two or more components (e.g. guide RNAs or dsRGN polypeptides) are each under regulatory control of another guide RNA/dsRGN polypeptide complex, the level of expression of one component of the network may affect the level of expression (e.g. may increase or decrease the expression) of another component of the network. Through this mechanism, the expression of one component may affect the expression of a different component in the same network, and the network may include a mix of components that increase the expression of other components. as well as components that decrease the expression of other components. As would be understood by one of skill in the art, the above examples whereby the level of expression of one component may affect the level of expression of one or more different component(s) are for illustrative purposes, and are not limiting. Optionally an additional layer of complexity may be introduced into a network when one or more components are modified (as described above) to be manipulable (i.e., under experimental control, e.g. temperature control; drug control, i.e., drug inducible control; light control; etc.).

As one non-limiting example, a first guide RNA can bind to the promoter of a second guide RNA, which controls the expression of a target therapeutic/metabolic gene. In such a case, conditional expression of the first guide RNA indirectly activates the therapeutic/metabolic gene. RNA cascades of this type are useful, for example, for converting a repressor into an activator, and can be used to control the logics or dynamics of expression of a target gene.

A transcription modulation method can also be used for drug discovery and target validation.

Various aspects of the invention make use of the following materials and methods and are illustrated by the following non-limiting examples.

### Genome Editing

Genome editing generally refers to the process of modifying the nucleotide sequence of a genome, such as in a precise or predetermined manner. Examples of methods of genome editing described herein include methods of using site-directed nucleases to cut DNA at precise target locations in the genome, thereby creating double-strand or single-strand DNA breaks at particular locations within the genome. Such breaks can be and regularly are repaired by natural, endogenous cellular processes such as homology-directed repair (HDR) and non-homologous end-joining (NHEJ), as recently reviewed in Cox et al., Nature Medicine 21 (2), 121-31 (2015). NHEJ directly joins the DNA ends resulting from a double-strand break sometimes with the loss or addition of nucleotide sequence which may disrupt or enhance gene expression. HDR utilizes a homologous sequence, or donor sequence, as a template for inserting a defined DNA sequence at the break point. The homologous sequence may be in the endogenous genome, such as a sister chromatid. Alternatively, the donor may be an exogenous nucleic acid such as a plasmid, a single-strand oligonucleotide, a duplex oligonucleotide or a virus, that has regions of high homology with the nuclease-cleaved locus, but which may also contain additional sequence or sequence changes including deletions that can be incorporated into the cleaved target locus. A third repair mechanism is microhomology-mediated end joining (MMEJ), also referred to as “Alternative NHEJ,” in which the genetic outcome is similar to NHEJ in that small deletions and insertions can occur at the cleavage site. MMEJ makes use of homologous sequences of a few base pairs flanking the DNA break site to drive a more favored DNA end joining repair outcome, and recent reports have further elucidated the molecular mechanism of this process; see, e.g. Cho and Greenberg, Nature 518, 174-76 (2015); Kent et al., Nature Structural and Molecular Biology, Adv. Online doi: 10.1038/nsmb.2961 (2015); Mateos-Gomez et al., Nature 518, 254-57 (2015); Ceccaldi et al., Nature 528, 258-62 (2015). In some instances likely repair outcomes can be predicted based on analysis of potential microhomologies at the site of the DNA break.

Each of these genome editing mechanisms can be used to create desired genomic alterations. The genome editing process creates generally one or two DNA breaks in the target locus as close as possible to the site of intended mutation. This can achieved via the use of site-directed polypeptides, as described and illustrated herein.

Site-directed polypeptides can introduce double-strand breaks or single-strand breaks in nucleic acid, (e.g., genomic DNA). The double-strand break can stimulate a cell's endogenous DNA-repair pathways (e.g., homology-dependent repair (HDR) and non-homologous end joining (NHEJ) or alternative non-homologous end joining (A-NHEJ) or microhomology-mediated end joining (MMEJ)). NHEJ can repair cleaved target nucleic acid without the need for a homologous template. This can sometimes result in small deletions or insertions (indels) in the target nucleic acid at the site of cleavage and can lead to disruption or alteration of gene expression. HDR can occur when a homologous repair template, or donor, is available. The homologous donor template comprises sequences that are homologous to sequences flanking the target nucleic acid cleavage site. The sister chromatid is generally used by the cell as the repair template. However, for the purposes of genome editing, the repair template is often supplied as an exogenous nucleic acid, such as a plasmid, duplex oligonucleotide, single-strand oligonucleotide or viral nucleic acid. With exogenous donor templates it is common to introduce additional nucleic acid sequence (such as a transgene) or modification (such as a single base change or a deletion) between the flanking regions of homology so additional or altered nucleic acid sequence also becomes incorporated into the target locus. MMEJ results in a genetic outcome that is similar to NHEJ in that small deletions and insertions can occur at the cleavage site. MMEJ makes use of homologous sequences of a few basepairs flanking the cleavage site to drive a favored end-joining DNA repair outcome. In some instances it may be possible to predict likely repair outcomes based on analysis of potential microhomologies in the nuclease target regions. Thus, in some cases, homologous recombination is used to insert an exogenous polynucleotide sequence into the target nucleic acid cleavage site. An exogenous polynucleotide sequence is termed a donor polynucleotide herein. In some embodiments, the donor polynucleotide, a portion of the donor polynucleotide, a copy of the donor polynucleotide, or a portion of a copy of the donor polynucleotide is inserted into the target nucleic acid cleavage site. In some embodiments, the donor polynucleotide is an exogenous polynucleotide sequence, i.e., a sequence that does not naturally occur at the target nucleic acid cleavage site.

The modifications of the target DNA due to NHEJ and/or HDR can lead to, for example, mutations, deletions, alterations, integrations, gene correction, gene replacement. gene tagging, transgene insertion, nucleotide deletion, gene disruption, translocations and/or gene mutation. The processes of deleting genomic DNA and integrating non-native nucleic acid into genomic DNA are examples of genome editing.

### EXAMPLES

### Example 1a: Confirmation of the Activity of sRGN Polypeptide Gib11

Gib11 and SluCas9 were tested in a fluorescence polarization-based biochemical cleavage assay.

### Protocol Fluorescence Polarization Assay:

Oligonucleotide duplexes were prepared in 10 mM Tris (pH=7.8) 50 mM NaCl as 10 μM solutions (from 100 μM stocks) and annealed at 95° C. for 5 minutes and then slowly cooled down in thermo cycler (6° C. per minute). The stocks were subsequently diluted in 10 mM Tris (pH=7.8) 50 mM NaCl+0.05% pluronic. 20 nM oligo (20 μL) were immobilized on streptavidin coated plate, washed twice after 5 minutes and then incubated with a 20 μL sample for a kinetics of 60 minutes (excitation wavelength: 635 nm; emission wavelength: 670 nm). Prior to the cleavage, the reaction RNP was formed. Nuclease and sgRNA_{VEGFA} (SEQ ID NO: 114), transcribed from pCas606 (SEQ ID NO: 118), were added to 817.6 μl 1×PBS MgCl₂, and incubated at 37° C. for 10 minutes. 20 μl RNP was added to each well and the polarization was measured for 60 minutes at 37° C. See, for example, Methods Enzymol. 2014; 546:1-20.

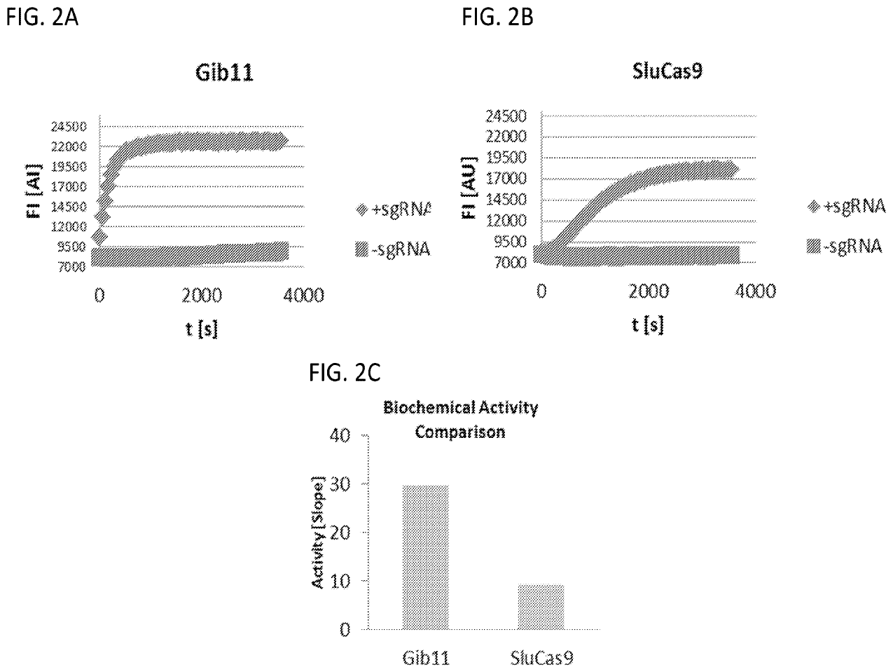

The amount of protein was titrated against a constant level of sgRNA. Upon incubation of the Gib11- or SluCas9-RNP complex targeting the VEGFA oligonucleotide sequence, cleavage can be observed by changes in the fluorescence polarization and fluorescence intensity signal (decrease of polarization values and increase in fluorescence intensity over time upon successful cleavage). As a quantitative estimate of the cleavage reaction, the initial slope of the graph was analyzed. The results (represented in FIGS. 2A and 2B) show that Gib11 and SluCas9 successfully cleave an oligonucleotide substrate. The cleavage kinetics are shown in FIG. 2C.

### Example 1B: Confirmation of the Gib11 “NNGG” PAM Motif

The activity of Gib11 was confirmed in liquid cultures and on agar plates.

### In Liquid Culture:

BL21DE3 origami cells harboring a toxic reporter plasmid (pCas634, SEQ ID NO: 119, encoding an arabinose-inducible ccdB toxic gene with a VEGFA target site and a “NNGG” PAM) and a plasmid encoding an sgRNA targeting VEGFA (pCas606, SEQ ID NO: 118) were transformed with a plasmid encoding chimeric construct 1 (PI/WEDGE domain exchanged, pCas889 according to SEQ ID NO: 122) or a chimeric construct 2 (PI domain exchanged, pCas888 according to SEQ ID NO: 121) under an IPTG-inducible Trc promotor. After transformation, the cell suspension was rescued with 1 ml SOC and incubated at 37° C. with 1000 rpm shaking (Eppendorf Thermomix). 200 μl were used to inoculate LB medium (supplemented with kanamycin, chloramphenicol and ampicillin) and the liquid culture was incubated overnight at 37° C. with 190 rpm shaking. 200 μl of this overnight culture were used to inoculate 5 ml LB medium supplemented with kanamycin and ampicillin and with/without 0.5% L-arabinose, with/without 0.05 mM IPTG. After incubation at 37° C. overnight (18 h) with 190 rpm shaking, OD₆₀₀ was determined.

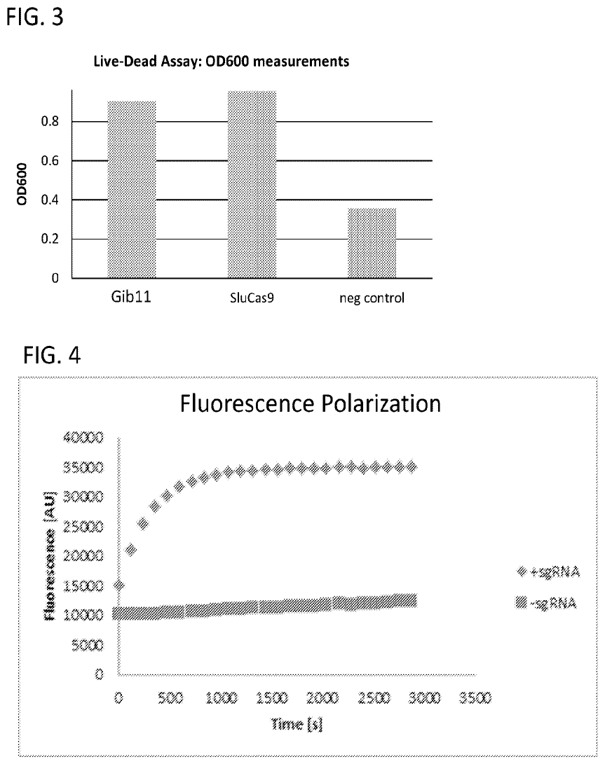

The OD₆₀₀ measurement is visualized in the bar chart in FIG. 3 with OD₆₀₀ values obtained for Gib11, SluCas9, and the negative control.

### On Plates:

BL21DE3 origami cells harboring a toxic reporter plasmid (pCas634, SEQ ID NO: 119, encoding an arabinose-inducible ccdB toxic gene with a VEGFA target site and an “NNGG” PAM) and a plasmid encoding an sgRNA targeting VEGFA (pCas606, SEQ ID NO: 118) were transformed with a plasmid encoding chimeric construct 1 (PI/WEDGE domain exchanged, pCas889, according to SEQ ID NO: 122) or chimeric construct 2 (PI domain exchanged, pCas888, according to SEQ ID NO: 121) under an IPTG-inducible Trc promotor (pCas81, SEQ ID NO: 120). After transformation, the cell suspension was rescued with 1 ml SOC medium and incubated at 37° C. with 1000 rpm shaking (Eppendorf Thermomix). 200 μl were plated in LB-Agar plates supplemented with kanamycin, ampicillin, 0.05 mM IPTG and 0.5% L-arabinose. After incubation at 37° C. for 18 h, colonies were counted.

BL21DE3 origami cells harboring a plasmid encoding Gib11 successfully cleaved a toxic reporter plasmid and survived the incubation after induction of the toxic reporter protein ccdB. The survival is measured by increased OD₆₀₀ values in liquid culture and by colony formation on agar plates.

TABLE 5

| | | SluCas9 | negative |
|---|---|---|---|
| Colony formation on agar plates | Gib11 | WT | control |
| normalized Growth to SluCas9 | 94 | 100 | 37 |
| wild-type [%] | | | |

### Example 1C: Confirmation of Gib11-Mediated Genome Editing in Mammalian Cells

Activity of Gib11 in mammalian cells was demonstrated in the following assays:

### BFP Disruption Assay Using Guide Targeting BFP Gene:

HEK293T cells harboring a gene encoding blue fluorescent protein (BFP) in the AAVS1 locus were transfected with plasmid expressing Gib11 and an sgRNA (BFP targeting guide RNA [SEQ ID NO: 117] and SluCas9 tracr RNA [SEQ ID NO: 123]) targeting the reverse strand of BFP genomic sequence as described. Transfection of plasmids was performed using Lipofectamine 3000 (Thermo Fischer Scientific, Cat no. L3000015) as described by the supplier. Flow cytometric analyses of BFP signal disruption was carried out 7 days post-transfection. Cells were prepared by washing with 1×PBS, trypsinization, and resuspension in 200 μl FACS buffer (1×PBS supplemented with 2% FCS). BFP signal from 10,000 cells was assayed using the V450 filter set in the BD FACS Canto® II (Glaser et al., 2016).

HEK293T cells were transfected with a plasmid encoding Gib11 and a sgRNA targeting the BFP gene. A significant decrease in blue fluorescence was observed (quantified as % BFP disruption). In line with the function of Cas9 proteins, the likely reason for this is a Gib11-assisted DNA cleavage followed by insertions/deletions at the BFP open-reading-frame (ORF) resulting in disruption of the BFP ORF. Gib11 lead to 34.35%+7.15% BFP disruption.

TABLE 6

| Samples | % BFP Disruption | Std Dev |
|---|---|---|
| Neg control | 4.1 | 0 |
| SluCas9 | 6.35 | 0.65 |
| Gib11 | 34.35 | 7.15 |

### Example 2a: Confirmation of the Activity of sRGN Polypeptide P2H12

### Protocol Fluorescence Polarization Assay:

Oligonucleotide duplexes were prepared in 10 mM Tris (pH=7.8) 50 mM NaCl as 10 μM solutions (from 100 μM stocks) and annealed at 95° C. for 5 minutes and then slowly cooled down in thermo cycler (6° C. per minute). The stocks were subsequently diluted in 10 mM Tris (pH=7.8) 50 mM NaCl+0.05% pluronic. 20 nM oligo (20 μL) were immobilized on streptavidin coated plate, washed twice after 5 minutes and then incubated with a 20 μL sample for a kinetics of 60 minutes (excitation wavelength: 635 nm; emission wavelength: 670 nm). Prior to the cleavage the reaction RNP was formed. Nuclease and sgRNA_{VEGFA} (SEQ ID NO: 114), transcribed from pCas606 (SEQ ID NO: 118), were added to 817.6 μl 1×PBS MgCl₂, and incubated at 37° C. for 10 minutes. 20 μl RNP was added to each well and the polarization was measured for 60 minutes at 37° C. See, for example, Methods Enzymol. 2014; 546:1-20.

Upon incubation of the P2H12-RNP complex targeting the VEGFA oligonucleotide sequence, cleavage was observed by marked changes in the fluorescence polarization and fluorescence intensity signal (decrease of polarization values and increase in fluorescence intensity over time upon successful cleavage) as represented in FIG. 4.

### Example 2B: Confirmation of P2H12-Mediated Genome Editing in Mammalian Cells

The activity of P2H12 in mammalian cells was demonstrated in the following assays:

### BFP Disruption Assay Using Guide Targeting BFP Gene:

HEK293T cells harboring a gene encoding BFP in the AAVS1 locus were transfected with plasmid expressing P2H12 and a sgRNA (BFP targeting guide RNA [SEQ ID NO: 117] and SluCas9 tracr RNA [SEQ ID NO: 123]) targeting the reverse strand of BFP genomic sequence as described. Transfection of plasmids was performed using Lipofectamine 3000 (Thermo Fischer Scientific, Cat no. L3000015) as described by the supplier. Flow cytometric analyses of BFP signal disruption was carried out 7 days post-transfection. Cells were prepared by washing with 1×PBS, trypsinization, and resuspension in 200 μl FACS buffer (1×PBS supplemented with 2% FCS). BFP signal from 10,000 cells was assayed using the V450 filter set in the BD FACS Canto II (Glaser et al., 2016).

HEK293T cells were transfected with a plasmid encoding P2H12 and an sgRNA targeting the BFP gene. A significant decrease in blue fluorescence was observed (quantified as % BFP disruption). In line with the function of Cas9 proteins, the likely reason for this is a P2H12-assisted DNA cleavage followed by insertions/deletions at the BFP open-reading-frame (ORF) resulting in disruption of the BFP ORF. P2H12 lead to 22.9%+4.4% BFP disruption.

TABLE 7

| Samples | % BFP Disruption |
|---|---|
| Neg Control | 4.1 |
| SluCas9 | 6.35 ± 0.65 |
| P2H12 | 22.9 ± 4.4 |

### Example 3: Confirmation of E2-Mediated Genome Editing in Mammalian Cells

The activity of E2 in mammalian cells was demonstrated in the following assays:

### Bfp Disruption Assay Using Guide Targeting Bfp Gene:

HEK293T cells harboring a gene encoding BFP in the AAVS1 locus were transfected with plasmid expressing E2 and a sgRNA (BFP targeting guide RNA [SEQ ID NO: 117] and SluCas9 tracr RNA [SEQ ID NO: 123]) targeting the reverse strand of BFP genomic sequence as described. Transfection of plasmids was performed using Lipofectamine 3000 (Thermo Fischer Scientific, Cat no. L3000015) as described by the supplier. Flow cytometric analyses of BFP signal disruption was carried out 7 days post-transfection. Cells were prepared by washing with 1×PBS, trypsinization, and resuspension in 200 μl FACS buffer (1×PBS supplemented with 2% FCS). BFP signal from 10,000 cells was assayed using the V450 filter set in the BD FACS Canto II (Glaser et al., 2016).

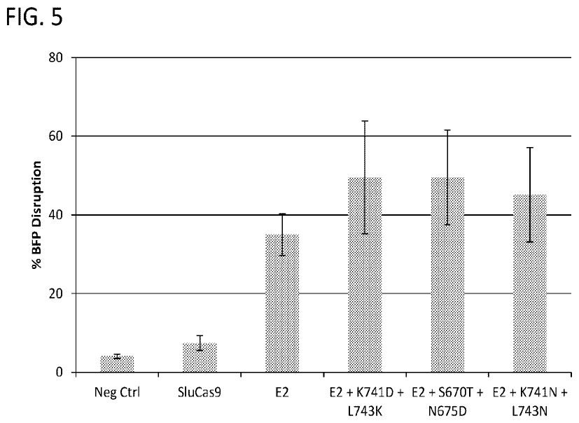

The results of three replicates of this experiment with E2, three E2 mutants, a negative control, and for reference SluCas9 (SEQ ID NO: 125) are shown in Table 8, and depicted in FIG. 5. These results show that E2 and the three mutants are able to mediate targeted DNA cleavage.

TABLE 8

| Cas9 Protein | % BFP Disruption | Std Dev |
|---|---|---|
| Neg Ctrl | 4.05 | 0.55 |
| SluCas9 | 7.4 | 1.9 |
| E2 | 35.0 | 5.3 |
| E2 + K741D + L743K | 49.5 | 14 |
| E2 + S670T + N675D | 49.5 | 12 |
| E2 + K741N + L743N | 45.1 | 12 |

### Example 4: Confirmation of Gib11Spa-Mediated Genome Editing in Mammalian Cells

The activity of Gib11Spa-1, Gib11Spa-2, and Gib11Spa-3 in mammalian cells was demonstrated in the following assay:

### Bfp Disruption Assay Using Guide Targeting Bfp Gene:

HEK293T cells harboring a gene encoding BFP in the AAVS1 locus were transfected with plasmid expressing Gib11Spa-1, Gib11Spa-2, or Gib11Spa-3 and a sgRNA (BFP targeting guide RNA (SEQ ID NO: 117) and SluCas9 tracr RNA (SEQ ID NO: 123)) targeting the reverse strand of BFP genomic sequence as described. Transfection of plasmids was performed using Lipofectamine 3000 (Thermo Fischer Scientific, Cat no. L3000015) as described by the supplier. Flow cytometric analyses of BFP signal disruption was carried out 7 days post-transfection. Cells were prepared by washing with 1×PBS, trypsinization, and resuspension in 200 μl FACS buffer (1×PBS supplemented with 2% FCS). BFP signal from 10,000 cells was assayed using the V450 filter set in the BD FACS Canto II (Glaser et al., 2016, Molecular Therapy Nucleic Acids 5, e334).

### Editing in Mammalian Cells Using Gib11Spa-1, Gib11Spa-2, and Gib11Spa-3

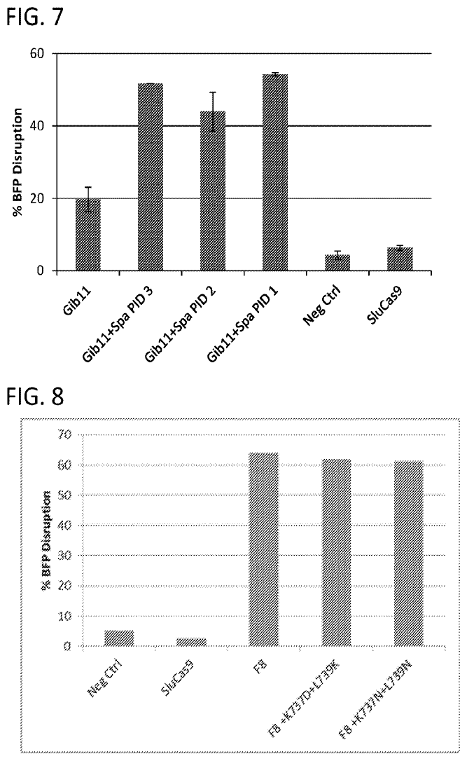

Upon transfecting HEK293T cells with a plasmid encoding Gib11Spa-1, Gib11Spa-2, or Gib11Spa-3 and a sgRNA targeting the BFP gene, a significant decrease in blue fluorescence was observed (quantified as % BFP disruption). In line with the function of Cas9 proteins, the likely reason for this is Gib11Spa-assisted DNA cleavage followed by insertions/deletions at the BFP open-reading-frame (ORF) and resulting disruption of the BFP ORF. Results are shown in Table 9 and in the bar graph in FIG. 7.

TABLE 9

| | % BFP | |
|---|---|---|
| Cas9 | Disruption | Std Dev |
| Gib11 | 19.7 | 3.4 |
| Gib11 + Spa PID 3 | 51.7 | — |
| Gib11 + Spa PID 2 | 44.0 | 5.4 |
| Gib11 + Spa PID 1 | 54.2 | 0.5 |
| Neg Ctrl | 4.4 | 1.1 |
| SluCas9 | 6.3 | 0.7 |

### Example 5: Confirmation of F8-Mediated Genome Editing in Mammalian Cells

The activity of F8 in mammalian cells was demonstrated in the following assay:

### Bfp Disruption Assay Using Guide Targeting Bfp Gene:

HEK293T cells harboring a gene encoding BFP in the AAVS1 locus were transfected with plasmid expressing F8 and a sgRNA (BFP targeting guide RNA [SEQ ID NO: 117] and SluCas9 tracr RNA [SEQ ID NO: 123]) targeting the reverse strand of BFP genomic sequence as described. Transfection of plasmids was performed using Lipofectamine 3000 (Thermo Fischer Scientific, Cat no. L3000015) as described by the supplier. Flow cytometric analyses of BFP signal disruption was carried out 7 days post-transfection. Cells were prepared by washing with 1×PBS, trypsinization, and resuspension in 200 μl FACS buffer (1×PBS supplemented with 2% FCS). BFP signal from 10,000 cells was assayed using the V450 filter set in the BD FACS Canto II (Glaser et al., 2016).

The results of three replicates of this experiment with F8, two F8 mutants, a negative control, and for reference SluCas9 (SEQ ID NO: 125) are shown in Table 10 and depicted in FIG. 8. These results show that F8 and the two mutants are able to mediate targeted DNA cleavage.

TABLE 10

| Cas9 | % BFP Disruption |
|---|---|
| Neg Ctrl | 5.2 |
| SluCas9 | 2.6 |
| F8 | 64 |
| F8 + K737D + L739K | 61.9 |
| F8 + K737N + L739N | 61.3 |

### Example 6: Determination of Biochemical Specificity Profile for sRGNs

To assess the specificity profile of Gib11, Gib11Spa-1, Gib11Spa-3, E2, and F8, biochemical cleavage of an on-target sequence and all 60-derived single nucleotide mismatched dsDNA substrates (according to SEQ ID NOs: 129-250 used as pairs, e.g., R01-1-A and R01-1-B with SEQ ID NOs: 129 and 130, respectively, represent one duplex pair) were determined. Oligonucleotide duplexes were prepared in 10 mM Tris (pH 7.8) 50 mM NaCl as 10 μM solutions (from 100 μM stocks) and annealed at 95° C. for 5 minutes then slowly cooled down in thermo cycler (6° C. per minute). The stocks were subsequently diluted in 10 mM Tris (pH 7.8), 50 mM NaCl, and 0.05% Pluronic. 20 μl of each oligonucleotide (20 nM) was immobilized on streptavidin coated plates, washed twice after 10 minutes and then incubated with a 20 μL sample for a kinetics of 60 minutes (excitation wavelength: 635 nm; emission wavelength: 670 nm). Prior to the cleavage the reaction RNP was formed. RNP was assembled by mixing 12.03 μl of the respective sRGN protein (1.21 μg/μl) in 3200 μl 1×PBS+5 mM MgCl2 with and without sgRNA (SEQ ID NO: 128; 60 nM final concentration), RNPs were incubated 5 min at 37° C. prior to the reaction. 20 μl RNP was added to each well and the polarization was measured for 60 minutes at 37° C.

The cleavage kinetics were analyzed by calculating the initial slope of the oligonucleotide cleavage reaction. The slopes were calculated for each of the 61 substrates and normalized to the value of the on-target substrate (defined as 1). The normalized cleavage values for all 60 off-target substrates where then grouped according to their position. For each of the 20 nucleotide positions in the target sequence, the normalized cleavage value of the three single nucleotide mismatches was blotted to illustrate the position-specific nucleotide tolerance of the tested nuclease.

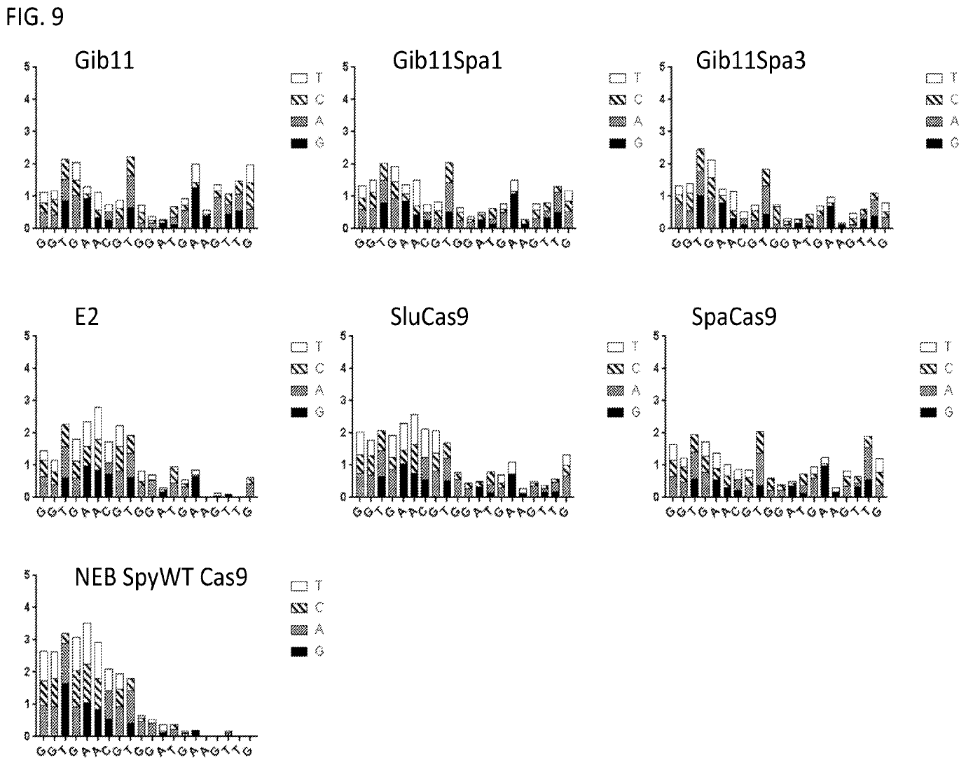

The results are shown in FIG. 9. The specificity profile of E2 was comparable to that of SluCas9 using this guide/target combination. Gib11, Gib11Spa-1, and Gib11Spa-3 had specificity profiles comparable to SpaCas9.

Overall Specificity of sRGNs:

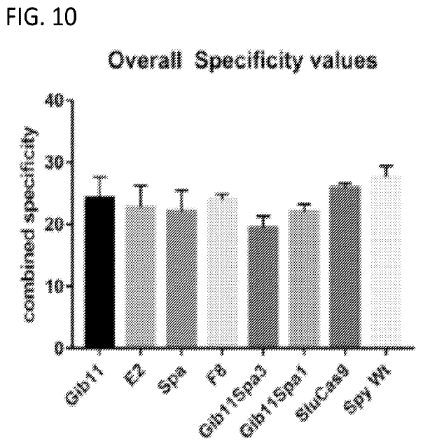

The cleavage kinetics for a) Gib11; b) Gib11Spa-1; c) Gib11Spa-2, d) E2, e) F8, f) SluCas9, g) Staphylococcus pyogenes wild type (New England Biolabs), and h) Staphylococcus aureus were analyzed by calculating the initial slope of the oligonucleotide cleavage reaction. The slopes were calculated for each of the 61 substrates and were normalized to the value of the on-target substrate (defined as 1). All normalized values across the entire panel of 60 off-target substrates were summed to yield an overall specificity value (as depicted in the bar graph in FIG. 10). The sRGNs were more specific than SpyCas9 using this guide/target combination.

### Example 7: Determination of Activity Profile for sRGNs

To assess the activity profile of Gib11, Gib11Spa-1, Gib11Spa-3, E2, and F8, biochemical cleavage of 96 on-target DNA substrates (according to SEQ ID NOs: 347-538 used as pairs, e.g., Ext-A-P-1-A and Ext-A-P-1-B with SEQ ID NOs: 347 and 348, respectively, represent one duplex pair) with varying GC-content was determined.

### Protocol Fluorescence Polarization Assay:

Oligonucleotide duplexes were prepared in 10 mM Tris (pH=7.8) 50 mM NaCl as 10 μM solutions (from 100 μM stocks) and annealed at 95° C. for 5 minutes and then slowly cooled down in thermo cycler (6° C. per minute). The stocks were subsequently diluted in 10 mM Tris (pH=7.8) 50 mM NaCl+0.05% pluronic. 20 nM oligo (20 μL) were immobilized on streptavidin coated plate, washed twice after 5 minutes and then incubated with a 20 μL sample for a kinetics of 60 minutes (excitation wavelength: 635 nm; emission wavelength: 670 nm). Prior to the cleavage the reaction RNP was formed. For each guide sequence of SEQ ID NOS: 251-346, nuclease and an sgRNA containing the guide sequence were added to 817.6 μl 1×PBS MgCl₂, and incubated at 37° C. for 10 minutes. 20 μl RNP was added to wells containing the respective DNA substrate for the sgRNA in the RNP, and the polarization was measured for 60 minutes at 37° C. See, for example, Methods Enzymol. 2014; 546:1-20.

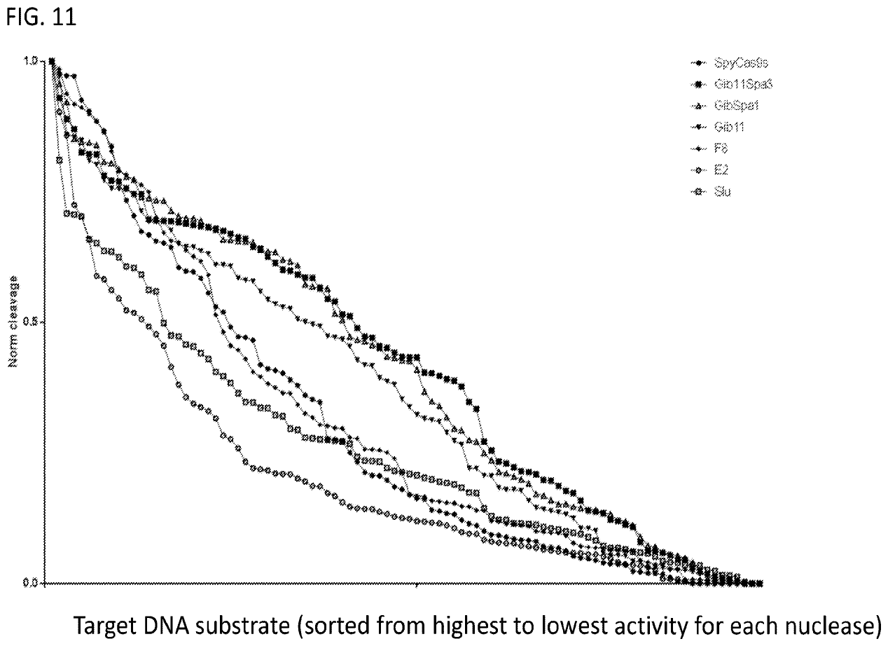

Indicated variants were tested in a fluorescence polarization-based biochemical cleavage assay. The amount of protein was titrated against a constant level of sgRNA. Upon incubation of the variant-RNP complex targeting the VEGFA oligonucleotide sequence, cleavage can be observed by changes in the fluorescence polarization and fluorescence intensity signal (decrease of polarization values and increase in fluorescence intensity over time upon successful cleavage). As a quantitative estimate of the cleavage reaction, the initial slope of the graph was analyzed. The data was normalized to the substrate with the highest activity for one RNA-guided nuclease, and results are shown in FIG. 11. Gib11Spa1/3 and Gib11 displayed a higher activity across a broader range of targets as compared to the other sRGNs and SpyCas9.

1-41. (canceled)

42. A synthetic RNA-guided nuclease (sRGN) polypeptide comprising an amino acid sequence having at least 95% identity to the amino acid sequence of SEQ ID NO: 3 or 4.

43. The sRGN of claim 42, wherein the amino acid sequence comprises a PAM-interacting domain that recognizes the PAM sequence NNGG.

44. The sRGN polypeptide of SEQ ID NO: 3 or 4, wherein the amino acid sequence has at least 96%, 97%, 98%, or 99% identity to the amino acid sequence of SEQ ID NO: 3 or 4, or wherein the amino acid sequence is SEQ ID NO: 3 or 4.

45. The sRGN of claim 44, wherein the sRGN polypeptide further comprises:
(i) one or more nuclear localization signals (NLSs);
(ii) one or more mutations that result in reduced nuclease activity as compared to the polypeptide of SEQ ID NO: 3 or 4, wherein the one or more mutations are selected from an amino acid substitution at a position corresponding to position 10 of the polypeptide of SEQ ID NO: 3 or 4, at a position corresponding to position 562 of the polypeptide of SEQ ID NO: 3 or 4, and at a position corresponding to position 585 of the polypeptide of SEQ ID NO: 3 or 4, and/or
(iii) a C- or N-terminally attached polypeptide with a nucleobase editing activity or deaminase activity, optionally attached via a linker peptide sequence.

46. A system for introducing a single-stranded or a double-stranded break in a target polynucleotide, the system comprising:
(a) a sRGN polypeptide comprising an amino acid sequence having at least 95% identity to the amino acid sequence of SEQ ID NO: 3 or 4; and
(b) a single guide RNA (sgRNA) comprising a DNA targeting segment capable of hybridizing to a target polynucleotide,
wherein the sgRNA combines with the sRGN polypeptide to induce a single-stranded or double-stranded break in the target polynucleotide.

47. The system of claim 46, further comprising a donor template comprising a heterologous polynucleotide, wherein the heterologous polynucleotide is capable of being inserted into the target polynucleotide.

48. The system of claim 46, wherein the sRGN polypeptide is pre-complexed with the sgRNA to form a ribonucleoprotein (RNP) complex.

49. The system of claim 46, wherein the sRGN polypeptide is formulated in a liposome or lipid nanoparticle, optionally wherein the liposome or lipid nanoparticle further comprises the sgRNA.

50. A pharmaceutical composition comprising the sRGN polypeptide of claim 42, and a pharmaceutically acceptable carrier.

51. A kit comprising:
(a) the sRGN polypeptide according to claim 42;
(b) a guide RNA (gRNA), wherein the gRNA is capable of guiding the sRGN polypeptide to a target polynucleotide, and
(c) instructions for targeting, editing, modifying, or manipulating a target DNA at a target locus.
